(19) United States
(12) Patent Application Publication
(10) Pub. No.: US 2026/0034245 A1
(43) Pub. Date: Feb. 5, 2026

(54) COMPOSITIONS AND METHODS FOR THE DIAGNOSIS AND TREATMENT OF RETINOPATHIES

(71) Applicant: Duke University, Durham, NC (US)

(72) Inventors: Jeremy KAY, Durham, NC (US); Thomas RAY, Durham, NC (US)

(21) Appl. No.: 19/299,144

(22) Filed: Aug. 13, 2025

Related U.S. Application Data
(62) Division of application No. 17/436,419, now Pat. No. 12,491,264.
(60) Provisional application No. 62/813,272, filed on Mar. 4, 2019.

(51) Int. Cl. A61K 48/00; C07K 14/47; C12N 15/86

(52) U.S. Cl. CPC A61K 48/005; C07K 14/4702; C12N 15/86; C12N 2750/14143

(57) ABSTRACT

The present invention provides compositions and methods related to the cell surface protein CRB1 for the treatment of retinopathies in a subject. In particular, isolated polynucleotides and recombinant vectors encoding a particular isoform called Crumbs 1-B (CRB1-B) are provided. The CRB1-B encoding polynucleotides may be operably linked to a heterologous promoter capable of expressing the isoform in a retinal cell. Kits employing such compositions are also provided.

### CROSS REFERENCE TO RELATED APPLICATIONS

This patent application is a divisional of U.S. patent application Ser. No. 17/436,419, filed Sep. 3, 2021, which application is a national stage filing under 35 U.S.C. § 371 of International Application No. PCT/US2020/020977, filed Mar. 4, 2020, which claims the benefit of priority of U.S. Provisional Application No. 62/813,272, filed on Mar. 4, 2019, the contents of which are incorporated herein by reference in their entireties.

### STATEMENT REGARDING FEDERALLY SPONSORED RESEARCH

This invention was made with Government support under Federal Grant number F32EY026344 awarded by the National Institutes of Health. The Government has certain rights to this invention.

### SEQUENCE LISTING

A Sequence Listing accompanies this application and is submitted as an xml of the sequence listing named “155554_00789_SL_ST26.xml” which is 244,530 bytes in size and was created on Aug. 8, 2025. The sequence listing is electronically submitted with the application and is incorporated herein by reference in its entirety.

### BACKGROUND

Loss-of-function mutations in the CRB1 gene cause a wide spectrum of retinal degenerative diseases. Recent advances in gene therapy have opened new possibilities for halting progressive vision loss in single-gene blinding diseases. If CRB1 disease is to become a strong candidate for such therapy, it is essential to understand the normal and pathobiological functions of CRB1 protein in the retina in vivo. The prevailing model of CRB1 function posits that it is required for structural integrity of the outer limiting membrane (OLM). CRB1 protein—a cell surface molecule with a large extracellular domain—has been localized to OLM cell-cell adhesions linking photoreceptors and Müller glia. Loss of CRB1 function is thought to weaken OLM adhesion, leading to structural deficits that ultimately cause photoreceptor death. According to this model, replacement of the CRB1 gene is a promising therapeutic strategy: Restoring adhesion would be expected to improve OLM integrity, thereby slowing or even halting photoreceptor death. To design an effective gene replacement strategy, it is important to know two critical pieces of information that are currently unclear. First, in which cell type should CRB1 be replaced? Is it needed on the glial or photoreceptor side of the OLM junction—or both? Second, which splice variant of the CRB molecule should be used for replacement? CRB1 is known to encode several alternative mRNA isoforms; moreover, since the true complexity of the human transcriptome remains surprisingly murky, there may still be additional isoforms that are not described. Because only one cDNA species can be chosen for inclusion in a gene therapy vector, it is critical to establish which isoform is most effective at halting degeneration when reintroduced into the mature retina.

### SUMMARY

In one aspect, the present disclosure provides an isolated polynucleotide comprising a polynucleotide sequence encoding a Crumbs 1-B (CRB1-B) isoform comprising SEQ ID NO:1 operably linked to a heterologous promoter capable of expressing the isoform in a retinal cell. In another aspect, the disclosure provides a vector comprising the isolated polynucleotide.

In another aspect, the disclosure provides a recombinant vector comprising a polynucleotide encoding a Crumbs 1-B (CRB1-B) isoform, wherein the CRB1-B isoform comprises an N-terminal signal peptide linked to an extracellular polypeptide comprising, from N-terminus-to-C-terminus: two EGF domains, a lamG domain, an EGF domain, a lamG domain, an EGF domain, a lamG domain, and four EGF domains; wherein the C terminus of the extracellular polypeptide is linked to a C-terminal domain comprising a transmembrane domain and intracellular domain.

In a further aspect, the present disclosure provides an isolated polypeptide made from the isolated polynucleotide or recombinant vector described herein.

In another aspect, the disclosure provides a pharmaceutical composition comprising the isolated polynucleotide or the recombinant vector described herein and a pharmaceutically acceptable carrier.

In another aspect, the present disclosure provides a method of treating an ocular disorder in a subject, the method comprising administering to the subject a therapeutically effective amount of the polynucleotide, the recombinant vector, the isolated polypeptide, or the pharmaceutical composition described herein such that the ocular disorder is treated in the subject.

In yet another aspect, the disclosure provides a method of reducing progression of loss of vision or maintaining vision function in a subject in need thereof, the method comprising administering to the subject a therapeutically effective amount of the polynucleotide, the recombinant vector, the isolated polypeptide, or the pharmaceutical composition described herein such that loss of vision is reduced.

In yet another embodiment, the disclosure provides a kit for treating an ocular disorder in a subject, the kit comprising the isolated polynucleotide, the recombinant vector, the isolated polypeptide, or the pharmaceutical composition described herein, a device for delivery of the isolated polynucleotide, recombinant vector, isolated polypeptide or pharmaceutical composition to the subject, and instructions for use.

In a further aspect, the disclosure provides a kit for reducing progression or reducing loss of vision or maintaining vision function in a subject, the kit comprising the isolated polynucleotide, the recombinant vector, the isolated polypeptide, or the pharmaceutical composition described herein and a device for delivery of the isolated polynucleotide, recombinant vector, isolated polypeptide, or pharmaceutical composition to the subject, and instructions for use.

In another aspect, the disclosure provides a system for the delivery of the isolated polynucleotide, the recombinant vector, the isolated polypeptide or the pharmaceutical composition to an eye of a subject, the system comprising a therapeutically effective amount of the isolated polynucleotide, the recombinant vector, the isolated polypeptide, or the pharmaceutical composition described herein, and a device for delivery to the subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts the strategy used herein for identifying cell surface receptors that exhibit high isoform diversity. (A) Screening strategy for selecting genes for lrCaptureSeq. Members of EGF, Ig, and adhesion GPCR families were tested for 1) expression during neural development, using RNA-seq data from retina and cortex; and 2) unannotated transcript diversity, based on RNA-seq read alignments compared to UCSC Genes public database. Thirty genes showing strong evidence for unannotated events such as alternative splicing, novel exons, and novel transcriptional start sites (asterisks) were selected for targeted sequencing of full length transcripts (B,C). (B) lrCaptureSeq workflow. cDNAs are 5′ tagged to enable identification of full-length reads. Biotinylated capture probes tiling known exons. To obtain sequencing libraries enriched for intact cDNAs, two rounds of amplification and size selection were used. (C) Size distribution of full-length reads for each lrCaptureSeq experiment. Mouse retinal transcripts were analyzed at P1, P6, P10 and adult; cortex data is from adult mice. The vast majority of reads are within expected size range for cDNAs of targeted genes. Dashed lines, quartiles of read length distribution.

FIG. 2 illustrates the mRNA isoform diversity revealed by lrCaptureSeq. (A) Total number of isoforms catalogued for each gene after completion of lrCaptureSeq bioinformatic pipeline. (B) UpSet plot comparing isoform numbers in the PacBio lrCaptureSeq dataset with public databases (RefSeq, UCSC Genes). Intersections show that 53.9% of NCBI RefSeq isoforms were detected in the PacBio dataset (255 RefSeq isoforms, 4^{rd}+6^{th} columns from left). For UCSC genes, 72.3% of isoforms annotated in this database were detected in the PacBio dataset (102 UCSC isoforms, 5^{th}+6^{th} columns). (C) Lorenz plots depicting total number of isoforms cataloged for each gene (right Y intercepts), and fraction of each gene's total reads represented by each of its isoforms (dots). Curves are cumulative functions, with isoforms displayed in order from highest (left) to lowest (right) fraction of total gene reads. Also see FIG. 10D. (D) Shannon diversity index was used to compare the relative diversity of each gene. Higher Shannon index reflects both higher isoform number and parity of isoform expression. (E) Treeplot depicting relative abundance of genes and isoforms (nested rectangles) within the entire dataset. Rectangle size is proportional to total read number. The most abundant isoform belonged to Crb1; the most abundant gene was Nrcam. (F,G) Unsupervised clustering applied at single gene level identifies families of related isoforms that share specific sequence elements. Ptprd gene is shown as an example. A subset of Ptprd isoforms cluster into 5 groups (F, bottom). These differ based upon 3 variables: length of 5′ UTR; length of 3′ UTR; and splicing of a variable exon cluster (F, top). The same groups segregate within principal components plot (G).

FIG. 3 demonstrates that transcript diversity contributes to a wealth of protein diversity. (A) Total number of transcripts and ORFs for each gene in the lrCaptureSeq dataset. ORF number typically scales with transcript number, as shown by similar line slopes across most genes. A minority of genes exhibit far fewer ORFs than transcript isoforms (steep slopes). (B) Lorenz plots of isoform ORF distributions, similar to FIG. 2C. Many predicted protein isoforms (dots) are expected to contribute to overall gene expression. (Also see FIG. 11A,B). (C) Shannon diversity index for unique predicted ORFs for each gene. Genes that encode trans-synaptic binding proteins are highlighted using gray text. (D) Treeplot depicting relative abundance of predicted ORFs within the dataset. For most genes, overall expression is distributed across many ORF isoforms. Genes with steep slopes in A (e.g. Cntn4) show differences here compared to transcript treeplot (FIG. 2E). (E) Schematic of proteomic techniques used to enrich for cell surface proteins. (F) Coomasie stained protein gel from biotin labeled and streptavidin-enriched cell surface proteins. Elution lane (E) shows enrichment of higher molecular weight proteins compared to total lysate input (I). Bands from 75 kDa-250 kDa were excised for mass spectrometry. (G) Plot depicting number of unannotated peptides discovered by mass spectrometry that do not exist in the UniProtKB database. Such peptides would have gone undetected if they had not been predicted to exist by lrCaptureSeq.

FIG. 4 demonstrates that the isoform diversity of Megf11 driven by modular alternative splicing. (A) Schematic of MEGF11 protein, showing how domain features correspond to exon boundaries. Most extracellular domain exons encode individual EGF or EGF-Laminin (Lam) repeats. Splicing that truncates EGF-Lam domains (e.g. skipping of exon 14) is predicted to leave behind an intact EGF domain, preserving modularity. Intracellular domain exons encoding canonical signaling motifs are noted: +, immunoreceptor tyrosine-based activation motif (YxxL/Ix₍₆₋₈₎YxxL/I); −, immunoreceptor tyrosine-based inhibitory motif (S/I/V/LxYxxI/V/L). TM, transmembrane domain; EMI, Emilin-homology domain. (B) Megf11 sashimi plot generated from combined PacBio dataset. The most variable exon clusters (13-17 and 19-23) are shown. Exons in these clusters can splice with any downstream exon within the cluster. Width of line corresponds to frequency of splicing event in isoform database. (C) Exon usage correlations across Megf11 isoforms. High Pearson correlation values (red) are seen at short range among exons that show minimal splicing, e.g. 1-8 and 17-19. Long range correlations are largely absent, suggesting that most splicing is stochastic. Strong long-range negative correlations are only observed in the trivial case of exons downstream from an alternative transcription stop site (asterisks). (D) Predicted protein structures of the 10 most abundant Megf11 isoforms. Alternative splicing varies number and identity of EGF and EGF-Lam domains on the extracellular portion of the protein, and produces 5 distinct cytoplasmic domains. Isoform 8 is the result of an alternative transcriptional stop site (C, exon 8b) and is predicted to encode a secreted isoform. *splicing from exon 19 to 20 results in a frameshift and early stop codon. **Retention of intron 24 results in a frameshift and early stop codon. (E) BaseScope in situ hybridization of P10 mouse retinal cross sections, using probes targeting indicated splice junctions (red). A constitutive junction (2-3, top left) shows full Megf11 expression pattern, in four cell types: ON and OFF starburst amacrine cells (blue arrows), horizontal cells (red arrow), and an unidentified amacrine cell (black arrow). Calbindin (green) marks starburst and horizontal cells. Staining intensity for each junctional probe is consistent with junction frequency in sequencing data (see Sashimi plot, B). All junctions are expressed by all individual cells of the starburst and horizontal populations. Scale bar=10 μm.

FIG. 5 demonstrates that Crb1-B is the most abundant Crb1 isoform in mouse and human retina. (A,B) Transcript maps of most abundant Crb1 isoforms from mouse retina (A) and cortex (B). A is the canonical isoform; A2 is a minor splice variant of A. These isoforms are shared between retina and cortex, whereas Cortex 1, Cortex 2, and Crb1-B are tissue-specific. Corresponding exon coverage and sashimi plots were generated from lrCaptureSeq dataset. Note prevalence of reads associated with Crb1-B isoform (A). (C) Assay for chromatin accessibility (ATAC-seq; GSE102092, GSE83312) identifies likely promoters of Crb1-A and -B isoforms. Bars indicate location of putative A and B promoters. Maps in A-C are aligned with each other. Crb1-A promoter is more open during development, but stays accessible in mature retina. Crb1-B promoter is open and presumed active in mature rods and both types of cones. DNase I hypersensitivity data from ENCODE project reveals distinct chromatin environment in frontal cortex, consistent with expression of A isoform, as well as shorter cortex isoforms (cortex 1 and 2; bar at top labeled “ctx”). (D) Retinal expression of top 3 Crb1 isoforms across mouse development, quantified from PacBio dataset. A isoforms predominate at P1 but Crb1-B becomes most abundant by P6. Data were normalized to total Crb1 read counts at each timepoint (P1=923 reads, P6=6,127 reads, P10=14,007 reads, Adult=10,975 reads). (E) Transcript maps of most abundant human retinal CRB1 isoforms, identified by lrCaptureSeq. A and B isoforms are highly homologous to mouse (A). CRB1-C encodes a putative secreted form of the protein; it was also identified in the mouse dataset but its relative abundance in mouse was much lower than A and B. Note that Crb1-A2 was not detected in the human dataset. Exon coverage and sashimi plots were generated from lrCaptureSeq data. (F) ATAC-seq (GSE99287) of human peripheral (per.) and macular (mac.) retina show open regulatory sites corresponding to putative promoters for CRB1-A (bar labeled “A”) and CRB1-B (bar labeled “B”). Two biological replicates are shown. Maps in E,F are aligned with each other. (G) Expression of top 3 human CRB1 isoforms, quantified from adult human retina lrCaptureSeq dataset. (H,I) Quantification of top 3 mouse (H) or human (I) (RBI isoforms using short-read RNA-seq data. Mouse dataset (GSE101986) confirms developmental regulation of each isoform observed in PacBio data (D). Human dataset (GSE94437) confirms (RBI-B is dominant isoform in adult retina. Lines (I) show measurements derived from same donor. Statistics (I): One-way ANOVA with Tukey's post-hoc comparison. ****P<1×10⁻⁷. ***P=1.6×10⁻⁶ (top); P=6.6×10⁻⁶ (bottom). Error bars, 95% confidence intervals (H) or S.D. (I).

FIG. 6 demonstrates that CRB1-B is expressed by photoreceptors. (A) Domain structures of CRB1-A and CRB1-B protein isoforms. Isoform-specific regions are highlighted using dashed rectangles. Each isoform has unique sequences at N-termini, predicted to encode signal peptides, and at C-termini, predicted to encode transmembrane (TM) and intracellular domains. (B) ClustalW alignment of unique CRB1-B sequences. Both N- and C-terminal regions are highly conserved across vertebrate species. The N-terminal region comprises a signal peptide (left) and the C-terminal region comprises a transmembrane domain (right). The illustrated sequences are as follows: SEQ ID NO:87 is the consensus signal peptide, SEQ ID NO:88 is the consensus transmembrane domain, SEQ ID NO:89 is the Homo sapiens signal peptide, SEQ ID NO:3 is the Homo sapiens transmembrane domain, SEQ ID NO:90 is the Bos taurus signal peptide, SEQ ID NO: 91 is the Bos taurus transmembrane domain, SEQ ID NO:92 is the Mus musculus signal peptide, SEQ ID NO:93 is the Mus musculus transmembrane domain, SEQ ID NO:94 is the Rattus norvegicus signal peptide, SEQ ID NO:95 is the Rattus norvegicus transmembrane domain, SEQ ID NO: 96 is the Danio rerio signal peptide, and SEQ ID NO:97 is the Danio rerio transmembrane domain. (C) Western blot verifying CRB1-B protein expression in retinal lysates. CRB1-B antibodies were generated against unique CRB1-B C-terminus. Deletion of Crb1-B first exon in mutant mice (Crb1^{delB} allele; see FIG. 7A) demonstrates antibody specificity and that unique first and last exons of Crb1-B are primarily used together, as predicted at transcript level (FIG. 5A). Photoreceptor protein ABCA4 is used as loading control. (D) Western blot on retinal lysates separated into soluble(S) and membrane-associated (M) protein fractions. CRB1-B is detected in the membrane fraction. Loading controls: Membrane fraction, ABCA4; soluble fraction, Phosducin. (E) Schematic showing anatomy of outer retinal region where CRB1 is expressed. Left, photomicrograph depicting photoreceptor nuclei; inner segment; and outer segment. The outer limiting membrane (OLM) separates nuclear layer from inner segment layer. Right, OLM anatomy schematic. OLM consists of junctions (dots) between photoreceptors and Müller cells. These junctions form selectively at particular subcellular domains of each cell type, i.e. glial apical membranes and photoreceptor inner segments. CRB1-A is expressed by Müller cells (F,G) where it localizes selectively to OLM junctions⁴⁹. CRB1-B is expressed throughout the photoreceptor, including inner and outer segments (F-H). Also see FIG. 14. (F) Mapping of Crb1 isoforms in scRNA-seq data⁴⁸. Heat map generated from gene profiles of >90,000 cells, showing normalized expression of Crb1 isoforms and retinal cell type marker genes. Unsupervised clustering was used to define genes co-expressed with Crb1 isoforms. Crb1-B clusters with known cone and rod photoreceptor genes, while Crb1-A clusters with known Müller glia genes. (G) BaseScope in situ hybridization of P20 mouse retinas using isoform-specific probes (red). Blue, Hoeschst nuclear counterstain. Crb1-A probe targeted exon 1-2 junction, which is also used by Crb1-A2 and Crb1-C (see FIG. 5A). Signal is primarily limited to central INL, where Müller cell bodies reside (left). Crb1-B probe targeted the junction between its unique 5′ exon and exon 6 (FIG. 5A). Signal is limited to photoreceptors within ONL. Abbreviations: ONL=outer nuclear layer; INL=inner nuclear layer; GCL=ganglion cell layer. Scale bar, 100 μm. (H) Subcellular localization of CRB1-B within rod photoreceptors, assessed by Western blotting of serial 10 μm tangential sections through mouse outer retina. Each lane corresponds to photoreceptor cellular compartment denoted by cartoon at top. Rhodopsin (Rho, center) is an outer segment marker; GAPDH (bottom) is excluded from outer segment but is present throughout the rest of the cell. CRB1-B protein (top) is present in all compartments; expression is strongest in lanes corresponding to outer and inner segments.

FIG. 7 demonstrates that Crb1 isoforms are required for outer limiting membrane integrity. (A) Schematic of Crb1 locus showing genetic lesions associated with mouse mutant alleles. Previously studied mutants: Crb1^{ex1}, a targeted deletion of exon 1 that does not impact the Crb1-B isoform; Crb1^{rd8}, a point mutation in exon 9. Mutant alleles generated for this study: Crb1^{delB}, a CRISPR-mediated deletion of the first Crb1-B exon and its promoter region, leaving the Crb1-A isoform intact; Crb1^{null}, a large CRISPR-mediated deletion of consecutive exons that are used in all Crb1 isoforms. Also see FIG. 15A for documentation of new alleles. (B,C) Assessment of OLM junctions by electron microscopy. B: schematic illustrating location of OLM junctions (dots) surrounding photoreceptor inner segments. C: Electron micrograph from wild-type mouse. All inner segments make OLM junctions with Müller cells. IS, inner segment. Arrowheads marked with plus sign (+), photoreceptor-glial junctions. Arrowheads marked with hash sign (#), glial-glial junctions. (D,E) OLM disruption phenotype in Crb1 mutants. D: electron micrograph from control (wild-type) mouse. OLM divides outer nuclear layer (ONL) from IS layer. In Crb1 mutants (E), gaps in OLM allow nuclei to penetrate into inner segment layer. Arrows demarcate region lacking OLM junctions. This image is from Crb1^{delB/null} mutant, but is representative of OLM phenotypes observed in null, delB, and rd8 mutants (FIG. 15D-F). (F-I) Higher power views of OLM gaps in Crb1 mutants, showing inner segments that lack OLM junctions (asterisks). In each allelic combination, photoreceptors lacking Müller contacts were observed. Marked arrowheads as in C. (J) Quantification of OLM gap frequency. No gaps were observed in wild-type or Crb1^{null+} heterozygotes. The frequency of OLM disruption was similar in rd8, null, and delB/null mutants, the latter of which lack Crb1-B but still express Crb1-A. Statistics, one-way ANOVA with Tukey's post-hoc test. Null, rd8, and delB/null were all significantly different from wild-type and heterozygous controls (respective P-values: 0.014; 0.005; 0.019), but did not differ significantly from each other (rd8 vs. null P=0.991; rd8 vs. delB/null P=0.784; null vs. delB/null P=0.967). Also see FIG. 15F for quantification of gap sizes. Scale bars, 2 μm C, D (bar also applies to E); 1 μm G (bar also applies to F), H, I.

FIG. 8 demonstrates that ablation of all Crb1 isoforms causes retinal degeneration. (A) Retinal histology in Crb1 mutant mice at P100. Thin plastic sections through inferior hemisphere are shown for homozygous mutants of indicated genotype, and wild-type controls. Arrow, ONL layer containing photoreceptor nuclei. Large focal region of photoreceptor loss is evident in Crb1^{null} retina, accompanied by retinal detachment. Areas outside the most aggressively degenerative patch show ONL thinning. Crb1^{delB} and Crb1^{rd8} mutants show no apparent loss of ONL cells. ONH, optic nerve head. (B) Higher magnification views of retinal histology, 450 μm inferior to ONH. Images from two different Crb1^{null} animals are shown to highlight variability in focal degeneration. Even the mild null case has thinner ONL (light gray line) with fewer nuclei than age-matched Crb1^{rd8}. Outer segment length (dark gray line) is also diminished in null mutants. (C,D) Quantification of ONL cell number at P100. C: Spider plot showing counts of ONL nuclei in 100 μm bins distributed uniformly across retinal sections (e.g. B). Left, inferior side. For Crb1^{delB} spider plot see FIG. 15. D: Total ONL nuclei counted in all 8 bins. Statistics (C): 2-way ANOVA with Sidak post-hoc test. P-values refer to WT vs. null comparison; rd8 was not significantly different from WT at any location. *P=0.015; **P=0.007, P=0.004; ****P<1×10⁻⁷. Statistics (D): 1-way ANOVA with Tukey's post-hoc test. Crb1^{null} was significantly different from all other groups. ****P<1×10⁻⁵ for all comparisons. None of the other Crb1 mutants differed from WT or each other. Sample sizes denoted by dots on graph (D).

FIG. 9 illustrates PacBio sequencing of captured cDNAs. (A) Histogram of PacBio read size distribution for a pilot lrCaptureSeq experiment, in which the second size selection after PCR amplification was not performed (see workflow, FIG. 1B. Profile demonstrates that this size selection is necessary for enrichment of long transcripts. Dotted line represents interquartile range. FLNC, full-length non-chimeric reads called by Iso-Seq software. (B) Percentage of on target reads per experiment, calculated as the number of high quality (HQ) reads corresponding to our targeted genes vs. all other reads. HQ reads called by Iso-Seq software. (C) Sequencing statistics from each individual lrCaptureSeq experiment and the combined dataset. (D,E) Validation of lrCaptureSeq isoform 5′ ends by CAGE. Three independent CAGE-seq replicates from adult mouse retina were mapped to the adult mouse retina lrCaptureSeq isoforms. D: Box and whiskers plot showing CAGE read coverage at the first exon of lrCaptureSeq isoforms. Coverage is extensive, supporting the accuracy of lrCaptureSeq 5′ ends. Box represents IQR, horizontal line represents median, whiskers equal to 1.5*IQR. E: Position along 5′-3′ axis of CAGE reads that mapped to lrCaptureSeq isoforms. CAGE coverage was exclusive to the 5′ end of transcripts.

FIG. 10 shows the isoform length and abundance in the lrCaptureSeq catalog. (A) UpSet plot comparing number of “ground-truth” isoforms in the lrCaptureSeq dataset with ones computationally predicted from retina and cortex RNA-seq datasets by Cufflinks or Stringtie. Many more isoforms were detected by lrCaptureSeq than were assembled by these two programs. Nevertheless, only a minority of predicted isoforms were validated by long-read sequencing: 186 isoforms predicted by Cufflinks (3^{rd}+5^{th} columns) were detected in the PacBio dataset (or 38% of Cufflinks isoforms), and 170 isoforms predicted by Stringtie (4^{th}+5^{th} columns) were detected (or 17.7% of Stringtie isoforms). (B) Box and whisker plot showing number of RNA-seq reads that mapped to lrCaptureSeq isoforms. Two classes of isoforms are compared: those for which all exon junctions were validated in RNA-seq data (Full), and those that were not 100% validated (Partial). Read counts were lower for the latter group, suggesting that failure to validate all junctions may have resulted at least in part from low expression levels and/or insufficient RNA-seq read coverage of those particular isoforms. Box represents IQR, horizontal line represents median, whiskers equal to 1.5*IQR. Split bar indicates 95% confidence interval of the mean. (C) Contribution of isoforms containing non-canonical splice junctions to overall isoform count. Curves show abundance rank ordering of all isoforms and the same rank ordering for only those isoforms that contain a non-canonical splice junction. Non-canonical junctions account for a small fraction of total isoforms. Successively removing the least abundant isoforms from each gene (i.e. moving along the X axis) yields a similar fraction of isoforms that use a non-canonical junction, suggesting some of these are abundantly expressed. (D) Plots depicting the number of isoforms that account for the top 50% (D) or 75% (E) of each gene's total read count (see FIG. 2C). These plots show that, even with strict abundance cutoffs, many isoforms exist and contribute to overall gene expression. (E,F) Isoforms vary substantially in their length. This is shown by a dotplot depicting the lengths of isoforms for each gene (F) and by a box and whiskers plot depicting the number of exons used across isoforms of each gene. Box represents IQR, horizontal line represents median, whiskers equal to 1.5*IQR. (G) t-SNE plot of all isoforms. Most isoforms segregate into their respective gene families, validating efficacy of clustering algorithms for comparing isoform similarity. Isoforms in center of plot that do not segregate well generally contain large genomic elements (i.e. retained introns) which impede clustering with other isoforms of the same gene. The spread of isoforms suggests significant variations in sequence composition. Plot was generated with 1,000 iterations and perplexity=35.

FIG. 11 shows coding and non-coding isoform variations. (A,B) Plots depicting the number of unique predicted ORFs that account for the top 50% (A) or 75% (B) of each gene's total read count (see FIG. 3B). (C) Intron retention is a major source of non-protein-coding isoform diversity, as exemplified here by Vldlr gene. The top 20 most abundant Vldlr isoforms are illustrated. Thick black bars, exons. Note extensive, combinatorial intron retention. Asterisks, introns that were detected in lrCaptureSeq isoforms (i.e. within polyadenylated transcripts). Intron retention creates a high degree of transcript diversity that does not translate to high ORF diversity. All of the retained introns introduce premature stop codons. (D) Non-coding transcript diversity can arise from variations in the 5′ UTR region of the gene, as exemplified here by Cntn4. Figure shows 5′ end of top 20 most abundant Cntn4 isoforms. Note alternative transcriptional start sites and differential exon usage within 5′ UTR. (E) The number of unique trypsin peptide products encoded by our 30 genes in the UniProtKb database (right bar), compared to the number of predicted trypsin peptide products that exist within the lrCaptureSeq dataset (left bar).

FIG. 12 shows the Megf11 isoform diversity uncovered by PacBio sequencing. (A) DNA electrophoresis gel image of Megf11 RT-PCR products. Primers were designed to amplify two different Megf11 variants (denoted long and short) by placing primers in exon 25 or alternative exon 23, respectively. PCR was performed on retinal (long) or cortex (short) cDNA. The size spread of RT-PCR products indicates that numerous Megf11 isoforms of different sizes can be readily amplified. (B) Lorenz plot profiles of Megf11 isoform abundance from lrCaptureSeq and

PCR datasets. All datasets suggest that many isoforms contribute to overall Megf11 expression. The rightward shift of the PCR dataset curves suggests overrepresentation of the most abundant isoforms, likely due to PCR-induced bias. (C) Transcript maps depicting the long and short forms of Megf11 (top) and corresponding exon coverage and sashimi plots from 3 different PacBio sequencing datasets. PCR1 dataset was generated by sequencing Megf11 long form PCR products, while PCR2 dataset was generated by sequencing short form PCR products. These are compared to the Megf11 reads from the 30-gene lrCaptureSeq experiment. All three experiments reveal extensive alternative splicing of Megf11 transcripts. Sashimi plots show remarkable similarity between the different datasets.

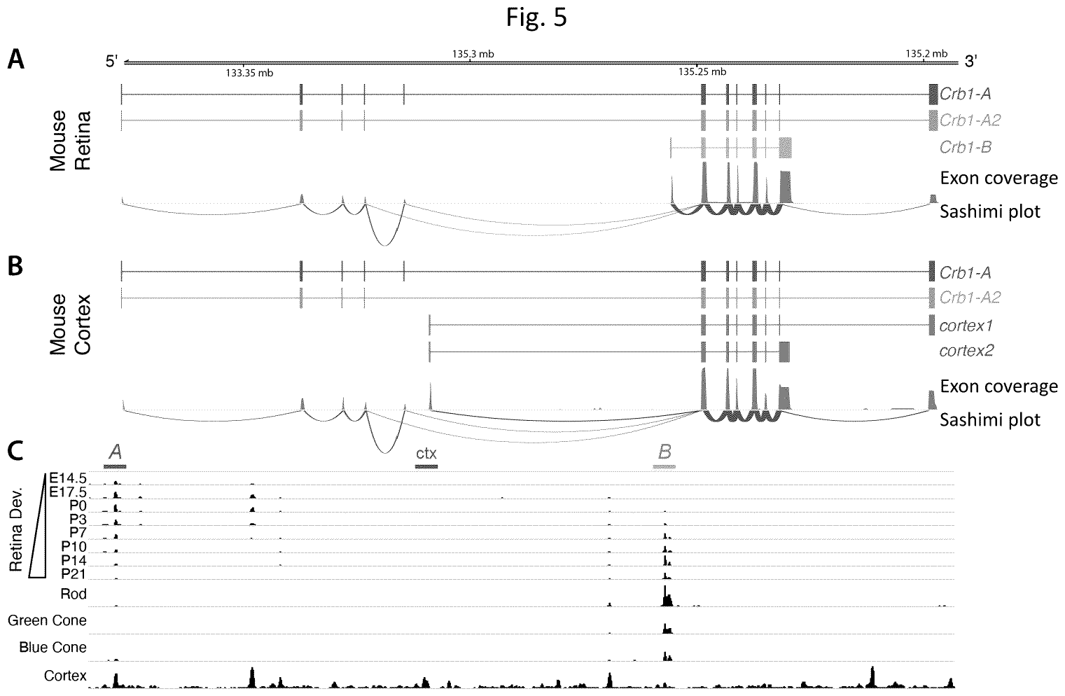

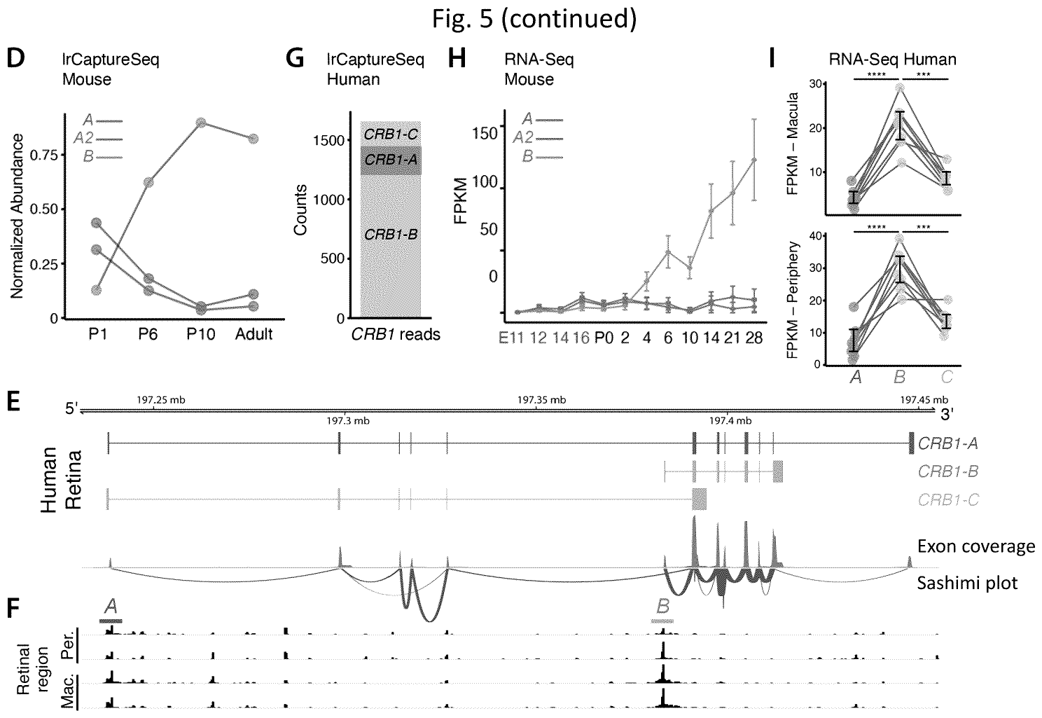

FIG. 13 demonstrates that the Crb1-B isoform is expressed across a variety of vertebrate species. (A) Quantification of Crb1 isoforms in bovine, rat, and zebrafish retina, based on publicly available RNA-seq data (bovine, GES59911; rat, GSE84932; zebrafish, GSE101544). Crb1-B is at least as abundant as Crb1-A in all species, and is more abundant in rat and zebrafish. Crb1-A2 was not detectable in bovine or zebrafish retina. Error bars represent 95% confidence intervals. (B) Quantitative (q) RT-PCR analysis of Crb1 isoforms in mouse retina confirm expression patterns identified using PacBio and short-read RNA-seq (FIG. 5). Crb1-A is most abundant at P1, while Crb1-B is most abundant in adulthood. PCR primers were designed to span splice junctions expressed by the indicated isoforms. Data were normalized to values obtained from pan-Crb1 primers. N=3 animals for each age. (C) RT-PCR on cDNA from mouse retina and cortex, using pan-Crb1 primers (pan), or primers targeting a Crb1-B splice junction (B). No Crb1-B band is detected in mouse cortex. Pan-Crb1 primers produce bands in both tissues. N=3 mice. L, ladder.

FIG. 14 shows the cell-type-specific expression of Crb1 isoforms. (A) Pearson correlation of Crb1 exons demonstrates that exons unique to Crb1-B (5c and 11b) are negatively correlated with exons unique to Crb1-A isoforms (1-5 and 12). The unique Crb1-B exons (5c and 11b) are strongly positively correlated suggesting that they are primarily used together. (B) Quantification of Crb1 isoforms from bulk RNA-seq of isolated cone (top) and rod (bottom) photoreceptors (dataset: GSE74660). Crb1-B is the only isoform expressed in photoreceptors. Error bars, 95% confidence intervals. (C) CRB1 isoforms expressed in K562 cells traffic to the plasma membrane. Images depict native fluorescence of CRB1-A and CRB1-B constructs tagged at C-terminus with YFP. (D) Mapping of Crb1 isoforms in single-cell RNA-seq data³¹. Jitter plot indicates relative transcript expression counts within individual cells. Each point represents one cell, shaded by the annotated cell type. Crb1-A is expressed by Müller glia whereas Crb1-B is expressed by rod and cone photoreceptors. Cell type-specific markers of Müller glia (Aqp4), rods (Gnat1), cones (Gnat2), and bipolar cells (Pcp2) are shown for comparison.

FIG. 15 depicts Crb1 mutant mice and OLM phenotypes. (A) Location of deletions within Crb1^{null} and Crb1^{delB} alleles, verified by Sanger sequencing. Delta symbol (Δ) indicates size of the deleted genomic fragment. The genomic region comprising the Crb1^{null} allele is SEQ ID NO:98 (top four sequences), while genomic region comprising the Crb1^{delB} allele is SEQ ID NO:99 (fifth, seventh, and eighth sequence). The sequence illustrating the Crb1^{delB} deletion is SEQ ID NO:100 (sixth sequence). (B) Confirmation that CRB1-B protein is eliminated in Crb1^{null} mutant mice.

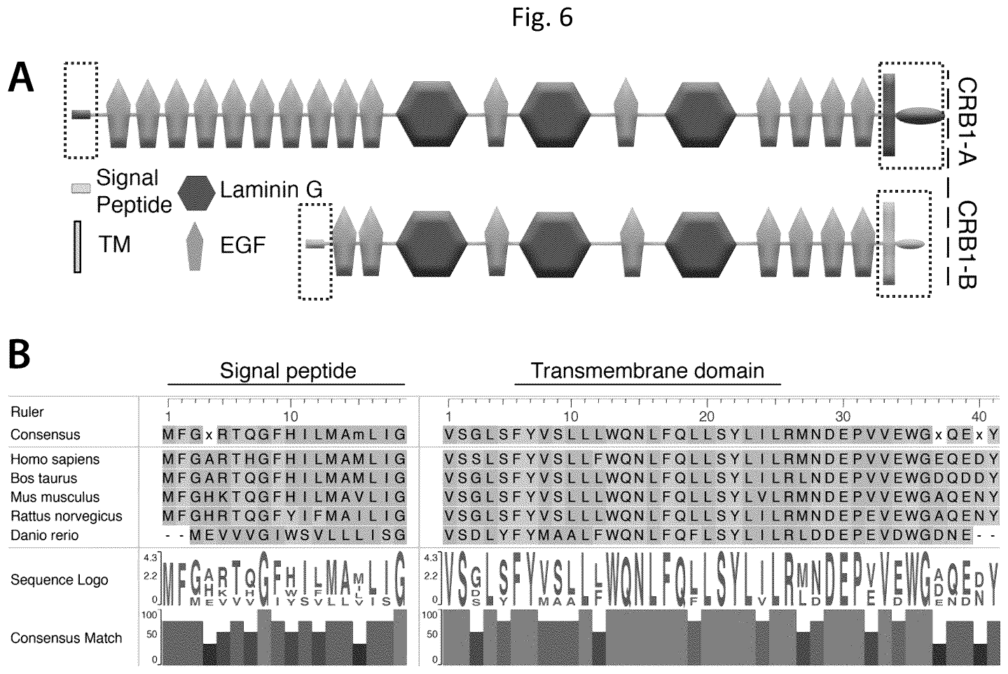

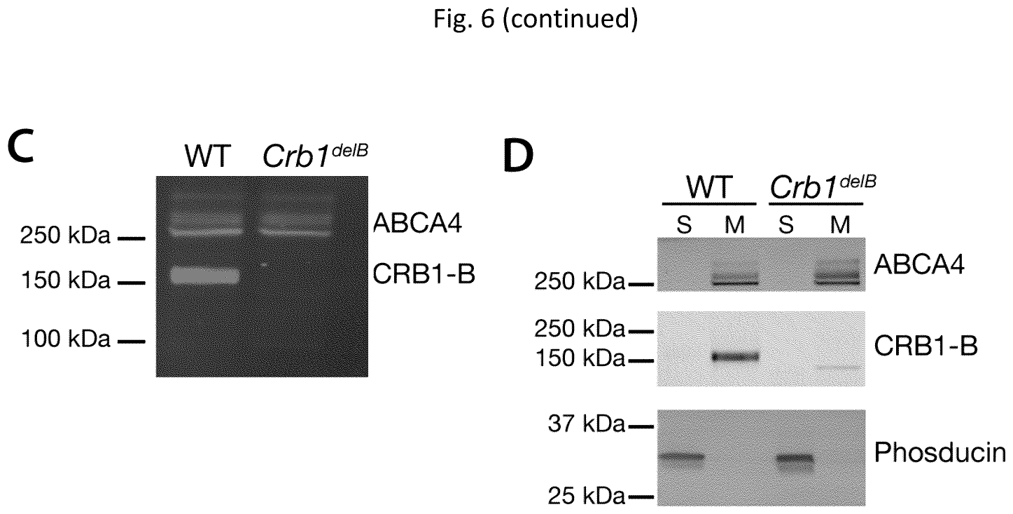

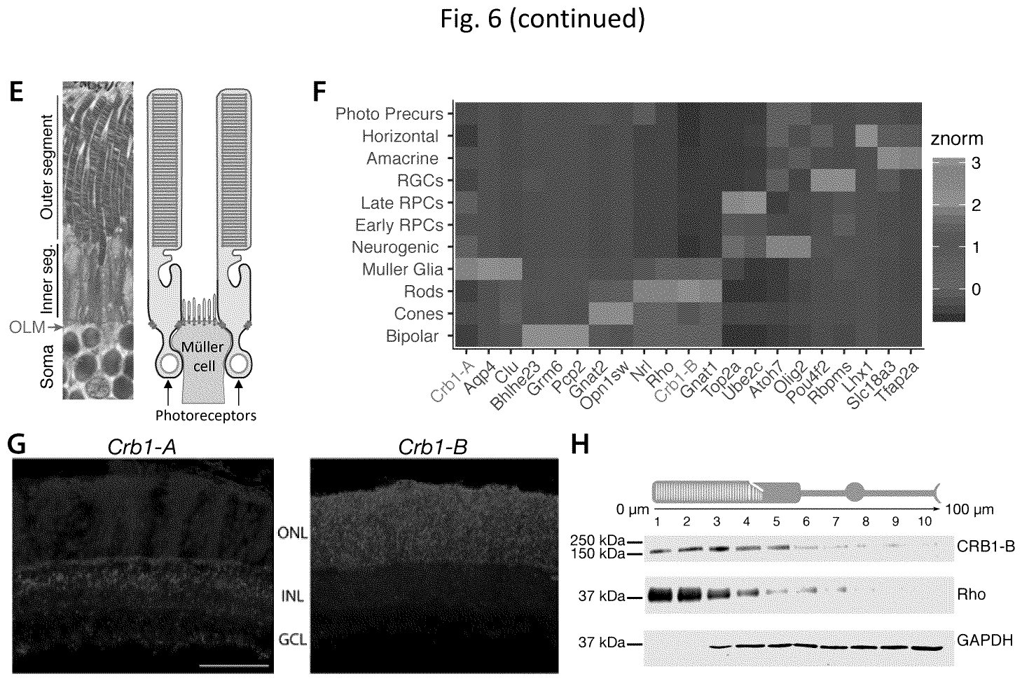

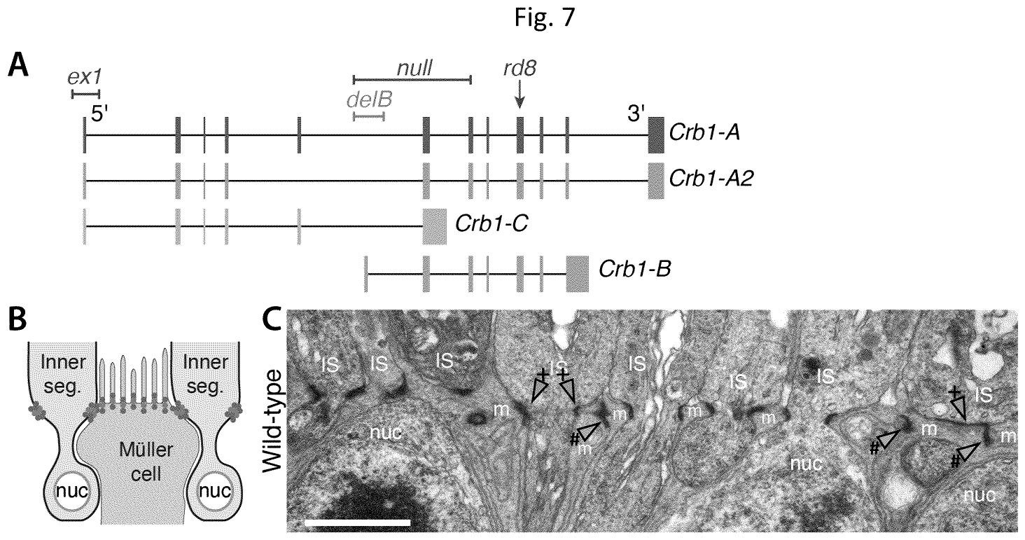

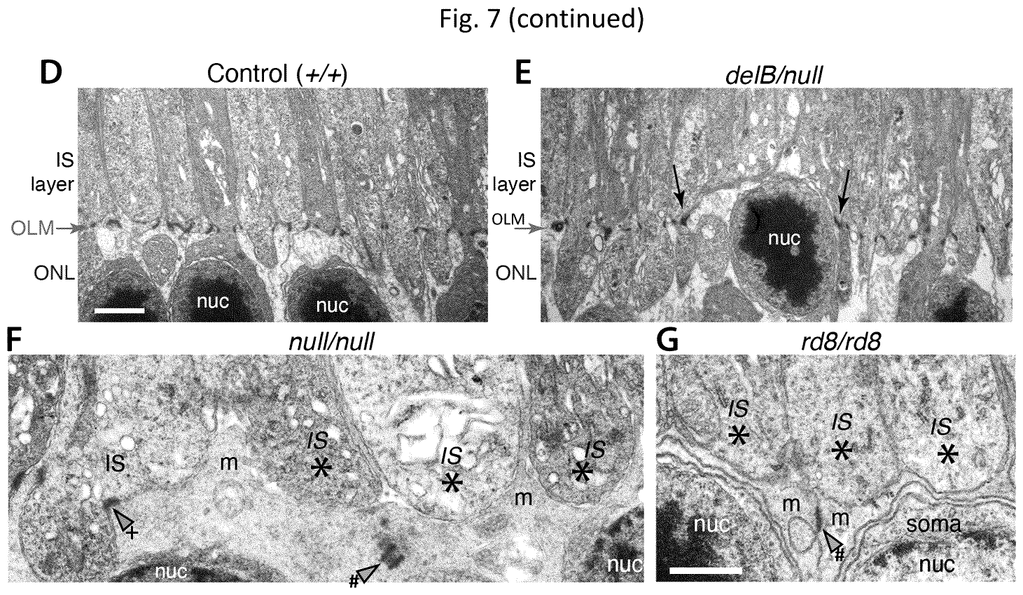

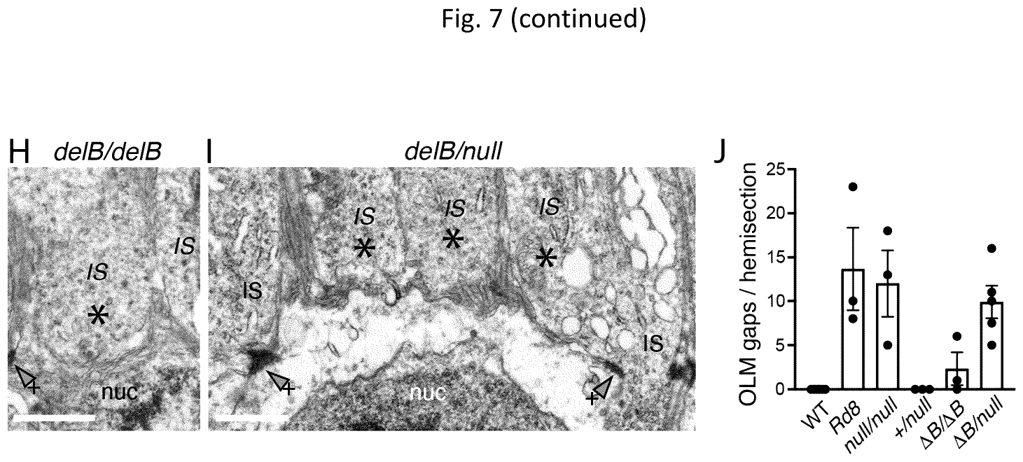

Western blots on retinal lysates were performed as in FIG. 6C. ABCA4, loading control. (C) Spider plot showing lack of photoreceptor loss in Crb1^{delB/delB} mice at P100. Gray, wild-type controls. (D,E) Representative electron micrographs showing OLM disruptions in Crb1^{null} and Crb1^{rd8} mutants. Images are similar in scale to FIG. 7D,E. Arrows demarcate region lacking OLM junctions. Anatomical disturbances are similar to those previously reported for rd8³⁶, and to those observed in Crb1^{delB/null} mice, which lack Crb1-B but still retain one copy of Crb1-A (FIG. 7). Scale bar, 5 μm. (F) OLM gap size in Crb1 mutants carrying various allele combinations. Size of OLM gaps was not significantly different across the various mutants. Statistics, one-way ANOVA (F=2.19; P=0.095).

FIG. 16 shows the polypeptide sequence of the CRB1-B isoform (SEQ ID NO:1) with the EGF domains highlighted in gray (residues 24-65, 68-109, 303-334, 516-550, 773-802, 804-839, 841-876 and 924-960) and the laminin G domains highlighted in red (residues 141-276, 370-487, and 607-732). A schematic depiction of the protein domains is shown below the sequence.

### DETAILED DESCRIPTION

Gene replacement is a promising therapeutic strategy for the wide spectrum of retinal degenerative diseases caused by loss-of-function mutations in the Crb1 gene. However, to design an effective gene replacement strategy, both the cell type in which this gene needs to be replaced and the proper Crb1 isoform to provide must be identified.

Crb1 is a member of the evolutionarily conserved Crumbs gene family, which encode cell-surface proteins that mediate apico-basal epithelial polarity³³. Notably, it is considered standard practice to refer to the mouse version of the gene as Crb1 and refer to human version of the gene as CRB1. However, in the present application, the nomenclatures Crb1 and CRB1 are used interchangeably to refer to the gene and are not necessarily used to indicate the species from which the gene is derived.

In the retina, CRB1 localizes to the outer limiting membrane (OLM), a set of structurally important junctions between photoreceptors and neighboring glial cells known as Müller glia²⁶. OLM junctions form at precise subcellular domains within each cell type, suggesting a high degree of molecular specificity in the establishment of these intercellular contacts³⁴. There is great interest in understanding the function of CRB1 at OLM junctions, because loss-of-function mutations in human CRB1 cause a spectrum of retinal degenerative disorders³⁵. It has been proposed that loss of OLM integrity might play a role in disease pathogenesis^{26,36}. Yet, studies in mice have yet to provide convincing support for this model. For example, in mice, deletion of the known Crb1 isoform neither disrupts the OLM nor causes significant photoreceptor degeneration³⁷.

In the present application, the inventors identify a new Crb1 isoform that is far more abundant—in both mouse and human retina—than the canonical isoform. Using a mouse model, they show that this new isoform is required for OLM integrity and that its removal is required to adequately phenocopy the human degenerative disease. These results call for a major revision to prevailing models of CRB1 disease genetics and pathobiology. Remarkably, the present inventors discover that the major isoform of the retinal degeneration gene Crb1 was previously overlooked. This isoform, Crb1-B, is the only one expressed by photoreceptors, the affected cells in CRB1 disease. Using a mouse model, the inventors identify a function for this isoform at photoreceptor-glial junctions and demonstrate that loss of this isoform accelerates photoreceptor death.

The present invention demonstrates that the major isoform Crb1-B, when presented in trans, is sufficient to retain photoreceptor function, allowing for its use to maintain vision and reduce vision loss. Specifically, introduction of the Crb1-B isoform into retinal photoreceptor cells is sufficient to maintain photoreceptor function and reduce loss of photoreceptor function.

### Isoform Annotation

Most genes generate multiple mRNA isoforms. As used herein the term “isoform” is used to describe mRNAs that are produced from the same locus but are different in their transcription start sites (TSSs), protein coding DNA sequences (CDSs) and/or untranslated regions (UTRs). Alternative isoforms are produced by mechanisms such as alternative splicing, intron retention, and alternative transcription start/stop sites. Alternative isoforms often differ in their protein-coding capacity¹⁻⁴, which sometimes results in altered gene function. These mechanisms are especially common in the central nervous system (CNS), where the use of alternative isoforms is particularly prevalent^{1,5}. Moreover, dysregulation of isoform expression is implicated in several neurological disorders⁹⁻¹¹.

Despite the clear importance of isoform diversity, information about the number and the identity of CNS mRNA isoforms remains surprisingly scarce—even within the major transcriptome annotation databases¹². RNA-sequencing (RNA-seq) has generated an explosion of new information about alternative splicing However, because typical RNA-seq read lengths are less than 200 bp, this method is not able to resolve the full-length sequence of multi-kilobase transcripts. Therefore, by relying on RNA-seq alone, it is impossible to determine the number of isoforms produced by any given gene, or their full-length sequences. In the absence of reliable full-length transcript annotations, the design and interpretation of genetic experiments becomes exceedingly difficult. For example, unless transcript sequences are known, it is difficult to be certain that a “knockout” mouse allele has been properly designed such that it fully eliminates expression of all isoforms. Unannotated isoforms can also be problematic for understanding how mutations lead to pathology in human genetic disease. Hidden isoforms may possess uncharacterized protein-coding sequences or novel expression patterns, which could cause the molecular and cellular consequences of disease-linked mutations to be misinterpreted. Thus, a lack of comprehensive isoform sequence information remains a major impediment to our understanding of both normal gene function and the phenotypic consequences of gene dysfunction¹².

In the present application, the inventors devised a strategy that leverages Pacific Biosciences (PacBio) long-read sequencing technology to generate comprehensive catalogs of CNS cell-surface molecules. Long-read sequencing is ideal for full-length transcript identification; however, the available sequencing depth is not sufficient to reveal the full scope of isoform diversity²⁷⁻³⁰. To overcome this limitation, the inventors adapted a strategy from short-read sequencing, in which targeted cDNAs are pulled down with biotinylated probes against known exons^{31,32}. This approach yielded major improvements in long-read coverage, revealing an unexpectedly rich diversity of isoforms encoded by the targeted genes. To make sense of these complex datasets, the inventors developed bioinformatics tools for the classification and comparison of isoforms, and for determining their expression patterns using short-read RNA-seq data. Using these methods, the inventors were able to identify a novel Crb1 isoform that offers great potential for the treatment of retinopathies.

### Compositions

### i. Polynucleotide sequences, Vectors and Isolated Proteins

Gene therapy protocols for disorders of the eye require the localized delivery of the polynucleotide or vector to the cells in the eye (e.g., cells of the retina) for local expression. The cells that will be the treatment target in these diseases may include, inter alia, one or more cells of the eye (e.g., photoreceptors, ocular neurons, etc.). The polynucleotides, vectors, polypeptides, compositions, methods, systems and kits of the present disclosure are based, at least in part, on the discovery that a certain unknown isoform of the gene Crb1, termed Crb11 B is, exclusively expressed in retinal photoreceptors. The Crb1-B isoform has been found by the inventors to be an attractive candidate for Crb1 gene replacement therapy for numerous reasons, including for example: (i) size; (ii) their localized expression in retinal photoreceptors-the cell type that degenerates in retinal dystrophies; (iii) the presence of a unique promoter as well as unique first and last coding exons making them functionally distinct from other isoforms; and (iv) increased expression (e.g., Crb1-B is expressed ˜10 fold higher) than other Crb1 isoforms in the retina, suggesting their function may be the most important to replace to rescue vision. As demonstrated in the examples, CRB1-B is the majority isoform expressed in retinal photoreceptors, while the other isoforms are expressed in other retinal cell types (e.g. CRB1-A is found expressed in Müller cells). As such, in trans expression of CRB1-B protein within photoreceptors in a subject is sufficient by itself to retain photoreceptor function and maintain vision in the subject.

In one embodiment, the present technology provides an isolated polynucleotide comprising a polynucleotide sequence encoding a Crumbs 1-B (CRB1-B) isoform comprising SEQ ID NO:1(the human CRB1-B protein) operably linked to a heterologous promoter capable of expressing the isoform in a retinal cell. CRB1-B isoform is specifically expressed in photoreceptor cells, predominantly within the inner and outer segments. This localization is in marked contrast to CRB1-A which has been localized to the apical tips of Müller cells, within the OLM (See FIG. 6E). In one embodiment, the polynucleotide sequence encoding the CRB1-B isoform is SEQ ID NO:2.

In other embodiments, the present technology provides isolated polynucleotides encoding other isoforms of the human Crumbs 1 gene. In one embodiment, the polynucleotide sequence (SEQ ID NO:4) encodes a Crumbs 1-A (CRB1-A) isoform comprising SEQ ID NO:5 (human CRB1-A protein). In another embodiment, the polynucleotide sequence (SEQ ID NO:6) encodes a Crumbs 1-C (CRB1-C) isoform comprising SEQ ID NO:7 (human CRB1-C protein).

In further embodiments, the isolated polynucleotides encode isoforms of the mouse Crumbs 1 gene. In one embodiment, the polynucleotide sequence (SEQ ID NO:8) encodes a Crumbs 1-A (CRB1-A) isoform comprising SEQ ID NO:9 (mouse CRB1-A protein). In another embodiment, the polynucleotide sequence (SEQ ID NO:10) encodes a Crumbs 1-B (CRB1-B) isoform comprising SEQ ID NO:11 (mouse CRB1-B protein). In yet another embodiment, the polynucleotide sequence (SEQ ID NO:12) encodes a Crumbs 1-C (CRB1-C) isoform comprising SEQ ID NO:13 (mouse CRB1-C protein). In yet a further embodiment, the polynucleotide sequence encodes a Crumbs 1-A2 (CRB1-A2) protein.

The terms “polynucleotide” or “nucleic acid” are used interchangeably herein and refer to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be an oligodeoxynucleoside phosphoramidate (P—NH₂) or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer. Polynucleotide sequences provided herein are provided as the cDNA encoding for the CRB1 isoform of interest.

As used herein, a “therapeutic” agent (e.g., a therapeutic polypeptide, nucleic acid, or transgene) is one that provides a beneficial or desired clinical result, such as the exemplary clinical results described above. As such, a therapeutic agent may be used in a treatment as described herein. In some embodiments, the polynucleotide comprises a Crb1 isoform. In a preferred embodiment, the Crb1 isoform is Crb1-B. In another embodiment, the isoform is selected from the group consisting of Crb1-A, Crb1-A2, Crb1-B, Crb1-C and combinations thereof.

“Heterologous” means derived from a genotypically distinct entity from that of the rest of the entity to which it is compared or into which it is introduced or incorporated. For example, a polynucleotide introduced by genetic engineering techniques into a different cell type is a heterologous polynucleotide (and, when expressed, can encode a heterologous polypeptide). Similarly, a cellular sequence (e.g., a gene or portion thereof) that is incorporated into a viral vector is a heterologous nucleotide sequence with respect to the vector. The term “transgene” refers to a polynucleotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome. In another aspect, it may be transcribed into a molecule that mediates RNA interference, such as miRNA, siRNA, or shRNA. The transgene for use in the present invention is an isoform of Crb1, preferably Crb1-B.

As used herein, the term “isolated” means that the material is removed from its original environment (e.g., the natural environment if it is naturally occurring). For example, a naturally occurring polynucleotide or polypeptide present in a living microorganism is not isolated, but the same polynucleotide or polypeptide, separated from some or all of the coexisting materials in the natural system, is isolated. Such polynucleotides could be part of a vector and/or such polynucleotides or polypeptides could be part of a composition and still be isolated in that such vector or composition is not part of its natural environment.

Accordingly, in another aspect of the present disclosure provides a recombinant vector comprising, consisting of, or consisting essentially of a polynucleotide comprising a Crb1 isoform and encoding the CRB1-B protein.

The terms “vector” or “recombinant vector” are used interchangeably herein and refer to a recombinant plasmid or virus that comprises a nucleic acid to be delivered into a host cell, either in vitro or in vivo. The vector can be a nucleic acid molecule capable of propagating another nucleic acid to which it is linked, and include the term “expression vectors.” Vectors also include any pharmaceutical compositions thereof (e.g., a recombinant vector and a pharmaceutically acceptable carrier/excipient as provide herein). The term vector includes the vector as a self-replicating nucleic acid structure as well as the vector incorporated into the genome of a host cell into which it has been introduced. Vectors, including expression vectors, comprise the nucleotide sequence encoding the CRB1-B isoform described herein and a heterogeneous sequence necessary for proper propagation of the vector and expression of the encoded polypeptide. The heterogeneous sequence (i.e., sequence from a difference species than the polypeptide) can comprise a heterologous promoter or heterologous transcriptional regulatory region that allows for expression of the polypeptide. As used herein, the terms “heterologous promoter,” “promoter,” “promoter region,” or “promoter sequence” refer generally to transcriptional regulatory regions of a gene, which may be found at the 5′ or 3′ side of the polynucleotides described herein, or within the coding region of the polynucleotides, or within introns in the polynucleotides. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site, as well as protein binding domains (consensus sequences) responsible for the binding of RNA polymerase. Any promoter capable of expressing CRB1-B in a retinal cell are contempated to be used in the practice of the present invention.

In some embodiments, the recombinant vector comprises a polynucleotide encoding a Crumbs 1-B (CRB1-B) isoform, wherein the CRB1-B isoform comprises an N-terminal signal peptide linked to an extracellular polypeptide comprising or consisting of, from N-terminus-to-C-terminus: two EGF domains, a lamG domain, an EGF domain, a lamG domain, an EGF domain, a lamG domain, and four EGF domains (see FIG. 16 for the CRB1-B protein sequence with these domains annotated); wherein the C terminus of the extracellular polypeptide is linked to a C-terminal domain comprising a transmembrane domain and intracellular domain. In a preferred embodiment, the polynucleotide is operably linked to a heterologous promoter capable of expressing the isoform in a retinal cell. In some embodiments, the extracellular polypeptide extends from the N-terminus of the ninth EGF domain of a CRB1-A isoform to the C-terminus of the sixteenth EGF domain of the CRB1-A isoform. In some embodiments, the C-terminal domain comprises the amino acid sequence of VSSLSFYVSLLFWQNLFQLLSYLILRMNDEPVVEWGEQEDY (SEQ ID NO: 3).

As used herein, the term “EGF domain” (also referred to as an “EGF-like domain”) is an evolutionary conserved protein domain, which derives its name from the epidermal growth factor where it was first described. Most occurrences of the EGF-like domain are found in the extracellular domain of membrane-bound proteins or in proteins known to be secreted. The main structure of EGF-like domains is a two-stranded B-sheet followed by a loop to a short C-terminal, two-stranded β-sheet. EGF-like domains frequently occur in numerous tandem copies within proteins, which typically fold together to form a single, linear solenoid domain block. Suitable EGF domains include, without limitation, SEQ ID NO: 14-20 and SEQ ID NO:52 which are the EGF domains found within the human CRB1-B isoform.

As used herein, the terms “laminin globular (G) domain” and “lamG domain” are used interchangeably to refer to a domain found in various members of the laminin protein family as well as in a large number of other extracellular proteins. Suitable lamG domains include, without limitation, SEQ ID NO:21-23, which are the lamG domains found within the human CRB1-B isoform.

The term “N-terminal signal peptide” (also commonly referred to as a “signal peptide”, “signal sequence”, or “leader peptide”) refers to a short peptide present at the N-terminus of a protein that directs the cellular localization of a protein by targeting it within the cell's secretory pathway. The term “extracellular polypeptide” refers to a polypeptide or portion thereof that localizes outside of the cell in the extracellular space (i.e., outside of the plasma membrane).

Accordingly, another aspect of the present disclosure provides a recombinant vector comprising, consisting of, or consisting essentially of a polynucleotide comprising a Crb1 isoform selected from the group consisting of Crb1-A, Crb1-A2, Crb1-B, Crb1-C and combinations thereof. In one embodiment, the Crb1 isoform comprises Crb1-A. In another embodiment, the Crb1 isoform comprises Crb1-A2. In another embodiment, the Crb1 isoform comprises Crb1-B. In yet another embodiment, the Crb1 isoform comprises Crb1-C.

In some embodiments, the vector comprises a viral vector. The term viral vector as used herein also include the virus particles containing the viral vector produced by expression of viral vectors within a cell (e.g. a cell line), wherein the cell produces the viral vector containing viral particles (i.e. virions). The virus particles comprise a viral DNA or RNA that encodes and is capable of expression of the isoform of interest in a cell to which it is introduced. Thus, the term “viral vector” includes the mature viral particles containing the viral vector that are capable of expressing the isoform of interest in a host cell, preferably a retinal cell. Introduction or transduction of the viral vector into a host cell, preferably a retinal cell, allows for the expression of the encoded CRB1-B isoform within the host cell. Methods of packaging the viral vector into virions (i.e. particles) are known in the art. In a preferred embodiment, the viral vector is an adeno-associated virus (AAV). It is understood that other gene delivery vectors, including retroviruses, lentiviruses, HSV vectors, or Semliki-Forrest-Virus vectors and adenoviruses may also be used and are contemplated to be part of the present invention. The advantage of AAV vectors is that they can generally be concentrated to titers of about 10¹⁴ viral particles per ml, a level of vector that has the potential to transduce a greater number of target cells, e.g., retinal cells, in a patient. Moreover, AAV-based vectors have a well-established record of safety and do not integrate at significant levels into the target cell genome, thus avoiding the potential for insertional activation of deleterious genes or deactivation of necessary genes. Accordingly, in certain embodiments the viral vector comprises an AAV vector.

In some embodiments, the polynucleotide is under the control of a promoter sequence that is expressed in the retina. In other embodiments, the polynucleotide is operably linked to a promoter suitable for expression of the polynucleotide in one or more retina cell types. In some embodiments, the retina cell is selected from the group consisting of a photoreceptor cells, a retinal pigmented epithelial cell, a bipolar cell, a horizontal cell, an amacrine cell, a Müller cell, and/or a ganglion cell. In certain embodiments, the retinal cell comprises a photoreceptor cell. In some embodiments, the promoter is selected from the group consisting of a rhodopsin kinase (RK) promoter, an opsin promoter, a Cytomegalovirus (CMV) promoter, and a chicken β-actin (CBA promoter), among others.

For example, in one embodiment, the target cell of the isolated polynucleotide or recombinant vector encoding CRB1-B is a photoreceptor cell in the retina. In another example, the isolated polynucleotide or recombinant vector encodes CRB1-A and the target cell is a Mueller cell. In some embodiments, one or more vectors may be used in combination, wherein one vector encodes the CRB1-B isoform, and the one or more other vectors encodes one of the other Crb isoforms, for example, CRB1-A, CRB1-A2, or CRB-C.

A “recombinant viral vector” refers to a recombinant polynucleotide vector comprising one or more heterologous sequences (i.e., nucleic acid sequence not of viral origin). In the case of recombinant AAV vectors, the recombinant nucleic acid is flanked by at least one inverted terminal repeat sequence (ITR). In some embodiments, the recombinant nucleic acid is flanked by two ITRs.

A “recombinant AAV vector (rAAV vector)” refers to a polynucleotide vector comprising one or more heterologous sequences (i.e., nucleic acid sequence not of AAV origin) that are flanked by at least one AAV inverted terminal repeat sequence (ITR). Such rAAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins). When a rAAV vector is incorporated into a larger polynucleotide (e.g., in a chromosome or in another vector such as a plasmid used for cloning or transfection), then the rAAV vector may be referred to as a “pro-vector” which can be “rescued” by replication and encapsidation in the presence of AAV packaging functions and suitable helper functions. A rAAV vector can be in any of a number of forms, including, but not limited to, plasmids, linear artificial chromosomes, complexed with lipids, encapsulated within liposomes, and encapsidated in a viral particle, e.g., an AAV particle. A rAAV vector can be packaged into an AAV virus capsid to generate a “recombinant adeno-associated viral particle (rAAV particle)”. Methods and kits for making AAV are known in the art, for example, but not limited to, AdEasy® cloning system (e.g., available from QBiogene GmbH, Heidelberg, Germany). Corresponding vectors and helper vectors are extensively known in the art (Nicklin S A, Baker A H, Curr Gene Ther., 2002, 2: 273-93; Mah et al., Clin Pharmacokinet., 2002, 41: 901-11).

An “rAAV virus” or “rAAV viral particle” refers to a viral particle composed of at least one AAV capsid protein and an encapsidated rAAV vector genome.

In some embodiments, the vector comprises a recombinant AAV (rAAV) vector. In some embodiments, the vector comprises a transgene flanked by one or two AAV inverted terminal repeats (ITRs). The nucleic acid is encapsidated in the AAV particle. The AAV vector may also comprise capsid proteins. In some embodiments, the nucleic acid comprises the coding sequence(s) of interest (e.g., Crb1-A, Crb1-A2, Crb1-B, Crb1-C, preferably Crb1-B) operatively linked components in the direction of transcription, control sequences including transcription initiation and termination sequences, thereby forming an expression cassette.

In some embodiments, the expression cassette is flanked on the 5′ and 3′ end by at least one functional AAV ITR sequences. By “functional AAV ITR sequences” it is meant that the ITR sequences function as intended for the rescue, replication and packaging of the AAV virion. See Davidson et al., PNAS, 2000, 97 (7) 3428-32; Passini et al., J. Virol., 2003, 77 (12): 7034-40; and Pechan i., Gene Ther., 2009, 16:10-16, all of which are incorporated herein in their entirety by reference. For practicing some aspects of the present disclosure, the recombinant vectors comprise at least all of the sequences of AAV essential for encapsidation and the physical structures for infection by the rAAV. AAV ITRs for use in the vectors of the present disclosure need not have a wild-type nucleotide sequence (e.g., as described in Kotin, Hum. Gene Ther., 1994, 5:793-801), and may be altered by the insertion, deletion or substitution of nucleotides or the AAV ITRs may be derived from any of several AAV serotypes. More than 40 serotypes of AAV are currently known, and new serotypes and variants of existing serotypes continue to be identified. See Gao et al., PNAS, 2002, 99(18): 11854-6; Gao et al., PNAS, 2003, 100(10):6081-6; and Bossis et al., J. Virol., 2003, 77(12):6799-810.

Use of any AAV serotype is considered within the scope of the present disclosure. In some embodiments, a rAAV vector is a vector derived from an AAV serotype, including without limitation, AAV ITRs are AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV DJ, a goat AAV, bovine AAV, or mouse AAV ITRs or the like. In some embodiments, the nucleic acid in the AAV comprises an ITR of AAV ITRs are AAV1, AAV2, AAV3, AAV4, AAV5, AAV6, AAV7, AAV8, AAVrh8, AAVrh8R, AAV9 (Aschauer et al., 2013), AAV10, AAVrh10, AAV11, AAV12,AAV2R471A, AAV DJ, a goat AAV, bovine AAV, or mouse AAV or the like. In certain embodiments, the nucleic acid in the AAV comprises an AAV2 ITR. In some embodiments, a vector may include a stuffer nucleic acid. In some embodiments, the stuffer nucleic acid may encode a green fluorescent protein. In some embodiments, the stuffer nucleic acid may be located between the promoter and the nucleic acid encoding the CRB1-B isoform.

Numerous methods are known in the art for production of viral vectors, including rAAV vectors, including transfection, stable cell line production, and infectious hybrid virus production systems. Some of those systems include, but are not limited to, for example, adenovirus-AAV hybrids, herpesvirus-AAV hybrids (Conway, J E et al., (1997) J. Virology 71(11): 8780-8789) and baculovirus-AAV hybrids. rAAV production cultures for the production of rAAV virus particles all require; 1) suitable host cells, including, for example, human-derived cell lines such as HeLa, A549, or 293 cells, or insect-derived cell lines such as SF-9, in the case of baculovirus production systems; 2) suitable helper virus function, provided by wild-type or mutant adenovirus (such as temperature sensitive adenovirus), herpes virus, baculovirus, or a plasmid construct providing helper functions; 3) AAV rep and cap genes and gene products; 4) a transgene (such as a therapeutic transgene) flanked by at least one AAV ITR sequences; and 5) suitable media and media components to support rAAV production. Suitable media known in the art may be used for the production of rAAV vectors. These media include, without limitation, media produced by Hyclone Laboratories and JRH including Modified Eagle Medium (MEM), Dulbecco's Modified Eagle Medium (DMEM), custom formulations such as those described in U.S. Pat. No. 6,566,118, and Sf-900 II SFM media as described in U.S. Pat. No. 6,723,551, each of which is incorporated herein by reference in its entirety, particularly with respect to custom media formulations for use in production of recombinant AAV vectors.

The vectors according to the present disclosure can be produced using methods known in the art. See, e.g., U.S. Pat. Nos. 6,566,118; 6,989,264; and 6,995,006. In practicing the invention, host cells for producing rAAV particles include mammalian cells, insect cells, plant cells, microorganisms and yeast. Host cells can also be packaging cells in which the AAV rep and cap genes are stably maintained in the host cell or producer cells in which the AAV vector genome is stably maintained. Exemplary packaging and producer cells are derived from 293, A549 or HeLa cells. AAV vectors are purified and formulated using standard techniques known in the art.

In some embodiments, vectors according to the present disclosure may be produced by a triple transfection method, such as the exemplary triple transfection method provided infra. Briefly, a plasmid containing a rep gene and a capsid gene, along with a helper adenoviral plasmid, may be transfected (e.g., using the calcium phosphate method) into a cell line (e.g., HEK-293 cells), and virus may be collected and optionally purified.

In some embodiments, the vectors may be produced by a producer cell line method, such as the exemplary producer cell line method provided infra (see also (referenced in Martin et al., (2013)) Human Gene Therapy Methods 24:253-269). Briefly, a cell line (e.g., a HeLa cell line) may be stably transfected with a plasmid containing a rep gene, a capsid gene, and a promoter-transgene sequence. Cell lines may be screened to select a lead clone for vector production, which may then be expanded to a production bioreactor and infected with an adenovirus (e.g., a wild-type adenovirus) as helper to initiate vector production. Virus may subsequently be harvested, adenovirus may be inactivated (e.g., by heat) and/or removed, and the vectors may be purified.

The terms “genome particles (gp),” “genome equivalents,” or “genome copies” as used in reference to a viral titer, refer to the number of virions containing the recombinant AAV DNA genome, regardless of infectivity or functionality. The number of genome particles in a particular vector preparation can be measured by procedures such as described in, for example, Clark et al. (1999) Hum. Gene Ther., 10:1031-1039; Veldwijk et al. (2002) Mol. Ther., 6:272-278. The term “vector genome (vg)” as used herein may refer to one or more polynucleotides comprising a set of the polynucleotide sequences of a vector, e.g., a viral vector. A vector genome may be encapsidated in a viral particle. Depending on the particular viral vector, a vector genome may comprise single-stranded DNA, double-stranded DNA, or single-stranded RNA, or double-stranded RNA. A vector genome may include endogenous sequences associated with a particular viral vector and/or any heterologous sequences inserted into a particular viral vector through recombinant techniques. For example, a recombinant AAV vector genome may include at least one ITR sequence flanking a promoter, a stuffer, a sequence of interest (e.g., an RNAi), and a polyadenylation sequence. A complete vector genome may include a complete set of the polynucleotide sequences of a vector. In some embodiments, the nucleic acid titer of a viral vector may be measured in terms of vg/mL. In another embodiment, for example in the use of AAV vectors, the viral titer may be measured in terms of DNase resistant particles (DRP) as mature, enveloped AAV particles are counted from not fully formed AAV particles. Methods suitable for measuring this titer are known in the art (e.g., quantitative PCR).

### Promoters

In some embodiments, the nucleic acids (polynucleotides) of the present disclosure (e.g., Crb1 isoform B, and in other embodiments, Crb1 isoforms A, A2 and/or C) are operably linked to a promoter. The promoter can be a constitutive, inducible, or repressible promoter. Preferably, the promoter is capable of expression of the isoform encoded in the polynucleotide in the target cell. Exemplary promoters include, but are not limited to, the cytomegalovirus (CMV) immediate early promoter, the RSV LTR, the MoMLV LTR, the phosphoglycerate kinase-1 (PGK) promoter, a simian virus 40 (SV40) promoter and a CK6 promoter, a transthyretin promoter (TTR), a TK promoter, a tetracycline responsive promoter (TRE), an HBV promoter, an hAAT promoter, a LSP promoter, chimeric liver-specific promoters (LSPs), the E2F promoter, the telomerase (hTERT) promoter; the cytomegalovirus enhancer/chicken β-actin/Rabbit β-globin promoter (CAG promoter; Niwa et al., Gene, 1991, 108(2):193-9) and the elongation factor 1-α promoter (EF1-α) promoter (Kim et al., Gene, 1990, 91(2):217-23 and Guo et al., Gene Ther., 1996, 3(9):802-10).

As used herein, a promoter is “operably connected to” or “operably linked to” when it is placed into a functional relationship with a second polynucleotide sequence. For instance, a promoter is operably connected to a polynucleotide if the promoter is connected to the polynucleotide such that it may effect transcription of the polynucleotide coding sequence. In various embodiments, the polynucleotides may be operably linked to at least 1, at least 2, at least 3, at least 4, at least 5, or at least 10 promoters.

Advantageously, the promoter is a tissue-specific promoter that drives gene expression in retinal cells. Numerous retinal-specific promoters are known in the art. For example, the rhodopsin kinase (RK) promoter (SEQ ID NO:24), which is derived from the human rhodopsin kinase gene (GenBank Entrez Gene ID 6011), has been shown to drive expression specifically in rod and cone photoreceptor cells, as well as retinal cell lines such as WERI Rb-1 (Khani, S. C., et al. (2007) Invest. Ophthalmol. Vis. Sci. 48(9):3954-61). As used herein, “rhodopsin kinase promoter” may refer to an entire promoter sequence or a fragment of the promoter sequence sufficient to drive photoreceptor-specific expression, such as the sequences described in Khani, S. C., et al. (2007) Invest. Ophthalmol. Vis. Sci. 48(9):3954-61 and Young, J. E., et al. (2003) Invest. Ophthalmol. Vis. Sci. 44(9):4076-85. In some embodiments, the RK promoter spans from −112 to +180 relative to the transcription start site.

Opsin promoters and derivatives thereof are also commonly used to drive retinal-specific gene expression. For example, a minimal promoter has been derived from the mouse opsin gene (SEQ ID NO:25) has been shown to drive robust expression in photoreceptors (Pawlyk, B. S., et al. (2005) Invest Ophthalmol Vis Sci, 46 (9), 3039-45). Thus, in some embodiments, the promoter is a rhodopsin kinase (RK) promoter or an opsin promoter.

Alternatively, the promoter may be a constitutive promoter that is not tissue-specific. Use of such promoters may be advantageous when a high-level of gene expression is desirable. For example, the cytomegalovirus (CMV) promoter (SEQ ID NO:26) is commonly included in vectors used to genetically engineering mammalian cells, as it is well-characterized as a strong constitutive promoter (Boshart et al., Cell, 41:521-530 (1985)). Another example of a commonly used constitutive promoter is the chicken β-actin promoter (SEQ ID NO:27), which is also known as the “CAG promoter” (see Definitions; Miyazaki, J., et al. (1989) Gene 79(2):269-77)). The CAG promoter is a strong synthetic promoter that was formed by combining the cytomegalovirus (CMV) early enhancer element, the promoter, first exon and the first intron of chicken beta-actin gene, and the splice acceptor of the rabbit beta-globin gene. Thus, in some embodiments, the promoter is a cytomegalovirus (CMV) promoter or a chicken β-actin (CAG promoter). 69502412As used herein, the term “CAG promoter” may be used interchangeably with “CBA promoter.”

In some embodiments, the promoter comprises a human β-glucuronidase promoter or a cytomegalovirus enhancer linked to a chicken β-actin (CBA) promoter. In some embodiments, the invention provides a recombinant vector comprising nucleic acid encoding a heterologous transgene of the present disclosure operably linked to a CBA promoter. Exemplary promoters and descriptions may be found, e.g., in U.S. PG Pub. 20140335054.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the SV40 promoter, the dihydrofolate reductase promoter, the 13-actin promoter, the phosphoglycerol kinase (PGK) promoter, and the EF1α promoter [Invitrogen].

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

Suitable promoters for use in AAV vectors capable of expression in retinal cells are known in the art, for example, as found in “Targeting neuronal and glial cell types with synthetic promoter AAVs in mice, non-human primates, and humans” see, Table S1 in Jüttner et. al, bioRxiv 434720; doi: doi.org/10.1101/434720 (October 2018), Now published in Nature Neuroscience doi: 10.1038/s41593-019-0431-2, incorporated by reference in its entirety.

In another embodiment, the native promoter, or fragment thereof, for the transgene will be used. The native promoter can be used when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some aspects, the present disclosure provides an isolated polypeptide comprising or consisting of the CRB1-B isoform (e.g., SEQ ID NO:1). Suitably, the isolated polypeptide may be expressed from the polynucleotide or vector described herein. The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like.

Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

The term “substantial identity” of polynucleotide sequences means that a polynucleotide comprises a sequence that has at least 95% sequence identity to the polynucleotide encoding the polypeptide of interest described herein. Alternatively, percent identity can be any integer from 95% to 100%. In one embodiment, the sequence identity is at least 95%, alternatively at least 99%. More preferred embodiments include at least: 96%, 97%, 98%, 99% or 100% compared to a reference sequence using the programs described herein; preferably BLAST using standard parameters, as described. These values can be appropriately adjusted to determine corresponding identity of proteins encoded by two nucleotide sequences by taking into account codon degeneracy, amino acid similarity, reading frame positioning and the like.

In some preferred embodiments, the term “substantial identity” of amino acid sequences for purposes of this invention means polypeptide sequence identity of at least 95%, preferably 98%, most preferably 99% or 100%. Preferred percent identity of polypeptides can be any integer from 95% to 100%. More preferred embodiments include at least 96%, 97%, 98%, 99%, or 100%.

### iii. Pharmaceutical Compositions

The present disclosure further provides a pharmaceutical composition. The pharmaceutical composition may comprise or consists of the isolated polynucleotide encoding the CRB1 isoform, the recombinant vector encoding the CRB1 isoform, preferably the CRB1-B isoform described herein and a pharmaceutically acceptable carrier. In one example, the pharmaceutical composition may comprise viral vectors encoding the CRB1-B isoform. The pharmaceutical composition can comprise viral vectors, for example rAAV viral vectors, at a concentration of about 1×10⁶ DNase-resistant particles (DRP)/ml to about 1×10¹⁴ DRP/ml. The pharmaceutical composition of claim 14 or 15, further comprising a second vector encoding CRB1-A, CRB1-A2, CRB1-C, or combinations thereof.

The vectors according to the present disclosure may further be in the form of a pharmaceutical composition. Accordingly, in some embodiments, the vectors provided herein may further contain buffers and/or pharmaceutically acceptable excipients and/or pharmaceutically acceptable carriers. As is well known in the art, pharmaceutically acceptable excipients and/or carriers are relatively inert substances that facilitate administration of a pharmacologically effective substance and can be supplied as liquid solutions or suspensions, as emulsions, or as solid forms suitable for dissolution or suspension in liquid prior to use. The pharmaceutically acceptable carrier may be selected based upon the route of administration desired. For example, an excipient can give form or consistency, or act as a diluent. Suitable excipients include but are not limited to stabilizing agents, wetting and emulsifying agents, salts for varying osmolarity, encapsulating agents, pH buffering substances, and buffers. Such excipients include any pharmaceutical agent suitable for direct delivery to the eye which may be administered without undue toxicity. Suitably the pharmaceutically acceptable carrier helps maintain the viral particle integrity of the viral vector prior to administration, e.g., provide a suitable pH balanced solution. Pharmaceutically acceptable excipients include, but are not limited to, sorbitol, any of the various TWEEN® (surfactant/detergent) compounds, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in REMINGTON'S PHARMACEUTICAL SCIENCES (Mack Pub. Co., N.J. 1991). The compositions can be sterilized by conventional, well known sterilization techniques prior to administration (e.g., filtration, addition of sterilizing agent, etc.). The compositions may contain pharmaceutically acceptable additional substances as required to approximate physiological conditions such as a pH adjusting and buffering agent, toxicity adjusting agents, such as, sodium acetate, sodium chloride, potassium chloride, calcium chloride, sodium lactate, and the like.

In some embodiments related to ocular delivery, pharmaceutically acceptable carriers include, for example, sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. Additional ingredients may also be used, for example preservatives, buffers, tonicity agents, antioxidants and stabilizers, nonionic wetting or clarifying agents, viscosity-increasing agents, and the like.

In some embodiments, the pharmaceutical compositions of the present disclosure are formulated for administration by subretinal injection. Accordingly, these compositions can be combined with pharmaceutically acceptable vehicles such as saline, Ringer's balanced salt solution (pH 7.4), and the like. Although not required, the compositions may optionally be supplied in unit dosage form suitable for administration of a precise amount.

In other embodiments, the pharmaceutical compositions of the present disclosure are formulated for topical administration to the eye. In such embodiments, conventional intraocular delivery reagents can be used. For example, pharmaceutical compositions of the present disclosure for topical intraocular delivery can comprise saline solutions as described above, corneal penetration enhancers, insoluble particles, petrolatum or other gel-based ointments, polymers which undergo a viscosity increase upon instillation in the eye, or mucoadhesive polymers. Preferably, the intraocular delivery reagent increases corneal penetration, or prolongs preocular retention of the siRNA through viscosity effects or by establishing physicochemical interactions with the mucin layer covering the corneal epithelium.

### Methods of Treatment

The present disclosure further provides methods of treating and/or preventing ocular disorders in a subject using the polynucleotides, vectors and pharmaceutical compositions according to the present disclosure. In some embodiments, the method of treating is a gene therapy protocol for such ocular disorders and requires the localized delivery of the polynucleotide or vectors according to the present disclosure to the cells in the retina. The cells that will be the treatment target in such embodiments are either the photoreceptor cells in the retina or the cells of the RPE underlying the neurosensory retina. Hence, in one embodiment, the delivery of the polynucleotides and vectors according to the present disclosure are achieved by injection into the subretinal space between the retina and the RPE. Accordingly, one aspect of the present disclosure provides a method of treating and/or preventing an ocular disorder in a subject, the method comprising, consisting of, or consisting essentially of administering the subject a therapeutically effective amount of a polynucleotide, recombinant vector or pharmaceutical composition according to the present disclosure such that the ocular disorder is treated in the subject. Preferably, the polynucleotide or recombinant vector or pharmaceutical composition comprising the same encodes the CRB1-B isoform described herein.

The present disclosure provides a method of reducing progression of loss of vision or maintaining vision function in a subject in need thereof. The method comprises administering the subject a therapeutically effective amount of the isolated polynucleotide, the recombinant vector, the isolated polypeptide, or pharmaceutical composition described herein such that progression of loss of vision or is reduced. In some embodiments, the loss of vision is maintained at a level similar to the vision level when treatment was started, for example, vision is maintained within about 10% of the vision at the start of treatment. Not to be bound by any theory, but maintainance of the level of CRB1-B expression in trans in photoreceptor cells within the retina of subject in need of treatment may allow for the reduction in the death of photoreceptor cells and the maintenance of the photoreceptor-glial junctions, maintain the vision in the subject. In some embodiments, the isolated polynucleotide, recombinant vector or pharmaceutical composition is administered intravitreally, subretinally, or topically.

In some embodiments, the method described herein can further comprise monitoring the visual function of the subject, wherein the vision function in the subject is maintained and not reduced after administration. Methods of monitoring visual function are known in the art (described further below) and include, for example, monitoring visual acuity of the subject.

In some examples, the function for this isoform at photoreceptor-glial junctions is maintained after treatment with the isolated polynucleotide, the recombinant vector, the isolated polypeptide, or pharmaceutical composition described herein. The term “administering” encompasses methods of delivering the isolated polypeptide, vector or pharmaceutical composition to one or more cells within the retina of the subject. In a preferred embodiment, the isoform is CRB1-B and the one or more cells are photoreceptor cells within the retina. Suitable techniques for delivering the isolated polynucleotide, vector or pharmaceutical composition according to the present disclosure to a subject may include numerous methods known in the art, such as by gene gun, electroporation, nanoparticles, transduction by viral particles, micro-encapsulation, gene editing, and the like, or by parenteral and enteral administration routes. Suitable parenteral administration routes include, for example, peri-and intra-tissue administration (e.g., intra-retinal injection or subretinal injection); direct (e.g., topical) application to the area at or near the site of neovascularization, for example by a catheter or other placement device (e.g., a corneal pellet or a suppository, eyedropper, or an implant comprising a porous, non-porous, or gelatinous material). Suitable placement devices include the ocular implants described in U.S. Pat. Nos. 5,902,598 and 6,375,972, and the biodegradable ocular implants described in U.S. Pat. No. 6,331,313, the entire disclosures of which are herein incorporated by reference. Such ocular implants are available from Control Delivery Systems, Inc. (Watertown, Mass.) and Oculex Pharmaceuticals, Inc. (Sunnyvale, Calif.). In certain embodiments, the parenteral administration route comprises intraocular administration. It is understood that intraocular administration of the isolated polynucleotides, vectors and pharmaceutical compositions according to the present disclosure can be accomplished by injection or direct (e.g., topical) administration to the eye, as long as the administration route allows the isolated polynucleotides, vectors or pharmaceutical compositions to enter the eye. In addition to the topical routes of administration to the eye described above, suitable intraocular routes of administration include intravitreal, intraretinal, subretinal, subtenon, peri-and retro-orbital, trans-corneal and trans-scleral administration. Such intraocular administration routes are within the skill in the art; see, e.g., and Acheampong A A et al, 2002, supra; and Bennett et al. (1996), Hum. Gene Ther. 7: 1763-1769 and Ambati J et al., 2002, Progress in Retinal and Eye Res. 21: 145-151, the entire disclosures of which are herein incorporated by reference.

As used herein, the term “topically” means application to the surface of the eye.

In some embodiments, the isolated polynucleotides, vectors or pharmaceutical compositions according to the present disclosure are administered to a subject via subretinal delivery. Methods of subretinal delivery are known in the art. For example, see WO 2009/105690, incorporated herein by reference. Briefly, the general method for delivering a vector according to the present disclosure to the subretina of the macula and fovea may be illustrated by the following brief outline. This example is merely meant to illustrate certain features of the method, and is in no way meant to be limiting.

Generally, they can be delivered in the form of a composition injected intraocularly (subretinally) under direct observation using an operating microscope. This procedure may involve vitrectomy followed by injection of the vector suspension using a fine cannula through one or more small retinotomies into the subretinal space.

Briefly, an infusion cannula can be sutured in place to maintain a normal globe volume by infusion (of e.g., saline) throughout the operation. A vitrectomy is performed using a cannula of appropriate bore size (for example 20 to 27 gauge), wherein the volume of vitreous gel that is removed is replaced by infusion of saline or other isotonic solution from the infusion cannula. The vitrectomy is advantageously performed because (1) the removal of its cortex (the posterior hyaloid membrane) facilitates penetration of the retina by the cannula; (2) its removal and replacement with fluid (e.g., saline) creates space to accommodate the intraocular injection of vector, and (3) its controlled removal reduces the possibility of retinal tears and unplanned retinal detachment.

In some embodiments, the isolated polynucleotide, vector or pharmaceutical composition according to the present disclosure is directly injected into the subretinal space outside the central retina, by utilizing a cannula of the appropriate bore size (e.g., 27-45 gauge), thus creating a bleb in the subretinal space. In other embodiments, the subretinal injection of the isolated polynucleotide, vector or pharmaceutical composition according to the present disclosure is preceded by subretinal injection of a small volume (e.g., about 0.1 to about 0.5 ml) of an appropriate fluid (such as saline or Ringer's solution) into the subretinal space outside the central retina. This initial injection into the subretinal space establishes an initial fluid bleb within the subretinal space, causing localized retinal detachment at the location of the initial bleb. This initial fluid bleb can facilitate targeted delivery of the isolated polynucleotide, vector and/or pharmaceutical composition to the subretinal space (by defining the plane of injection prior to vector and/or pharmaceutical composition delivery), and minimize possible isolated polynucleotide, vector and/or pharmaceutical composition administration into the choroid and the possibility of isolated polynucleotide, vector and/or pharmaceutical composition injection or reflux into the vitreous cavity. In some embodiments, this initial fluid bleb can be further injected with fluids comprising one or more isolated polynucleotidevector and/or pharmaceutical compositions and/or one or more additional therapeutic agents by administration of these fluids directly to the initial fluid bleb with either the same or additional fine bore cannulas.

Intraocular administration of the isolated polynucleotide, vector or pharmaceutical composition according to the present disclosure and/or the initial small volume of fluid can be performed using a fine bore cannula (e.g., 27-45 gauge) attached to a syringe. In some embodiments, the plunger of this syringe may be driven by a mechanized device, such as by depression of a foot pedal. The fine bore cannula is advanced through the sclerotomy, across the vitreous cavity and into the retina at a site pre-determined in each subject according to the area of retina to be targeted (but outside the central retina). Under direct visualization the isolated polynucleotide, vector or pharmaceutical composition suspension is injected mechanically under the neurosensory retina causing a localized retinal detachment with a self-sealing non-expanding retinotomy. As noted above, the isolated polynucleotide, vector or pharmaceutical composition can be either directly injected into the subretinal space creating a bleb outside the central retina or the isolated polynucleotide, vector or pharmaceutical composition can be injected into an initial bleb outside the central retina, causing it to expand (and expanding the area of retinal detachment). In some embodiments, the injection of the isolated polynucleotide, vector or pharmaceutical composition according to the present disclosure is followed by injection of another fluid into the bleb.

Without wishing to be bound by theory, the rate and location of the subretinal injection(s) can result in localized shear forces that can damage the macula, fovea and/or underlying RPE cells. The subretinal injections may be performed at a rate that minimizes or avoids shear forces. In some embodiments, the isolated polynucleotide, vector or pharmaceutical composition according to the present disclosure is injected over about 15-17 minutes. In some embodiments, the vector is injected over about 17-20 minutes. In some embodiments, the isolated polynucleotide, vector and/or pharmaceutical composition is injected over about 20-22 minutes. In some embodiments, the isolated polynucleotidevector and/or pharmaceutical composition is injected at a rate of about 35 to about 65 μl/min. In some embodiments, the isolated polynucleotide, vector and/or pharmaceutical composition is injected at a rate of about 35 μl/min. In some embodiments, the isolated polynucleotide, vector and/or pharmaceutical composition is injected at a rate of about 40 μl/min. In some embodiments, the isolated polynucleotide, vector and/or pharmaceutical composition is injected at a rate of about 45 μl/min. In some embodiments, the isolated polynucleotide, vector and/or pharmaceutical composition is injected at a rate of about 500 min. In some embodiments, the isolated polynucleotide, vector and/or pharmaceutical composition is injected at a rate of about 55 μl/min. In some embodiments, the isolated polynucleotide, vector and/or pharmaceutical composition is injected at a rate of about 60 μl/min. In some embodiments, the isolated polynucleotide, vector and/or pharmaceutical composition is injected at a rate of about 65 μl/min. One of ordinary skill in the art would recognize that the rate and time of injection of the bleb may be directed by, for example, the volume of the vector and/or pharmaceutical composition or size of the bleb necessary to create sufficient retinal detachment to access the cells of central retina, the size of the cannula used to deliver the isolated polynucleotide, vector and/or pharmaceutical composition, and the ability to safely maintain the position of the cannula of the invention.

In some embodiments of the present disclosure, the volume of the isolated polynucleotide or vector (in solution or in a pharmaceutical composition as provided herein) injected to the subretinal space of the retina is more than about any one of 1 μl, 2 μl, 3 μl, 4 μl, 5 μl, 6 μl, 7 μl, 8 μl, 9 μl, 10 μl, 15 μl, 20 μl, 25 μl, 50 μl, 75 μl, 100 μl, 200 μl, 300 μl, 400 μl, 500 μl, 600 μl, 700 μl, 800 μl, 900 μl, or 1 mL, or any amount therebetween.

In some embodiments, the methods comprise administration to the eye (e.g., by subretinal and/or intravitreal administration) an effective amount of an isolated polynucleotide, vector or pharmaceutical composition according to the present disclosure. In some embodiments, a viral vector is used in a pharmaceutical composition, and the viral titer of the composition is at least about any of 5×10¹², 6×10¹², 7×10^{12,}8×10¹², 9×10¹², 10×10¹², 11×10¹², 15×10¹², 20×10¹², 25×10¹², 30×10¹², or 50×10¹² genome copies/mL. In some embodiments, the viral titer of the composition is about any of 5×10¹² to 6×10¹², 6×10¹² to 7×10¹², 7×10¹² to 8×10¹², 8×10¹² to 9×10¹², 9×10¹² to 10×10¹², 10×10¹² to 11×10¹², 11×10¹² to 15×10¹², 15×10¹² to 20 ×10¹², 20×10¹² to 25×10¹², 25×10¹² to 30×10¹², 30×10¹² to 50×10¹², or 50×10¹² to 100×10¹² genome copies/mL In some embodiments, the viral titer of the composition is about any of 5×10¹² to 10×10¹², 10×10¹² to 25×10^{12,} or 25×10¹² to 50×10¹² genome copies/mL In some embodiments, the viral titer of the composition is at least about any of 5×10⁹, 6×10⁹, 7×10⁹, 8×10⁹, 9×10⁹, 10×10⁹, 11×10⁹, 15×10⁹, 20×10⁹, 25×10⁹, 30×10⁹, or 50×10⁹ transducing units/mL. In some embodiments, the viral titer of the composition is about any of 5×10⁹ to 6×10⁹, 6×10⁹ to 7×10⁹, 7×10⁹ to 8×10⁹, 8×10⁹ to 9×10⁹, 9×10⁹ to 10×10⁹, 10×10⁹ to 11×10⁹, 11×10⁹ to 15×10⁹, 15×10⁹ to 20×10⁹, 20×10⁹ to 25×10⁹, 25×10⁹ to 30×10⁹, 30×10⁹ to 50×10⁹ or 50×10⁹ to 100×10⁹ transducing units/mL. In some embodiments, the viral titer of the composition is about any of 5×10⁹ to 10×10⁹, 10×10⁹ to 15×10⁹, 15×10⁹ to 25×10⁹, or 25×10⁹ to 50×10⁹ transducing units/mL In some embodiments, the viral titer of the composition is at least any of about 5×10¹⁰, 6×10¹⁰, 7×10¹⁰, 8×10¹⁰, 9×10¹⁰, 10×10¹⁰, 11×10¹⁰, 15×10¹⁰, 20×10¹⁰, 25×10¹⁰, 30×10¹⁰, 40×10¹⁰, or 50×10¹⁰ infectious units/mL In some embodiments, the viral titer of the composition is at least any of about 5×10¹⁰ to 6×10¹⁰, 6×10¹⁰ to 7×10¹⁰, 7×10¹⁰ to 8×10¹⁰, 8×10¹⁰ to 9×10¹⁰, 9×10¹⁰ to 10×10¹⁰, 10×10¹⁰ to 11×10¹⁰, 11×10¹⁰ to 15×10¹⁰, 15×10¹⁰ to 20×10¹⁰, 20×10¹⁰ to 25×10¹⁰, 25×10¹⁰ to 30×10¹⁰, 30×10¹⁰ to 40×10¹⁰, 40×10¹⁰ to 50×10¹⁰, or 50×10¹⁰ to 100×10¹⁰ infectious units/mL In some embodiments, the viral titer of the composition is at least any of about 5×10¹⁰ to 10×10¹⁰, 10×10¹⁰ to 15×10¹⁰, 15×10¹⁰ to 25×10¹⁰, or 25×10¹⁰ to 50×10¹⁰ infectious units/mL

One or multiple (e.g., 2, 3, or more) blebs can be created. Generally, the total volume of bleb or blebs created by the methods and systems of the invention cannot exceed the fluid volume of the eye, for example about 4 ml in a typical human subject. The total volume of each individual bleb can be at least about 0.3 ml, or at least about 0.5 ml in order to facilitate a retinal detachment of sufficient size to expose the cell types of the central retina and create a bleb of sufficient dependency for optimal manipulation. One of ordinary skill in the art will appreciate that in creating the bleb according to the methods and systems of the invention that the appropriate intraocular pressure must be maintained in order to avoid damage to the ocular structures. The size of each individual bleb may be, for example, about 0.5 to about 1.2 ml, about 0.8 to about 1.2 ml, about 0.9 to about 1.2 ml, about 0.9 to about 1.0 ml, about 1.0 to about 2.0 ml, about 1.0 to about 3.0 ml. Thus, in one example, to inject a total of 3 ml of isolated polynucleotide, vector and/or pharmaceutical composition suspension, 3 blebs of about 1 ml each can be established. The total volume of all blebs in combination may be, for example, about 0.5 to about 3.0 ml, about 0.8 to about 3.0 ml, about 0.9 to about 3.0 ml, about 1.0 to about 3.0 ml, about 0.5 to about 1.5 ml, about 0.5 to about 1.2 ml, about 0.9 to about 3.0 ml, about 0.9 to about 2.0 ml, about 0.9 to about 1.0 ml.

In order to safely and efficiently transduce areas of target retina (e.g., the central retina) outside the edge of the original location of the bleb, the bleb may be manipulated to reposition the bleb to the target area for transduction. Manipulation of the bleb can occur by the dependency of the bleb that is created by the volume of the bleb, repositioning of the eye containing the bleb, repositioning of the head of the human with an eye or eyes containing one or more blebs, and/or by means of a fluid-air exchange. This is particularly relevant to the central retina since this area typically resists detachment by subretinal injection. In some embodiments fluid-air exchange is utilized to reposition the bleb; fluid from the infusion cannula is temporarily replaced by air, e.g., from blowing air onto the surface of the retina. As the volume of the air displaces vitreous cavity fluid from the surface of the retina, the fluid in the vitreous cavity may flow out of a cannula. The temporary lack of pressure from the vitreous cavity fluid causes the bleb to move and gravitate to a dependent part of the eye. By positioning the eye globe appropriately, the bleb of subretinal vector and/or pharmaceutical composition position is manipulated to involve adjacent areas (e.g., the macula and/or fovea). In some cases, the mass of the bleb is sufficient to cause it to gravitate, even without use of the fluid-air exchange. Movement of the bleb to the desired location may further be facilitated by altering the position of the subject's head, so as to allow the bleb to gravitate to the desired location in the eye. Once the desired configuration of the bleb is achieved, fluid is returned to the vitreous cavity. The fluid is an appropriate fluid, e.g., fresh saline. Generally, the subretinal vector and/or pharmaceutical composition may be left in situ without retinopexy to the retinotomy and without intraocular tamponade, and the retina will spontaneously reattach within about 48 hours.

The term “bleb” as used herein refers to a fluid space within the subretinal space of an eye. A bleb of the invention may be created by a single injection of fluid into a single space, by multiple injections of one or more fluids into the same space, or by multiple injections into multiple spaces, which when repositioned create a total fluid space useful for achieving a therapeutic effect over the desired portion of the subretinal space.

By safely and effectively transducing ocular cells (e.g., RPE and/or photoreceptor cells of e.g., the macula and/or fovea) with a vector comprising a therapeutic polypeptide (e.g., CRB1-B), the methods of the invention may be used to treat an individual; e.g., a human, having an ocular disorder, wherein the transduced cells produce the therapeutic polypeptide CRB1-B or RNA sequence in an amount sufficient to treat the ocular disorder.

An effective amount of an isolated polynucleotide, vector or pharmaceutical composition according to the present disclosure is administered, depending on the objectives of treatment. For example, in use of a viral vector where a low percentage of transduction can achieve the desired therapeutic effect, then the objective of treatment is generally to meet or exceed this level of transduction. In some instances, this level of transduction can be achieved by transduction of only about 1 to 5% of the target cells, in some embodiments at least about 20% of the cells of the desired tissue type, in some embodiments at least about 50%, in some embodiments at least about 80%, in some embodiments at least about 95%, in some embodiments at least about 99% of the cells of the desired tissue type. The isolated polynucleotide, vector and/or pharmaceutical compositions may be administered by one or more subretinal injections, either during the same procedure or spaced apart by days, weeks, months, or years. In some embodiments, multiple vectors may be used to treat the human. For example, in one embodiment, multiple vectors, each encoding a different CRB1 isoform or other retinal therapeutic agent can be used. For example, a vector encoding for CRB1-B can be used and targeted to photoreceptor cells within the retina alone or in combination with a second vector encoding a CRB1 isoform selected from CRB1-A and CRB1-A2 which can be targeted to Müller cells within the retina.

In some embodiments, the administration to the retina of an effective amount of an isolated polynucleotide, vector or pharmaceutical composition according to the present disclosure transduces photoreceptor cells at or near the site of administration. In some embodiments, when a viral vector is used, more than about any of 5%, 10%, 15%, 20%, 25%, 30%, 35%, 40%, 45%, 50%, 55%, 60%, 65%, 70%, 75% or 100% of photoreceptor cells incorporate the isolated polynucleotide or vector and express the CRB1 isoform. In some embodiments, when a viral vector is used, more than about any of 5%, 10%, 15%, 20%, 25%, 30%, 35%, 40%, 45%, 50%, 55%, 60%, 65%, 70%, 75% or 100% of photoreceptor cells incorporate the isolated polynucleotide or vector and express the CRB1 isoform, are transduced. In some embodiments, about 5% to about 100%, about 10% to about 50%, about 10% to about 30%, about 25% to about 75%, about 25% to about 50%, or about 30% to about 50% of the photoreceptor cells are targeted (e.g. transduced with a viral vector). Methods to identify photoreceptor cells transduced by AAV viral particles comprising a vector or targeted with the pharmaceutical composition are known it the art and include, for example, immunohistochemistry or the use of a marker within the polynucleotide or vector such as enhanced green fluorescent protein can be used to detect incorporation or transduction of the vectors or pharmaceutical compositions.

In some embodiments of the present disclosure, the methods comprise administration to the subretina (e.g., the subretinal space) of a mammal an effective amount of an isolated polynucleotide, vector or pharmaceutical composition according to the present disclosure for treating an individual with an ocular disorder; e.g., a human with an ocular disorder. In some embodiments, the isolated polynucleotide, vector or pharmaceutical composition is injected to one or more locations in the subretina to allow expression of the polynucleotide in photoreceptor cells. In some embodiments, the isolated polynucleotide, vector or pharmaceutical composition is injected into any one of one, two, three, four, five, six, seven, eight, nine, ten or more than ten locations in the subretina.

In some embodiments the isolated polynucleotide, vector or pharmaceutical composition according to the present disclosure are administered to more than one location simultaneously or sequentially. In some embodiments, multiple injections of isolated polynucleotide, vector or pharmaceutical composition are no more than one hour, two hours, three hours, four hours, five hours, six hours, nine hours, twelve hours or 24 hours apart.

In other embodiments, the isolated polynucleotide, vector or pharmaceutical composition according to the present disclosure may be administered to the subject intravitreally. The general method for intravitreal injection may be illustrated by the following brief outline. This example is merely meant to illustrate certain features of the method, and is in no way meant to be limiting. Procedures for intravitreal injection are known in the art (see, e.g., Peyman, G. A., et al. (2009) Retina 29(7):875-912 and Fagan, X. J. and Al-Qureshi, S. (2013) Clin. Experiment. Ophthalmol. 41(5):500-7).

Briefly, a subject for intravitreal injection may be prepared for the procedure by pupillary dilation, sterilization of the eye, and administration of anesthetic. Any suitable mydriatic agent known in the art may be used for pupillary dilation. Adequate pupillary dilation may be confirmed before treatment. Sterilization may be achieved by applying a sterilizing eye treatment, e.g., an iodide-containing solution such as Povidone-Iodine (BETADINE™). A similar solution may also be used to clean the eyelid, eyelashes, and any other nearby tissues (e.g., skin). Any suitable anesthetic may be used, such as lidocaine or proparacaine, at any suitable concentration. Anesthetic may be administered by any method known in the art, including without limitation topical drops, gels or jellies, and subconjuctival application of anesthetic.

Prior to injection, a sterilized eyelid speculum may be used to clear the eyelashes from the area. The site of the injection may be marked with a syringe. The site of the injection may be chosen based on the lens of the patient. For example, the injection site may be 3-3.5 mm from the limus in pseudophakic or aphakic patients, and 3.5-4 mm from the limbus in phakic patients. The patient may look in a direction opposite the injection site.

In some embodiments, the methods comprise administration to the eye (e.g., by subretinal and/or intravitreal administration) an effective amount of an isolated polynucleotide, vector or pharmaceutical composition according to the present disclosure. In some embodiments, a viral vector is administered in a pharmaceutical composition, the viral titer of the composition is at least about any of 5×10¹², 6×10¹², 7×10¹², 8×10¹², 9×10¹², 10×10¹², 11×10¹², 15×10¹², 20×10¹², 25×10¹², 30×10¹², or 50×10¹² genome copies/mL In some embodiments, the viral titer of the vector and/or pharmaceutical composition is about any of 5×10¹² to 6×10¹², 6×10¹² to 7×10¹², 7×10¹² to 8×10¹², 8×10¹² to 9×10¹², 9×10¹² to 10×10¹², 10×10¹² to 11×10¹², 11×10¹² to 15×10¹², 15×10¹² to 20×10¹², 20×10¹² to 25×10¹², 25×10¹² to 30×10¹², 30×10¹² to 50×10¹², or 50×10¹² to 100×10¹² genome copies/mL. In some embodiments, the viral titer of the composition is about any of 5×10⁹ to 10×10⁹, 10×10⁹ to 15×10⁹, 15×10⁹ to 25×10⁹, or 25×10⁹ to 50×10⁹ transducing units/mL In some embodiments, the viral titer of the composition is at least any of about 5×10¹⁰, 6×10¹⁰, 7×10¹⁰, 8×10¹⁰, 9×10¹⁰, 10×10¹⁰, 11×10¹⁰, 15×10¹⁰, 20×10¹⁰, 25×10¹⁰, 30×10¹⁰, 40×10¹⁰, or 50×10¹⁰ infectious units/mL In some embodiments, the viral titer of the composition is at least any of about 5×10¹⁰ to 6×10¹⁰, 6×10¹⁰ to 7×10¹⁰, 7×10¹⁰ to 8×10¹⁰, 8×10¹⁰ to 9×10¹⁰, 9×10¹⁰ to 10×10¹⁰, 10×10¹⁰ to 11×10¹⁰, 11×10¹⁰ to 15×10¹⁰, 15×10¹⁰ to 20×10¹⁰, 20×10¹⁰ to 25×10¹⁰, 25×10¹⁰ to 30×10¹⁰, 30×10¹⁰ to 40×10¹⁰, 40×10¹⁰ to 50×10¹⁰, or 50×10¹⁰ to 100×10¹⁰ infectious units/mL In some embodiments, the viral titer of the composition is at least any of about 5×10¹⁰ to 10×10¹⁰, 10×10¹⁰ to 15×10¹⁰, 15×10¹⁰ to 25×10¹⁰, or 25×10¹⁰ to 50×10¹⁰ infectious units/mL of 5×10¹² to 10×10¹², 10×10¹² to 25×10¹², or 25×10¹² to 50×10¹² genome copies/mL In some embodiments, the methods comprise administration to the eye (e.g., by subretinal and/or intravitreal administration) of an individual (e.g., a human) an effective amount of a vector according to the present disclosure. In some embodiments, the dose of vectors and/or pharmaceutical compositions administered to the individual is at least about any of 1×10⁸ to about 1×10¹³ genome copies/kg of body weight. In some embodiments, the dose of vectors and/or pharmaceutical compositions administered to the individual is about any of 1×10⁸ to about 1×10¹³ genome copies/kg of body weight.

During injection, the needle may be inserted perpendicular to the sclera and pointed to the center of the eye. The needle may be inserted such that the tip ends in the vitreous, rather than the subretinal space. Any suitable volume known in the art for injection may be used. After injection, the eye may be treated with a sterilizing agent such as an antibiotic. The eye may also be rinsed to remove excess sterilizing agent.

Other embodiments of the present disclosure provides a means to determine the effectiveness of delivery of a vector or pharmaceutical composition according to the present disclosure. The effectiveness of delivery by subretinal or intravitreal injection of a vector or pharmaceutical composition according to the present disclosure can be monitored by several criteria as described herein. For example, after treatment in a subject using methods of the present invention, the subject may be assessed for e.g., an improvement and/or stabilization and/or delay in the progression of one or more signs or symptoms of the disease state by one or more clinical parameters including those described herein. Examples of such tests are known in the art, and include objective as well as subjective (e.g., subject reported) measures. For example, to measure the effectiveness of a treatment on a subject's visual function, one or more of the following may be evaluated: the subject's subjective quality of vision or improved central vision function (e.g., an improvement in the subject's ability to read fluently and recognize faces), the subject's visual mobility (e.g., a decrease in time needed to navigate a maze), visual acuity (e.g., an improvement in the subject's Log MAR score), microperimetry (e.g., an improvement in the subject's dB score), dark-adapted perimetry (e.g., an improvement in the subject's dB score), fine matrix mapping (e.g., an improvement in the subject's dB score), Goldmann perimetry (e.g., a reduced size of scotomatous area (i.e. areas of blindness) and improvement of the ability to resolve smaller targets), flicker sensitivities (e.g., an improvement in Hertz), autofluorescence, and electrophysiology measurements (e.g., improvement in ERG). In some embodiments, the visual function is measured by the subject's visual mobility. In some embodiments, the visual function is measured by the subject's visual acuity. In some embodiments, the visual function is measured by microperimetry. In some embodiments, the visual function is measured by dark-adapted perimetry. In some embodiments, the visual function is measured by ERG. In some embodiments, the visual function is measured by the subject's subjective quality of vision.

In the case of diseases resulting in progressive degenerative visual function, treating the subject at an early age may not only result in a slowing or halting of the progression of the disease, it may also ameliorate or prevent visual function loss due to acquired amblyopia. Amblyopia may be of two types. In studies in nonhuman primates and kittens that are kept in total darkness from birth until even a few months of age, the animals even when subsequently exposed to light are functionally irreversibly blind despite having functional signals sent by the retina. This blindness occurs because the neural connections and “education” of the cortex is developmentally is arrested from birth due to stimulus arrest. It is unknown if this function could ever be restored. In the case of diseases of retinal degeneration, normal visual cortex circuitry was initially “learned” or developmentally appropriate until the point at which the degeneration created significant dysfunction. The loss of visual stimulus in terms of signaling in the dysfunctional eye creates “acquired” or “learned” dysfunction (“acquired amblyopia”), resulting in the brain's inability to interpret signals, or to “use” that eye. It is unknown in these cases of “acquired amblyopia” whether with improved signaling from the retina as a result of gene therapy of the amblyopic eye could ever result in a gain of more normal function in addition to a slowing of the progression or a stabilization of the disease state. In some embodiments, the human treated is less than 30 years of age. In some embodiments, the human treated is less than 20 years of age. In some embodiments, the human treated is less than 18 years of age. In some embodiments, the human treated is less than 15 years of age. In some embodiments, the human treated is less than 14 years of age. In some embodiments, the human treated is less than 13 years of age. In some embodiments, the human treated is less than 12 years of age. In some embodiments, the human treated is less than 10 years of age. In some embodiments, the human treated is less than 8 years of age. In some embodiments, the human treated is less than 6 years of age.

In some ocular disorders, there is a “nurse cell” phenomena, in which improving the function of one type of cell improves the function of another. For example, transduction of the etinal pigment epithelium (RPE) of the central retina by an isolated polynucleotide, vector and/or pharmaceutical composition of the present disclosure may then improve the function of the rods, and in turn, improved rod function results in improved cone function. Accordingly, treatment of one type of cell may result in improved function in another.

The selection of a particular isolated polynucleotide, vector or pharmaceutical composition according to the present disclosure depend on a number of different factors, including, but not limited to, the individual human's medical history and features of the condition and the individual being treated. The assessment of such features and the design of an appropriate therapeutic regimen is ultimately the responsibility of the prescribing physician.

As used herein, the term “individual,” “subject,” and “patient” are used interchangeably herein and refer to both human and nonhuman animals. The term “nonhuman animals” of the disclosure includes all vertebrates, e.g., mammals and non-mammals, such as, domesticated animals (e.g., cows, sheep, cats, dogs, and horses), primates (e.g., humans and non-human primates such as monkeys), rabbits, and rodents (e.g., mice and rats), amphibians, reptiles, and the like. In some embodiments, the individual or subject comprises a human. In certain embodiments, the subject comprises a human suffering from, or is at risk of suffering from, an ocular disorder.

In some embodiments, the subject to be treated has a genetic ocular disorder, but has not yet manifested clinical signs or symptoms. In some embodiments, the human to be treated has an ocular disorder. In some embodiments, the human to be treated has manifested one or more signs or symptoms of an ocular disorder. In some embodiments, the subject to be treated has a mutation in one or both alleles of the crb1 gene.

An “allele” refers to one of several alternative forms of a gene occupying a given locus on a chromosome. The length of an allele can be as small as one nucleotide, but is often larger. As used herein, a “mutation” refers to an alteration in the DNA sequence of a gene, such that the sequence differs from what is found in most people. A mutation may comprise a substitution of one or more nucleotides, an insertion of one or more nucleotides, or a deletion of one or more nucleotides.

In some embodiments, the ocular disorder comprises a retinopathy. As used herein, the term “retinopathy” refers to any damage to the retina of the eyes. This term often refers to retinal vascular disease, or damage to the retina caused by abnormal blood flow. Non-limiting examples of ocular disorder or retinopathies which may be treated by the systems and methods of the invention include: autosomal recessive severe early-onset retinal degeneration (Leber's Congenital Amaurosis), congenital achromatopsia, Stargardt's disease, Best's disease, Doyne's disease, cone dystrophy, retinitis pigmentosa, X-linked retinoschisis, Usher's syndrome, age related macular degeneration, atrophic age related macular degeneration, neovascular AMD, diabetic maculopathy, proliferative diabetic retinopathy (PDR), cystoid macular oedema, central serous retinopathy, retinal detachment, intra-ocular inflammation, glaucoma, posterior uveitis, choroideremia, and Leber hereditary optic neuropathy.

The isolated polynucleotide, vector, or pharmaceutical composition according to the present disclosure can be used either alone or in combination with one or more additional therapeutic agents for treating ocular disorders. The interval between sequential administration can be in terms of at least (or, alternatively, less than) minutes, hours, or days.

In some embodiments, one or more additional therapeutic agents may be administered to the subretina or vitreous (e.g., through intravitreal administration). Non-limiting examples of the additional therapeutic agent include polypeptide neurotrophic factors (e.g., GDNF, CNTF, BDNF, FGF2, PEDF, EPO), polypeptide anti-angiogenic factors (e.g., sFlt, angiostatin, endostatin), anti-angiogenic nucleic acids (e.g., siRNA, miRNA, ribozyme), for example anti-angiogenic nucleic acids against VEGF, anti-angiogenic morpholinos, for example anti-angiogenic morpholinos against VEGF, anti-angiogenic antibodies and/or antibody fragments (e.g., Fab fragments), for example anti-angiogenic antibodies and/or antibody fragments against VEGF.

In another embodiment, the therapeutic agent used may be the use of stem cell therapy to be used in the retina of the eye in order to restore cell loss. Suitable stem cells for use in combination may be known in the art, and include administering progenitor stem cells that are capable of differentiating into retinal photoreceptor cells.

In some embodiments of the above aspects and embodiments, the isolated polynucleotide, the recombinant vector, the isolated polypeptide, or pharmaceutical composition described herein according to the present disclosure is delivered by stereotactic delivery. In some embodiments, the isolated polynucleotide, isolated polypeptide, vector and/or pharmaceutical compositions according to the present disclosure is delivered by convection enhanced delivery. In some embodiments, the isolated polynucleotide, isolated polypeptide, vector and/or pharmaceutical compositions according to the present disclosure is administered using a CED delivery system. In some embodiments, the cannula is a reflux-resistant cannula or a stepped cannula. In some embodiments, the CED delivery system comprises a cannula and/or a pump. In some embodiments, the isolated polynucleotide, isolated polypeptide, vector and/or pharmaceutical compositions according to the present disclosure is administered using a CED delivery system. In some embodiments, the pump is a manual pump. In some embodiments, the pump is an osmotic pump. In some embodiments, the pump is an infusion pump.

An “effective amount” or “therapeutically effective amount” is an amount sufficient to effect beneficial or desired results, including clinical results (e.g., amelioration of symptoms, achievement of clinical endpoints, and the like). An effective amount can be administered in one or more administrations. In terms of a disease state, an effective amount is an amount sufficient to ameliorate, stabilize, or delay development of a disease.

As used herein, “treatment,” “treating,” “therapy,” and/or “therapy regimen” are used interchangeably and refer to an approach for obtaining beneficial or desired clinical results. For purposes of the present disclosure, beneficial or desired clinical results include, but are not limited to, alleviation of symptoms, diminishment of extent of disease, stabilized (e.g., not worsening) state of disease, preventing spread (e.g., additional loss of photoreceptors and vision) of disease, delay or slowing of disease progression, amelioration or palliation of the disease state, and remission (whether partial or total), whether detectable or undetectable. “Treatment” can also mean prolonging vision as compared to expected loss of vision if not receiving treatment.

As used herein, the term “prophylactic treatment” or “preventative treatment” refers to treatment, wherein an individual is known or suspected to have or be at risk for having a disorder but has displayed no symptoms or minimal symptoms of the disorder. An individual undergoing prophylactic treatment may be treated prior to onset of symptoms. In some embodiments, a subject having an inheritable genetic ocular disease may be treated prior to showing signs and/or symptoms of the ocular disease.

The term “central retina” as used herein refers to the outer macula and/or inner macula and/or the fovea. The term “central retina cell types” as used herein refers to cell types of the central retina, such as, for example, RPE and photoreceptor cells.

The term “macula” refers to a region of the central retina in primates that contains a higher relative concentration of photoreceptor cells, specifically rods and cones, compared to the peripheral retina. The term “outer macula” as used herein may also be referred to as the “peripheral macula”. The term “inner macula” as used herein may also be referred to as the “central macula”.

The term “fovea” refers to a small region in the central retina of primates of approximately equal to or less than 0.5 mm in diameter that contains a higher relative concentration of photoreceptor cells, specifically cones, when compared to the peripheral retina and the macula.

The term “subretinal space” as used herein refers to the location in the retina between the photoreceptor cells and the retinal pigment epithelium cells. The subretinal space may be a potential space, such as prior to any subretinal injection of fluid. The subretinal space may also contain a fluid that is injected into the potential space. In this case, the fluid is “in contact with the subretinal space.” Cells that are “in contact with the subretinal space” include the cells that border the subretinal space, such as RPE and photoreceptor cells.

### Systems and Kits

The isolated polynucleotide(s), vector(s) or pharmaceutical composition(s) according to the present disclosure may be contained within a system designed for use in one of the methods of the present disclosure as provided herein. In such aspects, the system comprises, consists of, or consists essentially of a therapeutically effective amount of a vector as provided herein, and a device for delivery of the vector to the subject.

In some embodiments, the system is designed for subretinal delivery of a vector according to the present disclosure to an eye of an individual. In other embodiments, the system is designed for intravitreal delivery of a vector according to the present disclosure to the eye of an individual. In yet other embodiments, the system is designed for topical delivery of a vector according to the present disclosure to the eye of an individual.

In general, for the intravitreal or subretinal delivery of a vector according to the present disclosure, the system comprises a fine-bore cannula, wherein the cannula is 27 to 45 gauge, one or more syringes (e.g., 1, 2, 3, 4 or more), and one or more fluids (e.g., 1, 2, 3, 4 or more) suitable for use in the methods of the present disclosure. The fine bore cannula is suitable for subretinal injection of the vector and/or other fluids to be injected into the subretinal space. In some embodiments, the cannula is 27 to 45 gauge. In some embodiments, the fine-bore cannula is 35-41 gauge. In some embodiments, the fine-bore cannula is 40 or 41 gauge. In some embodiments, the fine-bore cannula is 41-gauge. The cannula may be any suitable type of cannula, for example, a de-Juan™ cannula or an Eagle™ cannula.

The syringe may be any suitable syringe, provided it is capable of being connected to the cannula for delivery of a fluid. In some embodiments, the syringe is an Accurus™ system syringe. In some embodiments, the system has one syringe. In some embodiments, the system has two syringes. In some embodiments, the system has three syringes. In some embodiments, the system has four or more syringes.

The system may further comprise an automated injection pump, which may be activated by, e.g., a foot pedal.

The fluids suitable for use in the methods of the present disclosure include those described herein, for example, one or more fluids each comprising an effective amount of one or more vectors as described herein, one or more fluids for creating an initial bleb (e.g., saline or other appropriate fluid), and one or more fluids comprising one or more therapeutic agents.

The fluids suitable for use in the methods of the present disclosure include those described herein, for example, one or more fluids each comprising an effective amount of one or more vectors as described herein, one or more fluids for creating an initial bleb (e.g., saline or other appropriate fluid), and one or more fluids comprising one or more therapeutic agents.

In some embodiments, the volume of the fluid comprising an effective amount of the vector is greater than about 0.8 ml. In some embodiments, the volume of the fluid comprising an effective amount of the vector is at least about 0.9 ml. In some embodiments, the volume of the fluid comprising an effective amount of the vector is at least about 1.0 ml. In some embodiments, the volume of the fluid comprising an effective amount of the vector is at least about 1.5 ml. In some embodiments, the volume of the fluid comprising an effective amount of the vector is at least about 2.0 ml. In some embodiments, the volume of the fluid comprising an effective amount of the vector is greater than about 0.8 to about 3.0 ml. In some embodiments, the volume of the fluid comprising an effective amount of the vector is greater than about 0.8 to about 2.5 ml. In some embodiments, the volume of the fluid comprising an effective amount of the vector is greater than about 0.8 to about 2.0 ml. In some embodiments, the volume of the fluid comprising an effective amount of the vector is greater than about 0.8 to about 1.5 ml. In some embodiments, the volume of the fluid comprising an effective amount of the vector is greater than about 0.8 to about 1.0 ml. In some embodiments, the volume of the fluid comprising an effective amount of the vector is about 0.9 to about 3.0 ml. In some embodiments, the volume of the fluid comprising an effective amount of the vector is about 0.9 to about 2.5 ml. In some embodiments, the volume of the fluid comprising an effective amount of the vector is about 0.9 to about 2.0 ml. In some embodiments, the volume of the fluid comprising an effective amount of the vector is about 0.9 to about 1.5 ml. In some embodiments, the volume of the fluid comprising an effective amount of the vector is about 0.9 to about 1.0 ml. In some embodiments, the volume of the fluid comprising an effective amount of the vector is about 1.0 to about 3.0 ml. In some embodiments, the volume of the fluid comprising an effective amount of the vector is about 1.0 to about 2.0 ml.

The fluid for creating the initial bleb may be, for example, about 0.1 to about 0.5 ml. In some embodiments, the total volume of all fluids in the system is about 0.5 to about 3.0 ml.

In some embodiments, the system comprises a single fluid (e.g., a fluid comprising an effective amount of the vector). In some embodiments, the system comprises 2 fluids. In some embodiments, the system comprises 3 fluids. In some embodiments, the system comprises 4 or more fluids.

The systems of the present disclosure may further be packaged into kits, wherein the kits may further comprise instructions for use. In some embodiments, the kits further comprise a device for delivery of a vector according to the present disclosure. In some embodiments, the delivery comprises subretinal delivery. In other embodiments, the delivery comprises topical delivery. In yet other embodiments, the delivery comprises intravitreal delivery. In some embodiments, the instructions for use include instructions according to one of the methods described herein. In some embodiments, the instructions for use include instructions for subretinal, intravitreal and/or topical delivery of a vector according to the present disclosure.

In another embodiment, the present disclosure provides a kit for treating an ocular disorder in a subject, the kit comprising a the isolated polynucleotide, the recombinant vector, the isolated polypeptide, or pharmaceutical composition described herein and a device for delivery of the isolated polynucleotide, recombinant vector, or isolated polypeptide or pharmaceutical composition to the subject, and instructions for use. In some embodiments, the device for delivery is designed for subretinal delivery. In another embodiment, the device for delivery is designed for intravitreal delivery. In a further embodiment, the device for delivery is designed for topical delivery.

In another aspect, the disclosure provides a kit for reducing progression or reducing loss of vision or maintaining vision function in a subject, the kit comprising the isolated polynucleotide, the recombinant vector, the isolated polypeptide, or pharmaceutical composition and a device for delivery of the isolated polynucleotide, recombinant vector isolated polypeptide, or pharmaceutical composition to the subject, and instructions for use. In a preferred embodiment, the kit comprises a first vector encoding CRB1-B and a second vector encoding a CRB1-A, CRB1-A2, or CRB1-C, and instructions for use.

The kits described herein can be packaged in single unit dosages or in multidosage forms. The contents of the kits are generally formulated as sterile and substantially isotonic solution.

Yet another aspect of the present disclosure provides all that is disclosed and illustrated herein.

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of” and “consisting of” those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by one of ordinary skill in the art to which this disclosure belong.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

All publications, patent applications, patents, and other references mentioned herein are incorporated by reference in their entirety. In the case of conflict, the present specification, including definitions, will control.

Other features and advantages of the invention will be apparent from the description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting.

### EXAMPLES

Genes encoding cell surface proteins control nervous system development and are implicated in neurological disorders. These genes produce alternative mRNA isoforms, which remain poorly characterized, impeding our understanding of how disease-associated mutations cause pathology. Here we introduce a strategy to reveal complete full-length isoform portfolios encoded by individual genes. We use this strategy to catalog a diversity of neural cell-surface molecules, identifying thousands of unannotated isoforms expressed in the retina and brain. By mass spectrometry, we confirm expression of newly discovered proteins on the cell surface in vivo. Remarkably, we discover that the major isoform of the retinal degeneration gene CRB1 was previously overlooked. This isoform is the only one expressed by photoreceptors, the affected cells in CRB1 disease. Using a mouse model, we identify a function for this isoform at photoreceptor-glial junctions and we demonstrate that loss of this isoform accelerates photoreceptor death.

### Materials and Methods

### Resources and Reagents

All key reagents used in this study, including antibodies, primers, datasets, and animal strains, are listed in a table of key resources (Table 1).

TABLE 1
Key resources. Provides the name and source of key reagents and resources used in this
study, such as antibodies, mouse strains, datasets, primers, and chemicals.

| Reagent Type | Reagent | Source or reference | Identifier | Additional information |
|---|---|---|---|---|
| Antibody | Alexa ® Fluor 488 | Jackson | 711-545-152 | |
| | AffiniPure Donkey | ImmunoResearch | | |
| | Anti-rabbit: 1:1000 | | | |
| Antibody | Calbindin | Swant | CB-38 | |
| Antibody | CRB1 B: 1:500 WB | this study | | |
| Antibody | ABCA4 | Santa Cruz | SC21460 | |
| Antibody | rhodopsin | Abcam | ab5417 | |
| Antibody | Sheep anti-phosducin | Sokolov et al., 2004 | | |
| Antibody | IRDye ® 800CW Donkey | Li-Cor Biosciences | 925-32213 | |
| | anti-Rabbit IgG | | | |
| | (H + L): 1:1000 | | | |
| Antibody | IRDye ® 680RD Donkey | Li-Cor Biosciences | 925-68072 | |
| | anti-Mouse IgG | | | |
| | (H + L): 1:1000 | | | |
| biological | KAPA HiFi ™ DNA | Kapa Biosystems | KK2602 | |
| reagent | Polymerase | | | |
| biological | Takara LA Taq ™ | Takara Bio | RR002A | |
| reagent | (polymerase mixture) | | | |
| biological | Nimblegen's SeqCap | Nimblegen | | Capture probes |
| reagent | EZ Developer | | | |
| | (≤200 Mb) custom | | | |
| | baits | | | |
| biological | Twist Bioscience ® | Twist Bioscience | | Capture probes |
| reagent | NGS Taret | | | |
| | Enrichment | | | |
| biological | Phusion ® High- | New England Biolabs | M05305 | |
| reagent | Fidelity DNA | | | |
| | Polymerase | | | |
| biological | Ttypsin/Lys-C Mix, | Promega | v072 | |
| reagent | Mass Spec Grade | | | |
| Chemical | 16% | Electron Microscopy | 15710 | |
| compound | Paraformaldehyde | Sciences | | |
| Chemical | 50% Glutaraldehyde | Electron Microscopy | G5882 | |
| compound | | Sciences | | |
| Chemical | Normal Donkey | Jackson | 017-000-121 | |
| compound | Serum | ImmunoResearch | | |
| Chemical | TriReagent ® | Thermo Fisher | AM9738 | |
| compound | (lysis reagent) | Scientific | | |
| Chemical | Hank's balanced salt | Sgma Aldrich | H8264 | |
| compound | solution (HBSS) | | | |
| Chemical | Fetal Bovine Serum | Life Technologies | 16250-078 | |
| compound | | | | |
| Chemical | Opti-MEM ™ I Reduced | Thermo Fisher | 31985070 | |
| compound | Serum Medium | Scientific | | |
| Chemical | Vecta-Mount ® | Vector Laboraories | H-5000 | |
| compound | (mounting medium) | | | |
| Chemical | ammonium bicarbonate | Sigma-Aldrich | 11213 | |
| compound | | | | |
| Chemical | Iodoacetamide (IAA) | Sigma-Aldrich | I1149 | |
| compound | | | | |
| Chemical | Dithiothreitol (DTT) | Sigma-Aldrich | 43815 | |
| compound | | | | |
| Chemical | Pierce ™ | Thermo Fischer | 88816 | |
| compound | Streptavidin | Scientific | | |
| | Magnetic Beads | | | |
| Chemical | EZLink ™ Sulfo-NHS- | Thermo Fischer | 21328 | |
| compound | SS-Biotin | Scientific | | |
| Chemical | Hoechst 33258 | Invitrogen | H21491 | |
| compound | | | | |
| Chemical | Isothesia: | Henry Schein | 11695-6776 | |
| compound | Isoflurane | | | |
| Chemical | Tissue Freezing | VWR | 15148-031 | |
| compound | Medium | | | |
| Chemical | 4x Laemmli Sample | Bio-Rad ® (company) | 1610747 | |
| compound | Buffer | | | |
| Chemical | Odyssey ™ Blocking | Li-Cor Biosciences | 927-40000 | |
| compound | Buffer | | | |
| Chemical | cOmplete ™, Mini, | Roche | 4693159001 | |
| compound | EDTA-free Protease | | | |
| | Inhibitor Cocktail | | | |
| | Tablets | | | |
| Other | Immun-Blot Low | Bio-Rad ® | 1620264 | |
| | Fluorescence PVDF | | | |
| | membrane | | | |
| commercial | Bio-Rad ® DC Protein | Bio-Rad ® | 5000112 | |
| assay or kit | Assay Kit | | | |
| recombinant | CAG-Crb1-B-YFP | this study | | |
| DNA | | | | |
| recombinant | CAG-Crb1-A-YFP | this study | | |
| DNA | | | | |
| recombinant | CAG-YFP | Addgene | 11180 | |
| DNA | | | | |
| Cell line | K562 | ATCC | CCL-243 | |
| model organism | Mouse: C57B16/J | Jackson Labs | 000664 | |
| model organism | Mouse: Cd1 | Charles River | 022 | |
| model organism | Mouse: Crb1null | this study | | |
| model organism | Mouse: Crb1delB | this study | | |
| model organism | Mouse: B6SJLF1/J | Jackson Labs | 100012 | |
| model organism | Mouse: C57B16/N | Charles River | 027 | |
| Software | Fiji/ImageJ | Schindelin et al. | | |
| | | (2012) | | |
| Software | Cufflinks | Trapnell et al., 2012 | | |
| Software | CummeRbund | Trapnell et al., 2012 | | |
| Software | StringTie | Perteaet al., 2016 | | |
| Software | Hisat2 | Kim et al., 2015 | | |
| Software | SQANTI | Tardaguila et al., | | |
| | | 2018 | | |
| Software | NIS Elements | Nikon Instruments | | |
| Software | Image Studio ™ | LI-COR Biosciences | | |
| | (image anaylsis | | | |
| | software) | | | |
| Software | Photoshop ® | Adobe | | |
| | (image editing | | | |
| | software) | | | |
| Software | IGV | Robinson et al., 2011 | | |
| Software | STAR | Dobin et al., 2013 | | |
| Software | Gviz | hahne et al., 2016 | | |
| Software | Iso-Seq | Pacific Biosciences | | |
| Software | SMART embl | smart.embl- | | |
| | | heidelberg.de/ | | |
| Software | text2vec | text2vec.org | | |
| Software | treemapify | https://github.com/ | | |
| | | wilkox/treemapify | | |
| Software | UpSetR | github.com/ | | |
| | | hms-dbmi/UpSetR | | |
| Software | GMAP | research- | | |
| | | pub.gene.corn/gmap/ | | |
| Software | R v3.3.3 | stat.ethz.ch/ | | |
| | | pipermail/r-help/2008- | | |
| | | May/161481.html | | |
| Software | Tidyverse R packages | tidyverse.org/ | | |
| | ggp10t2, dplyr, | | | |
| | stringr, magrittr | | | |
| Software | reshape2 R package | CRAN.R- | | used in making correlation |
| | | project.org/package= | | heatmaps |
| | | reshape2 | | |
| Software | plotly R package | plot.ly/r/ | | used for 3D plots |
| Software | dendextend R package | github.com/ | | used in clustering |
| | | talalili/dendextend | | dendrogram tree plots |
| Software | Rtsne R package | github.com/ | | used for t-SNE |
| | | jkrijthe/Rtsne | | |
| Software | vegan R package | github.com/ | | used to calculate Shannon |
| | | vegandevs/vegan | | Index |
| Software | text2vec | text2vec.org/ | | used for k-mer counting |
| Software | IsoPops | github.com/ | | Analysis and visualization |
| | | kellycochran/IsoPops | | of long-read data. |
| | | | | Introduced in this study |
| GEO dataset | P2_rep1 | PMID: 27326930 | SRR2936836 | mouse photoreceptor bulk |
| GSE74660 | | | | RNA-seq (FIG. 14B) |
| GEO dataset | P2_rep2 | PMID: 27326930 | SRR2936837 | mouse photoreceptor bulk |
| GSE74660 | | | | RNA-seq (FIG. 14B) |
| GEO dataset | P4_repl | PMID: 27326930 | SRR2936838 | mouse photoreceptor bulk |
| GSE74660 | | | | RNA-seq (FIG. 14B) |
| GEO dataset | P4_rep2 | PMID: 27326930 | SRR2936839 | mouse photoreceptor bulk |
| GSE74660 | | | | RNA-seq (FIG. 14B) |
| GEO dataset | P6_rep1 | PMID: 27326930 | SRR2936840 | mouse photoreceptor bulk |
| GSE74660 | | | | RNA-seq (FIG. 14B) |
| GEO dataset | P6_rep2 | PMID: 27326930 | SRR2936841 | mouse photoreceptor bulk |
| GSE74660 | | | | RNA-seq (FIG. 14B) |
| GEO dataset | P6_rep3 | PMID: 27326930 | SRR2936842 | mouse photoreceptor bulk |
| GSE74660 | | | | RNA-seq (FIG. 14B) |
| GEO dataset | P10_rep1 | PMID: 27326930 | SRR2936843 | mouse photoreceptor bulk |
| GSE74660 | | | | RNA-seq (FIG. 14B) |
| GEO dataset | P10_rep2 | PMID: 27326930 | SRR2936844 | mouse photoreceptor bulk |
| GSE74660 | | | | RNA-seq (FIG. 14B) |
| GEO dataset | P10_rep3 | PMID: 27326930 | SRR2936845 | mouse photoreceptor bulk |
| GSE74660 | | | | RNA-seq (FIG. 14B) |
| GEO dataset | P14_rep1 | PMID: 27326930 | SRR2936846 | mouse photoreceptor bulk |
| GSE74660 | | | | RNA-seq (FIG. 14B) |
| GEO dataset | P14_rep2 | PMID: 27326930 | SRR2936847 | mouse photoreceptor bulk |
| GSE74660 | | | | RNA-seq (FIG. 14B) |
| GEO dataset | P28_repl | PMID: 27326930 | SRR2936848 | mouse photoreceptor bulk |
| GSE74660 | | | | RNA-seq (FIG. 14B) |
| GEO dataset | P28_rep2 | PMID: 27326930 | SRR2936849 | mouse photoreceptor bulk |
| GSE74660 | | | | RNA-seq (FIG. 14B) |
| GEO dataset | P28_rep3 | PMID: 27326930 | SRR2936850 | mouse photoreceptor bulk |
| GSE74660 | | | | RNA-seq (FIG. 14B) |
| GEO dataset | P28_rep4 | PMID: 27326930 | SRR2936851 | mouse photoreceptor bulk |
| GSE74660 | | | | RNA-seq (FIG. 14B) |
| GEO dataset | P2_KO_rep1 | PMID: 27326930 | SRR2936852 | mouse photoreceptor bulk |
| GSE74660 | | | | RNA-seq (FIG. 14B) |
| GEO dataset | P2_KO_rep2 | PMID: 27326930 | SRR2936853 | mouse photoreceptor bulk |
| GSE74660 | | | | RNA-seq (FIG. 14B) |
| GEO dataset | P4_KO_repl | PMID: 27326930 | SRR2936854 | mouse photoreceptor bulk |
| GSE74660 | | | | RNA-seq (FIG. 14B) |
| GEO dataset | P4_KO_rep2 | PMID: 27326930 | SRR2936855 | mouse photoreceptor bulk |
| GSE74660 | | | | RNA-seq (FIG. 14B) |
| GEO dataset | P6_KO_rep1 | PMID: 27326930 | SRR2936856 | mouse photoreceptor bulk |
| GSE74660 | | | | RNA-seq (FIG. 14B) |
| GEO dataset | P6_KO_rep2 | PMID: 27326930 | SRR2936857 | mouse photoreceptor bulk |
| GSE74660 | | | | RNA-seq (FIG. 14B) |
| GEO dataset | P6_KO_rep3 | PMID: 27326930 | SRR2936858 | mouse photoreceptor bulk |
| GSE74660 | | | | RNA-seq (FIG. 14B) |
| GEO dataset | P10_KO_rep1 | PMID: 27326930 | SRR2936859 | mouse photoreceptor bulk |
| GSE74660 | | | | RNA-seq (FIG. 14B) |
| GEO dataset | P10_KO_rep2 | PMID: 27326930 | SRR2936860 | mouse photoreceptor bulk |
| GSE74660 | | | | RNA-seq (FIG. 14B) |
| GEO dataset | P10_KO_rep3 | PMID: 27326930 | SRR2936861 | mouse photoreceptor bulk |
| GSE74660 | | | | RNA-seq (FIG. 14B) |
| GEO dataset | P14_KO_rep1 | PMID: 27326930 | SRR2936862 | mouse photoreceptor bulk |
| GSE74660 | | | | RNA-seq (FIG. 14B) |
| GEO dataset | P14_KO_rep2 | PMID: 27326930 | SRR2936863 | mouse photoreceptor bulk |
| GSE74660 | | | | RNA-seq (FIG. 14B) |
| GEO dataset | P28_KO_rep1 | PMID: 27326930 | SRR2936864 | mouse photoreceptor bulk |
| GSE74660 | | | | RNA-seq (FIG. 14B) |
| GEO dataset | P28_KO_rep2 | PMID: 27326930 | SRR2936865 | mouse photoreceptor bulk |
| GSE74660 | | | | RNA-seq (FIG. 14B) |
| GEO dataset | E14.5 | Ref [68] | SRR5884802 | ATAC-seq (FIG. 5C) |
| GSE102092 | | | | |
| GEO dataset | E17.5 | Ref [68] | SRR5884803 | ATAC-seq (FIG. 5C) |
| GSE102092 | | | | |
| GEO dataset | P0 | Ref [68] | SRR5884804 | ATAC-seq (FIG. 5C) |
| GSE102092 | | | | |
| GEO dataset | p3 | Ref [68] | SRR5884805 | ATAC-seq (FIG. 5C) |
| GSE102092 | | | | |
| GEO dataset | p7 | Ref [68] | SRR5884807 | ATAC-seq (FIG. 5C) |
| GSE102092 | | | | |
| GEO dataset | P10 | Ref [68] | SRR5884808 | ATAC-seq (FIG. 5C) |
| GSE102092 | | | | |
| GEO dataset | P14 | Ref [68] | SRR5884810 | ATAC-seq (FIG. 5C) |
| GSE102092 | | | | |
| GEO dataset | P21 | Ref [68] | SRR5884811 | ATAC-seq (FIG. 5C) |
| GSE102092 | | | | |
| GEO dataset | Rod | Ref [69] | SRR3662499 | ATAC-seq (FIG. 5C) |
| GSE83312 | | | | |
| GEO dataset | Green Cone | Ref [69] | SRR3662503 | ATAC-seq (FIG. 5C) |
| GSE83313 | | | | |
| GEO dataset | Blue Cone | Ref [69] | SRR3662509 | ATAC-seq (FIG. 5C) |
| GSE83314 | | | | |
| ENCODE | Frontal Cortex | Ref [71] | ENCFF018VSA.bam | DNAse footprinting (FIG. 5C) |
| dataset | | | | |
| GEO dataset | Retina-Macula 1 | Ref [70] | SRR5601846 | Human ATAC-seq (FIG. 5F) |
| GSE99287 | | | | |
| GEO dataset | Retina-Macula 2 | Ref [70] | SRR5601851 | Human ATAC-seq (FIG. 5F) |
| GSE99287 | | | | |
| GEO dataset | Retina-Periphery 1 | Ref [70] | SRR5601847 | Human ATAC-seq (FIG. 5F) |
| GSE99287 | | | | |
| GEO dataset | Retina-Periphery 2 | Ref [70] | SRR5601850 | Human ATAC-seq (FIG. 5F) |
| GSE99287 | | | | |
| GEO dataset | E12.1 | Ref [38] | SRR5877174 | Mouse whole retina RNA-seq |
| GSE101986 | | | | (FIG. 5H) |
| GEO dataset | E12.2 | Ref [38] | SRR5877175 | Mouse whole retina RNA-seq |
| GSE101986 | | | | (FIG. 5H) |
| GEO dataset | E14.1 | Ref [38] | SRR5877176 | Mouse whole retina RNA-seq |
| GSE101986 | | | | (FIG. 5H) |
| GEO dataset | E14.2 | Ref [38] | SRR5877177 | Mouse whole retina RNA-seq |
| GSE101986 | | | | (FIG. 5H) |
| GEO dataset | E16.1 | Ref [38] | SRR5877178 | Mouse whole retina RNA-seq |
| GSE101986 | | | | (FIG. 5H) |
| GEO dataset | E16.2 | Ref [38] | SRR5877179 | Mouse whole retina RNA-seq |
| GSE101986 | | | | (FIG. 5H) |
| GEO dataset | P0.1 | Ref [38] | SRR5877180 | Mouse whole retina RNA-seq |
| GSE101986 | | | | (FIG. 5H) |
| GEO dataset | P0.2 | Ref [38] | SRR5877181 | Mouse whole retina RNA-seq |
| GSE101986 | | | | (FIG. 5H) |
| GEO dataset | P2.1 | Ref [38] | SRR5877182 | Mouse whole retina RNA-seq |
| GSE101986 | | | | (FIG. 5H) |
| GEO dataset | P2.2 | Ref [38] | SRR5877183 | Mouse whole retina RNA-seq |
| GSE101986 | | | | (FIG. 5H) |
| GEO dataset | P4.1 | Ref [38] | SRR5877184 | Mouse whole retina RNA-seq |
| GSE101986 | | | | (FIG. 5H) |
| GEO dataset | P4.2 | Ref [38] | SRR5877185 | Mouse whole retina RNA-seq |
| GSE101986 | | | | (FIG. 5H) |
| GEO dataset | P6.1 | Ref [38] | SRR5877186 | Mouse whole retina RNA-seq |
| GSE101986 | | | | (FIG. 5H) |
| GEO dataset | P6.2 | Ref [38] | SRR5877187 | Mouse whole retina RNA-seq |
| GSE101986 | | | | (FIG. 5H) |
| GEO dataset | P10.1 | Ref [38] | SRR5877188 | Mouse whole retina RNA-seq |
| GSE101986 | | | | (FIG. 5H) |
| GEO dataset | P10.2 | Ref [38] | SRR5877189 | Mouse whole retina RNA-seq |
| GSE101986 | | | | (FIG. 5H) |
| GEO dataset | P14.1 | Ref [38] | SRR5877190 | Mouse whole retina RNA-seq |
| GSE101986 | | | | (FIG. 5H) |
| GEO dataset | P14.2 | Ref [38] | SRR5877191 | Mouse whole retina RNA-seq |
| GSE101986 | | | | (FIG. 5H) |
| GEO dataset | P21.1 | Ref [38] | SRR5877192 | Mouse whole retina RNA-seq |
| GSE101986 | | | | (FIG. 5H) |
| GEO dataset | P21.2 | Ref [38] | SRR5877193 | Mouse whole retina RNA-seq |
| GSE101986 | | | | (FIG. 5H) |
| GEO dataset | P28.1 | Ref [38] | SRR5877194 | Mouse whole retina RNA-seq |
| GSE101986 | | | | (FIG. 5H) |
| GEO dataset | P28.2 | Ref [38] | SRR5877195 | Mouse whole retina RNA-seq |
| GSE101986 | | | | (FIG. 5H) |
| GEO dataset | 11-1516 Peripheral | PMID: 4634144 | SRR5225761 | Human retina RNA-seq (FIG. |
| GSE94437 | Retina | | | 5I) |
| GEO dataset | 11-1556 Peripheral | PMID: 4634144 | SRR5225765 | Human retina RNA-seq (FIG. |
| GSE94437 | Retina | | | 5I) |
| GEO dataset | 11-1614 Peripheral | PMID: 4634144 | SRR5225769 | Human retina RNA-seq (FIG. |
| GSE94437 | Retina | | | 5I) |
| GEO dataset | 11-1624 Peripheral | PMID: 4634144 | SRR5225773 | Human retina RNA-seq (FIG. |
| GSE94437 | Retina | | | 5I) |
| GEO dataset | 11-1648 Peripheral | PMID: 4634144 | SRR5225777 | Human retina RNA-seq (FIG. |
| GSE94437 | Retina | | | 5I) |
| GEO dataset | 11-1833 Peripheral | PMID: 4634144 | SRR5225781 | Human retina RNA-seq (FIG. |
| GSE94437 | Retina | | | 5I) |
| GEO dataset | 11-1875 Peripheral | PMID: 4634144 | SRR5225785 | Human retina RNA-seq (FIG. |
| GSE94437 | Retina | | | 5I) |
| GEO dataset | 11-2043 Peripheral | PMID: 4634144 | SRR5225789 | Human retina RNA-seq (FIG. |
| GSE94437 | Retina | | | 5I) |
| GEO dataset | 11-1516 Macular | PMID: 4634144 | SRR5225763 | Human retina RNA-seq (FIG. |
| GSE94437 | Retina | | | 5I) |
| GEO dataset | 11-1556 Macular | PMID: 4634144 | SRR5225767 | Human retina RNA-seq (FIG. |
| GSE94437 | Retina | | | 5I) |
| GEO dataset | 11-1614 Macular | PMID: 4634144 | SRR5225771 | Human retina RNA-seq (FIG. |
| GSE94437 | Retina | | | 5I) |
| GEO dataset | 11-1624 Macular | PMID: 4634144 | SRR5225775 | Human retina RNA-seq (FIG. |
| GSE94437 | Retina | | | 5I) |
| GEO dataset | 11-1648 Macular | PMID: 4634144 | SRR5225779 | Human retina RNA-seq (FIG. |
| GSE94437 | Retina | | | 5I) |
| GEO dataset | 11-1833 Macular | PMID: 4634144 | SRR5225783 | Human retina RNA-seq (FIG. |
| GSE94437 | Retina | | | 5I) |
| GEO dataset | 11-1875 Macular | PMID: 4634144 | SRR5225787 | Human retina RNA-seq (FIG. |
| GSE94437 | Retina | | | 5I) |
| GEO dataset | 11-2043 Macular | PMID: 4634144 | SRR5225791 | Human retina RNA-seq (FIG. |
| GSE94437 | Retina | | | 5I) |
| GEO dataset | Cortex_CC1 | Ref [39] | SRR3269772 | Bulk RNA-seq |
| GSE79416 | | | | |
| GEO dataset | Cortex_CC2 | Ref [39] | SRR3269773 | Bulk RNA-seq |
| GSE79416 | | | | |
| GEO dataset | Cortex_CC3 | Ref [39] | SRR3269774 | Bulk RNA-seq |
| GSE79416 | | | | |
| GEO dataset | zf_retina_1 | | SRR5833542 | Bulk RNA-seq |
| GSE101544 | | | | |
| GEO dataset | zf_retina_2 | | SRR5833543 | Bulk RNA-seq |
| GSE101544 | | | | |
| GEO dataset | Bovine_rep1 | | SRR1532566 | Bulk RNA-seq |
| GES59911 | | | | |
| GEO dataset | Bovine_rep2 | | SRR1532567 | Bulk RNA-seq |
| GES59911 | | | | |
| GEO dataset | Bovine_rep3 | | SRR1532568 | Bulk RNA-seq |
| GES59911 | | | | |
| GEO dataset | rat_rep1 | | SRR3957262 | Bulk RNA-seq |
| GSE84932 | | | | |
| GEO dataset | rat_rep2 | | SRR3957263 | Bulk RNA-seq |
| GSE84932 | | | | |
| DDBJ SRA | Sham1 | Ref [42] | DRR021692 | Adult mouse retina CAGE |
| dataset | | | | RNA-seq |
| DRA002410 | | | | |
| DDBJ SRA | Sham2 | Ref [42] | DRR021693 | Adult mouse retina CAGE |
| dataset | | | | RNA-seq |
| DRA002410 | | | | |
| DDBJ SRA | Sham3 | Ref [42] | DRR021694 | Adult mouse retina CAGE |
| dataset | | | | RNA-seq |
| DRA002410 | | | | |
| CRISPR gRNA | Crb1 guide 5'4 | | GAATAAGTACCC | 5′ guide for making Crb1 AB |
| | | | GTTCCTTG (SEQ | and B mouse |
| | | | ID NO: 28) | |
| CRISPR gRNA | Crb1 guide 3'2 | | AAAGCGATTAGG | 3′ guide for making Crb1 B |
| | | | TGATGCCC (SEQ | mouse |
| | | | ID NO: 29) | |
| CRISPR gRNA | Crb1 guide 3'4 | | TGTCCGAACACG | 3′ guide for making Crb1 AB |
| | | | TCAACCCC (SEQ | mouse |
| | | | ID NO: 30) | |
| Primer | MegF11_1.1F | IDT | GCTTGCTCACTCG | RT-PCR primer |
| | | | TTCTCAGT (SEQ | |
| | | | ID NO: 31) | |
| Primer | Megf11_2.1R | IDT | AGCTCTCTCCTTC | RT-PCR primer |
| | | | CAAACCC (SEQ ID | |
| | | | NO: 32) | |
| Primer | Megf11_alt23_R | IDT | ACCCACAAGCGT | RT-PCR primer |
| | | | TTGCTAAG (SEQ | |
| | | | ID NO: 33) | |
| Primer | Crb1_delB F1 | IDT | CAGTATCCCAGG | genotyping primer |
| | | | AGCATTCC (SEQ | |
| | | | ID NO: 34) | |
| Primer | Crb1_delB F2 | IDT | TTTTTCAGTGTGC | genotyping primer |
| | | | CAGGAAGT (SEQ | |
| | | | ID NO: 35) | |
| Primer | Crb1_delB R | IDT | AAGACTTTCCGA | genotyping primer |
| | | | AGCCATGA (SEQ | |
| | | | ID NO: 36) | |
| Primer | Crb1_delAB F | IDT | CAAGACACCCAG | genotyping primer |
| | | | GACCAAGT (SEQ | |
| | | | ID NO: 37) | |
| Primer | Crb1_delAB F2 | IDT | CTTCCCTCTTTGG | genotyping primer |
| | | | ACATTGC (SEQ ID | |
| | | | NO: 38) | |
| Primer | Crb1_delAB R | IDT | AACTTGGGAGAG | genotyping primer |
| | | | CCTGGAGT (SEQ | |
| | | | ID NO: 39) | |
| Primer | Crb1_5Fq.seq | IDT | GCCTCGGGCTAT | qPCR primer |
| | | | GTGTGTAT (SEQ | |
| | | | ID NO: 40) | |
| Primer | Crb1_5cFq.seq | IDT | AAACGGTTCCTG | qPCR primer |
| | | | TCGACCTA (SEQ | |
| | | | ID NO: 41) | |
| Primer | Crb1_6Rq.seq | IDT | Ggcaagggtgcag | qPCR primer |
| | | | taaacat (SEQ ID | |
| | | | NO: 42) | |
| Primer | Crb1_11Fq.seq | IDT | Tgcatcaatggagg | qPCR primer |
| | | | actgtg (SEQ ID | |
| | | | NO: 43) | |
| Primer | Crb1_11UTRRq.seq | IDT | Tcatgcgcagtacg | qPCR primer |
| | | | aggtag (SEQ ID | |
| | | | NO: 44) | |
| Primer | Crb1_12Rq.seq | IDT | TGAAGAACAGGG | qPCR primer |
| | | | CCAAAGTT (SEQ | |
| | | | ID NO: 45) | |
| Primer | Crb1_6Fq.seq | IDT | AGAGGACGCTGC | qPCR primer |
| | | | ATCAACTT (SEQ | |
| | | | ID NO: 46) | |
| Primer | Crb1_7Rq.seq | IDT | TCATCTTGGCCAA | qPCR primer |
| | | | ATCTTCC (SEQ ID | |
| | | | NO: 47) | |
| Primer | Crb1_8Fq.seq | IDT | GCTCCCTCAAGG | qPCR primer |
| | | | GTTTGAAT (SEQ | |
| | | | ID NO: 48) | |
| Primer | Crb1_9Rq.seq | IDT | CCATCAGGTGCA | qPCR primer |
| | | | GCGTATAA (SEQ | |
| | | | ID NO: 49) | |
| Base Scope | BA-Mm-Megf11-E14E17 | Advanced Cell | 720881 | 1999-2304 |
| Probe | | Diagnostics | | |
| Base Scope | BA-Mm-Megf11-E16bE17 | Advanced Cell | 720891 | 2210-2247 |
| Probe | | Diagnostics | | |
| Base Scope | BA-Mm-Megfl1-E16E17 | Advanced Cell | 720901 | 2264-2302 |
| Probe | | Diagnostics | | |
| Base Scope | BA-Mm-Megf11-E19E23 | Advanced Cell | 720911 | 2699-2744 |
| Probe | | Diagnostics | | |
| Base Scope | BA-Mm-Megf11-E20E23 | Advanced Cell | 720921 | 2822-2864 |
| Probe | | Diagnostics | | |
| Base Scope | BA-Mm-Megf11-E22E23 | Advanced Cell | 720931 | 3050-3086 |
| Probe | | Diagnostics | | |
| Base Scope | BA-Mm-Megf11- | Advanced Cell | 720941 | 3030-3070 |
| Probe | E23E23alt | Diagnostics | | |
| Base Scope | BA-Mm-Megf11-E23123 | Advanced Cell | 720951 | 64700141-64700189 |
| Probe | | Diagnostics | | |
| Base Scope | BA-Mm-Megf11-E24E25 | Advanced Cell | 720961 | 3329-3370 |
| Probe | | Diagnostics | | |
| Base Scope | BA-Mm-Megf11-E24124 | Advanced Cell | 720971 | 3024-3072 |
| Probe | | Diagnostics | | |
| Base Scope | BA-Mm-Megf11-E2E3 | Advanced Cell | 720981 | 273-314 |
| Probe | | Diagnostics | | |
| Base Scope | BA-Mm-Crb1-004-E5CE6 | Advanced Cell | 704351 | CACAAGGTTTTCACATTTT |
| Probe | | Diagnostics | | AATGGCAGTGCTCATAGG |
| | | | | AATTCACTGTG (SEQ ID |
| | | | | NO: 50) |
| Base Scope | BA-Mm-Crb1-E1E2 | Advanced Cell | 704341 | ACCTCAGCTCCTCACTGCT |
| Probe | | Diagnostics | | CATCTGCATAAAGAATTC |
| | | | | ATTTTGCA (SEQ ID NO: 51) |

### Animals

The use of mice in this study was approved by the Duke University Institutional Animal Care and Use Committee. All experimental procedures followed the guidelines outlined in the National Institute of Health Guide for the Care and Use of Laboratory Animals. The mice were housed under a 12 hr light-dark cycle with ad lib access to food and water.

### Knockout Mouse Generation

For the generation of Crb1^{delB}, CRISPR guides were designed to target genomic coordinates chr1: 139,256,486 and 139,254,837 and validated in vitro on genomic DNA prior to injection. A C57B16J/SJL F1 hybrid mouse line was used for injection; both strains are wild-type at the Crb1 locus (i.e. they do not carry rd8). Founders were genotyped using PCR primers to distinguish the alleles (see Table 1 for primer sequences). Two founder lines with genomic deletions were maintained. One carrying the deletion 139,254,836-139,256,488 (Δ1,652 bp) plus two additional cytosines, and the other 139,254,836-139,256,488 (41,652 bp). Both alleles effectively delete the entire first exon of Crb1-B and the promoter region and are currently phenotypically indistinguishable. For the generation of Crb1^{null}, CRISPR guides were designed to target genomic coordinates chr1: 139,256,486-139,243,407 and validated in vitro on genomic DNA prior to injection. A C57B16J/SJL F1 hybrid mouse line was used for injection and founders were genotyped using PCR primers (Table 1) to distinguish the alleles. Two founder lines with genomic deletions were maintained. One carrying the deletion chr1: 139,256,844-139,243,411 (413,433 bp) and the other 139,257,194-139,243,411 (Δ13,783 bp). Both alleles effectively delete the entire first exon of Crb1-B and the promoter region in addition to exon 6 and part of exon 7 of Crb1-A. This deletion would eliminate the exon 7 splice acceptor and is predicted to exclude exon 7 altogether. Splicing from exons 5 to 8 (as in Crb1-A) and 4 to 8 (as in Crb1-A2) would result in frameshifts. The Crb1-C-specific retained intron after exon 6 is also entirely deleted. Founder animals were backcrossed with C57B16J mice for at least two generations before analysis and genotyped to ensure they were not carrying rd1 mutation from the SJL background. Animals generated in this study will be made available to the research community for non-commercial use.

### Human Retina Tissue

Human donor eyes were obtained from Miracles in Sight® (eye bank; Winston Salem, NC), which were distributed by BioSight (Duke University Shared Resource) under the Institutional Review Board protocol #PRO-00050810. Postmortem human donor eyes were enucleated and stored on ice in PBS until dissection. Retinas were dissected from posterior poles and proceeded to RNA isolation. Donors with a history of retinal disease were excluded from the study.

### CRB1-B Antibody

We used Pierce Custom antibody service (Thermo Fisher Scientific) to generate a CRB1-B specific antibody. The antigen was the last 16 amino acids (RMNDEPVVEWGAQENY; SEQ ID NO: 53) of CRB1-B, which are predicted to be exclusive to this isoform at the protein level. Antibodies were made in rabbit according to their 90-day protocol with initial inoculation followed by 3 boosts. The antibody was affinity purified and validated by western blot with a Crb1^{delB} knockout control. CRB1-B produces a band of approximately 150 kDa, larger than the predicted size of 110 kDa. This discrepancy in experimental vs predicted size is likely due to post translational modifications such as glycosylation, since addition of PNGase F lowered the band size (not shown). Antibodies generated in this study will be made available to the research community for non-commercial use.

### RNA Extraction

For PacBio sequencing experiments and qRT-PCR, C57B16/J mice were used. Mice were anesthetized with isoflurane or cryoanesthesia (neonates only) followed by decapitation. Eyes were enucleated and retinas were dissected out, or brain was dissected from the skull and the cerebral cortex was removed. Total RNA was isolated using TRI Reagent® (lysis reagent; ThermoFisher Scientific AM9738) according to the manufacturer's protocol. Tissue was mechanically homogenized in TRI Reagent® followed by phase separation with chloroform and isopropanol precipitation. RNA samples were stored at −80° C. RIN number was calculated using a Bioanalyzer. Only RIN values above 9 were used for sequencing.

### PacBio Library Preparation for Mouse Samples

Reverse transcription was carried out using the Clontech SMARTer™ cDNA kit according to the manufacturer's protocol. cDNA was amplified with KAPA HiFi™ DNA Polymerase for 12 cycles followed by size selection (4.5 to 10 Kb). For capture, 1 ug of cDNA was denatured and blocked with DTT primer and Clontech primer then mixed with Nimblegen's SeqCap EZ Developer (≤200 Mb) custom baits at 47° C. for 20 hrs. Biotynaylated cDNAs were pulled down with streptavidin beads and washed with Nimblegen hybridization buffers to minimize non-specific binding. Targeted cDNA library was amplified 11 cycles with Takara LA Taq™ (polymerase mixture). SMRT bell library was constructed then additional size selection was performed (4.5 to 10 Kb) followed by binding of Polymerase with P6-C4 chemistry (RSII). Library was loaded onto SMRT cell using MagBead loading at 80 pM (RSII). For PacBio Sequel library, sequencing primer version 2.1 was annealed and bound using polymerase version 2.0. The bound complex was cleaned with PB Ampure beads and loaded by diffusion at 6 PM with 120 min pre-extension.

### PacBio Library Prep for Human Retina

Reverse transcription was carried out using Clontech SMARTer cDNA kit according to the manufacturer's protocol. cDNA was amplified with PrimeSTAR® GXL DNA Polymerase for 14 cycles followed by Blue Pippin size selection (4.5 to 10 Kb). For capture, lug denatured cDNA was used then incubated with Twist Custom Probes at 70° C. for 20 hrs. Biotynaylated cDNAs were pulled down with streptavidin beads and washed with Twist hybridization buffers to reduce non-specific binding. Targeted cDNA library was amplified 11 cycles with Takara LA Taq™ (polymerase mixture) yielding 650 ng of enriched cDNA for library prep. SMRTbell® Template Prep Kit 1.0 post exonuclease was used for library prep followed by a Blue Pippin size selection (4 Kb to 50 KB). Post size selection yielded 120 ng of DNA. Sequencing primer version 3.0 was annealed and bound using polymerase version 2.0. The bound complex was cleaned with PB Ampure beads and loaded onto PacBio Sequel instrument by diffusion at 6 pM.

### Processing of PacBio Raw Data

Iso-Seq software was used for initial post-processing of raw PacBio data. For lrCaptureSeq experiments, reads of insert were generated from PacBio raw reads using ConsensusTools.sh with the parameters --minFullPasses 1--minPredictedAccuracy 80 --parameters/smrtanalysis/current/analysis/etc/algorithm_parameters/2014-09/. From the reads of insert full-length, non-chimeric reads (FLNC reads) were generated using pbtranscript.py classify with the parameters --min_seq_len 500 and presence of 5′ and 3′ Clontech primers in addition to a poly A tail preceding the 3′ primer. For Megf11 PCR product sequencing, parameters were the same except that full length reads were distinguished by the presence of Megf11-specific primer sequences (5′ GGCTCCGGGGTATAGGA (SEQ ID NO:54); 3′ sequence CTGGCTGCATTGCATTGG (SEQ ID NO:55) for Megf11 long or GGTGTCCAATAAAGTC (SEQ ID NO:56) for Megf11 short).

### Isoform Level Clustering

Clustering of FLNC reads into isoforms was performed using ToFU, which consists of two parts: 1) Isoform-level clustering algorithm ICE (Iterative Clustering for Error Correction), used to generate consensus isoforms; and 2) Quiver, used to polish consensus isoforms. Transcript isoforms were generated using the ToFU_wrap script with the parameters --bin_manual “(0,4,6,9,30)” --quiver --hq_quiver_min_accuracy 0.99 (0.98 for Megf11 PCR data). This generated high-quality full-length transcripts with ≥99% post correction accuracy (≥98% for Megf11 PCR data). Isoforms were aligned to the mouse genome mm10 using GMAP (version 1.3.3b) with default values of alignment accuracy (0.85) and coverage (0.99). To prevent over clustering based on 5′ end lengths, redundant clusters were removed by collapsing all transcripts that share exactly the same exon structure. To minimize the impact truncated mRNAs may have on inflating isoform numbers, we set a threshold of ≥2 independent full-length reads that must cluster together in order to define an isoform.

To generate the entire isoform catalog, the complete dataset (all timepoints, retina and cortex) was analyzed using the cluster function of Iso-Seq (version 3), with default parameters. Only the highest-quality full-length reads (≥99% accuracy or QV≥20) from each experiment were passed to this analysis. At the conclusion of Iso-Seq 8,287 isoforms of our 30 genes were identified. HQ reads were mapped to the genome (mm10 for mouse, hg19 for human) Cupcake ToFU (github.com/Magdoll/cDNA_Cupcake) in order to further reduce overclustering of isoform subdivisions.

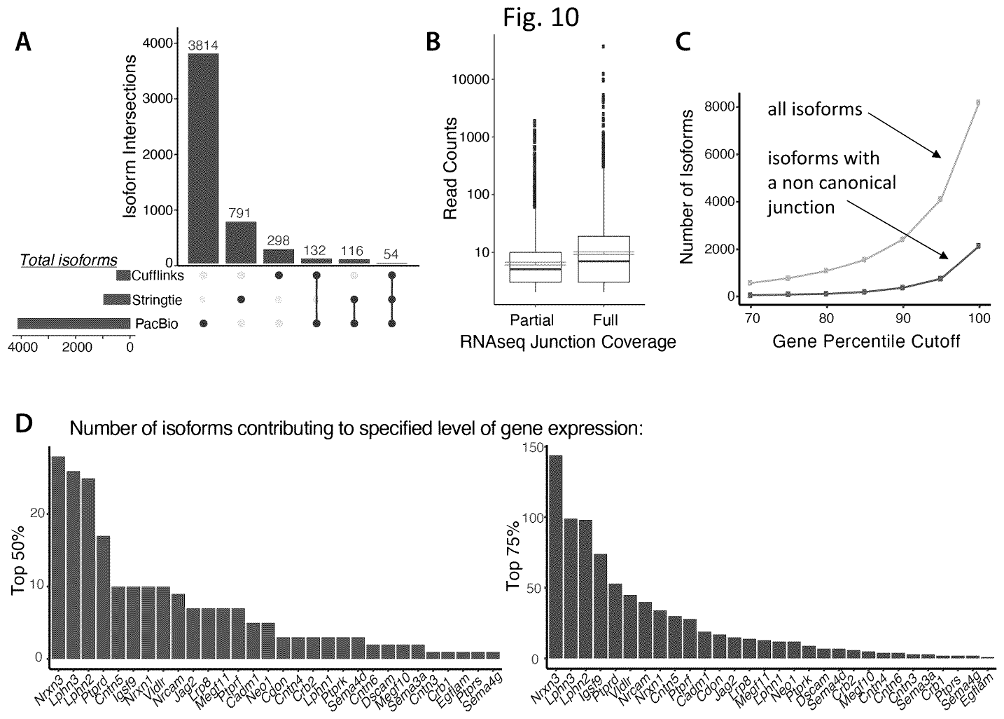

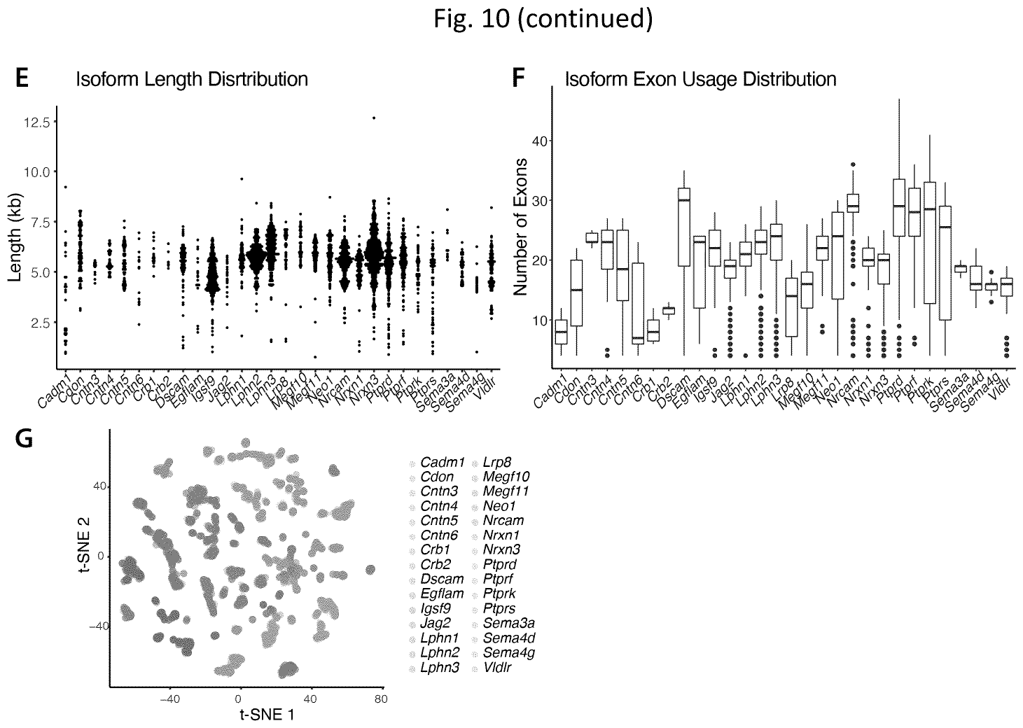

Finally, additional filtering of putative spurious isoforms was performed with our IsoPops software. The goal of this filtering was to remove artifacts arising from cDNA truncations or poly-A mispriming within genomic DNA. Details of the filtering methodology are provided below in the section describing the software package. Applying these filters yielded the final catalog of 4,116 isoforms. We did not exclude isoforms that contained non-canonical junctions, because many such isoforms were highly abundant; however, even if they were excluded, overall isoform counts would be only slightly reduced (FIG. 10C).

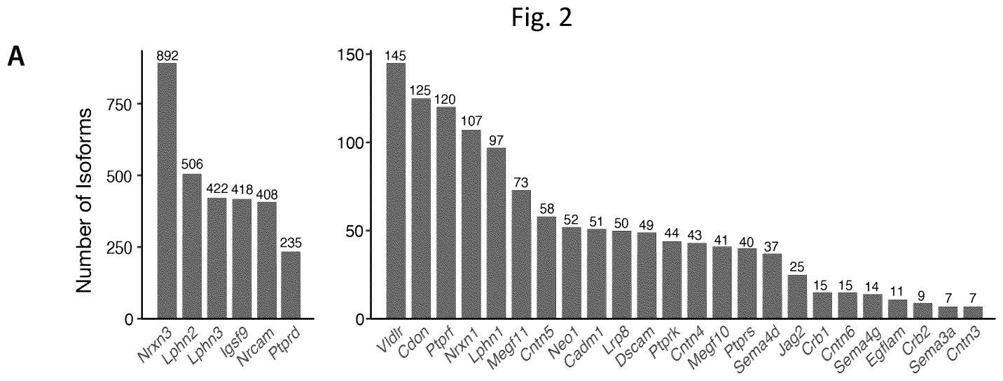

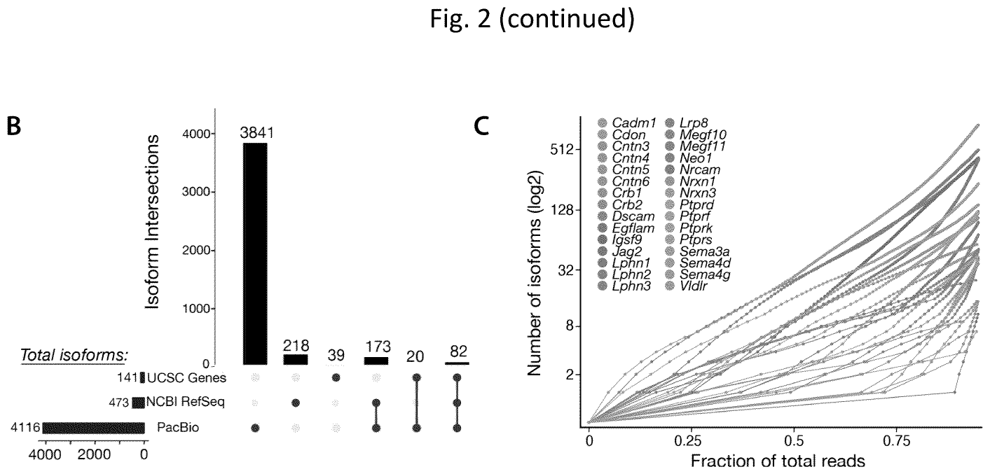

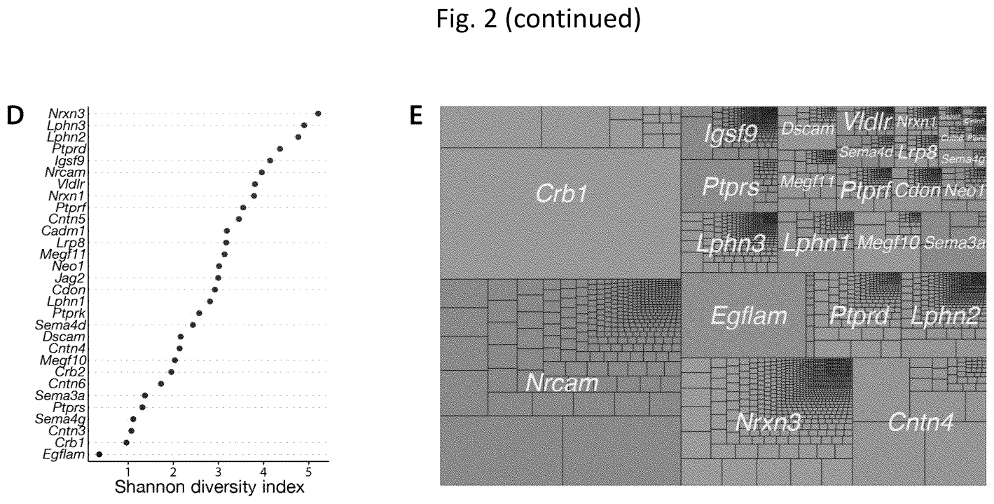

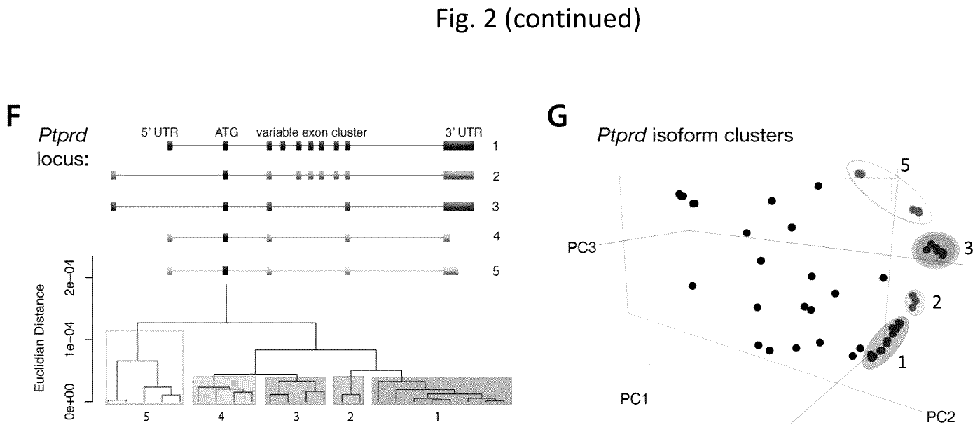

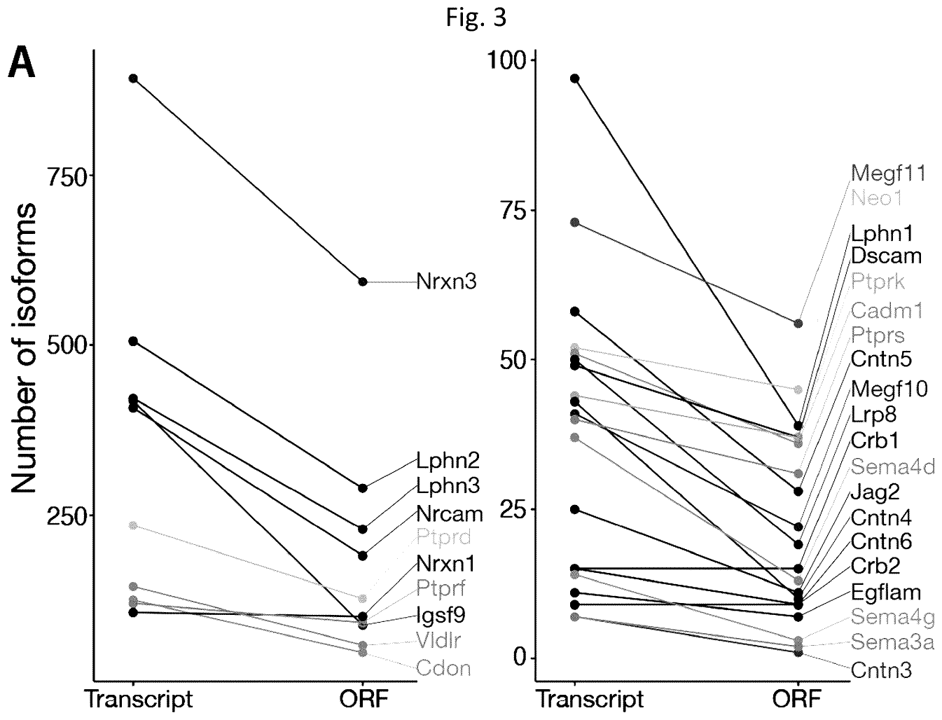

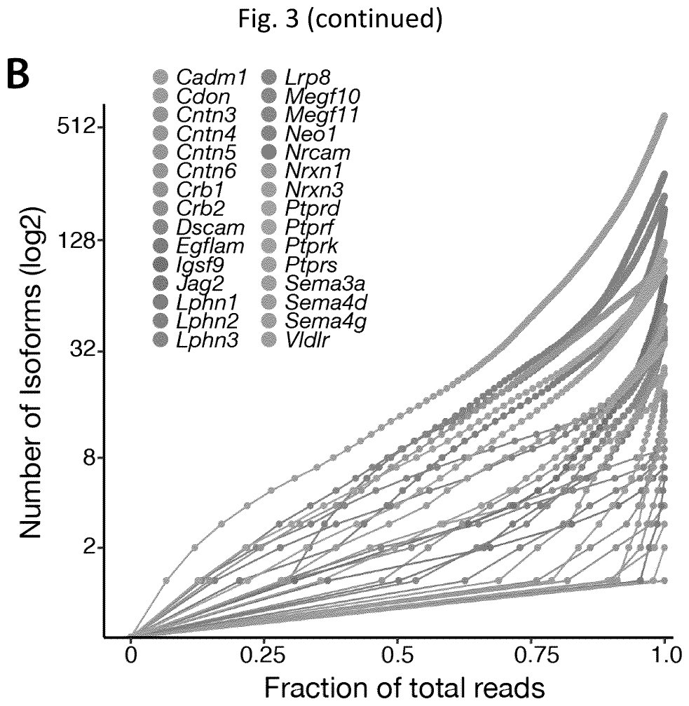

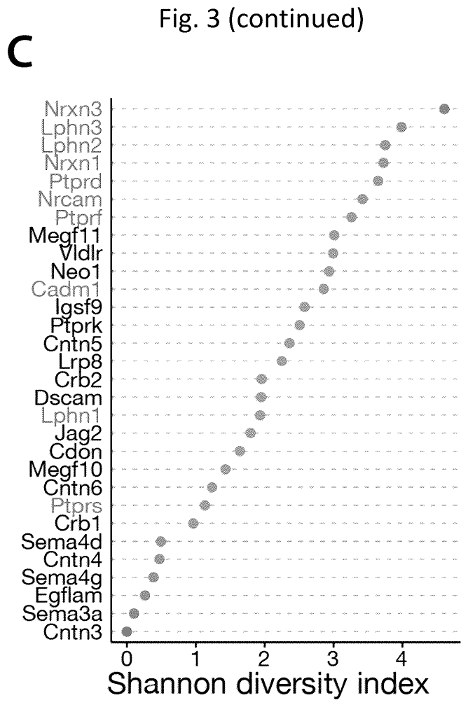

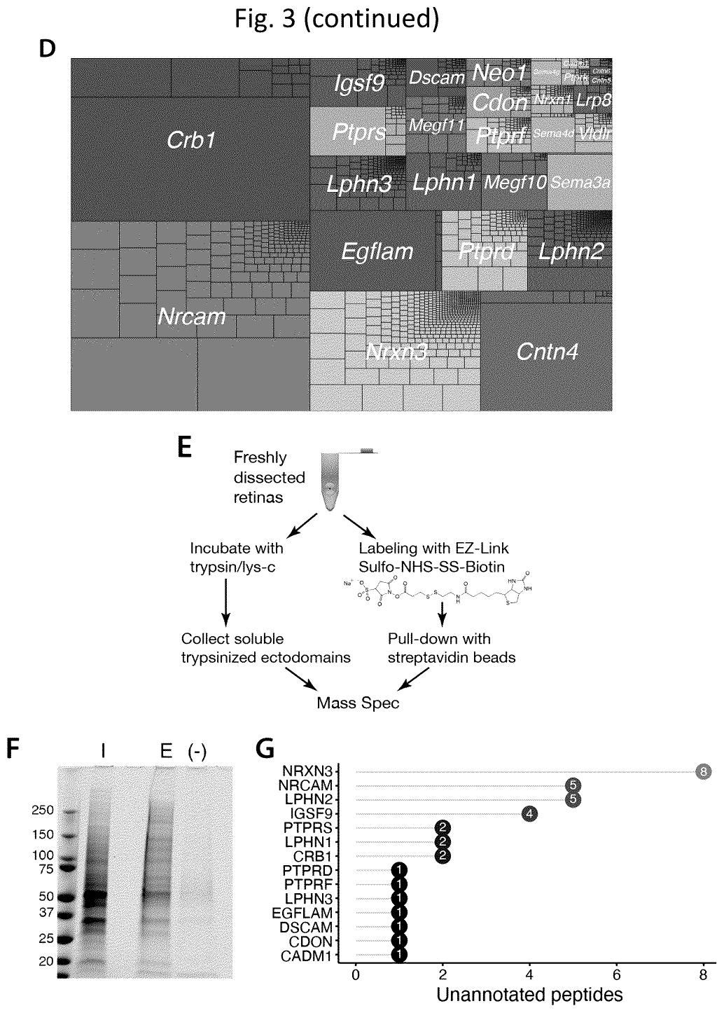

The final isoform catalog specified not only the number of isoforms, but also the number of full-length reads obtained for each isoform. We have reported these read counts for some of our analyses (e.g. FIG. 2C,E; FIG. 3B,D). These data aid in understanding how the overall expression of a particular gene is distributed across its isoform portfolio. We have avoided making conclusions about the expression level of particular isoforms, unless the PacBio data are supported by independent short-read RNA-seq data (e.g. FIG. 5D,G,H,I).

### IsoPops R Package

We developed a package of R software for convenient analysis and viewing of PacBio transcriptome sequence output. The IsoPops R package allows users to perform many of the analyses described in this study on their own long-read data.

The package offers the following features. First, it permits filtering of truncated and spurious isoforms to facilitate downstream analysis. Second, it displays maps of exon usage enabling the user to visually compare how isoforms differ. Third, it generates plots summarizing expression levels of isoforms within an individual gene and across a dataset. These include tree plots (FIG. 2E) and a variant on the Lorenz plot that we have termed a jellyfish plot (FIG. 2C). Fourth, it clusters similar isoforms and displays the data in various dimension-reducing plots such as dendrograms and 3-dimensional PCA plots. Fifth, it provides summary statistics such as the length distribution of a gene's isoforms or the number of exons used in each isoform. Finally, it performs cross-correlations, enabling the user to ask if certain exons tend to appear together in the same transcripts. Methods relevant to these features are described below.

### Filtering

The IsoPops isoform filtering process consists of 3 steps: First, transcripts containing fewer than n exons are removed. For our study, n was set to 4, because we did not expect any such short isoforms for the genes in our dataset. To quantify exon number, we did not reference exon annotations, but instead defined the number of non-contiguous genomic segments (or the number of junctions plus one) as the exon count for each isoform. This filtering step removed most spurious transcripts arising from genomic poly-A mispriming, as these sequences typically mapped to a single “exon.”

Second, we filtered out truncation artifacts. To identify truncated isoforms, we developed an algorithm designed to filter as thoroughly as possible without discarding potentially valuable unique transcripts. In particular, we wanted to preserve all unique splicing events and tolerate unique transcription start sites (TSS) and transcription termination sites (TTS) modestly. The algorithm compares the set of exon boundaries (coordinates of acceptor and donor splice sites) for an isoform pair A and B and applies the following two rules. Rule 1: If all the exon boundaries in B form a contiguous subset of the exon boundaries in A, then B is a truncation of A. We required the subset to be contiguous to avoid filtering transcripts with retained introns. Rule 2: If all 3 of the following conditions are met, B is a truncation of A. 1) The TSS of B falls within an exon in A; 2) the TTS of B is either found in A or within/beyond the 3′-most exon of the gene; 3) internal exon boundaries of B (i.e. excluding the 5′- and 3′-most exon boundaries of B) are a contiguous subset of A.

Third, the least abundant 5% of isoforms for each gene were filtered out, on the assumption that these extremely low-abundance isoforms might constitute experimental or biological noise.

### Pearson Correlation

This function enables analysis of exon co-occurrence across isoforms. Each isoform in a given gene was labeled with a series of binary values representing the exons called within its cDNA sequence. Exon calls were determined by searching for exact matches of either the first 30 bp or last 30 bp of each exon within the transcript. Exon definitions were derived from PacBio isofom GFF file. Isoforms were weighted by their full-length read counts before pairwise Pearson correlations between exon calls were calculated.

### K-mer Vectorization

IsoPops enables quantification of sequence differences between isoforms. To quantify relative differences between isoforms, we calculated the Euclidean distances between vectorizations of each isoform's cDNA sequence (or their predicted ORF amino acid sequence). We used the text2vec R package to generate a vector for each isoform, where each element in the vector equals the number of times a certain k-mer (sequence fragment) appears within the isoform. We counted all possible 6-mers within isoforms, choosing k=6 to maximize k-mer count uniqueness between isoforms without requiring excessive computational resources. Each isoform's vector of k-mer counts was then normalized to sum to 1, so that isoform distances calculated from these vectors would not be dominated by differences in length between transcripts.

### Isoform Clustering

To cluster isoforms, we calculated pairwise euclidean distances between isoforms' k-mer count vectorizations. We then performed hierarchical agglomerative clustering using the R base algorithm hclust using default settings and the “complete” agglomeration method. Dendrogram plots of clusterings were generated by the dendextend R package.

### Dimension Reduction

PCA and t-SNE were performed directly on the k-mer count vectorizations. We used the R base algorithm prcomp for PCA with default settings. For t-SNE, we ran the Rtsne package's algorithm for exact t-SNE (theta=0, maximum iterations=1000, perplexity=35), which includes a round of PCA for data pre-processing. t-SNE results are plotted in the same number of dimensions as output by the algorithm (i.e. 3D t-SNE plots were generated with ndim=3).

### Lorenz (Jellyfish) Plot

Cumulative percent abundance was calculated independently for the isoforms of each gene. First, full-length read counts were normalized across the gene and labeled “percent abundance.” Next, isoforms for a given gene were rank ordered by percent abundance in descending order. Finally, a cumulative percent abundance was calculated for each isoform, via partial summation of percent abundances in descending order. Isoforms were then plotted in this order along the y-axis and positioned according to cumulative percent abundance along the x-axis.

### ORF Prediction

Sqanti⁶⁷ was used for ORF prediction and genomic correction of PacBio isoforms.

### RNA-seq Analysis

RNA-seq fastq files were downloaded from NCBI GEO and the data was mapped with Hisat2 (version 2.1.0) to reference build mm 10 (for mouse), hg19 (for human), bosTau8 (bovine), danRer11 (zebrafish), and rn6 (rat). Dataset GSE101986 and GSE74660 were quantified with

Cufflinks (version 2.2.1). Datasets GSE94437, GSE101544, GES49911, and GSE84932 were quantified with StringTie (version 1.3.3b). All reference annotations for isoform quantification analysis were generated from corresponding reference GTF files merged with the Iso-Seq GFF output using the top 3 most abundant isoforms for each of the 30 genes.

### Isoform Predictions from RNA-seq Data

Computational prediction of isoforms was performed on the RNA-seq data set GSE101986 and GSE79416 using Cufflinks (version 2.2.1) or Stringtie (version 1.3.3b) without a reference assembly. Resulting assemblies were merged using Cuffmerge to create the final reference assembly. Isoform matching between datasets was performed using Sqanti. Isoforms were considered a match if they were identified as “full-splice match” by Sqanti. All other isoforms were considered non-matching.

### Matching of lrCaptureSeq Isoforms to Other Databases

Sqanti was used for validation of isoforms in public databases, as well as Cufflinks/Stringtie predicted isoform databases. Validation was performed using the reference GTF (either from computational assembly, NCBI RefSeq, or UCSC Genes) as input. Isoforms were validated if they were “full-splice_match” to the reference. All other isoforms were considered distinct.

### Validation of Splice Junctions and 5′ Ends of lrCaptureSeq Isoforms

Junction coverage of PacBio isoforms by RNA-seq data was assessed using Sqanti software. The junction input file for Sqanti was generated using STAR (STAR_2.6.0a) by mapping mouse retina and cortex RNA-seq data (GSE101986 and GSE79416) to the mm10 genome with a custom index made using the PacBio GFF output. Junctions were classified as either canonical (GT-AG, GC-AG, and AT-AC) or noncanonical (all other combinations).

CAGE RNA-seq data from adult mouse retina (DRA002410; samples Sham1, Sham2, and Sham3) were aligned to the genome (mm10) using Hisat2. Read coverage at exon 1 of the lrCaptureSeq isoforms was determined using BedTools (version 2.29.2). CAGE data coverage across normalized isoform lengths was performed using Qualimap (version 2.2.1).

### Chromatin Accessibility

Publicly available ATAC-seq data was used to assess chromatin accessibility (i.e. putative promoter sites) in mouse and human retina⁶⁸⁻⁷⁰. DNAse I hypersensitivity data from the ENCODE project was used for assessment of mouse cortex⁷¹. All raw fastq files were downloaded from SRA or aligned bam files from ENCODE data portal. Reads were trimmed using fastqc (version 0.11.3) and trim galore (version 0.4.1) and mapped to either the mm9 or hg19 genomes using bowtie2 (version 2.2.5). Aligned bam files were filtered for quality (>Q30) and mitochondrial and blacklisted regions were removed. Files were converted to bigwigs using deeptools (version 3.1.0) and visualized in IGV (version 2.4.16). All tracks from the same experiment are group scaled.

### Shannon Diversity Index

The Shannon index was calculated with the R package Vegan (github.com/vegandevs/vegan) according to the following equation

$${H}^{′} = - \sum {p}_{i} ln {p}_{i}$$

where pᵢ is the proportion of isoforms found in a gene (pᵢ=nᵢ/N) and nᵢ is the number of reads for isoform i and N is the total number of reads for a gene.

### Sashimi Plots

Sashimi plots were generated using Gviz (version 1.24.0) with the PacBio generated GFF file. The reads for the plot were generated by mapping the PacBio FLNC.fastq (≥85% accuracy) file to the genome (mm 10, hg19) with GMAP (version 2014 Sep. 30). Because the FLNC reads had relatively high error rates that had not been filtered out like in our final datasets, and because expression varied by gene, minimum junction coverage was variable for each plot. Minimum junction coverage was set to 60 for Crb1 mouse retina, 4 for Crb1 Cortex, 11 for human CRB1, and 4 for Megf11.

### scRNA-seq

Raw scRNAseq data profiling mouse retinal development⁴⁸ were aligned to a custom mm10 mouse genome/transcriptome using CellRanger (v3.0, 10X Genomics). mm10 reference genome and transcriptomes were downloaded from 10X Genomics and the GTF file was modified to identify the dominant Crb1 isoforms (Crb1-A and Crb1-B) as independent genes. As the CellRanger count function only considers alignments that uniquely map to a single gene, output files only report reads that map within the independent 3′ exons or splice into these from the most distal last shared exon.

Data was subsequently analyzed exactly as previously reported⁴⁸. Each cell barcode of this new analysis was assigned to a cell type based on the classifications in the original manuscript. Monocle (v3.0) 72.73 and custom R scripts were used for data visualization and plotting.

### BaseScope In Situ Hybridization

Eyes were enucleated and retinas were dissected from the eyecup, washed in PBS, and fixed at RT for 24 hours in PBS supplemented with 4% formaldehyde. Retinas were cryoprotected by osmotic equilibrium overnight at 4 degrees in PBS supplemented with 30% sucrose. Retinas were imbedded in Tissue Freezing Medium and flash frozen in 2-methyl butane chilled by dry ice. Retina tangential sections were cut to 18 μm on a Thermo Scientific Microm HM 550 Cryostat and adhered to Superfrost® Plus slides.

Probes were designed against splice junctions to detect various splicing events (see Table 1 for sequences). Probe detection was performed using the Red detection kit. BaseScope in situ hybridization was performed according to the manufacturers protocol with slight modifications. Fixed frozen retinas were baked in an oven at 60° C. for 1 hr then proceeded with standard fixed frozen pretreatment conditions with the following exceptions: Incubation in Pretreatment 2 was reduced to 2 minutes and Pretreatment 3 was reduced to 13 minutes at RT. BaseScope probes were added to the tissue and hybridized for 2 hours at 40° C. Slides were washed with wash buffer and probes were detected using the Red Singleplex detection kit. Immunostaining was performed after probe detection by incubation with primary antibodies overnight. For Megf11 BaseScope, α-Calbindin antibodies were used to label starburst amacrine cells and horizontal cells. Tissue was washed 3 times with PBS and secondary antibodies were applied and incubated for 1 hour at RT. Slides were washed once again and coverslips mounted.

### Expression of CRB1 Isoforms in K562 Cells

Tagged CRB1 constructs were built by cloning YFP in-frame at the C-terminus of CRB1-A and CRB1-B. The tagged constructs were cloned into the pCAG-YFP plasmid (Addgene #11180).

K562 cells (ATCC® CCL-243™) were obtained from, validated by, and mycoplasma tested by ATCC. The cells were cultured in Dulbecco's Modified Eagle's Medium (DMEM) with 10% bovine growth serum, 4.5 g/L D-glucose, 2.0 mM L-glutamine, 1% Penicillin/Streptomycin in 10 cm cell culture dishes. Cells were passaged every 2-3 days before reaching 2 million cells/ml. Cells were transfected using the Amaxa® Cell Line Nucleofector® Kit V following instructions in the K562 nucleofection manual. Specifically, aliquots of 1 million cells were pelleted through centrifuging at 200×g for 5 minutes at room temperature in Eppendorf® tubes. Supernatant was completely and cell pellets were suspend in 100 ul Nucleofector® solution per sample. 2 ug of plasmid DNA (pCAG:Crb1A-YFP, pCAG:Crb1B-YFP, or pCAG:YFP) were added and gently mixed with the suspended cells. Cell and DNA mixture were transfected into cuvettes, inserted into the Nucleofector® Cuvette Holder, and transfected with program T-016. Cuvettes were taken out of the holder after program is completed and immediately added with 500 ul of pre-equilibrated cultured medium. These transfected cells were then divided and transferred into two wells of the 24-well glass bottom dish (MatTek Corporation). Cells were imaged 24-hour post transfection with an inverted confocal microscope (Nikon).

### Retina Thin Sectioning and Electron Microscopy

Mice were anesthetized with isoflurane followed by decapitation. Superior retina was marked with a low temperature cautery to track orientation. Eyes were enucleated and fixed overnight at RT in Glut Buffer (40 mM MOPS, 0.005% CaCl₂, 2% formaldehyde, 2% glutaraldehyde in H₂O). The dorsal-ventral axis was marked at the time of dissection so that superior and inferior retina could subsequently be identified in thin sections. Eyes were transferred to a fresh tube containing PBS for storage 4° C. until prepped for embedding.

For thin sections, the cornea was removed from the eyecup and the eyecup was immersed in 2% osmium tetroxide in 0.1% cacodylate buffer. The eyecup was then dehydrated and embedded in Epon 812 resin. Semi-thin sections of 0.5 um were cut through the optic nervehead from superior to inferior retina. The sections were counterstained with 1% methylene blue and imaged on an Olympus IX81 bright-filed microscope.

For electron microscopy, tissue was processed and imaged as described⁷⁴. Briefly, far peripheral retina was trimmed and 65-75 um sections were prepared on a Leica ultramicrotome.

Sections were prepared separately from superior and inferior hemisections of each retina, and counterstained with a solution of 2% uranyl acetate +3.5% lead citrate. Imaging was performed on a JEM-1400 electron microscope equipped with an Orius® 1000 camera.

### Retina Nuclei Counting

Retina semi-thin sections were tile scanned on an Olympus IX81 bright-filed microscope with a 60× oil objective and stitched together with cellSens software. Using Fiji software⁷⁵, a segmented line was drawn from the optic nerve head to the periphery for both superior and inferior retina. At intervals of 500 μm, four boxes of 100 μm were drawn encapsulating the outer nuclear layer so that the center of the box was a factor of 500 μm from the optic nerve head. For each hemisphere of the retina, four boxes were made. Using the count function in ImageJ, the total number of nuclei encapsulated by each box were counted at each position. Counts were averaged across each position and plotted as well as total counts for all 8 measurements for each retina.

### Assessment of OLM Junctions by Electron Microscopy

Each section, comprising ˜90% of one retinal hemisection (far peripheral retina was trimmed during sectioning), was evaluated on the electron microscope for OLM gaps. Each potential gap was imaged and gaps were subsequently confirmed offline by evaluating the presence of electron-dense OLM junctions on the inner segments of imaged photoreceptors. The number of gaps per section was quantified, along with the size of each gap, using Fiji software. For quantification and statistics, wild-type and null/+ heterozygous controls were grouped together, since neither genotype showed any OLM gaps.

### Retina Serial Sectioning with Western Blotting

Serial sectioning was performed as described^{50,51}. Briefly, mice were anesthetized with isoflurane followed by decapitation. Eyes were enucleated and dissected in ice-cold Ringer's solution. A retina punch (2 mm diameter) was cut from the eyecup with a surgical trephine positioned adjacent next to the optic disc, transferred onto PVDF membrane with the photoreceptor layer facing up, flat mounted between two glass slides separated by plastic spacers (ca. 240 μm) and frozen on dry ice. The retina surface was aligned with the cutting plane of a cryostat and uneven edges were trimmed away. Progressive 10-μm or 20-μm tangential sections were collected—depending upon endpoint of sectioning (photoreceptors or inner retina, respectively).

### Proteomics

### Retina Trypsin Ectodomain Extraction

Juvenile P14 Mice were anesthetized with isoflurane followed by decapitation. Eyes were enucleated and dissected out of the eyecup in Ringers solution (154 mM NaCl, 5.6 mM KCl, 1 mM MgCl₂, 2.2 mM CaCl₂, 10 mM glucose, 20 mM HEPES). Retinas were placed in 100 μl Ringers solution containing 5 μg trypsin/lys-c. Solution with retina was incubated at RT for 10 minutes with periodic gentle mixing. Contents were then centrifuged at 300×G for 1.5 minutes and the supernatant was transferred to new tube. Urea was added to protein mixture to 8M then incubated at 50° C. After 1 hr incubation, DTT was added to a final concentration of 10 mM and incubated for 15 min at 50° C. Peptides were alkylated by adding 3.25 μl of 20 mM Iodoacetamide and incubated for 30 min at room temperature in the dark. Reaction was quenched by adding DTT to 50 mM final concentration. Mixture was diluted 1:3 with ˜270 μl of ammonium bicarbonate. Mixture was further digested overnight by adding 1 μg of trypsin/lys-c at 37° C.

### Cell Surface Protein Labeling and Pulldown

Cell surface labeling of membrane proteins was performed based on a described protocol⁷⁶. Mice were anesthetized with isoflurane followed by decapitation. Eyes were enucleated and retinas were dissected out of the eyecup into ice cold HBSS. Retinas were washed with HBSS followed by incubation in HBSS supplemented with EZ-Link™ Sulfo_NHS-SS-Biotin (biotinylation reagent; 0.5 mg/ml in HBSS) for 45 min on ice. Retinas were then washed 3× with HBSS+100 μM lysine to quench remaining reactive esters. Retinas were then collected in 400 μl (200 μl/retina) lysis buffer (1% Triton® X-100 (detergent), 20 mM Tris, 50 mM NaCl, 0.1% SDS, 1 mM EDTA). Retinas were homogenized using short pulses on a sonicator. The lysate centrifuged at 21,000×G for 20 min at 4° C. and the soluble fraction was collected. For immunoprecipitation, 75 μg of protein lysate was mixed with 100 μl of Streptavidin Magnetic Beads (Pierce™) and incubated at room temperature while rotating. Streptavidin/biotin complex was sequestered using a magnet and washed with lysis buffer. Proteins were eluted from the beads by incubation with elution buffer (PBS with 0.1% SDS 100 mM DTT) at 50° C. for 30 min. Experimental samples (input, biotin enriched, and non-biotin labeled negative control) were mixed with 4× SDS-PAGE sample buffer and incubated on a heat block at 90° C. for 10 min. Samples were then loaded on a 4-15% Mini-PROTEAN® TGX™ Stain-Free protein gel. Electrophoresis was carried out at 65 V through the stacking gel then adjusted to 100 V until the dye front reached the end of the gel.

### In-Gel Tryptic Digestion

After electrophoresis, the gel was washed twice with H₂O, fixed with 50% methanol, 7% acetic acid for 20 min and stained with colloidal Coomassie based GelCode™ Blue Stain reagent (Thermo Fischer Scientific, cat #24590) for 30 min. The gel was destained with distilled water at 4° C. for 2 h while rocking. Protein bands were imaged on a Bio-Rad® ChemiDoc™ Touch imager. Using a clean razor blade, bands between 75-250 kDa were excised, cut into ˜1×1 mm pieces and collected in 0.5 ml siliconized (low retention) centrifuge tube. Gel pieces were destained with 200 ul of Destaining Solution (50 mM ammonium bicarbonate, NH₄HCO₃ in 50:50 acetonitrile: water) at 37° C. for 30 min with shaking. Solution was removed and replaced with 200 μl of Destaining Solution and incubated again at 37° C. for 30 min with shaking. Solution was removed from the gel pieces and peptides were reduced with 20 μl of 20 mM DTT in 50 mM ammonium bicarbonate buffer (pH 7.8) at 60° C. for 15 min. Cysteines were alkylated by adding 50 μl of the alkylation buffer (ammonium bicarbonate buffer with 50 mM Iodoacetamide) and incubated in the dark at room temperature for 1 h. Alkylation buffer was removed from tubes and replaced with 200 μl destaining buffer. Samples were incubated for 30 min at 37° C. with shaking, buffer removed, and washed again with destaining buffer. Gel pieces were dehydrated with 75 μl of acetonitrile and incubated at room temperature for 15 min. Acetonitrile was removed from tubes and shrunken gel pieces were left to dry for 15 min. Trypsin/lys-c (5 ng/μl in 25μl of ammonium bicarbonate buffer) was added to gel pieces and incubated for 1 h at room temperature. An additional 25 μl of ammonium bicarbonate buffer was added to the tubes and incubated overnight at 37° C. Sample volume was brought to 125 μl with distilled water, and liquid containing trypsinized peptides was placed in a clean siliconized 0.5 ml tube.

### Generating lrCaptureSeq peptide library for Mass Spec

Sqanti software was used on the Iso-seq output from retina samples to predict ORFs and amino acid sequences of isoforms. Amino acid sequences were trypsinized in silico using the python program trypsin with default settings. The proline rule was followed which did not cut lysine or arginine if it immediately preceded a proline.

### Mass Spectrometry Analysis of Retinal Samples

2 μl aliquots of tryptic digests were analyzed by LC-MS/MS using a nanoAcquity UPLC system coupled to a Synapt® G2 HDMS™ mass spectrometer (Waters Corp, Milford, MA). Peptides were initially trapped on a 180 μm×20 mm Symmetry C18 column (at the 5 μl/min flow rate for 3 min in 99.9% water, 0.1% formic acid). Peptide separation was then performed on a 75 μm×150 mm column filled with the 1.7 μm C18 BEH resin (Waters) using the 6 to 30% acetonitrile gradient with 0.1% formic acid for 90 min at the flow rate of 0.3 μl/min at 35° C. Eluted peptides were sprayed into the ion source of Synapt® G2 using the 10 μm PicoTip® emitter (Waters) at the voltage of 3.0 kV.

Each sample was subjected to a data-independent analysis (HDMSE) using ion mobility workflow for simultaneous peptide quantitation and identification. For robust peak detection and alignment of individual peptides across all HDMSE runs we performed automatic alignment of ion chromatography peaks representing the same mass/retention time features using Progenesis QI software. To perform peptide assignment to the ion features, PLGS 2.5.1 (Waters) was used to generate searchable files that were submitted to the IdentityE search engine incorporated into Progenesis QI for Proteomics. For peptide identification we searched against the Iso-Seq custom database described above. To identify novel peptides, all peptides identified were cross-referenced with UniProtKb mouse database. Protein and peptide false discovery rates were determined using Protein and Peptide Prophet software (Scaffold 4.4) with a decoy database-reversed mouse UniProt 2016 database. Protein and peptide FDRs were less than 1% and 5%, respectively. To distinguish newly discovered peptides from known peptides containing posttranslational modifications, we conducted an additional database search using the most common protein modifications, including phosphorylation at S, T and Y; glutamylation at E; acetylation at K; methylation at D and E. No potential false identifications were found.

### Western Blotting

Retinas from littermate WT and Crb1 mutant mice were briefly sonicated and vortexed in 400 μl of the lysis buffer containing 2% SDS in PBS plus protease inhibitor cocktail (complete™; Roche). The lysates were spun at 20,000×g for 10 min at 22° C., supernatants collected and total protein concentration determined by the DC protein assay kit (Bio-Rad®). Using lysis buffer, the volumes were adjusted to normalize the lysates by total protein concentration before adding 4×SDS-PAGE buffer containing 400 mM DTT and heating the lysates for 10 min at 90° C. Equal volumes of the lysates, each containing 15 μg total protein, were subjected to SDS-PAGE and proteins were transferred to polyvinylidene fluoride membranes (Bio-Rad®). The membranes were blocked in the Odyssey™ blocking buffer (LiCor Bioscience) and incubated with the appropriate primary antibodies and Alexa® Fluor 680 or 800 (fluorescent dye) conjugated secondary antibodies (Invitrogen). Protein bands were imaged by the Odyssey™ CLx infrared imaging system (LiCor Bioscience).

To separate soluble and insoluble proteins, mouse retinas were briefly sonicated and hypotonically shocked in 300 μl of water on ice. The lysed retinal suspensions were spun at 20,000×g at 4° C. for 20 min, the resulting supernatant was collected and the pellet was rinsed once with water. The pellet and supernatant were reconstituted in a final volume of 400 μL lysis buffer, containing 2% SDS, 1×PBS, and protease inhibitor cocktail (complete™; Roche) Equal volume aliquots of these lysates were used as described above for Western blotting.

### Data Availability

Long-read sequencing data is available in the NCBI BioProject repository (accession number PRJNA547800). Table 2 specifies the sequence, genomic location, and read number for all isoforms of Crb1 within the lrCaptureSeq dataset.

Mass spectrometry proteomics data have been deposited at the ProteomeXchange Consortium via the PRIDE partner repository with the dataset identifier PXD017290 (DOI: 10.6019/PXD017290).

### Code Availability

IsoPops code is available at kellycochran.github.io/IsoPops/index.html, licensed under the GNU General Public License v3.0.

TABLE 2
mRNA and ORF isoforms of Crb1 identified in this study.

| | Transcript | | | | Protein | | | | |
|---|---|---|---|---|---|---|---|---|---|
| PBID | (SEQ ID NO:) | Length | Prefix | FL_reads | (SEQ ID NO:) | ORFLength | ExonCount | Chr | % Abund |
| PB.338.150 | 57 | 5764 | PB.338 | 36872 | 58 | 1003 | 7 | chr1 | 0.7217916 |
| PB.338.8 | 59 | 6170 | PB.338 | 4869 | 60 | 1405 | 12 | chr1 | 0.0953136 |
| PB.338.10 | 61 | 5894 | PB.338 | 3303 | 62 | 1344 | 11 | chr1 | 0.0646582 |
| PB.338.154 | 63 | 5554 | PB.338 | 1093 | 64 | 574 | 7 | chr1 | 0.0213961 |
| PB.338.17 | 65 | 4783 | PB.338 | 506 | 66 | 1033 | 8 | chr1 | 0.0099053 |
| PB.338.165 | 67 | 6739 | PB.338 | 329 | 68 | 1314 | 10 | chr1 | 0.0064404 |
| PB.338.162 | 69 | 5481 | PB.338 | 286 | 70 | 528 | 6 | chr1 | 0.0055986 |
| PB.338.174 | 71 | 6950 | PB.338 | 255 | 72 | 1375 | 11 | chr1 | 0.0049918 |
| PB.338.156 | 73 | 5434 | PB.338 | 234 | 74 | 844 | 6 | chr1 | 0.0045807 |
| PB.338.12 | 75 | 5678 | PB.338 | 162 | 76 | 874 | 10 | chr1 | 0.0031712 |
| PB.338.194 | 77 | 5277 | PB.338 | 158 | 78 | 520 | 8 | chr1 | 0.0030929 |
| PB.338.151 | 79 | 5530 | PB.338 | 121 | 80 | 960 | 6 | chr1 | 0.0023686 |
| PB.338.160 | 81 | 5692 | PB.338 | 101 | 82 | 576 | 7 | chr1 | 0.0019771 |
| PB.338.339 | 83 | 5801 | PB.338 | 95 | 84 | 761 | 6 | chr1 | 0.0018597 |
| PB.338.163 | 85 | 5547 | PB.338 | 95 | 86 | 420 | 8 | chr1 | 0.0018597 |

PBID: Iso-Seq isoform identifier.
Transcript: full sequence of isoform as determined by PacBio sequencing.
Lenth: length of sequence in base pairs.
Prefix: Iso-Seq gene identifier.
FL_reads: number of reads across all of our mouse experiments.
Protein: ORF predicted within transcript.
ORFLength: length of predicted protein in amino acids.
ExonCount: number of exons comprising transcript.
Chr: mouse chromosome location of gene.
% Abund: fraction of total gene reads for this isoform.

### Results

Workflow for cataloguing isoforms via long-read capture sequencing

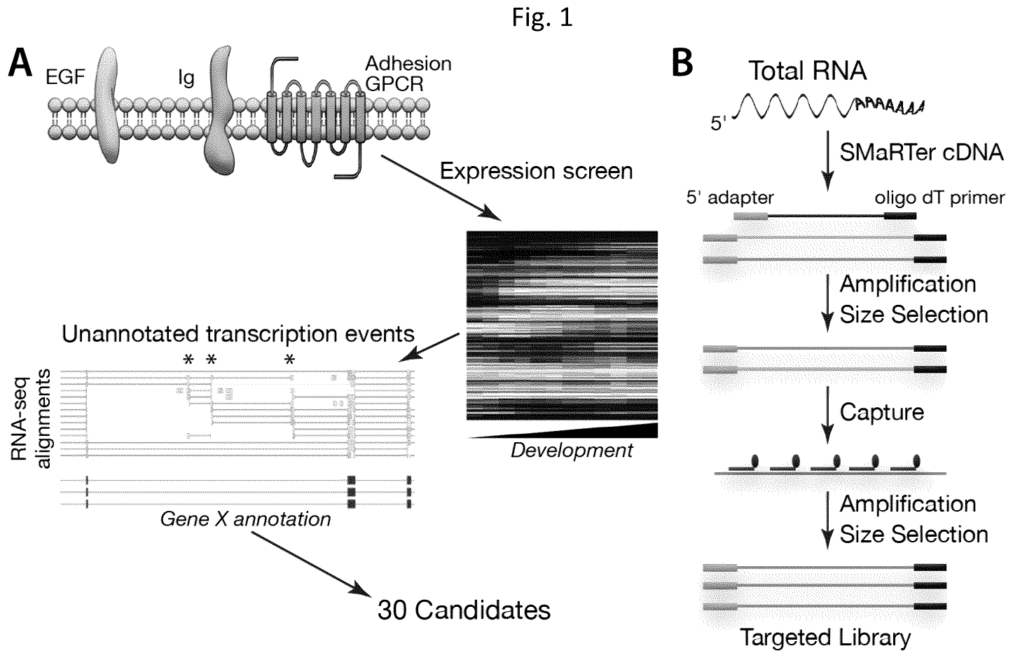

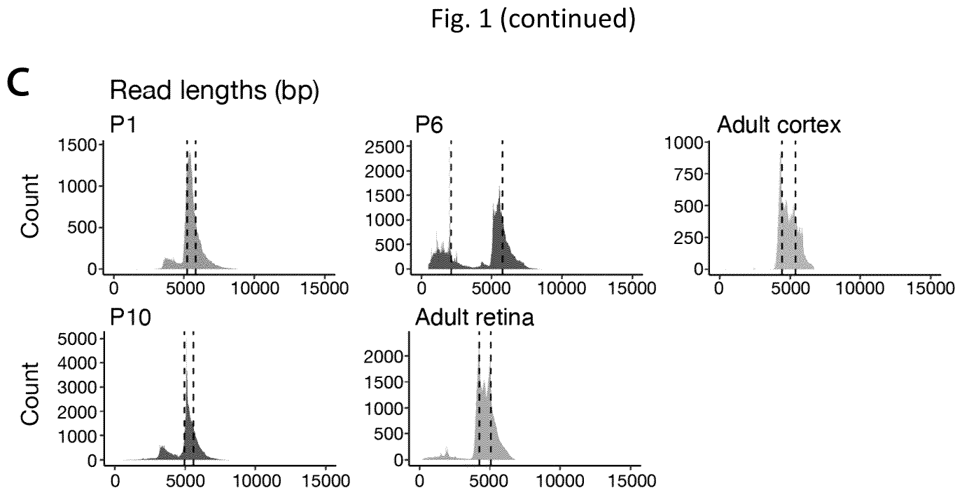

To catalog the isoform diversity of CNS cell surface molecules, we first manually screened RNA-seq data from mouse retina and brain^{38,39} to identify genes that showed substantial unannotated mRNA diversity. We focused on cell surface receptors of the epidermal growth factor (EGF), Immunoglobulin (Ig), and adhesion G-protein coupled receptor superfamilies, as these genes have many known roles in cell-cell recognition. For each gene screened (n=402), we assessed whether it was expressed during CNS development, and if so, whether the RNA-seq reads supported existence of unannotated exons or splice junctions (FIG. 1A). We found that ˜15% of genes (60/402) showed strong evidence of multiple unannotated features. These genes were selected as candidates for long-read sequencing.

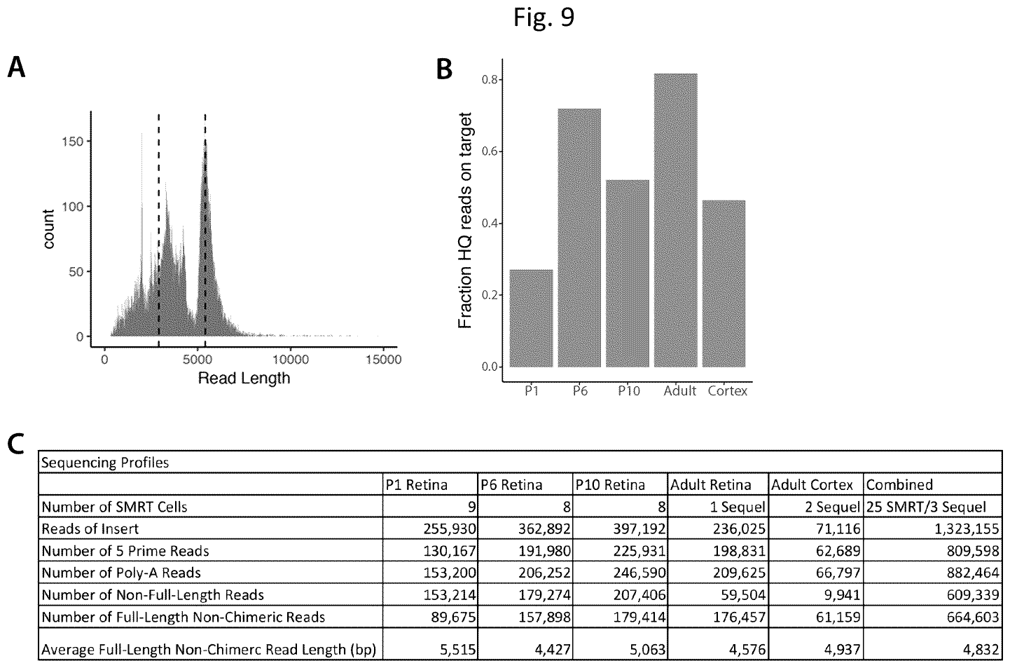

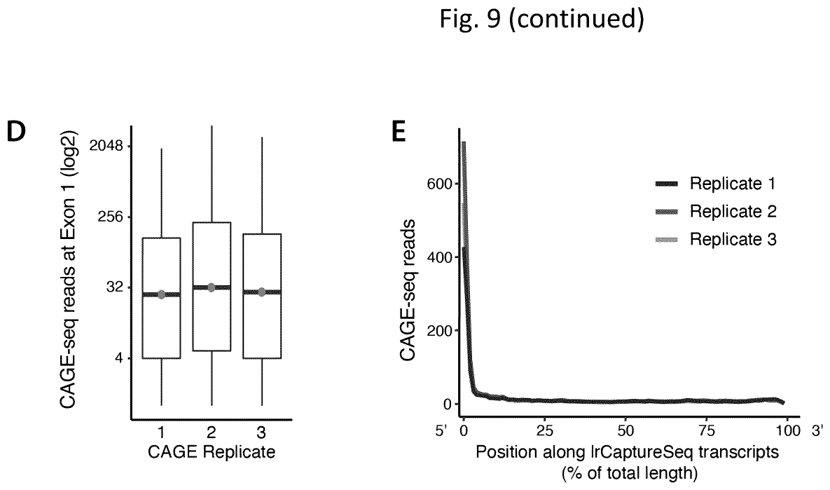

To comprehensively identify these genes' transcripts, we developed a method to improve PacBio sequencing depth for large (>4 kb) and moderately expressed cDNAs, such as the ones on our candidate gene list. We term this strategy long-read capture sequencing (lrCaptureSeq), because we adapted prior CaptureSeq approaches^{31,32,40} to enable characterization of protein-coding cDNAs with the long-read PacBio platform. In lrCaptureSeq (FIG. 1B,C), biotinylated probes are designed to tile known exons without crossing splice junctions, so as to avoid biasing the pool of captured transcripts towards particular isoforms. These probes are used to pull down cDNAs from libraries that have been size-selected to filter truncated cDNAs. In pilot experiments we found that size selection was essential to obtaining full-length reads (FIG. 9A), because shorter fragments tend to dominate the sequencing output¹⁵.

To implement lrCaptureSeq, we first filtered the initial list of 60 candidates down to 30 that were predicted to encode cDNAs of similar length (4-8 kb). The final target list included genes involved in axon guidance, synaptogenesis, and neuron-glial interactions; it also included the retinal disease gene Crb1, which is implicated in inherited photoreceptor degeneration. Some of the target genes were known to generate many isoforms (Nrxn1, Nrxn3), but in most cases isoform diversity had not previously been characterized. When captured cDNAs were sequenced on the PacBio platform, ˜132,000 full-length reads were generated per experiment (FIG. 9C). These reads were strongly enriched for the targeted genes (FIG. 9B), and the vast majority of reads were within the targeted length range (FIG. 1C). Thus, lrCaptureSeq can achieve deep full-length coverage of larger cDNAs that are underrepresented in other long-read datasets.

### A Comprehensive Isoform Catalog Generated by lrCaptureSeq

To catalog isoforms for all 30 genes across development and across CNS regions, we performed lrCaptureSeq at a variety of timepoints in mouse retina and brain (FIG. 1C; FIG. 9C). The number of isoforms, and reads comprising each, were determined using PacBio Iso-Seq software, together with custom software we developed for the analysis of isoform populations (IsoPops; github.com/kellycochran/IsoPops). After this processing pipeline, the lrCaptureSeq catalog contained 4, 116 isoforms of the 30 targeted genes (FIG. 2A,B; Table 2;)—approximately one order of magnitude greater than the number of isoforms currently annotated for this gene set in public databases (FIG. 2B). It was also far higher than the number of isoforms predicted by popular short-read transcriptome assembly software (FIG. 10A). Only 9% of lrCaptureSeq isoforms appeared in any of the databases we examined, suggesting most of them are novel.

To ensure that these novel isoforms are real, we used independent datasets to validate their transcription start sites and exon junctions. Start sites were identified using cap analysis of gene expression (CAGE), a short-read method for identifying sequences associated with the 5′ cap⁴¹. CAGE-seq reads from adult mouse retina+2 corroborated 97.5% of transcription start sites identified by lrCaptureSeq (1051/1078 adult retina isoforms had CAGE-seq coverage at their 5′ end; FIG. 9D). Moreover, CAGE-seq reads mapped selectively to 5′ ends of lrCaptureSeq isoforms (FIG. 9D,E), further supporting the accuracy of our transcription start site annotations. To validate splice junctions we first verified that the vast majority (98.9%) of lrCaptureSeq exon junctions occurred at canonical splice sites (n=80,590 junctions). Next, we tested for the existence of lrCaptureSeq exon junctions in short-read datasets from retina and brain^{38,39}. The vast majority (98.1%) of lrCaptureSeq junctions (n=79,020) were corroborated by these short-read datasets, providing independent confirmation of their validity. This included complete junction coverage for 71% of lrCaptureSeq isoforms (n=2,925). The unconfirmed junctions were likely absent from the RNA-seq data due to low expression levels, since the isoforms that did not show complete coverage were significantly less abundant (FIG. 10B). Consistent with this interpretation, unconfirmed junctions could be detected by sequencing of RT-PCR products, suggesting that they were simply below RNA-seq detection threshold (n=9/12 absent RNA-seq junctions in Megf11 gene were detected by RT-PCR). Together, these analyses strongly support the validity of our lrCaptureSeq isoform catalog.

### Efficient Isoform Detection by lrCaptureSeq

To probe the accuracy and sensitivity of isoform detection, we compared our lrCaptureSeq data to previous long-read sequencing studies cataloguing isoform diversity of the Nrxn1 and Nrxn3 genes. In these studies, PCR was used to generate isoform libraries of the a and B classes of Nrxn transcripts, which were then characterized using PacBio sequencing^{15,16}. The total number of Nrxn1 and Nrxn3 isoforms we identified was similar in scale to the previous studies (FIG. 2A), despite radically different library preparation methods and bioinformatic workflows. Patterns of exon usage in alternative splice sites (AS)1-AS4 were also similar (data not shown). For example, a deterministic AS4 splicing event identified in the previous work, wherein Nrxn3 exon 24 always splices to exon 25a, was confirmed in our data (n=76 exon 24-containing isoforms, all spliced to exon 25). These findings suggest that our Nrxn 1 and 3 isoform catalog largely matches those generated by past studies. Nevertheless, we were able to find new features of the neurexin genes not noted in the previous catalogs. Because our method was not biased by PCR primer placement, we found isoforms that did not contain canonical α or β transcript start/termination sites. For example, 64% of our Nrxn3α reads contained a distinct first exon, upstream of the annotated a transcriptional start site that lengthens the 5′ UTR. Further, we identified 7 novel transcription termination sites, used by 16 different Nrxn3α isoforms, that truncate the mRNA upstream of the transmembrane domain (data not shown). All 7 of these new sites were corroborated with junction coverage from RNA-seq data. Together, these findings demonstrate the utility of lrCaptureSeq in recovering isoform diversity with high efficiency.

### Many Isoforms Contribute to Overall Gene Expression

Given the large number of isoforms identified in our lrCaptureSeq dataset, we next sought to learn the extent to which isoform diversity is positioned to impact gene function. For diversity to be functionally significant, two conditions must be met: 1) multiple isoforms of individual genes should be expressed at meaningful levels; and 2) the sequences of the isoforms must differ enough to encode functional differences. To investigate isoform expression levels, we assessed how each gene's overall expression was distributed across its isoform portfolio (FIG. 2C,E; FIG. 10D). Some genes—for example, Egflam and Crb1—were dominated by a small number of isoforms. However, for the genes with the largest number of isoforms, expression levels were distributed far more equitably across isoforms (FIG. 2C,E). Using the Shannon diversity index⁴³, we rank-ordered genes based on the diversity of their expressed mRNA species. Nrnx3, which is known to generate extensive diversity, was the top-ranked gene. However, several other genes of the latrophilin and protein tyrosine phosphatase receptor (PTPR) families scored nearly as high (FIG. 2D). Thus, Nrxn3 is far from unique in expressing a large number of isoforms. We conclude that, for the genes in our dataset, much of the isoform diversity is expressed at appreciable levels.

### Predicted functional diversity of lrCaptureSeq isoforms

We next investigated the extent of sequence differences across the isoforms of each gene in our dataset. Most of the 30 genes encoded isoforms that varied widely in length and number of exons (FIG. 10E,F), suggesting the potential for great functional diversity. To identify isoforms that are most likely to diverge functionally, unsupervised clustering methods were used to group isoforms based on their sequence similarity (FIG. 2F,G; FIG. 10G). For most genes, isoforms clustered into distinct groups of related isoforms that made similar choices among alternative mRNA elements (FIG. 2F,G). Thus, major sequence differences exist within the isoform portfolio of individual genes, which can be traced to the inclusion of specific exon sequences by families of related isoforms.

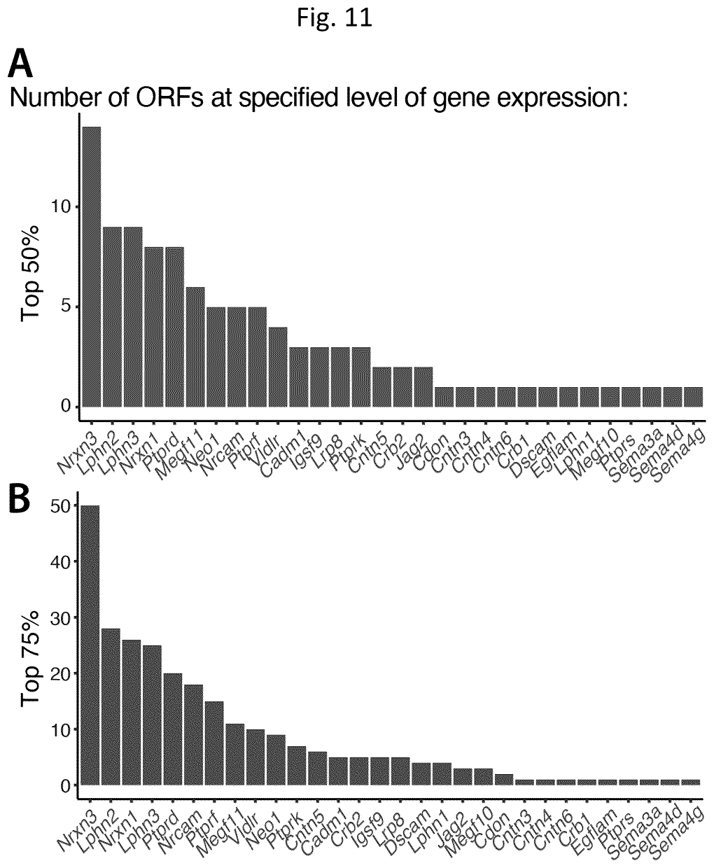

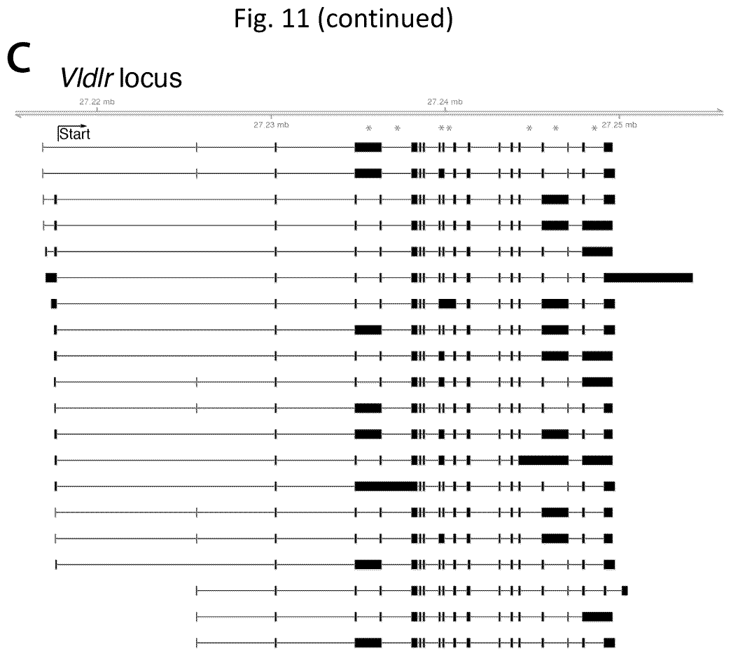

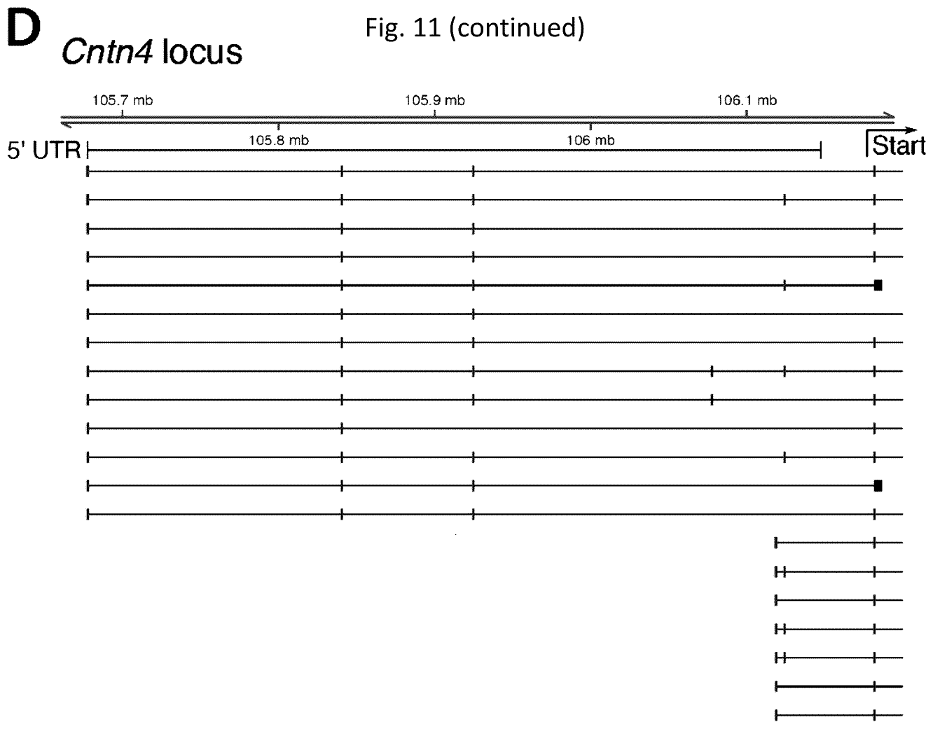

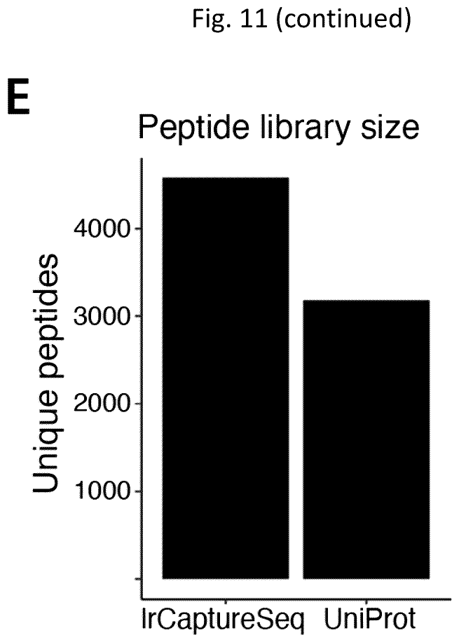

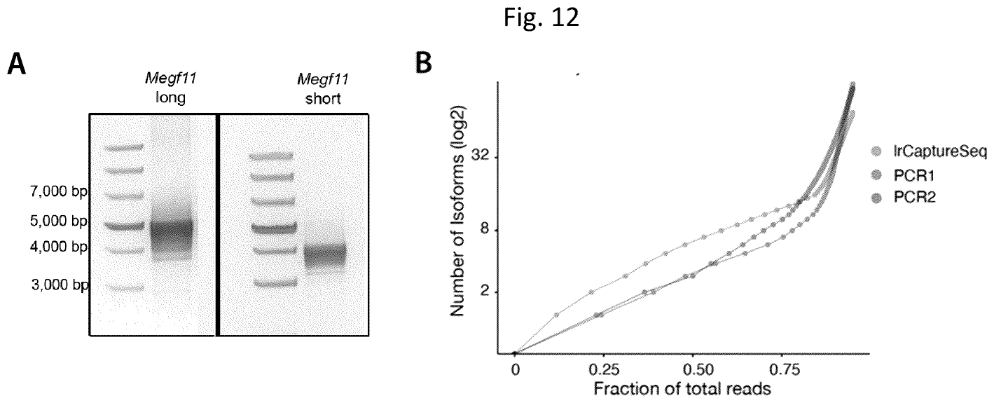

To learn whether these sequence differences might diversify protein output, we analyzed predicted open reading frames (ORFs; Table 2). Over half of the 4,116 isoforms in our dataset were found to contain unique ORFs (2,247; 54.6%). A small subset of genes expressed great mRNA diversity but no equivalent ORF diversity (FIG. 3A); this was largely due to variations in 5′ UTRs or systematic intron retention (FIG. 11C,D). Overall, however, there was a strong correlation between the number of isoforms and the number of predicted ORFs (FIG. 3A). The amount of expressed ORF diversity varied by gene; but similar to mRNAs, a large amount of this predicted protein diversity was expressed at appreciable levels (FIG. 3B-D; FIG. 11A,B). Remarkably, the genes with the most ORF diversity tended to encode a specific type of cell-surface protein: The top genes by Shannon diversity index all encode trans-synaptic adhesion molecules (FIG. 3C). This result indicates that a major function of mRNA diversity is the generation of protein variants that are positioned to influence formation or stability of synaptic connections.

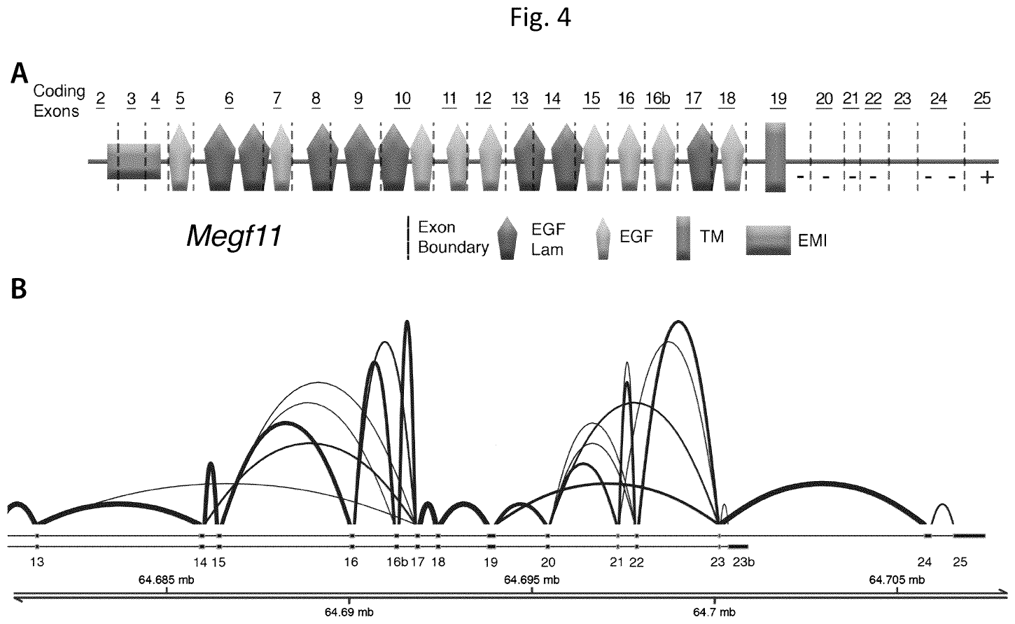

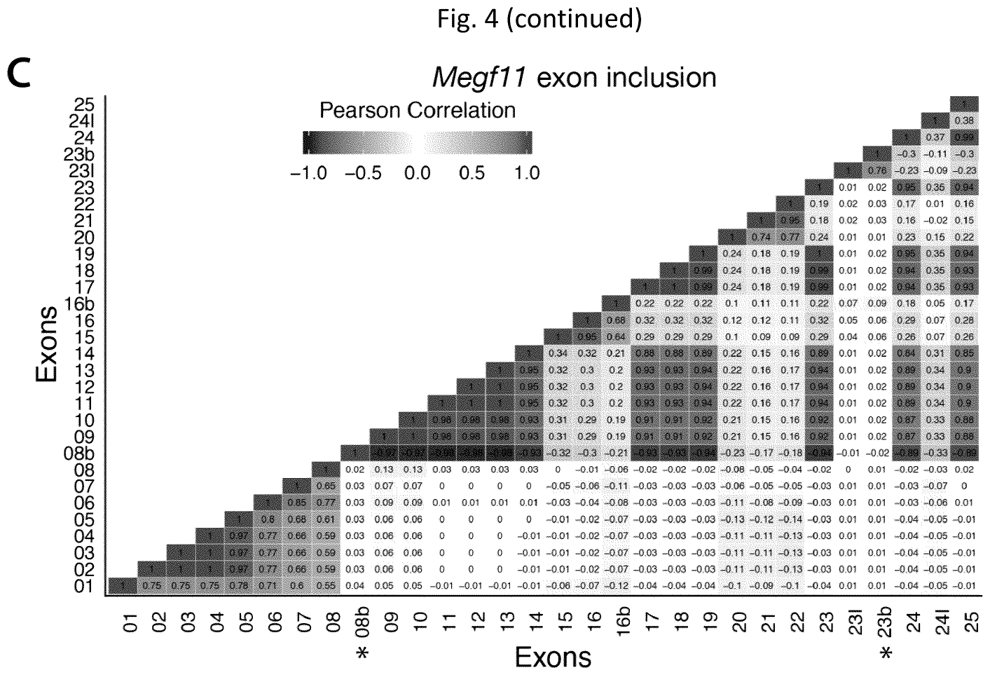

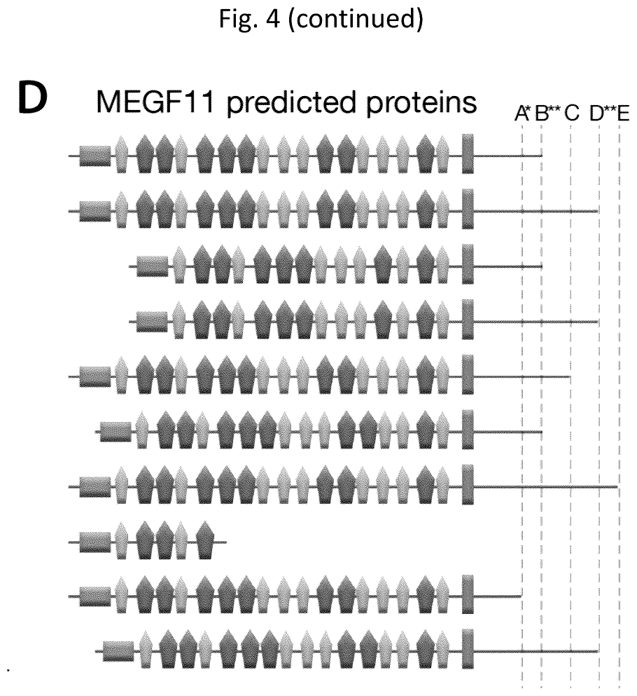

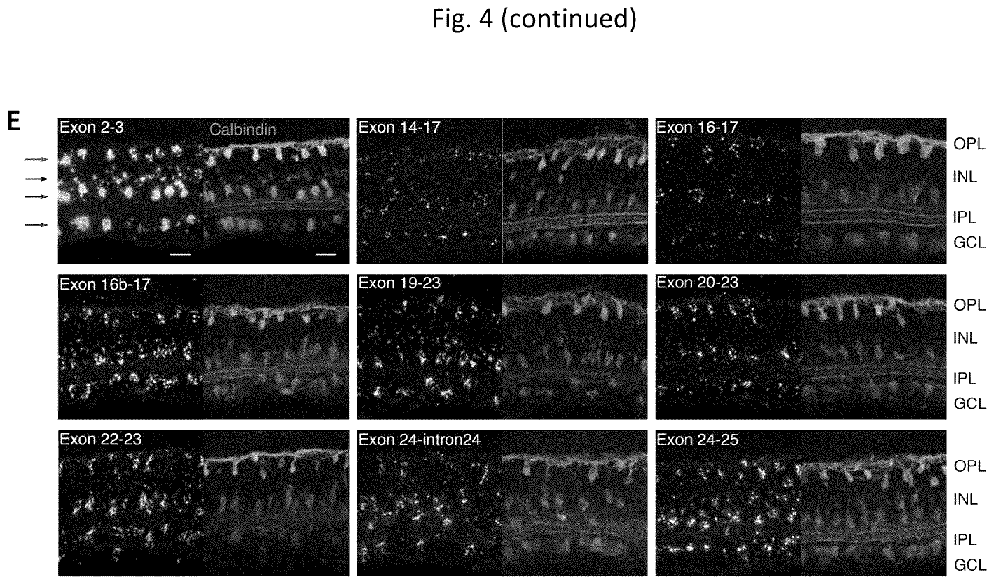

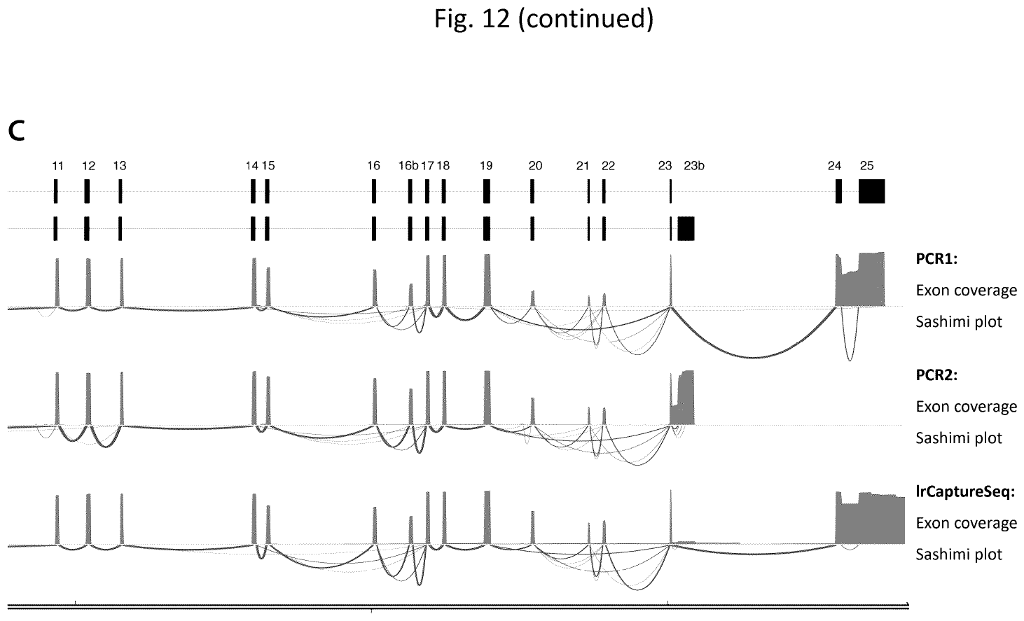

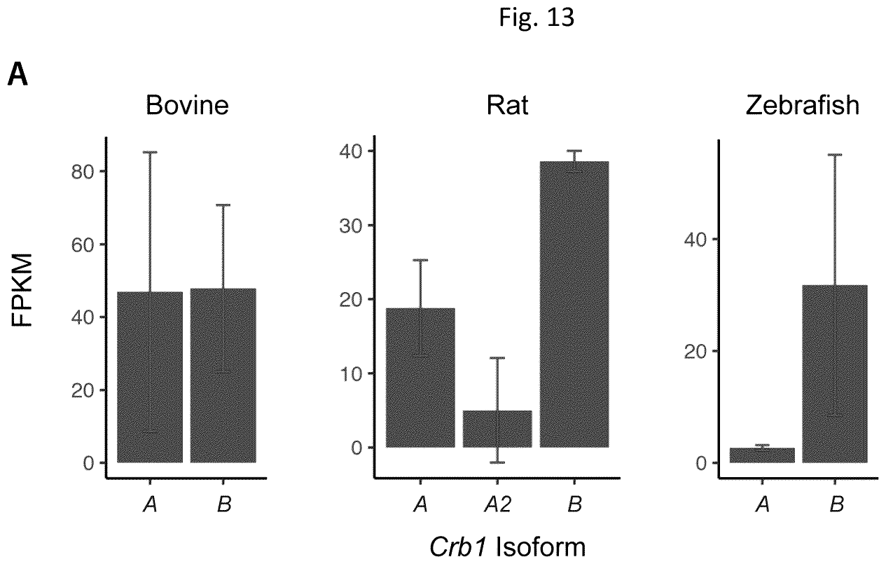

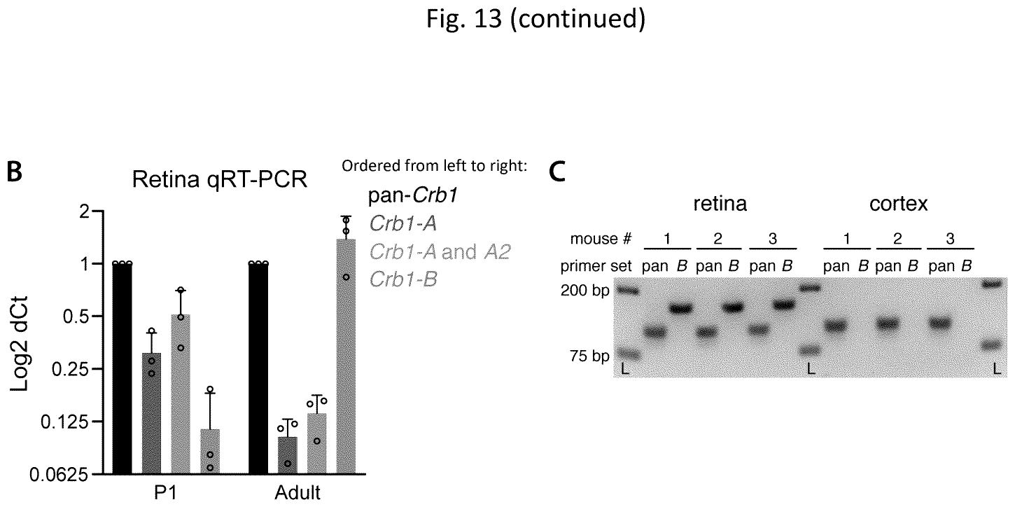

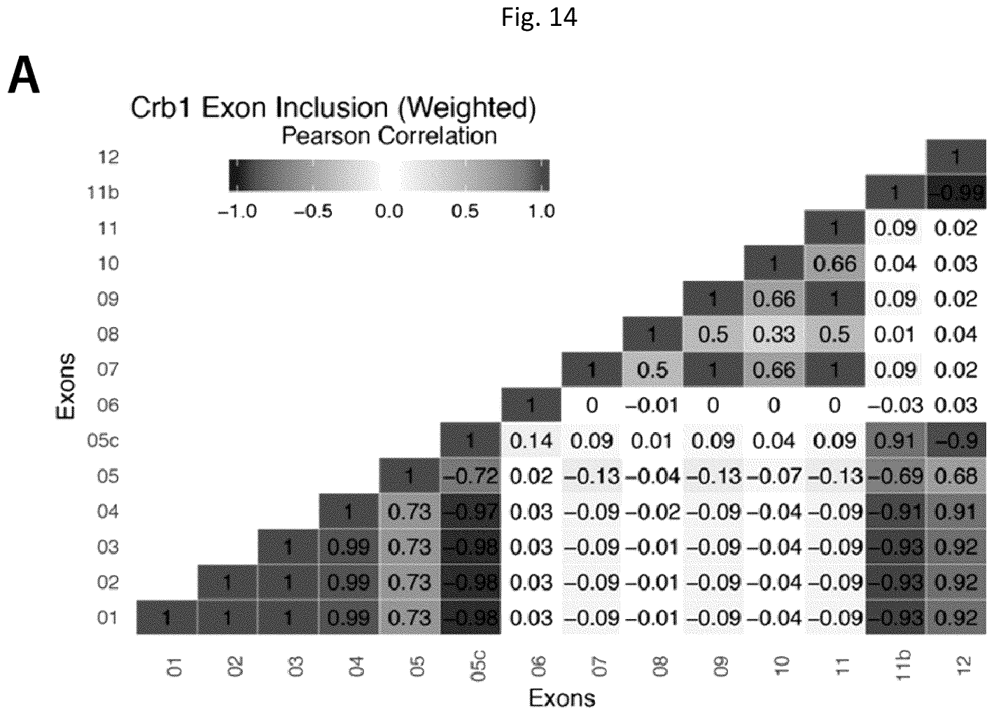

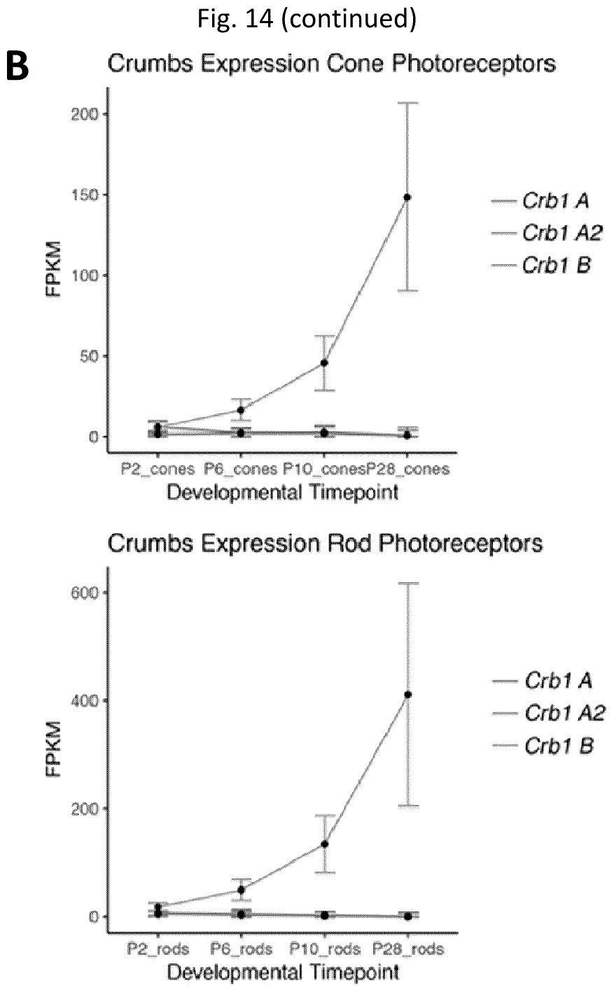

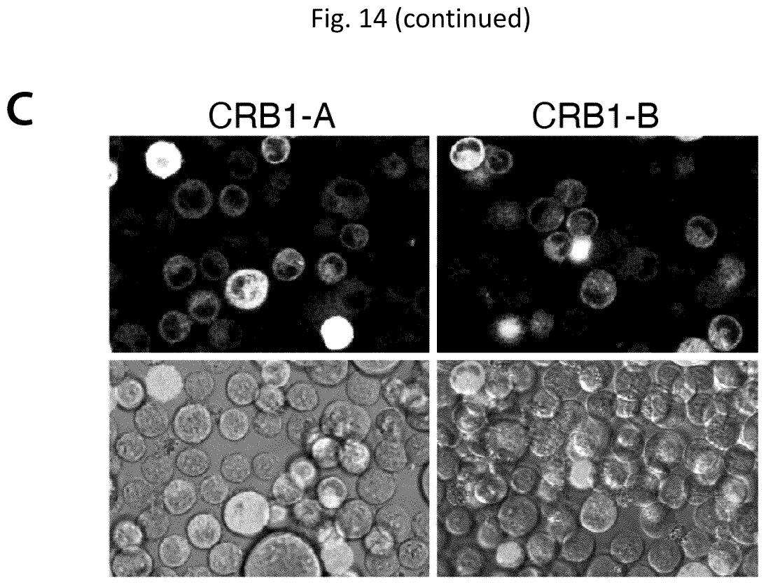

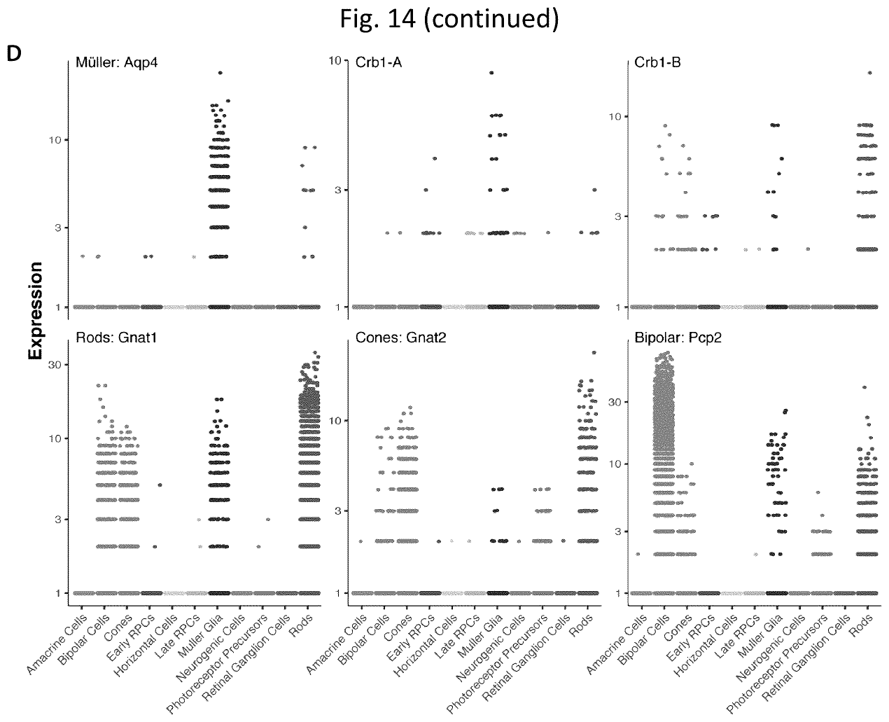

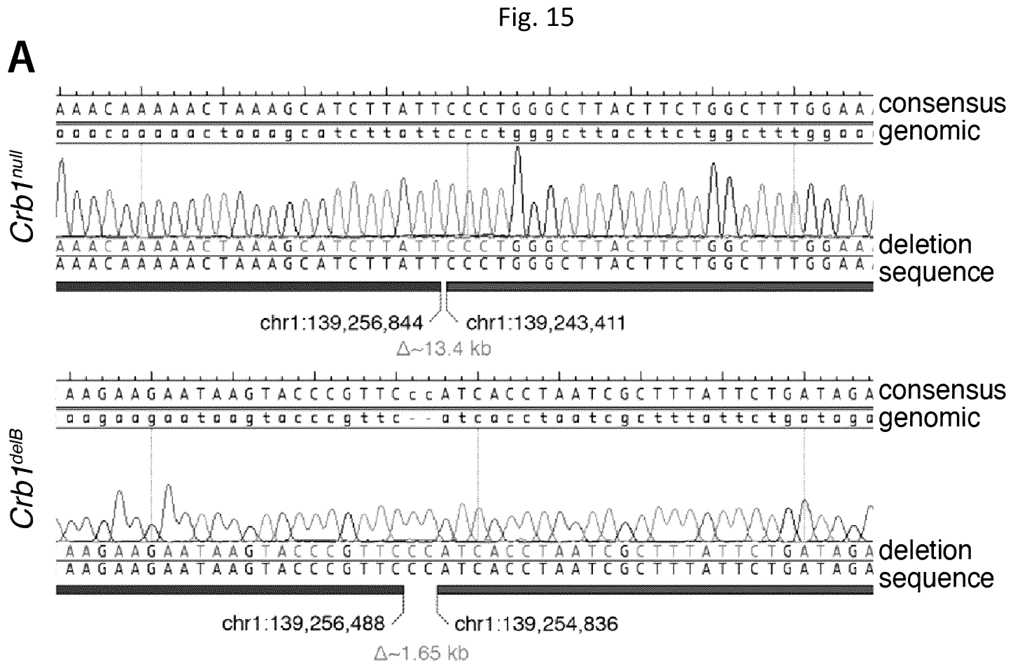

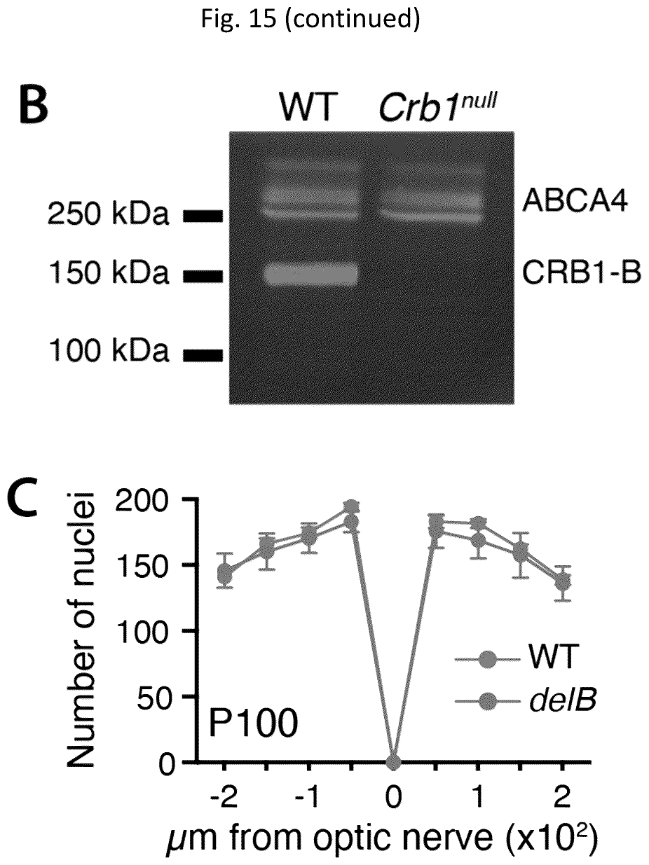

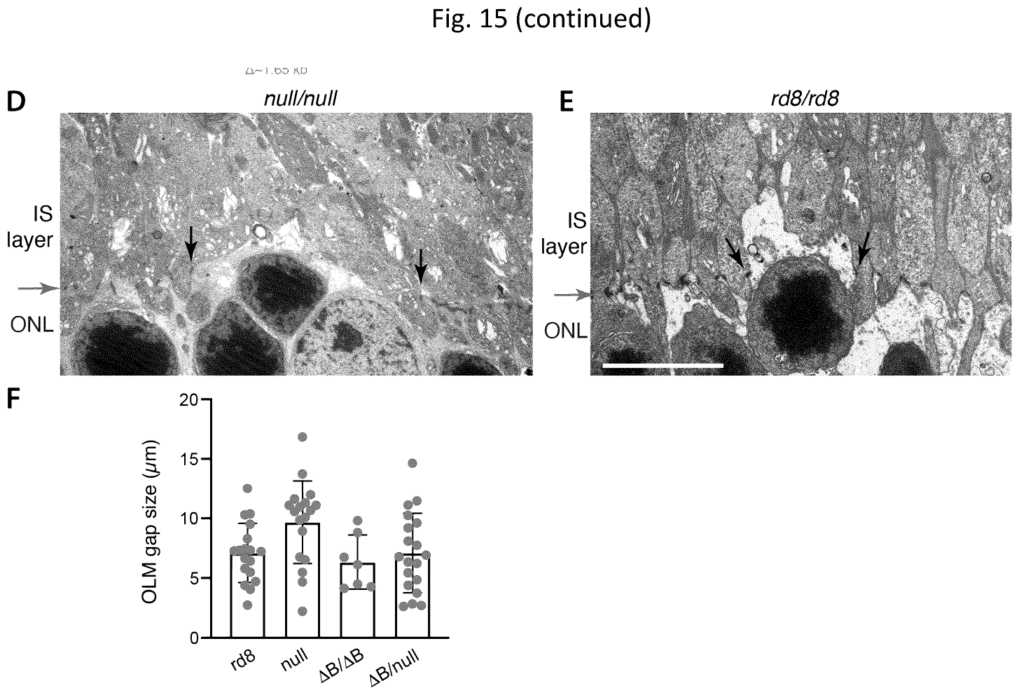

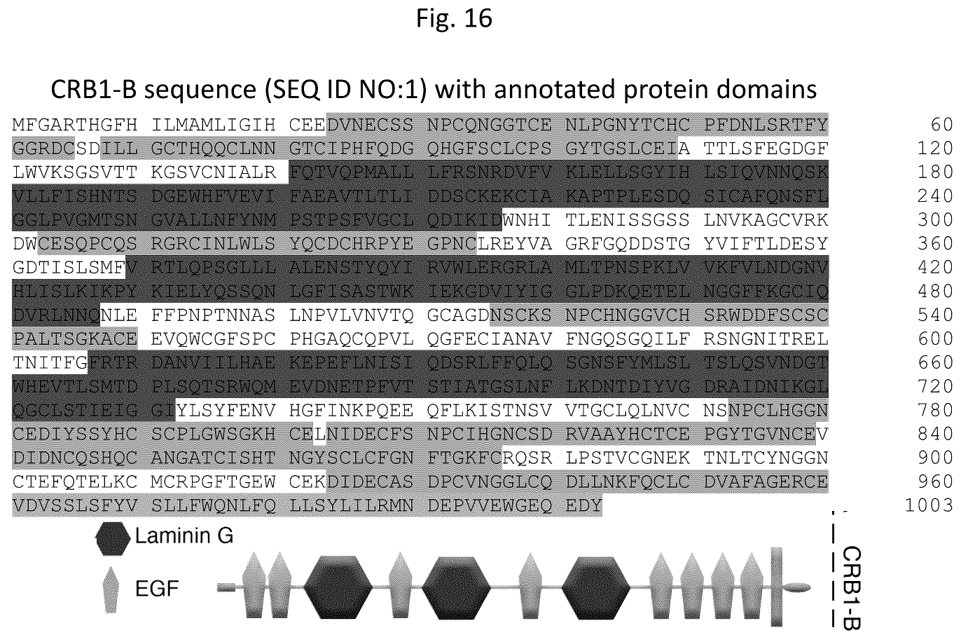

To determine whether mRNA diversity has a significant impact on protein sequences, we studied the predicted protein output of individual genes. In many cases, predicted proteins varied substantially in their inclusion of well-characterized features or functional domains. This phenomenon is exemplified by the Megf11 gene, which encodes a transmembrane EGF repeat protein implicated in cell-cell recognition during retinal development⁴⁴. Megf11 is subject to extensive alternative splicing: Out of 26 protein-coding exons, 21 are alternatively spliced (81%). In fact, we documented only 10 constitutive splice junctions within the 234 Megf11 isoforms identified in three independent long-read sequencing experiments (FIG. 4A,B; FIG. 12). Examination of predicted proteins revealed a potential reason for such extensive splicing: Most of the EGF repeats comprising the extracellular domain are encoded by individual exons, such that alternative splicing causes them to be deployed in a modular fashion (FIG. 4A-D). Intracellular domain exons also showed potential for modularity in the use of ITAM or ITIM signaling motifs (FIG. 4A-D), similar to the situation in its Drosophila homolog Draper⁴⁵. As a result of this modular organization, predicted MEGF11 proteins showed substantial variability in the number and/or identity of included EGF repeats (FIG. 4D). The most variable EGF repeats were encoded by exons 14-16b (FIG. 4B); however, most of the EGF repeats were subject to alternative usage. Using BaseScope™ in situ hybridization^{46,47}, we confirmed that each of the most variable exon junctions are expressed by retinal neurons in vivo (FIG. 4E). Remarkably, individual Megf11-expressing cells were found to use all of the exon junctions we tested, suggesting that extensive Megf11 isoform diversity is present even within individual neurons (FIG. 4E). Therefore, similar to insect Dscam1, Megf11 uses alternative splicing of modular extracellular domain features to create a large family of isoforms encoding distinct cell-surface molecules. Together with our analysis of the full lrCaptureSeq dataset, these findings strongly suggest that isoform diversity serves to diversify the neuronal cell-surface proteome in vivo.

### Cell-Surface Proteins Predicted by lrCaptureSeq are Expressed in Developing Retina

To determine whether novel lrCaptureSeq isoforms are translated into proteins, we performed mass spectrometry on cell-surface protein samples obtained from developing retina. Cell-surface proteins were captured using cell-impermeant reagents that either cleaved or biotinylated extracellular epitopes (FIG. 3E,F). To learn whether any of the captured peptides came from novel protein isoforms, we generated a database of possible trypsin peptide products derived from the isoforms within the lrCaptureSeq catalog. This was essential because protein identification requires comparison of raw mass spectrometry data to a reference peptide database. On generation of this new predicted peptide database, we found that it contained ˜25% more putative peptides for our 30 genes than the UniProt Mouse Reference Database typically used in most proteomics experiments (FIG. 11E). The extra putative peptides represent novel protein regions predicted by lrCaptureSeq.

Using this new database as a reference, we found 686 total peptides corresponding to 28 of the genes. 35 of these peptides were absent from the UniProt standard reference, and were present only in our new reference database (FIG. 3G). This fraction represents novel peptides, predicted from our lrCaptureSeq isoform catalog, that would have gone undetected in a typical mass spectrometry experiment. Novel peptides were found for 14 of our 30 genes, validating novel exonic sequences, splice junctions, and splice acceptor sites (data not shown). These findings demonstrate that at least some of the predicted proteins are expressed on the surface of retinal cells in vivo. Thus, the mRNA diversity we describe here contributes to the diversity of the retinal cell surface proteome.

### The Most Abundant Transcript in the lrCaptureSeq Database is a Novel Isoform of Crb1

To investigate whether newly-discovered isoforms could provide insight into gene function, we focused on Crb1, a well-known retinal disease gene. Our Crb1 catalog contained 15 isoforms, several of which were tissue-specific and developmentally-regulated (FIG. 5A,B; FIG. 13B,C). In mature retina, Crb1 expression was dominated by a single isoform—but not the one that has been the subject of virtually all previous Crb1 studies. Instead, the dominant isoform was a retina-specific variant bearing unique 5′ and 3′ exons (FIG. 5A,D; FIG. 14A) and a unique promoter site just upstream of the novel 5′ exon (FIG. 5C). We named this isoform Crb1-B, to distinguish it from the canonical Crb1-A isoform.

Even though Crb1-B was the most abundant of the 4, 116 isoforms in our dataset (FIG. 2D), it was not annotated in the major genome databases (RefSeq, GENCODE, or, UCSC). Nor, to our knowledge, was it documented in the literature. CRB1-B is also the most abundant isoform in human retina, as shown by a lrCaptureSeq dataset generated from human retinal cDNA (FIG. 5E,G). A third variant, CRB1-C, was also expressed in human retina at moderate levels—much higher than in mouse—but it was still not as abundant as CRB1-B (FIG. 5E,G). As in mouse, ATAC-seq data revealed a putative B isoform promoter in human retina (FIG. 5C,F). Using short-read datasets, we corroborated the mouse and human findings and then extended them to several other vertebrate species (FIG. 5H,I; FIG. 13A). Together, these results demonstrate that the major retinal isoform of an important disease gene had previously been overlooked: Across a range of vertebrate species, CRB1-B is the predominant CRB1 isoform in the retina.

### Crb1-A and Crb1-B Encode Cell-Surface Proteins Expressed in Different Cell Types

Crb1-B is predicted to encode a transmembrane protein sharing significant extracellular domain overlap with CRB1-A, but an entirely different intracellular domain (FIG. 6A,B). We therefore asked whether this protein is expressed and, if so, where the protein is localized. Western blotting with an antibody raised against the CRB1-B intracellular domain demonstrated that the protein exists in vivo (FIG. 6C). Moreover, it exists in the configuration predicted by lrCaptureSeq (FIG. 6A), because intracellular domain expression was absent in mice engineered to lack the Crb1-B promoter and 5′ exon (FIG. 6C; see FIG. 7A for mouse design). Consistent with the notion that CRB1-B is a transmembrane protein, it was detected in the membrane fraction but not the soluble fraction of retinal lysates (FIG. 6D). Further, when expressed in heterologous cells, CRB1-B trafficked to the plasma membrane in a manner strongly resembling CRB1-A (FIG. 14C). These data suggest that both major CRB1 isoforms localize at the cell surface.

To determine the expression patterns of Crb1-A and Crb1-B, we developed a strategy to evaluate expression of lrCaptureSeq isoforms within single cell (sc)-RNA-seq datasets. Applying this strategy to scRNA-seq data from developing mouse retina⁴⁸, we found distinct expression patterns for each isoform. Crb1-A was expressed largely by Müller glia (FIG. 6E,F; FIG. 14D), consistent with previous immunohistochemical studies^{37,49}. Crb1-B, by contrast, was expressed by rod and cone photoreceptors (FIG. 6E,F; FIG. 14B,D). These cell-type-specific expression patterns were validated using two independent methods: First, ATAC-seq data from rods and cones showed that photoreceptors selectively use the Crb1-B promoter (FIG. 5C). Second, BaseScope staining confirmed mutually exclusive expression of the two isoforms, with Crb1-A localizing to Müller cells and Crb1-B to photoreceptors (FIG. 6G).

To examine CRB1-B protein localization, we initially attempted immunohistochemistry but found that our antibody was not suitable. Therefore, we turned to a technique that combines serial tangential cryosectioning of the retina with Western blotting^{50,51}. Each tangential section contains a specific subset of cellular and subcellular structures that can be recognized by representative protein markers (FIG. 6H). This approach confirmed expression of CRB1-B in the photoreceptor layer, predominantly within the inner and outer segments. This localization is in marked contrast to CRB1-A which has been localized to the apical tips of Müller cells, within the OLM (FIG. 6E), using antibodies specific to this isoform^{37,49}.

### CRB1-B is Required for Integrity of the Outer Limiting Membrane

We next investigated the function of the CRB1-B isoform. Photoreceptors and Müller glia, the two cell types that express the major CRB1 isoforms (FIG. 6F,G), engage in specialized cell-cell junctions that form the OLM (FIG. 6E; FIG. 7B,C). It has been suggested that degenerative pathology in CRB1 disease may result from disruption of these junctions, but mouse studies have failed to clarify whether CRB1 is in fact required for OLM integrity. The two existing Crb1 mutant strains have conflicting OLM phenotypes: Mice bearing a Crb1 point mutation known as rd8 show sporadic OLM disruptions³⁶, whereas a Crb1 “knockout” allele, here denoted Crb1^{ex1}, fails to disturb OLM junctions³⁷. Our lrCaptureSeq data revealed a key difference between these two alleles: rd8 affects both Crb1-A and Crb1-B isoforms, whereas the “knockout” ex1 allele leaves Crb1-B intact (FIG. 7A). Therefore, we hypothesized that Crb1-B has a key role in the integrity of photoreceptor-Müller junctions at the OLM. To test this hypothesis, we generated two new mutant alleles (FIG. 7A; FIG. 15A,B). The first, Crb1^{delB}, abolishes Crb1-B while preserving other isoforms including Crb1-A. The second, Crb1^{null}, is a large deletion designed to disrupt all Crb1 isoforms.

Using electron microscopy to evaluate OLM integrity, we found that Crb1^{null} mutants exhibit disruptions at the OLM whereby photoreceptor nuclei invaded the inner segment layer, disturbing the structure of the outer retina (FIG. 7B-E; FIG. 15D). Within the disrupted regions, photoreceptor inner segments lacked their characteristic electron-dense junctions with apical Müller processes, indicating that OLM gaps arose due to disruption of photoreceptor-Müller contacts (FIG. 7F). A similar phenotype was also observed in Crb1^{rd8} mutants, as previously reported³⁶ (FIG. 7F,G,J; FIG. 15D-F). To explore the contribution of each isoform to the OLM phenotype, we examined mice bearing various combinations of the Crb1^{null} and Crb1^{delB} alleles. In Crb1^{delB/delB} mice, which lack Crb1-B but retain two copies of Crb1-A, the OLM phenotype was still evident but was weaker than in rd8 or null homozygotes (FIG. 7H,J). By contrast, the OLM phenotype was equivalent to rd8 and null mutants in Crb1^{delB} null mice, which lack Crb1-B but retain one copy of Crb1-A (FIG. 7E,J; FIG. 15F). These findings indicate that both Crb1 isoforms are needed for OLM junctional integrity, but the role of Crb1-B is particularly important, given that severe OLM disruptions can arise even when Crb1-A remains present.

### Retinal Degeneration in Mice Lacking All Crb1 Isoforms

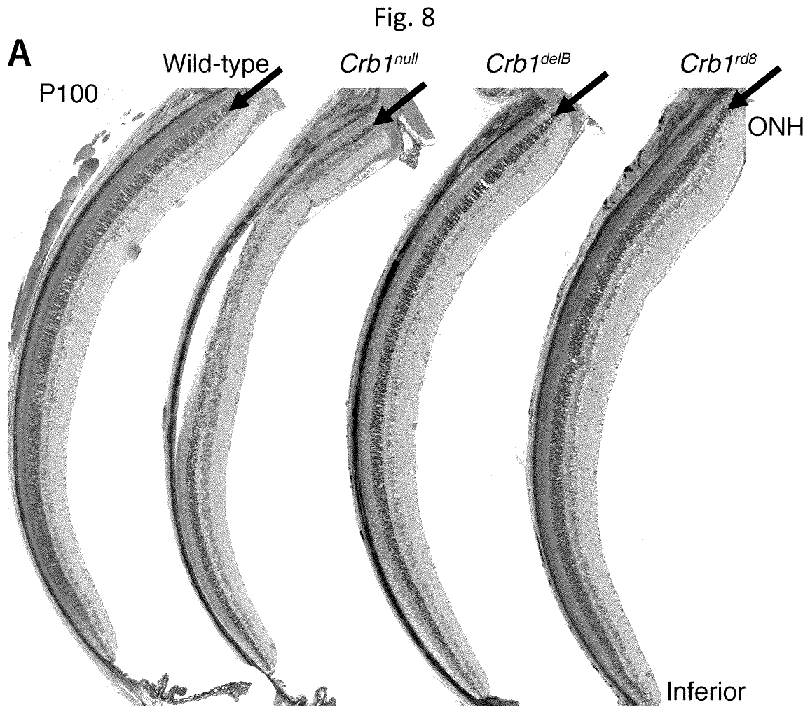

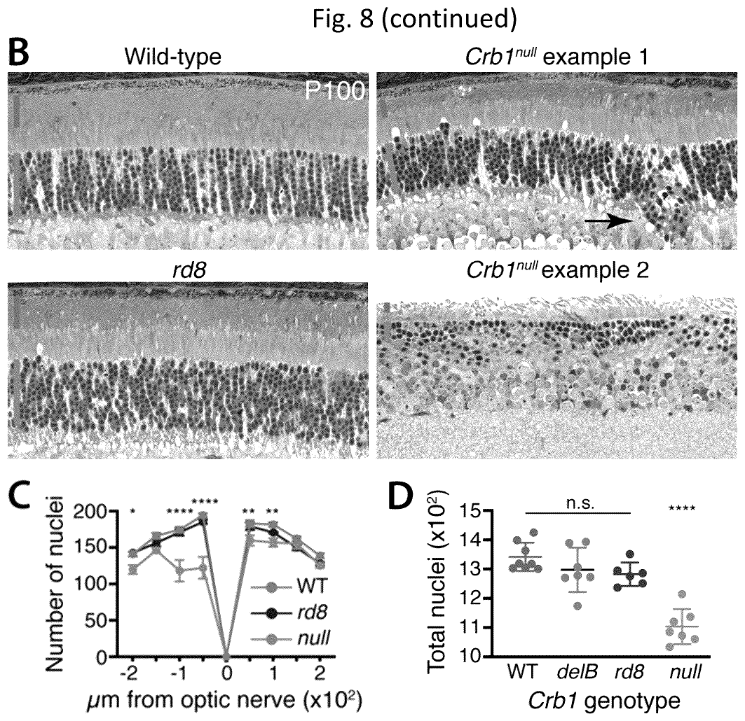

Finally, we asked whether insight into CRB1 isoforms could be used to improve animal models of CRB1 degenerative disease. Photoreceptor degeneration is absent or extremely slow in existing Crb1 loss-of-function mice, making them poor models of human degenerative phenotypes^{36,37,52}. We hypothesized that previously unannotated Crb1 isoforms, such as Crb1-B, might help explain these mild phenotypes. Consistent with this possibility, we noted that neither of the existing Crb1 mutant alleles completely eliminates all Crb1 isoforms (FIG. 7A). To test the contribution of new Crb1 isoforms to photoreceptor degeneration, we took advantage of our newly-generated Crb1^{delB} and Crb1^{null} strains (FIG. 7A). Analysis of photoreceptor numbers in young adult mice (P100) revealed that both Crb1-A and Crb1-B isoforms are required for photoreceptor survival. Crb1^{delB} mutants had normal photoreceptor numbers (FIG. 8A,D; FIG. 15C), similar to the previously-reported Crb1^{ex1} mutant³⁷. Therefore, removing either major isoform by itself has minimal degenerative effects. By contrast, deletion of all isoforms in Crb1^{null} mice caused marked photoreceptor degeneration (FIG. 8A-D). Thus, significant cell loss requires compromise of both Crb1-A and Crb1-B. No degeneration was evident yet at P100 in Crb1^{rd8} mutants (FIG. 8B-D), consistent with previous reports that significant degeneration takes ˜2 years^{36,52,53}. Together, these genetic experiments support the conclusion that multiple Crb1 isoforms contribute to photoreceptor survival—including the novel Crb1-B isoform. Thus, modeling of human disease can be achieved by rational design of mutant alleles guided by lrCaptureSeq isoform catalogs.

### Discussion

Despite recent advances in sequencing technology, the true diversity of the CNS transcriptome remains surprisingly murky¹². For most genes, only a small subset of the full isoform portfolio has been documented. Here we show that lrCaptureSeq can unveil isoform diversity with an unprecedented level of detail. LrCaptureSeq is accurate and efficient, with sufficient depth to reveal the full-length sequence of even low-abundance isoforms. To facilitate interpretation of lrCaptureSeq data we provide a companion R software package for analyzing and visualizing isoform catalogs. Applying these new tools to the developing nervous system, we uncovered a vast diversity of isoforms encoding cell surface proteins, most of which were novel. Many were predicted to alter functional protein domains. Further, we found that the most abundant isoform in our entire dataset—a novel isoform of the Crb1 disease gene—has a distinct expression pattern and function from the canonical isoform, endowing it with disease-relevant functions. CRB1 therefore serves as a striking example of the value of comprehensive full-length isoform identification. We propose that lrCaptureSeq can be applied to generate full-length isoform catalogs for many different CNS regions and cell types, an approach that is likely to unlock many new insights into CNS gene function and dysfunction.

Isoform identification requires substantial sequencing depth. Even with short read RNA-seq, complete isoform portfolios are likely detectable only for the most abundant genes, given that the least abundant 44% of transcripts garner only 1% of the reads^{31,54}. Targeted CaptureSeq approaches have been used to improve short-read detection of low-abundance transcripts^{31,55} Here, applying this strategy for long-read sequencing of protein-coding mRNAs, we obtained deep full-length coverage for a group of genes that would be poorly represented in existing PacBio transcriptomes, due to their cDNA size and expression levels. It is clear from the distribution of isoform abundances (FIG. 2C) that only the least abundant isoforms escaped detection. Some isoforms smaller than 4.5 kb may also have evaded detection, given the size selection step of our library preparation protocol (FIG. 1B). For these reasons we suspect that we have not detected every last isoform. However, even with our enrichment for long transcripts, we still obtained a large sample of shorter reads (FIG. 10E) and identified many smaller isoforms—including Crb1-B (3.0 kb). Thus, while the lrCaptureSeq catalogs may lack certain short and/or rare transcripts, we conclude that we have detected most of the isoforms expressed in our targeted tissues. We achieved this depth by targeting 30 genes for parallel sequencing, but higher-throughput PacBio instruments are now available; these should allow substantially more targeted genes to be sequenced in parallel without sacrificing isoform coverage.

Our results suggest many potential uses for lrCaptureSeq in transcriptome annotation. One particularly exciting use case is identification of cell-type-specific isoform expression patterns. We show that lrCaptureSeq data can be integrated with existing short-read RNA-seq datasets, including single-cell data, to reveal the time and place of isoform expression. As of now, this expression mapping works best for isoforms that differ at their 3′ ends, due to 3′ bias inherent in most single-cell library preparation methods. In the future, as scRNA-seq methods are refined to improve depth and coverage, we expect that other types of isoforms will be amenable to mapping in this way. With this methodology, it will not be necessary to generate lrCaptureSeq catalogs for each cell type in the nervous system; rather, cell-type-specific isoform expression can be determined bioinformatically by combining different types of sequencing data.

How many mRNA isoforms are produced by any given gene? For the 30 genes in our dataset the median number of RefSeq isoforms was 11.5, and no gene had more than 51. By contrast, the median number of isoforms in our lrCaptureSeq catalog was 50, while the most diverse gene, Nrxn3, had nearly 900. Overall, the number of lrCaptureSeq isoforms exceeded the number annotated in reference transcriptomes by nearly an order of magnitude (FIG. 2B). By contrast, a previous CaptureSeq study of long noncoding RNAs found only two-fold more isoforms⁴⁰. Thus, even though it is widely recognized that most genes generate multiple isoforms, the scale of diversity we uncovered for cell-surface molecules was still surprising. Our 30 genes probably have more isoforms than the average gene, given that they were selected because they showed evidence of transcript diversity (FIG. 1A). Whether such diversity is typical of other gene classes and other tissues remains to be determined-perhaps through broader application of the lrCaptureSeq methodology.

It has long been suspected that extensive diversity of cell-surface proteins might be involved in formation of precise neuronal connections^{5,8,56}. However, the need for numerous cell-surface cues has recently been called into question⁵⁷. In this view, extensive diversity would be required only in certain select contexts, such as during the self vs. non-self recognition mediated by Dscam1 and clustered protocadherins^{58,59}. Here we show that extensive isoform diversity is widespread across many cell-surface receptor genes, and that individual neurons most likely express numerous of isoforms of certain genes (e.g. Megf11; FIG. 4E). Thus, the molecular prerequisite for the “numerous cues” model is in place. Strikingly, the genes that have the most predicted protein diversity share a common function as trans-synaptic cell adhesion molecules (FIG. 3C). Many of these genes have known roles in synapse formation^{19,60,61}. Therefore, these diverse molecular cues are likely positioned in exactly the right place to influence the precision of synaptic connections. It will be interesting to learn the extent to which isoforms described here function in synapse specificity.

A striking feature of Dscam1 isoform diversity is the modular deployment of Ig repeats to modify binding specificity. Other genes with equivalently high potential for modular swapping of extracellular domain motifs have not previously been identified. Here we show that Megf11, a recognition molecule that mediates homotypic cell-cell repulsion during retinal development⁴⁴, diversifies its extracellular domain through extensive modular use of EGF-like repeats. The phenomenon of modular EGF-repeat swapping through alternative splicing has been observed before, albeit at smaller scale, for Netrin-G proteins⁶². Therefore, it is possible that many EGF-repeat genes may generate large families of cell surface proteins using a similar modular strategy.

Our studies of CRB1 illustrate the value and importance of documenting the complete isoform output of individual genes. CRB1 is a major causal gene for inherited retinal degenerative diseases, including Leber's congenital amaurosis, retinitis pigmentosa, and macular dystrophy⁶³⁻⁶⁵. As such, both mouse Crb1 and human CRB1 have been studied intensively. Nevertheless, the major CRB1 isoform in mature human retina—CRB1-B—had evaded detection until now. CRB1-B may have been overlooked because its 5′ and 3′ exons are the only parts of the transcript that distinguish it from CRB1-A. With short-read sequencing it is difficult to tell that these two distant exons are typically used together in the same transcript. By contrast, lrCaptureSeq clearly showed that the most abundant retinal CRB1 isoform was a novel variant containing these unconventional 5′ and 3′ exons.

Due to their distinct 5′ and 3′ ends (FIG. 6A), Crb1-A and-B differ in crucial ways that likely endow them with distinct functions. Their 5′ exons have different promoters that drive expression in different cell types-Crb1-A in Müller glia and Crb1-B in photoreceptors-while their 3′ exons encode different intracellular domains. The CRB1-A intracellular domain, like other vertebrate homologs of Drosophila Crumbs, contains two highly-conserved motifs mediating interactions with polarity proteins known as the Crumbs complex⁶⁶. These motifs localize Crumbs homologs to apical junctions, where they are required for maintaining epithelial structural integrity and apico-basal polarity³³. CRB1-B lacks these conserved motifs, suggesting a model whereby CRB1-A and -B operate in different cell types through different intracellular interaction partners.

Our findings have implications for the prevailing model of CRB1 disease, which posits that CRB1 and the Crumbs complex are required for integrity of OLM junctions between Müller glia and photoreceptors²⁶. A major challenge for this model has been the lack of OLM phenotypes or photoreceptor degeneration in Crb1^{ex1} mutants³⁷, which lack CRB1-A (FIG. 7A). As this mutant mouse was thought to be a null allele, its weak phenotype suggested that CRB1 might be dispensable for photoreceptor survival in mice²⁶. Here we show that CRB1 is indeed required for OLM integrity and photoreceptor survival, but the mechanism critically involves the photoreceptor-specific CRB1-B isoform. Moreover, we show a genetic interaction between the two isoforms, revealing OLM integrity and pro-survival functions for CRB1-A that were obscured in the Crb1^{ex1} mutant strain. We propose that the concerted action of CRB1-A in glia and CRB1-B in photoreceptors controls OLM integrity and photoreceptor health, perhaps through the assembly or maintenance of the junctional protein complex in each respective cell type.

The notion of concerted Crb1-A and Crb1-B function is further supported by the fact that Crb1^{rd8}, a point mutation affecting both isoforms (FIG. 7A), exhibits more degeneration than Crb1^{ex1 36,37,53}. However, Crb1^{rd8} is clearly less severe than Crb1^{null} (FIG. 8), even though both A and B isoforms are affected in both mutants. One possible reason for this difference is that Crb1^{rd8} may not be a mRNA or protein null⁵³. Another possibility is that the Crb1-C isoform may play a compensatory role, as it is unaffected by Crb1^{rd8} (FIG. 7A). Either way, our results show that the design of mouse disease models is significantly enhanced when a complete isoform catalog is available.

Overall, our work highlights the value of building complete and accurate full-length isoform catalogs. Lack of such information can cause key gene functions to be overlooked and can lead to misinterpretation of genetic experiments and disease phenotypes. We expect the transcriptomic “ground truth” provided by deep long-read capture sequencing will be an important addition to the transcriptome annotation toolbox, enabling discovery of specific mRNA isoforms that contribute to a wide range of normal and disease processes.

### References

1. Raj, B. & Blencowe, B. J. Alternative Splicing in the Mammalian Nervous System: Recent Insights into Mechanisms and Functional Roles. Neuron 87, 14-27 (2015).
2. Reyes, A. & Huber, W. Alternative start and termination sites of transcription drive most transcript isoform differences across human tissues. Nucleic Acids Res. 46, 582-592 (2018).
3 Taliaferro, J. M. et al. Distal Alternative Last Exons Localize mRNAs to Neural Projections. Mol. Cell 61, 821-33 (2016).
4. Tushev, G. et al. Alternative 3′ UTRs Modify the Localization, Regulatory Potential, Stability, and Plasticity of mRNAs in Neuronal Compartments. Neuron 98, 495-511.e6 (2018).
5. Furlanis, E., Traunmüller, L., Fucile, G. & Scheiffele, P. Landscape of ribosome-engaged transcript isoforms reveals extensive neuronal-cell-class-specific alternative splicing programs. Nat. Neurosci. 22, 1709-1717 (2019).
6. Takahashi, H. & Craig, A. M. Protein tyrosine phosphatases PTPδ, PTPσ, and LAR: presynaptic hubs for synapse organization. Trends Neurosci. 36, 522-34 (2013).
7. Lipscombe, D. & Lopez Soto, E. J. Alternative splicing of neuronal genes: new mechanisms and new therapies. Curr. Opin. Neurobiol. 57, 26-31 (2019).
8. Zipursky, S. L. & Sanes, J. R. Chemoaffinity revisited: dscams, protocadherins, and neural circuit assembly. Cell 143, 343-53 (2010).
9. Gandal, M. J. et al. Transcriptome-wide isoform-level dysregulation in ASD, schizophrenia, and bipolar disorder. Science (80-.). 362, eaat8127 (2018).
10. Taylor, J. P., Brown, R. H. & Cleveland, D. W. Decoding ALS: from genes to mechanism. Nature 539, 197-206 (2016).
11. Li, Y. I. et al. RNA splicing is a primary link between genetic variation and disease. Science 352, 600-4 (2016).
12. Morillon, A. & Gautheret, D. Bridging the gap between reference and real transcriptomes. Genome Biol. 20, 112 (2019).
13. Schmucker, D. et al. Drosophila Dscam is an axon guidance receptor exhibiting extraordinary molecular diversity. Cell 101, 671-84 (2000).
14. Chen, W. V. & Maniatis, T. Clustered protocadherins. Development 140, 3297-3302 (2013).
15. Schreiner, D. et al. Targeted Combinatorial Alternative Splicing Generates Brain Region-Specific Repertoires of Neurexins. Neuron 1-13 (2014). doi: 10.1016/j.neuron.2014.09.011
16. Treutlein, B., Gokce, O., Quake, S. R. & Südhof, T. C. Cartography of neurexin alternative splicing mapped by single-molecule long-read mRNA sequencing. Proc. Natl. Acad. Sci. U. S. A. 111, E1291-9 (2014).
17. Rubinstein, R. et al. Molecular Logic of Neuronal Self-Recognition through Protocadherin Domain Interactions. Cell 163, 629-642 (2015).
18. Wojtowicz, W. M. et al. A vast repertoire of Dscam binding specificities arises from modular interactions of variable Ig domains. Cell 130, 1134-45 (2007).
19. Furlanis, E. & Scheiffele, P. Regulation of Neuronal Differentiation, Function, and Plasticity by Alternative Splicing. Annu. Rev. Cell Dev. Biol. 34, 451-469 (2018).
20. Südhof, T. C. Neuroligins and neurexins link synaptic function to cognitive disease. Nature 455, 903-11 (2008).
21. Mulley, J. C., Scheffer, I. E., Petrou, S. & Berkovic, S. F. Channelopathies as a genetic cause of epilepsy. Curr. Opin. Neurol. 16, 171-6 (2003).
22. Pederick, D. T. et al. Abnormal Cell Sorting Underlies the Unique X-Linked Inheritance of PCDH19 Epilepsy. Neuron 97, 59-66.e5 (2018).
23. Hammond, T. R., Marsh, S. E. & Stevens, B. Immune Signaling in Neurodegeneration. Immunity 50, 955-974 (2019).
24. Hollingworth, P. et al. Common variants at ABCA7, MS4A6A/MS4A4E, EPHA1, CD33 and CD2AP are associated with Alzheimer's disease. Nat. Genet. 43, 429-35 (2011).
25. Naj, A. C. et al. Common variants at MS4A4/MS4A6E, CD2AP, CD33 and EPHA1 are associated with late-onset Alzheimer's disease. Nat. Genet. 43, 436-441 (2011).
26. Quinn, P. M., Pellissier, L. P. & Wijnholds, J. The CRB1 Complex: Following the Trail of Crumbs to a Feasible Gene Therapy Strategy. Front. Neurosci. 11, 175 (2017).
27. Au, K. F. et al. Characterization of the human ESC transcriptome by hybrid sequencing. Proc. Natl. Acad. Sci. U. S. A. 110, E4821-30 (2013).
28. Byrne, A. et al. Nanopore long-read RNAseq reveals widespread transcriptional variation among the surface receptors of individual B cells. Nat. Commun. 8, 16027 (2017).
29. Gupta, I. et al. Single-cell isoform RNA sequencing characterizes isoforms in thousands of cerebellar cells. Nat. Biotechnol. 36, (2018).
30. Karlsson, K. & Linnarsson, S. Single-cell mRNA isoform diversity in the mouse brain. BMC Genomics 18, 126 (2017).
31. Bussotti, G. et al. Improved definition of the mouse transcriptome via targeted RNA sequencing. Genome Res. 26, 705-716 (2016).
32. Mercer, T. R. et al. Targeted RNA sequencing reveals the deep complexity of the human transcriptome. Nat. Biotechnol. 30, 99-104 (2012).
33. Thompson, B. J., Pichaud, F. & Röper, K. Sticking together the Crumbs—an unexpected function for an old friend. Nat. Rev. Mol. Cell Biol. 14, 307-14 (2013).
34. Vecino, E., Rodriguez, F. D., Ruzafa, N., Pereiro, X. & Sharma, S. C. Glia-neuron interactions in the mammalian retina. Prog. Retin. Eye Res. 51, 1-40 (2016).
35. Ehrenberg, M., Pierce, E. A., Cox, G. F. & Fulton, A. B. CRB1: one gene, many phenotypes. Semin. Ophthalmol. 28, 397-405 (2013).
36. Mehalow, A. K. et al. CRB1 is essential for external limiting membrane integrity and photoreceptor morphogenesis in the mammalian retina. Hum. Mol. Genet. 12, 2179-89 (2003).
37. van de Pavert, S. a et al. Crumbs homologue 1 is required for maintenance of photoreceptor cell polarization and adhesion during light exposure. J. Cell Sci. 117, 4169-77(2004).
38. Hoshino, A. et al. Molecular Anatomy of the Developing Human Retina. Dev. Cell 43, 763-779.e4 (2017).
39. Peng, J. et al. High-Throughput Sequencing and Co-Expression Network Analysis of IncRNAs and mRNAs in Early Brain Injury Following Experimental Subarachnoid Haemorrhage. Sci. Rep. 7, 46577 (2017).
40. Lagarde, J. et al. High-throughput annotation of full-length long noncoding RNAs with capture long-read sequencing. Nat. Genet. 49, 1731-1740 (2017).
41. Shiraki, T. et al. Cap analysis gene expression for high-throughput analysis of transcriptional starting point and identification of promoter usage. Proc. Natl. Acad. Sci. 100, 15776-15781 (2003).
42. Yasuda, M. et al. Retinal transcriptome profiling at transcription start sites: a cap analysis of gene expression early after axonal injury. BMC Genomics 15, 982 (2014).
43. Magurran, A. Measuring Biological Diversity. (Wiley-Blackwell, 2004).
44. Kay, J. N., Chu, M. W. & Sanes, J. R. MEGF10 and MEGF11 mediate homotypic interactions required for mosaic spacing of retinal neurons. Nature 483, 465-9 (2012).
45. Logan, M. a et al. Negative regulation of glial engulfment activity by Draper terminates glial responses to axon injury. Nat. Neurosci. 15, 722-30 (2012).
46. Baker, A.-M. et al. Robust RNA-based in situ mutation detection delineates colorectal cancer subclonal evolution. Nat. Commun. 8, 1998 (2017).
47. Erben, L., He, M.-X., Laeremans, A., Park, E. & Buonanno, A. A Novel Ultrasensitive In Situ Hybridization Approach to Detect Short Sequences and Splice Variants with Cellular Resolution. Mol. Neurobiol. 55, 6169-6181 (2018).
48. Clark, B. S. et al. Single-Cell RNA-Seq Analysis of Retinal Development Identifies NFI Factors as Regulating Mitotic Exit and Late-Born Cell Specification. Neuron 102, 1111-1126.e5 (2019).
49. van Rossum, A. G. S. H. et al. Pals1/Mpp5 is required for correct localization of Crb1 at the subapical region in polarized Muller glia cells. Hum. Mol. Genet. 15, 2659-72 (2006).
50. Lobanova, E. S. et al. Transducin gamma-subunit sets expression levels of alpha- and beta-subunits and is crucial for rod viability. J. Neurosci. 28, 3510-20 (2008).
51. Sokolov, M. et al. Massive light-driven translocation of transducin between the two major compartments of rod cells: a novel mechanism of light adaptation. Neuron 34, 95-106 (2002).
52. Moore, B. A. et al. A Population Study of Common Ocular Abnormalities in C57BL/6N rd8 Mice. Investig. Opthalmology Vis. Sci. 59, 2252 (2018).
53. Luhmann, U. F. O. et al. The severity of retinal pathology in homozygous Crb1rd8/rd8 mice is dependent on additional genetic factors. Hum. Mol. Genet. 24, 128-141 (2015).
54. Jiang, L. et al. Synthetic spike-in standards for RNA-seq experiments. Genome Res. 21, 1543-51 (2011).
55. Clark, M. B. et al. Quantitative gene profiling of long noncoding RNAs with targeted RNA sequencing. Nat. Methods 12, 339-42 (2015).
56. Sperry, R. W. Chemoaffinity in the orderly growth of nerve fiber patterns and connections. Proc. Natl. Acad. Sci. U. S. A. 50, 703-10 (1963).
57. Hassan, B. A. & Hiesinger, P. R. Beyond Molecular Codes: Simple Rules to Wire Complex Brains. Cell 163, 285-291 (2015).
58. Lefebvre, J. L., Sanes, J. R. & Kay, J. N. Development of dendritic form and function. Annu. Rev. Cell Dev. Biol. 31, 741-77 (2015).
59. Zipursky, S. L. & Grueber, W. B. The molecular basis of self-avoidance. Annu. Rev. Neurosci. 36, 547-68 (2013).
60 Li, Y. et al. Splicing-Dependent Trans-synaptic SALM3-LAR-RPTP Interactions Regulate Excitatory Synapse Development and Locomotion. Cell Rep. 12, 1618-1630 (2015).
61. Sando, R., Jiang, X. & Südhof, T. C. Latrophilin GPCRs direct synapse specificity by coincident binding of FLRTs and teneurins. Science 363, eaav7969 (2019).
62. Yin, Y., Miner, J. H. & Sanes, J. R. Laminets: laminin-and netrin-related genes expressed in distinct neuronal subsets. Mol. Cell. Neurosci. 19, 344-58 (2002).
63. den Hollander, A. I. et al. Mutations in a human homologue of Drosophila crumbs cause retinitis pigmentosa (RP12). Nat. Genet. 23, 217-21 (1999).
64. den Hollander, A. I. et al. Leber Congenital Amaurosis and Retinitis Pigmentosa with Coats-like Exudative Vasculopathy Are Associated with Mutations in the Crumbs Homologue 1 (CRB1) Gene. Am. J. Hum. Genet. 69, 198-203 (2001).
65. Khan, K. N. et al. A clinical and molecular characterisation of CRB1-associated maculopathy. Eur. J. Hum. Genet. 26, 687-694 (2018).
66. den Hollander, A. I. et al. CRB1 has a cytoplasmic domain that is functionally conserved between human and Drosophila. Hum. Mol. Genet. 10, 2767-2773 (2001).
67. Tardaguila, M. et al. SQANTI: extensive characterization of long-read transcript sequences for quality control in full-length transcriptome identification and quantification. Genome Res. 28, 396-411 (2018).
68. Aldiri, I. et al. The Dynamic Epigenetic Landscape of the Retina During Development, Reprogramming, and Tumorigenesis. Neuron 94, 550-568.e10 (2017).
69. Hughes, A. E. O., Enright, J. M., Myers, C. A., Shen, S. Q. & Corbo, J. C. Cell Type-Specific Epigenomic Analysis Reveals a Uniquely Closed Chromatin Architecture in Mouse Rod Photoreceptors. Sci. Rep. 7, 43184 (2017).
70. Wang, J. et al. ATAC-Seq analysis reveals a widespread decrease of chromatin accessibility in age-related macular degeneration. Nat. Commun. 9, 1364 (2018).
71. Davis, C. A. et al. The Encyclopedia of DNA elements (ENCODE): data portal update. Nucleic Acids Res. 46, D794-D801 (2018).
72. Qiu, X. et al. Single-cell mRNA quantification and differential analysis with Census. Nat. Methods 14, 309-315 (2017).
73. Trapnell, C. et al. The dynamics and regulators of cell fate decisions are revealed by pseudotemporal ordering of single cells. Nat. Biotechnol. 32, 381-386 (2014).
74. Puñal, V. M. et al. Large-scale death of retinal astrocytes during normal development mediated by microglia. bioRxiv 593731 (2019). doi: 10.1101/593731
75. Schindelin, J. et al. Fiji: an open-source platform for biological-image analysis. Nat. Methods 9, 676-82 (2012).
76. Smolders, K., Lombaert, N., Valkenborg, D., Baggerman, G. & Arckens, L. An effective plasma membrane proteomics approach for small tissue samples. Sci. Rep. 5, 10917 (2015).

What is claimed:

1. A recombinant vector comprising a polynucleotide encoding a Crumbs 1-B (CRB1-B) isoform, wherein the CRB1-B isoform comprises an N-terminal signal peptide linked to an extracellular polypeptide comprising, from N-terminus-to-C-terminus: two EGF domains, a lamG domain, an EGF domain, a lamG domain, an EGF domain, a lamG domain, and four EGF domains; wherein the C terminus of the extracellular polypeptide is linked to a C-terminal domain comprising a transmembrane domain and intracellular domain.

2. The recombinant vector of claim 1, wherein the polynucleotide is operably linked to a heterologous promoter capable of expressing the isoform in a retinal cell.

3. The recombinant vector of claim 1, wherein the extracellular polypeptide extends from the N-terminus of the ninth EGF domain of a CRB1-A isoform to the C-terminus of the sixteenth EGF domain of the CRB1-A isoform.

4. The recombinant vector of claim 1, wherein the C-terminal domain comprises SEQ ID NO: 3.

5. The recombinant vector of claim 1, wherein the retinal cell is selected from the group consisting of a photoreceptor cells, a retinal pigmented epithelial cell, a bipolar cell, a horizontal cell, an amacrine cell, a Müller cell, and/or a ganglion cell.

6. The recombinant vector according to claim 5, wherein the retinal cell comprises a photoreceptor cell. The recombinant vector of claim 2, wherein the promoter is selected from the group consisting of a rhodopsin kinase (RK) promoter, an opsin promoter, a Cytomegalovirus (CMV) promoter, and a chicken β-actin (CBA promoter).

8. The recombinant vector of claim 1, wherein the vector is a viral vector.

9. The recombinant vector of claim 8, wherein the viral vector is an AAV vector.

10. An isolated polypeptide made from the recombinant vector of claim 1.

11. A pharmaceutical composition comprising the recombinant vector of claim 1 and a pharmaceutically acceptable carrier.

12. The pharmaceutical composition of claim 11, further comprising a second vector encoding CRB1-A, CRB1-A2, CRB1-C, or combinations thereof.

13. A method of treating an ocular disorder, reducing progression of loss of vision or maintaining vision function in a subject, the method comprising administering the subject a therapeutically effective amount of the recombinant vector of claim 1 such that the ocular disorder is treated, the loss of vision is reduced or vision function is maintained in the subject.

14. The method of claim 13, wherein the subject has a mutation in one or more alleles of CRB1.

15. The method of claim 13, wherein the subject has a retinopathy.

16. The method of claim 13, wherein the recombinant vector is administered intravitreally, subretinally or topically.

17. The method of claim 13, further comprising monitoring the vision function of the subject after administration.

18. The method of claim 17, wherein the vision function of the subject is monitored by microperimetry, dark-adapted perimetry, assessment of visual nobility, visual acuity, ERG, or reading assessment.

19. A kit for treating an ocular disorder, reducing progression of an ocular disorder, reducing loss of vision, or maintaining vision function in a subject, the kit comprising the recombinant vector of claim 1, a device for delivery of the recombinant vector to the subject, and instructions for use.

20. The kit according to claim 19, in which the delivery comprises subretinal delivery, intravitreal delivery or topical delivery.


SEQUENCE LISTING (100 sequences)

SEQ ID NO: 1 - protein, 1003 aa, Homo sapiens
MFGARTHGFHILMAMLIGIHCEEDVNECSSNPCQNGGTCENLPGNYTCHCPFDNLSRTFYGGRDCSDILLGCTHQQCLNNGTCIPHFQDGQHGFSCLCPSGYTGSLCEIATTLSFEGDGFLWVKSGSVTTKGSVCNIALRFQTVQPMALLLFRSNRDVFVKLELLSGYIHLSIQVNNQSKVLLFISHNTSDGEWHFVEVIFAEAVTLTLIDDSCKEKCIAKAPTPLESDQSICAFQNSFLGGLPVGMTSNGVALLNFYNMPSTPSFVGCLQDIKIDWNHITLENISSGSSLNVKAGCVRKDWCESQPCQSRGRCINLWLSYQCDCHRPYEGPNCLREYVAGRFGQDDSTGYVIFTLDESYGDTISLSMFVRTLQPSGLLLALENSTYQYIRVWLERGRLAMLTPNSPKLVVKFVLNDGNVHLISLKIKPYKIELYQSSQNLGFISASTWKIEKGDVIYIGGLPDKQETELNGGFFKGCIQDVRLNNQNLEFFPNPTNNASLNPVLVNVTQGCAGDNSCKSNPCHNGGVCHSRWDDFSCSCPALTSGKACEEVQWCGFSPCPHGAQCQPVLQGFECIANAVFNGQSGQILFRSNGNITRELTNITFGFRTRDANVIILHAEKEPEFLNISIQDSRLFFQLQSGNSFYMLSLTSLQSVNDGTWHEVTLSMTDPLSQTSRWQMEVDNETPFVTSTIATGSLNFLKDNTDIYVGDRAIDNIKGLQGCLSTIEIGGIYLSYFENVHGFINKPQEEQFLKISTNSVVTGCLQLNVCNSNPCLHGGNCEDIYSSYHCSCPLGWSGKHCELNIDECFSNPCIHGNCSDRVAAYHCTCEPGYTGVNCEVDIDNCQSHQCANGATCISHTNGYSCLCFGNFTGKFCRQSRLPSTVCGNEKTNLTCYNGGNCTEFQTELKCMCRPGFTGEWCEKDIDECASDPCVNGGLCQDLLNKFQCLCDVAFAGERCEVDVSSLSFYVSLLFWQNLFQLLSYLILRMNDEPVVEWGEQEDY

SEQ ID NO: 2 - DNA, 5621 bp, Homo sapiens
  misc_feature 204..206: note = Start codon
  source 1..5621: mol_type = genomic DNA
agcacaaatgagtcttacactaagccacactgatccagcttggagggaaatgagtcaaaaccaagtccccatactctctacctgcgagaaagaggtgacattgaagaacaagcccacacttctggatatggtccaaaatgatcagaaactcactctgtcaacctagtaggtgtttggatggatacctcttttttaacagaaagatgtttggagccaggacacatggttttcacattttaatggcaatgctcataggaatccactgcgaagaagacgtcaatgaatgttcttcaaacccttgccaaaatggtggtacttgtgagaacttgcctgggaattatacttgccattgcccatttgataacctttctagaactttttatggaggaagggactgttctgatattctcctgggctgtacccatcagcaatgtctaaataatggaacatgcatccctcacttccaagatggccagcatggattcagctgcctatgtccatctggctacaccgggtccctgtgtgaaatcgcaaccacactttcatttgagggcgatggcttcctgtgggtcaaaagtggctcagtgacaaccaagggctcagtttgtaacatagccctcaggtttcagactgttcagccaatggctcttctacttttccgaagcaacagggatgtgtttgtgaagctggagctgctaagtggctacattcacttatcaattcaggtcaataatcagtcaaaggtgcttctgttcatttcccacaacaccagcgatggagagtggcatttcgtggaggtaatatttgcagaggctgtgacccttaccttaatcgacgactcctgtaaggagaaatgcatcgcgaaagctcctactccacttgaaagtgatcaatcaatatgtgcttttcagaactcctttttgggtggtttaccagtgggaatgaccagcaatggtgttgctctgcttaacttctataatatgccatccacaccttcgtttgtaggctgtctccaagacattaaaattgattggaatcacattaccctggagaacatctcgtctggctcatcattaaatgtcaaggcaggctgtgtgagaaaggattggtgtgaaagccaaccttgtcaaagcagaggacgctgcatcaacttgtggctgagttaccagtgtgactgccacaggccctatgaaggccccaactgtctgagagagtatgtggcaggcagatttggccaggatgactccactggttatgtcatctttactcttgatgagagctatggagacaccatcagcctctccatgtttgtccgaacgcttcaaccatcaggcttacttctagctttggaaaacagcacttatcaatatatccgtgtctggctagagcgcggcagactagcaatgctgactccaaactctcccaaattagtagtaaaatttgttcttaatgatggaaatgtccacttgatatctttgaaaatcaagccatataaaattgaactgtatcagtcttcacaaaacctaggatttatttctgcttctacgtggaaaatcgaaaagggagatgtcatctacattggtggcctacctgacaagcaagagactgaacttaatggtggattcttcaaaggctgtatccaagatgtaagactaaacaaccaaaatctggaattctttccaaatccaacaaacaatgcatctctcaatccagttcttgtcaatgtaacccaaggctgtgctggagacaacagctgcaagtccaacccctgtcacaatggaggtgtttgccattcccggtgggatgacttctcctgttcctgtcctgccctcacaagtgggaaagcctgtgaggaggttcagtggtgtggattcagcccgtgtcctcacggagcccagtgccagccggtgcttcaaggatttgaatgtattgcaaatgctgtttttaatggacaaagcggtcaaatattattcagaagcaatgggaatattaccagagaactcaccaatatcacatttggtttcagaacaagggatgcaaatgtaataatattgcatgcagaaaaagagcctgaatttcttaatattagcattcaagattccagattattctttcaattgcaaagtggcaacagcttttatatgctaagtctgacaagtttgcagtcagtgaatgatggcacatggcacgaagtgaccctttccatgacagacccactgtcccagacctccaggtggcaaatggaagtggacaacgaaacaccttttgtgaccagcacaattgctactggaagcctcaactttttgaaggataatacagatatttatgtgggagacagagctattgacaatataaagggcctgcaagggtgtctaagtacaatagaaatcggaggcatttatctctcttactttgaaaatgttcatggtttcattaataaacctcaggaagagcaatttctcaaaatctctaccaattcagtggtcactggctgtttgcagttaaatgtctgcaactccaacccctgtttgcatggaggaaactgtgaagacatctatagctcttatcattgctcctgtcccttgggatggtcagggaaacactgtgaactcaacatcgatgaatgcttttcaaacccctgtatccatggcaactgctctgacagagttgcagcctaccactgcacatgtgagcctggatacactggtgtgaactgtgaagtggatatagacaactgccagagtcaccagtgtgcaaatggagccacctgcattagtcatactaatggctattcttgcctctgttttggaaattttacaggaaaattttgcagacagagcagattaccctcaacagtctgtgggaatgagaagacaaatctcacttgctacaatggaggcaactgcacagagttccagactgaattaaaatgtatgtgccggccaggttttactggagaatggtgtgaaaaggacattgatgagtgtgcctctgatccgtgtgtcaatggaggtctgtgccaggacttactcaacaaattccagtgcctctgtgatgttgcctttgctggcgagcgctgcgaggtggacgtaagcagcctctccttttatgtctctctcttattctggcagaatctttttcagcttctttcttacctcattttgcgtatgaatgacgagccagttgttgagtggggtgaacaggaagattattaacatacatttgaacattcccaaatgaaaaaaaaagccattgaatttcaagaaatgccttgattcattttagatctctggggaagaaaaaggaaataaaaaccatctcaataattaaggtaaattcaaggcttattttaaacatatcagaagcactttgtctgtgtataaaatattttcctattctaactttaaatatgaaaaaagtgttcttaatataactagaaatatctccttattgtgtgtatttagtacaaacatattatcattctcaacacttctatatgtgaatgaccactgcaatttcttcccactccatttctgggtattttcacattttaagttgccctccatcactatgattctattttcatttctgttctttcattcttatctattatttatgacacaaaaattgagaattacaggccaggtgtggtggttcactcctataatcccagcactatgggaggctgaagtgggcggaacacctgaggccaggagtttgagaccagcctagccaacgtggtgaaaacctgtctctactaaaaatacaaaagtaactgggagtggtggcacatgcctgtaatcccagctactcaggagggtgaagcaggagacttgcttgaacccaggaggcggccgttgcaatgagccaagattgtgccactgcactccagcctgggcgacaggtgagattctgtctaaaaaaaaaaaaaaaaaaagagagaattaccgattaaaattactgattatattcatctatgtttttacatgaagctattcaaatgaattgttacgttttctctgatatatgattaaatatataaagagaaatcaggaatttacatcgagtccctaaattgtagaaaaacaattatctagtatcagtactcaaattatacctccctggtataatttctgattccataaaactgtctctctaacaaagttacaaataatcctttctctatttcctttcctgcaatactttcccttttcctaacaaatagaacaatttttctgttgtttctaaatttatgagctccttgacttttctatcaaatggactaatttcagttgcttttcaatgaatatttaataaaaataagcactgtagtttatacataaatttaaaagtatatattgtaaaacttgaattttcttagaagcatggttttctaagatttgcaagtaaatttattttcttaagtatctttcagaaaaaaatatgaaagcatagtatacatcataaccaaaatatatttgacattatgattttttaaaataaatgtatacctgaaataatggatctataaagtatactaagatatgcaaaaattaatatattctttattataaatatttcagagattataaaataataatttaaaaaaactttcttaatgtttttattgtttccaccagtacgttatcatttatgctaaatatctttgtgtagatatacccttccaaagaagacgttatttgtgttcatttaaaggaaaaatagtttgatcctatgaattaattcagaaagcaactaaaataacaatggcctgccaaatgtcattttgtaaatatacgtctatgactttaggagctgtcctggtttgaaaacatgaggacagtttatccattggatgccatctatttagtcccaattaagaaagttgtttttttgtgagaatgaccaaggtaaatttaaatataccattcaaacaaacaaggacaaaataatatccttgttatagagtacatgtagcatatagtatgaagtaatatactacaaaagcaaagaaagtgtattctatcttgcaatagtaatagacaatttttatatagcaaattcatatcctttggagtagtgacaatcatttcaaactgggagcaactaattgtgaagattttccttcttactcatccattttcttcacatccaaggctgaacgtgtgatgctgctgcttcagatgatttgttccaaagttaaattttgtgacaaaagacatggggaaaaccttcccatcaatatttacattcacaagtatttgcaataagcataaaatagattatagccagaccatatgtatagttttcacatttactcccttctagacatacctgtacttatgtacttacaggctgttccaaatgtaatatgttctctaccaaatgtggttaagaaatattcactcacaatttctttctgtgtacaattctgatgcctctgttgtcactgtaattgtcagttgcttttctgttttccaaatgtcttcttgtcataaggtatctgactttaaaaaatgttttcccttttctttttattcttctgtattttccagctgcatgtgtgtgactatggcttttacatatttgcacagaaaaataaaacctttgtttctgtatct

SEQ ID NO: 3 - protein, 41 aa, Homo sapiens
VSSLSFYVSLLFWQNLFQLLSYLILRMNDEPVVEWGEQEDY

SEQ ID NO: 4 - DNA, 5619 bp, Homo sapiens
  source 1..5619: mol_type = genomic DNA
acactattctaatgtaggcccttttgaggaggcagcatgaacagaagaaaactcgcagcaaaggcttgaggggggaatgaatccaatccagcctgaaaaaatctgcaccaggtttgaaaaatcaccccatcctcccgtgtaagtgatgctaagaagcacaaactgcattttgaatctaagtccctgtattttctgtgaaggagctgtaagtagggtgggacagagatggcacctgggggttctgaggcacccgctcctctctgagacagacagggatcaggagccggactgggaccagaccaccagcaacacaccagaggatgttctctaaataagaccatggcacttaagaacattaactaccttctcatcttctacctcagtttctcactgcttatctacataaaaaattccttttgcaataaaaacaacaccaggtgcctctcaaattcttgccaaaacaattctacatgcaaagatttttcaaaagacaatgattgttcttgttcagacacagccaataatttggacaaagactgtgacaacatgaaagacccttgcttctccaatccctgtcaaggaagtgccacttgtgtgaacaccccaggagaaaggagctttctgtgcaaatgtcctcctgggtacagtgggacaatctgtgaaactaccattggttcctgtggcaagaactcctgccaacatggaggtatttgccatcaggaccctatttatcctgtctgcatctgccctgctggatatgctggaagattctgtgagatagatcacgatgagtgtgcttccagcccttgccaaaatggggccgtgtgccaggatggaattgatggttactcctgcttctgtgtcccaggatatcaaggcagacactgcgacttggaagtggatgaatgtgcttcagatccctgcaagaacgaggctacatgcctcaatgaaataggaagatatacttgtatctgtccccacaattattctggtgtaaactgtgaattggaaattgacgaatgttggtcccagccttgtttaaatggtgcaacttgtcaggatgctctgggggcctatttctgcgactgtgcccctggattcctgggggatcactgtgaactcaacactgatgagtgtgccagtcaaccttgtctccatggagggctgtgtgtggatggagaaaacagatatagctgtaactgcacgggtagtggattcacagggacacactgtgagaccttgatgcctctttgttggtcaaaaccttgtcacaataatgctacatgtgaggacagtgttgacaattacacttgtcactgctggcctggatacacaggtgcccagtgtgagatcgacctcaatgaatgcaatagtaacccctgccagtccaatggggaatgtgtggagctgtcctcagagaaacaatatggacgcatcactggactgccttcttctttcagctaccatgaagcctcaggttatgtctgtatctgtcagcctggattcacaggaatccactgcgaagaagacgtcaatgaatgttcttcaaacccttgccaaaatggtggtacttgtgagaacttgcctgggaattatacttgccattgcccatttgataacctttctagaactttttatggaggaagggactgttctgatattctcctgggctgtacccatcagcaatgtctaaataatggaacatgcatccctcacttccaagatggccagcatggattcagctgcctatgtccatctggctacaccgggtccctgtgtgaaatcgcaaccacactttcatttgagggcgatggcttcctgtgggtcaaaagtggctcagtgacaaccaagggctcagtttgtaacatagccctcaggtttcagactgttcagccaatggctcttctacttttccgaagcaacagggatgtgtttgtgaagctggagctgctaagtggctacattcacttatcaattcaggtcaataatcagtcaaaggtgcttctgttcatttcccacaacaccagcgatggagagtggcatttcgtggaggtaatatttgcagaggctgtgacccttaccttaatcgacgactcctgtaaggagaaatgcatcgcgaaagctcctactccacttgaaagtgatcaatcaatatgtgcttttcagaactcctttttgggtggtttaccagtgggaatgaccagcaatggtgttgctctgcttaacttctataatatgccatccacaccttcgtttgtaggctgtctccaagacattaaaattgattggaatcacattaccctggagaacatctcgtctggctcatcattaaatgtcaaggcaggctgtgtgagaaaggattggtgtgaaagccaaccttgtcaaagcagaggacgctgcatcaacttgtggctgagttaccagtgtgactgccacaggccctatgaaggccccaactgtctgagagagtatgtggcaggcagatttggccaggatgactccactggttatgtcatctttactcttgatgagagctatggagacaccatcagcctctccatgtttgtccgaacgcttcaaccatcaggcttacttctagctttggaaaacagcacttatcaatatatccgtgtctggctagagcgcggcagactagcaatgctgactccaaactctcccaaattagtagtaaaatttgttcttaatgatggaaatgtccacttgatatctttgaaaatcaagccatataaaattgaactgtatcagtcttcacaaaacctaggatttatttctgcttctacgtggaaaatcgaaaagggagatgtcatctacattggtggcctacctgacaagcaagagactgaacttaatggtggattcttcaaaggctgtatccaagatgtaagactaaacaaccaaaatctggaattctttccaaatccaacaaacaatgcatctctcaatccagttcttgtcaatgtaacccaaggctgtgctggagacaacagctgcaagtccaacccctgtcacaatggaggtgtttgccattcccggtgggatgacttctcctgttcctgtcctgccctcacaagtgggaaagcctgtgaggaggttcagtggtgtggattcagcccgtgtcctcacggagcccagtgccagccggtgcttcaaggatttgaatgtattgcaaatgctgtttttaatggacaaagcggtcaaatattattcagaagcaatgggaatattaccagagaactcaccaatatcacatttggtttcagaacaagggatgcaaatgtaataatattgcatgcagaaaaagagcctgaatttcttaatattagcattcaagattccagattattctttcaattgcaaagtggcaacagcttttatatgctaagtctgacaagtttgcagtcagtgaatgatggcacatggcacgaagtgaccctttccatgacagacccactgtcccagacctccaggtggcaaatggaagtggacaacgaaacaccttttgtgaccagcacaattgctactggaagcctcaactttttgaaggataatacagatatttatgtgggagacagagctattgacaatataaagggcctgcaagggtgtctaagtacaatagaaatcggaggcatttatctctcttactttgaaaatgttcatggtttcattaataaacctcaggaagagcaatttctcaaaatctctaccaattcagtggtcactggctgtttgcagttaaatgtctgcaactccaacccctgtttgcatggaggaaactgtgaagacatctatagctcttatcattgctcctgtcccttgggatggtcagggaaacactgtgaactcaacatcgatgaatgcttttcaaacccctgtatccatggcaactgctctgacagagttgcagcctaccactgcacatgtgagcctggatacactggtgtgaactgtgaagtggatatagacaactgccagagtcaccagtgtgcaaatggagccacctgcattagtcatactaatggctattcttgcctctgttttggaaattttacaggaaaattttgcagacagagcagattaccctcaacagtctgtgggaatgagaagacaaatctcacttgctacaatggaggcaactgcacagagttccagactgaattaaaatgtatgtgccggccaggttttactggagaatggtgtgaaaaggacattgatgagtgtgcctctgatccgtgtgtcaatggaggtctgtgccaggacttactcaacaaattccagtgcctctgtgatgttgcctttgctggcgagcgctgcgaggtggacttggcagatgacttgatctccgacattttcaccactattggctcagtgactgtcgccttgttactgatcctcttgctggccattgttgcttctgttgtcacctccaacaaaagggcaactcagggaacctacagccccagccgtcaggagaaggagggctcccgagtggaaatgtggaacttgatgccaccccctgcaatggagagactgatttaggagcattgtgtcccttcgagatggggatccacacactgtgaatgtgatgactgtacttcaggtatctctgacatacctgacaatgttaatctgcaactgggattacactggaactacaggaatgattcctttgaccaccttaaaaactttcacagtggttccgctcgacaccattgttttattatattatatcagccaattgcaaaaaaagtctgtgccagtaatttcagccttataattagcaaaaacatcttccagagaataaagtcttctgtggctttagtggctatcactgaaactctttcctcttttcaacctgggaacaaattttagttttcattttaggtttctgtactttctgtagtttctgtgtaaactgccatatgtttacatggaaactacaggaaaaaattggctacatttctcacttctcctatcatgtggtcaaagttattgttgtataccagcgatgggatgtatacttttgtccttcattcatggattcagagaaagctctgggaatgacttatggtccaaaaaagtgacccaatggcaacaaataaaaattgaaatgcagttgttctcctttctgagtactttttgcatttttgtgacattatgtgtgacaaaagtaacctctaggaacatttgaagaacctgcttatgaattagaccttttacctaaatcatttcaagttggttacattttcaaattattactctttgtaaagggttggttaaggcaaaacgcttcctagatagaaatcaaaacggggaaaactcagattctcaagttcgaaaaatcgagttcttttcttccaactgcttttaggtaaatcagtgccaaacagtgacattgtttaaaggtaagaactccaaagttaaatgtatgcactttacggagtatgtgttttaagactatgggatatttggagaaaatgctggggtttctattcttatattttcttctacaaagcatctgattatatttttatatgtgctttgaaatatatgaaacatgctactgctgtagaatataaataaaacttaagaatagat

SEQ ID NO: 5 - protein, 1406 aa, Homo sapiens
MALKNINYLLIFYLSFSLLIYIKNSFCNKNNTRCLSNSCQNNSTCKDFSKDNDCSCSDTANNLDKDCDNMKDPCFSNPCQGSATCVNTPGERSFLCKCPPGYSGTICETTIGSCGKNSCQHGGICHQDPIYPVCICPAGYAGRFCEIDHDECASSPCQNGAVCQDGIDGYSCFCVPGYQGRHCDLEVDECASDPCKNEATCLNEIGRYTCICPHNYSGVNCELEIDECWSQPCLNGATCQDALGAYFCDCAPGFLGDHCELNTDECASQPCLHGGLCVDGENRYSCNCTGSGFTGTHCETLMPLCWSKPCHNNATCEDSVDNYTCHCWPGYTGAQCEIDLNECNSNPCQSNGECVELSSEKQYGRITGLPSSFSYHEASGYVCICQPGFTGIHCEEDVNECSSNPCQNGGTCENLPGNYTCHCPFDNLSRTFYGGRDCSDILLGCTHQQCLNNGTCIPHFQDGQHGFSCLCPSGYTGSLCEIATTLSFEGDGFLWVKSGSVTTKGSVCNIALRFQTVQPMALLLFRSNRDVFVKLELLSGYIHLSIQVNNQSKVLLFISHNTSDGEWHFVEVIFAEAVTLTLIDDSCKEKCIAKAPTPLESDQSICAFQNSFLGGLPVGMTSNGVALLNFYNMPSTPSFVGCLQDIKIDWNHITLENISSGSSLNVKAGCVRKDWCESQPCQSRGRCINLWLSYQCDCHRPYEGPNCLREYVAGRFGQDDSTGYVIFTLDESYGDTISLSMFVRTLQPSGLLLALENSTYQYIRVWLERGRLAMLTPNSPKLVVKFVLNDGNVHLISLKIKPYKIELYQSSQNLGFISASTWKIEKGDVIYIGGLPDKQETELNGGFFKGCIQDVRLNNQNLEFFPNPTNNASLNPVLVNVTQGCAGDNSCKSNPCHNGGVCHSRWDDFSCSCPALTSGKACEEVQWCGFSPCPHGAQCQPVLQGFECIANAVFNGQSGQILFRSNGNITRELTNITFGFRTRDANVIILHAEKEPEFLNISIQDSRLFFQLQSGNSFYMLSLTSLQSVNDGTWHEVTLSMTDPLSQTSRWQMEVDNETPFVTSTIATGSLNFLKDNTDIYVGDRAIDNIKGLQGCLSTIEIGGIYLSYFENVHGFINKPQEEQFLKISTNSVVTGCLQLNVCNSNPCLHGGNCEDIYSSYHCSCPLGWSGKHCELNIDECFSNPCIHGNCSDRVAAYHCTCEPGYTGVNCEVDIDNCQSHQCANGATCISHTNGYSCLCFGNFTGKFCRQSRLPSTVCGNEKTNLTCYNGGNCTEFQTELKCMCRPGFTGEWCEKDIDECASDPCVNGGLCQDLLNKFQCLCDVAFAGERCEVDLADDLISDIFTTIGSVTVALLLILLLAIVASVVTSNKRATQGTYSPSRQEKEGSRVEMWNLMPPPAMERLI

SEQ ID NO: 6 - DNA, 5541 bp, Homo sapiens
  source 1..5541: mol_type = genomic DNA
gtcttggcccaacttacaaacagcagaaatctgagttgtgggaatataatttatgaacagaaaagattacttgtctgtgaattattttctgaagatgaaagtaaatatacaggaacatacggtattcctttaaaagttgccagatcataattgtgtggcaaggcagttatcagaaattaatccctctattgagagcaattgaagacactattctaatgtaggcccttttgaggaggcagcatgaacagaagaaaactcgcagcaaaggcttgaggggggaatgaatccaatccagcctgaaaaaatctgcaccaggtttgaaaaatcaccccatcctcccgtgtaagtgatgctaagaagcacaaactgcattttgaatctaagtccctgtattttctgtgaaggagctgtaagtagggtgggacagagatggcacctgggggttctgaggcacccgctcctctctgagacagacagggatcaggagccggactgggaccagaccaccagcaacacaccagaggatgttctctaaataagaccatggcacttaagaacattaactaccttctcatcttctacctcagtttctcactgcttatctacataaaaaattccttttgcaataaaaacaacaccaggtgcctctcaaattcttgccaaaacaattctacatgcaaagatttttcaaaagacaatgattgttcttgttcagacacagccaataatttggacaaagactgtgacaacatgaaagacccttgcttctccaatccctgtcaaggaagtgccacttgtgtgaacaccccaggagaaaggagctttctgtgcaaatgtcctcctgggtacagtgggacaatctgtgaaactaccattggttcctgtggcaagaactcctgccaacatggaggtatttgccatcaggaccctatttatcctgtctgcatctgccctgctggatatgctggaagattctgtgagatagatcacgatgagtgtgcttccagcccttgccaaaatggggccgtgtgccaggatggaattgatggttactcctgcttctgtgtcccaggatatcaaggcagacactgcgacttggaagtggatgaatgtgcttcagatccctgcaagaacgaggctacatgcctcaatgaaataggaagatatacttgtatctgtccccacaattattctggtgtaaactgtgaattggaaattgacgaatgttggtcccagccttgtttaaatggtgcaacttgtcaggatgctctgggggcctatttctgcgactgtgcccctggattcctgggggatcactgtgaactcaacactgatgagtgtgccagtcaaccttgtctccatggagggctgtgtgtggatggagaaaacagatatagctgtaactgcacgggtagtggattcacagggacacactgtgagaccttgatgcctctttgttggtcaaaaccttgtcacaataatgctacatgtgaggacagtgttgacaattacacttgtcactgctggcctggatacacaggtgcccagtgtgagatcgacctcaatgaatgcaatagtaacccctgccagtccaatggggaatgtgtggagctgtcctcagagaaacaatatggacgcatcactggactgccttcttctttcagctaccatgaagcctcaggttatgtctgtatctgtcagcctggattcacaggaatccactgcgaagaagacgtcaatgaatgttcttcaaacccttgccaaaatggtggtacttgtgagaacttgcctgggaattatacttgccattgcccatttgataacctttctagaactttttatggaggaagggactgttctgatattctcctgggctgtacccatcagcaatgtctaaataatggaacatgcatccctcacttccaagatggccagcatggattcagctgcctatgtccatctggctacaccgggtccctgtgtgaaatcgcaaccacactttcatttgagggcgatggcttcctgtgggtcaaaagtggctcagtgacaaccaagggctcagtttgtaacatagccctcaggtttcagactgttcagccaatggctcttctacttttccgaagcaacagggatgtgtttgtgaagctggagctgctaagtggctacattcacttatcaattcaggtcaataatcagtcaaaggtgcttctgttcatttcccacaacaccagcgatggagagtggcatttcgtggaggtaatatttgcagaggctgtgacccttaccttaatcgacgactcctgtaaggagaaatgcatcgcgaaagctcctactccacttgaaagtgatcaatcaatatgtgcttttcagaactcctttttgggtggtttaccagtgggaatgaccagcaatggtgttgctctgcttaacttctataatatgccatccacaccttcgtttgtaggctgtctccaagacattaaaattgattggaatcacattaccctggagaacatctcgtctggctcatcattaaatgtcaaggcaggctgtgtgagaaaggattggtgtgaaagccaaccttgtcaaagcagaggacgctgcatcaacttgtggctgagttaccagtgtgactgccacaggccctatgaaggccccaactgtctgagaggtgagagaaagctgagtgctatggctaggagtgccatgcctcagagcagagcagaaacagcaaaaacagccagactgcttctgcctgctatgaaacataatgaccccacaagacttctgctgctggttgcccactgatgagaaagaaaagaagagggcagtgatgtgcgttaattaattttgagtggattcataggacatcagtttcactcatacagagaagtaaaaaaaataagcagatagctcttttccaaagaggttttcatctttgtgtttgcaaaatgctactgcaattttaccattggtcacatatcagaaatttattgtaaatcttatttgaaagagaaataatcttttgaaaaaaaaaaaccttagacataaaatttgtcagtgccacatactagcatgatatcttgtgcatagtaaattctcggtaaatattcatttccttgctctcctttccatgcaattcacacttgctccacaatcataattaagcatagcgtttttataaaacgccaattttattcaaaggtatcttttccaaggttgccctggagaagacagataatatactaggtgtcttaagaaaaaaaaaaaagaaaaaaaaatgtggaaagtaacaatagtagaacttggtagatgccataaactaattgatctaatttctctagtaaaccacagtttgcaaagtatttcaaaatccttaacttccaacattgttctagagattgctgttgatagtgatcaatatatacctgtttctttttattattattatcatacattgaaaaagtctgagaggtaattgtaacggactgctttgaagttcagctgtaatttaccccaagattttagagtaacttgaagtaggacaacatctggtaagtagtcttctctgctttatatgaagtaaaattaaaaccagtcttagcctatactctattccatgttcaaatgtctaagaattattagaaactactcaagggtttcccccgaccacaatattcattacaaatcacggacctgtttgacaatgaagtatggtatctactagctctacgtaatattttaagtagaatggcaagttgttttgtgattttttttaaggagaaaaagggaaattatgctgaggcaatgttcactttttgaagaaaaatttaattgacctaaggcaatatttttactacatatacaaaataacaaacagatgctgactcattttgactggcttccagatgcagtgacccatgaggttaatactgaccatggtgatgttaactgtcctagtaaatagattcaagagatgccaatccactgggttctggttttctttattttatccaacttggaaaaccatgaacttttcaaaccaagttcctgaattggttacccttcccccacttctcccagtttccttatactccaacattatttgaaactgcttaaccctcaagaaaatatttgacttcaaaagatcactttacagacagagagagttctgtgaaatcttttctttctgaaaattcttggtataaatttaaggaaaattatatttttcatatcacctcttcattataaaattaaaatattctgtattttctgttatcttgaaggaatgtttcctggagagtttattcccactaaaaactgcttttaagtggtaatgagaacatggagtctttctgctactgtacatatacgtgcaagagggaggagacatactaaaaagcacaaaggcaaacttcagcccattcatgacttgcactcatatttggctcaatgttttccaaccatggtatacattttaatgaacaaggagactggatagaggattttggtggctgactaaacacaaaagacatagtgatttccaatgtatcaaggttgatcttttttaaataatctaatttctcaacagaaagagtgctgaaaaggatttaactaagcttaggtcacagaagtgtccataaggttctctcagcatatcttgtgttgactgtttgtaagaacatgcagcattcaagatactactactcagaataaatggcatcacctacctcttgctctctggtgaacatttgacttacaaaagcaatttacatatctcttcttttcagccatatacctagattttgagaattttattctcttagaattatagtttcctagatatagaaaaagattcattctatgactgaatttttaacacaattgtacctgccaaatgtagtggctcaaatctctgctcaaacaccttcacaaatagagagatcactcactccatcttgccaaaaattgcccaaaagttatttccttatattgaaatagaataggatttgcccgtagctttccttcattgaccttgttgcatttctggaacacatgcaaataaaaatctcaacttgcctgctgcttgagaatacttaatatctgaagaaaagtatctcgagccatctaaaccatcacttctattcaggctaaacatcctgtttcttctaatgtttgccatatgacatagttttccgacacttcatcagctgcttgcaggttttggaatatatttgcttctttgtattccttctaagatgtgatgtctgtatgtgctggatgatacagtgacacacagggccataacctcccttattctagataatgtgcttctgtttttcagcttaatacaacattgactttttggcagccacacttcgtgattgcgtcactttgtatcattgtcactcatgatgccacatagtcctgtttctcccactctaatcttataaacgcacatttagatcgaagtggctcttatttattcttgttaaatttcatcttgttgtattccatcaatcattaaagcttgccaaaatt

SEQ ID NO: 7 - protein, 754 aa, Homo sapiens
MALKNINYLLIFYLSFSLLIYIKNSFCNKNNTRCLSNSCQNNSTCKDFSKDNDCSCSDTANNLDKDCDNMKDPCFSNPCQGSATCVNTPGERSFLCKCPPGYSGTICETTIGSCGKNSCQHGGICHQDPIYPVCICPAGYAGRFCEIDHDECASSPCQNGAVCQDGIDGYSCFCVPGYQGRHCDLEVDECASDPCKNEATCLNEIGRYTCICPHNYSGVNCELEIDECWSQPCLNGATCQDALGAYFCDCAPGFLGDHCELNTDECASQPCLHGGLCVDGENRYSCNCTGSGFTGTHCETLMPLCWSKPCHNNATCEDSVDNYTCHCWPGYTGAQCEIDLNECNSNPCQSNGECVELSSEKQYGRITGLPSSFSYHEASGYVCICQPGFTGIHCEEDVNECSSNPCQNGGTCENLPGNYTCHCPFDNLSRTFYGGRDCSDILLGCTHQQCLNNGTCIPHFQDGQHGFSCLCPSGYTGSLCEIATTLSFEGDGFLWVKSGSVTTKGSVCNIALRFQTVQPMALLLFRSNRDVFVKLELLSGYIHLSIQVNNQSKVLLFISHNTSDGEWHFVEVIFAEAVTLTLIDDSCKEKCIAKAPTPLESDQSICAFQNSFLGGLPVGMTSNGVALLNFYNMPSTPSFVGCLQDIKIDWNHITLENISSGSSLNVKAGCVRKDWCESQPCQSRGRCINLWLSYQCDCHRPYEGPNCLRGERKLSAMARSAMPQSRAETAKTARLLLPAMKHNDPTRLLLLVAH

SEQ ID NO: 8 - DNA, 6170 bp, Mus musculus
  source 1..6170: mol_type = genomic DNA
attgttcacggaagcctgagggggacacgaatccaatccaggctggaaaaatctgctccaggattgactggttaccgtcttcctgtgcctgtaaggtgctgtgaaagagaagtgctttctgattctctgtctgtggaggagccctgggaggggtgggacagagatggcatcctggctctctgaggcacctgctcttctctgaaccacacaggagtcaagagccaaacagggatagcttcagcagcacttcagagggtgttctctaagtaagaacatgaagctcaagagaactgcctaccttctcttcctgtacctcagctcctcactgctcatctgcataaagaattcattttgcaataaaaacaataccaggtgcctttcaggtccttgccaaaacaattctacgtgcaagcattttccacaagacaacaattgttgcttagacacagccaataatttggacaaagactgtgaagatctgaaagacccttgcttctcgagtccctgccaaggaattgccacttgtgtgaaaatcccaggggaagggaacttcctgtgtcagtgtcctcctgggtacagcgggctgaactgtgaaactgccaccaattcctgtggagggaacctctgccaacatggaggcacctgccgtaaagaccctgagcaccctgtctgtatctgccctcctggatatgctggaaggttctgtgagactgatcacaatgagtgtgcttctagcccttgccacaatggggctatgtgccaggatggaatcaatggctactcctgcttctgtgtgcctggataccaaggcaggcattgtgacttggaagtggatgaatgtgtttctgatccctgcaagaatgaggctgtgtgcctcaatgagataggaagatacacttgtgtctgccctcaagagttttctggcgtgaactgtgagttggaaattgatgaatgcagatcccagccttgtctccacggtgccacatgtcaggacgctccagggggctactcctgtgactgtgcacctggattccttggagagcactgtgaactcagcgttaatgaatgtgaaagtcagccgtgtctccatggaggtctatgtgtggatggaagaaacagttaccactgtgactgcacaggtagtggattcacagggatgcactgtgagtccttgattcctctttgttggtcaaagccttgtcacaacgacgcgacatgtgaagatactgttgacagctatatttgtcactgccggcctggatacacaggtgccctgtgtgagacagacataaatgaatgcagtagcaacccctgccaattttggggggaatgtgtcgagctgtcctcagagggtctatatggaaacactgctggcctgccttcctccttcagctatgttggagcctcgggctatgtgtgtatctgtcagcctggattcacaggaattcactgtgaagaagacgttgatgaatgtttactgcacccttgcctaaatggtggtacttgtgagaacctgcctgggaattatgcctgtcactgtccctttgatgacacttctaggacattttatggaggagaaaactgctcagaaattctcctgggctgcactcatcaccagtgtctgaacaatggaaaatgtatccctcatttccaaaatggccagcatggattcacttgccagtgtctttctggctatgcggggcccctgtgtgaaactgtcaccacactttcatttgggagcaatggcttcctatgggtcacaagtggctcccatacaggcatagggccagaatgtaacatatccttgaggtttcacactgttcaaccaaacgcacttctcctcatccgaggcaacaaggacgtgtctatgaagctggagttgctgaatggttgtgttcacttatcaattgaagtctggaatcagttaaaggtgctcctgtctatttctcacaacaccagtgatggagaatggcatttcgtggaggtaacaatcgcagaaactctaacccttgccctagttggcggctcctgcaaggagaagtgcaccaccaagtcttctgttccagttgagaatcatcaatcaatatgtgctttgcaggactcttttttgggtggcttaccaatggggacagccaacaacagtgtgtctgtgcttaacatctataatgtgccgtccacaccttcctttgtaggctgtctccaagacattagatttgatttgaatcacattactctggagaacgtttcatctggcctgtcatcaaatgttaaagcaggctgcctgggaaaggactggtgtgaaagtcaaccctgtcaaaacagaggacgctgcatcaacttgtggcagggttatcagtgtgaatgtgacaggccctatacaggctccaactgcctgaaagagtatgtagcgggaagatttggccaagatgactccacaggatatgcggcctttagtgttaatgataattatggacagaacttcagtctttcaatgtttgtccgaacacgtcaacccctgggcttacttctggctttggaaaatagtacttaccagtatgtcagtgtctggctagagcacggcagcctagcactgcagactccaggctctcccaagttcatggtaaacttttttctcagtgatggaaatgttcacttaatatctttgagaatcaaaccaaatgaaattgaactgtatcagtcttcacaaaacctaggattcatttctgttcctacatggacaattcgaagaggagacgtcatcttcattggtggcttacctgacagagagaagactgaagtttatggtggcttcttcaaaggctgtgttcaagatgtcagattaaacagccagactctggaattctttcccaattcaacaaacaatgcatacgatgacccaattcttgtcaatgtgactcaaggctgtcccggagacaacacatgtaagtccaacccctgtcataatggaggtgtctgccactccctgtgggatgacttctcctgctcctgccctacaaacacagcggggagagcctgcgagcaagttcagtggtgtcaactcagcccatgtcctcccactgcagagtgccagctgctccctcaagggtttgaatgtatcgcaaacgctgttttcagcggattaagcagagaaatactcttcagaagcaatgggaacattaccagagaactcaccaatatcacatttgctttcagaacacatgatacaaatgtgatgatattgcatgcagaaaaagaaccagagtttcttaatattagcattcaagatgccagattattctttcaattgcgaagtggcaacagcttttatacgctgcacctgatgggttcccaattggtgaatgatggcacatggcaccaagtgactttctccatgatagacccagtggcccagacctcccggtggcaaatggaggtgaacgaccagacaccctttgtgataagtgaagttgctactggaagcctgaactttttgaaggacaatacagacatctatgtgggtgaccaatctgttgacaatccgaaaggcctgcagggctgtctgagcacaatagagattggaggcatatatctttcttactttgaaaatctacatggtttccctggtaagcctcaggaagagcaatttctcaaagtttctacaaatatggtacttactggctgtttgccatcaaatgcctgccactccagcccctgtttgcatggaggaaactgtgaagacagctacagttcttatcggtgtgcctgtctctcgggatggtcagggacacactgtgaaatcaacattgatgagtgcttttctagcccctgtatccatggcaactgctctgatggagttgcagcctaccactgcaggtgtgagcctggatacaccggtgtgaactgtgaggtggatgtagacaattgcaagagtcatcagtgtgcaaatggggccacctgtgttcctgaagctcatggctactcttgtctctgctttggaaattttaccgggagattttgcagacacagcagattaccctcaacagtctgtgggaatgagaagagaaacttcacttgctacaatggaggcagctgctccatgttccaggaggactggcaatgtatgtgctggccaggtttcactggagagtggtgtgaagaggacatcaacgagtgtgcctccgatccctgcatcaatggaggactgtgcagggacttggtcaacaggttcctatgcatctgtgatgtggccttcgctggcgagcgctgtgagctggacctggctgatgacaggctcctgggcattttcaccgctgttggctccggaactttggccctgttcttcatcctcttgcttgctggggttgcttctcttattgcctccaacaaaagggcgactcaaggaacctacagccccagcggtcaggagaaggctggccctcgagtggaaatgtggatcaggatgccgcccccggcactggaaaggctcatctaggagactgctgctcttctcaggacagagaagaacatgatgagtaccgggtcgtgcctgagtgaagatggctttacatcactagagatacatacagctgggactgtgggaaggaccttcctgtggagtcactgagtagttatgtcatccattcacagaagagtgtccctgtgtttgcctgtcagcctcagaattagcaaaacatctagcagacagagaacacagtatttcagaagaactccagaggctgccccttaaactctttactggttgatccacataaaatgcttagtagccaagtgccattaattatacagagccaagaagaaaaattagaatacaactttcactttttattttgtagggaaggttttatgttttggtttgttgttgttgttgtgacagtgacagtgactcattacatagaccaagctggcctcaaaatcacatggaccctcgggattacatgtgtccgaccatgttcatcttatttttgaatcttctgtcatatggtaaaagattccagtgggacctgaggagtgactagctaggtaaagcaagggctgtgtaagtgccagaactggtgtttgtgtcctcattatccacataagtgccaagtgagtgtggcccctgcctgtcatcctaggcctcaggagatatcactgctcactggagcaagccggttaaactgttagggcaggtaagttttgacttcaagtgagagaccctgactcaatatgaaaggcaattagtgagtcaagatgaccctgtatgctaacctcttgcctatacatgcatatacacacatttacatatgtgcccaaacatgaggacacaagcacacgcgcgcgcgcacacacacacacacacacacacacacacacacacacacacacacacgagtctaattgtatatagtgataacagtacactttcctccttctatttcggatttagagaaagccatgagaagcgtgtatggtttaaaccatgacccaagcataacaaataaagttgaaatagttgttctcctgtccaagcttgtctttattgttgtgcattctgtaagctggttgcttggttggctgatggatggcttctgtttgtttgttgttttttgtttgtttgtttgtctgggatattacatgtaagaaaaataactggtaagaacaatcaaagaactttgttatgaattaaatcttttgtctaagtcacttagagtcattattctttatgtagatttgcttccagtcaggacatttcctagacagaatttaagacagtaagaaaatgatttgtcacgtctgaaagaggttctttactttcagggacttttgataatgcccaacagagatggcatcgaaagaggagctcatagcgagatgggcatttgtgcatcctcaaggagaaaatattgtaccttctgtttgtatattgtctattctgtgatggctgtatcttacatatgttttgatgcatgtaacaatagtatcatatgaaataaattatatatatatataatatataatatatatcacaagataaaaattgaaattacataaactttaaatctaaaagaagaaacctatccttcccaagtattatcagtgcagtcaccgagctttttttgtttttttgtattagccatttcttcataatacaggaagttctataacttcaa

SEQ ID NO: 9 - protein, 1405 aa, Mus musculus
MKLKRTAYLLFLYLSSSLLICIKNSFCNKNNTRCLSGPCQNNSTCKHFPQDNNCCLDTANNLDKDCEDLKDPCFSSPCQGIATCVKIPGEGNFLCQCPPGYSGLNCETATNSCGGNLCQHGGTCRKDPEHPVCICPPGYAGRFCETDHNECASSPCHNGAMCQDGINGYSCFCVPGYQGRHCDLEVDECVSDPCKNEAVCLNEIGRYTCVCPQEFSGVNCELEIDECRSQPCLHGATCQDAPGGYSCDCAPGFLGEHCELSVNECESQPCLHGGLCVDGRNSYHCDCTGSGFTGMHCESLIPLCWSKPCHNDATCEDTVDSYICHCRPGYTGALCETDINECSSNPCQFWGECVELSSEGLYGNTAGLPSSFSYVGASGYVCICQPGFTGIHCEEDVDECLLHPCLNGGTCENLPGNYACHCPFDDTSRTFYGGENCSEILLGCTHHQCLNNGKCIPHFQNGQHGFTCQCLSGYAGPLCETVTTLSFGSNGFLWVTSGSHTGIGPECNISLRFHTVQPNALLLIRGNKDVSMKLELLNGCVHLSIEVWNQLKVLLSISHNTSDGEWHFVEVTIAETLTLALVGGSCKEKCTTKSSVPVENHQSICALQDSFLGGLPMGTANNSVSVLNIYNVPSTPSFVGCLQDIRFDLNHITLENVSSGLSSNVKAGCLGKDWCESQPCQNRGRCINLWQGYQCECDRPYTGSNCLKEYVAGRFGQDDSTGYAAFSVNDNYGQNFSLSMFVRTRQPLGLLLALENSTYQYVSVWLEHGSLALQTPGSPKFMVNFFLSDGNVHLISLRIKPNEIELYQSSQNLGFISVPTWTIRRGDVIFIGGLPDREKTEVYGGFFKGCVQDVRLNSQTLEFFPNSTNNAYDDPILVNVTQGCPGDNTCKSNPCHNGGVCHSLWDDFSCSCPTNTAGRACEQVQWCQLSPCPPTAECQLLPQGFECIANAVFSGLSREILFRSNGNITRELTNITFAFRTHDTNVMILHAEKEPEFLNISIQDARLFFQLRSGNSFYTLHLMGSQLVNDGTWHQVTFSMIDPVAQTSRWQMEVNDQTPFVISEVATGSLNFLKDNTDIYVGDQSVDNPKGLQGCLSTIEIGGIYLSYFENLHGFPGKPQEEQFLKVSTNMVLTGCLPSNACHSSPCLHGGNCEDSYSSYRCACLSGWSGTHCEINIDECFSSPCIHGNCSDGVAAYHCRCEPGYTGVNCEVDVDNCKSHQCANGATCVPEAHGYSCLCFGNFTGRFCRHSRLPSTVCGNEKRNFTCYNGGSCSMFQEDWQCMCWPGFTGEWCEEDINECASDPCINGGLCRDLVNRFLCICDVAFAGERCELDLADDRLLGIFTAVGSGTLALFFILLLAGVASLIASNKRATQGTYSPSGQEKAGPRVEMWIRMPPPALERLI

SEQ ID NO: 10 - DNA, 5764 bp, Mus musculus
  source 1..5764: mol_type = genomic DNA
ttacagaagggaggcaccgtgtctcctgcggggtaggagctaagaatatagcaaagctgcttgggaagtggcacagctgactcttacattaagccccactgatccagcttgaagaggagtgaggcaaagctgaaccctcccactctccttgacaagtgcaagcccacacttttggaaaaaagcacaaagacgtcagaaacggttcctgtcgacctactaggctttggatggctaagtgtttttgctttgtatggaaatatgtttggacacaagacacaaggttttcacattttaatggcagtgctcataggaattcactgtgaagaagacgttgatgaatgtttactgcacccttgcctaaatggtggtacttgtgagaacctgcctgggaattatgcctgtcactgtccctttgatgacacttctaggacattttatggaggagaaaactgctcagaaattctcctgggctgcactcatcaccagtgtctgaacaatggaaaatgtatccctcatttccaaaatggccagcatggattcacttgccagtgtctttctggctatgcggggcccctgtgtgaaactgtcaccacactttcatttgggagcaatggcttcctatgggtcacaagtggctcccatacaggcatagggccagaatgtaacatatccttgaggtttcacactgttcaaccaaacgcacttctcctcatccgaggcaacaaggacgtgtctatgaagctggagttgctgaatggttgtgttcacttatcaattgaagtctggaatcagttaaaggtgctcctgtctatttctcacaacaccagtgatggagaatggcatttcgtggaggtaacaatcgcagaaactctaacccttgccctagttggcggctcctgcaaggagaagtgcaccaccaagtcttctgttccagttgagaatcatcaatcaatatgtgctttgcaggactcttttttgggtggcttaccaatggggacagccaacaacagtgtgtctgtgcttaacatctataatgtgccgtccacaccttcctttgtaggctgtctccaagacattagatttgatttgaatcacattactctggagaacgtttcatctggcctgtcatcaaatgttaaagcaggctgcctgggaaaggactggtgtgaaagtcaaccctgtcaaaacagaggacgctgcatcaacttgtggcagggttatcagtgtgaatgtgacaggccctatacaggctccaactgcctgaaagagtatgtagcgggaagatttggccaagatgactccacaggatatgcggcctttagtgttaatgataattatggacagaacttcagtctttcaatgtttgtccgaacacgtcaacccctgggcttacttctggctttggaaaatagtacttaccagtatgtcagtgtctggctagagcacggcagcctagcactgcagactccaggctctcccaagttcatggtaaacttttttctcagtgatggaaatgttcacttaatatctttgagaatcaaaccaaatgaaattgaactgtatcagtcttcacaaaacctaggattcatttctgttcctacatggacaattcgaagaggagacgtcatcttcattggtggcttacctgacagagagaagactgaagtttatggtggcttcttcaaaggctgtgttcaagatgtcagattaaacagccagactctggaattctttcccaattcaacaaacaatgcatacgatgacccaattcttgtcaatgtgactcaaggctgtcccggagacaacacatgtaagtccaacccctgtcataatggaggtgtctgccactccctgtgggatgacttctcctgctcctgccctacaaacacagcggggagagcctgcgagcaagttcagtggtgtcaactcagcccatgtcctcccactgcagagtgccagctgctccctcaagggtttgaatgtatcgcaaacgctgttttcagcggattaagcagagaaatactcttcagaagcaatgggaacattaccagagaactcaccaatatcacatttgctttcagaacacatgatacaaatgtgatgatattgcatgcagaaaaagaaccagagtttcttaatattagcattcaagatgccagattattctttcaattgcgaagtggcaacagcttttatacgctgcacctgatgggttcccaattggtgaatgatggcacatggcaccaagtgactttctccatgatagacccagtggcccagacctcccggtggcaaatggaggtgaacgaccagacaccctttgtgataagtgaagttgctactggaagcctgaactttttgaaggacaatacagacatctatgtgggtgaccaatctgttgacaatccgaaaggcctgcagggctgtctgagcacaatagagattggaggcatatatctttcttactttgaaaatctacatggtttccctggtaagcctcaggaagagcaatttctcaaagtttctacaaatatggtacttactggctgtttgccatcaaatgcctgccactccagcccctgtttgcatggaggaaactgtgaagacagctacagttcttatcggtgtgcctgtctctcgggatggtcagggacacactgtgaaatcaacattgatgagtgcttttctagcccctgtatccatggcaactgctctgatggagttgcagcctaccactgcaggtgtgagcctggatacaccggtgtgaactgtgaggtggatgtagacaattgcaagagtcatcagtgtgcaaatggggccacctgtgttcctgaagctcatggctactcttgtctctgctttggaaattttaccgggagattttgcagacacagcagattaccctcaacagtctgtgggaatgagaagagaaacttcacttgctacaatggaggcagctgctccatgttccaggaggactggcaatgtatgtgctggccaggtttcactggagagtggtgtgaagaggacatcaacgagtgtgcctccgatccctgcatcaatggaggactgtgcagggacttggtcaacaggttcctatgcatctgtgatgtggccttcgctggcgagcgctgtgagctggacgtaagcggcctttccttttatgtgtccctcttactatggcaaaacctctttcagctcctgtcctacctcgtactgcgcatgaatgatgagccagttgtagagtggggggcacaggaaaattattaatgtgcatgggagcattcacaagtgtaaaacattgacttgcaagaaacatcttgtctcagtgtaggtttctaggaaagacaaagggaacattagggaatagactccatctagagcactggttctcagtcttcctaatgctgcaaccctttagtacagctcttcctgttgtagtgatcgcagccataacattattttcattgccacttcataactgtaatccttctactgctgtgaatcacaatggaaatatttatgttttctgatggtcttaagcaacacctctgaaaaagtcattgaccccccccccaaaggggctgtgatccacaggttgagaaatgctcatctggaaggtaaccatgcatttaagtgtacctctagtagtttgggtctatagaagatattctcctattctacctttttagacacgccagaagagggcatctgattccattaaagatgattgggagccaccgtgtggttcctgagaactgtactcgggccctttggaagagcaatcagtgctctttccagcccctaagaatatttttaatacagccagaaaggtctcattacccagtgtactgagccctaaggcactttcatcctcaatcgttccatgttgaatggttttcattacatttggaaaatgttttctctccactctacctttacatgttcctattttcctattgacaatttgccccttcactgtaattctaatttggtgtggtccttcttctcataagtttatatgtgacatgaacatttaaaaatatctatgaatattttatagtcatgtatgtctttctgcaaagctattcaaatgaactatggacagttcttttctacacgaagaagagatgagtttaatccccagtaacatgagaaaaagatgagtgagggacagtgctcacagtatccctcactagcatcatttgtgattccatgggccatttttttccaccagcaaatagcagagagccctttccctattcgtttctcttacacttccccttttctgttacaactgaacactttacattagttactcctttgtagggggtttgacttttccaccgttttctctggttcactatttatgctaagtatctgtgcagggcgggtatatcagtccaacagaggtgtcattagtgttcattgaggaggaaatactttgcatgaattcatgacatcattgaagtagcagtggccagaaagatacccttctgcgaatgtgtctgtgtattcagaagctgccctggttagaaaacatgtgggtcacttttcctttgcatgttaccagtgctcactgggtcatgattgttttaagacagagcttttgctgtggcaatgaccaaggtgaatccagagatgcagatcagacaaaggacaagacaatgtactatctgagtaaaaccctgccttgacttactcctcagtacttagagattttacatagcaacctccaccctgtggcaacccgttcacactagcagtgatgctgagatttgcccttccttctcatcatcttcctcacatccaaagcattttgtgtccacactgctgtttcagataactgtttctaaagtgggattgttgtagccagaaaggtagggaaaatgttccccaaaatatttgcattcttaagtatgtgaagtaagtagattatagtcagagacaatatgtaaggtttcaggttcactcccttctacacatatcttcaactgtgtatttgcagaatattctgaatgtgacatactcccaacagaatatatttaaggagtatttatccacagtattgttctctgtacagttctagtgcttctattgtcactgcaattgtcaattgtttttctgctttccaactgtcttattatcatttaatagcatcttgctaaatgccctctttctattctccttatttctccatagttcatgtgtgtctgtgtgactaaggattctcctcatttttgcagaaaaataaaatcttttcttctttatgtcctgcttgtcattctctggtgacacatgtctttgcttacttggactgagggttgtacagtaagtacagaagcaggctcagtcacacagacagagacacaccaccaccagcagcagcagcaccaccaccaccaccaccaccaccagaaaacagtatgagtactcatctcttgattacatgtcatttcaagtaagcaccatgacaccgagggccaggttccatggactttctctgttaggcacgtgattctttagctgacctttgagaacagactccaacaacctcacttatttttactgttgacttatatcatctctgacaacactggacttcgtttgagctagtcaagaggaaagaccatgacacctaagggacagaaattcacacactcggtttttcataattcacacacattcctatgtatcaaatctctgtaatagatgacatttacttgaataaaaagtcatttccctttgctgatgtttcatcttt

SEQ ID NO: 11 - protein, 1003 aa, Mus musculus
MFGHKTQGFHILMAVLIGIHCEEDVDECLLHPCLNGGTCENLPGNYACHCPFDDTSRTFYGGENCSEILLGCTHHQCLNNGKCIPHFQNGQHGFTCQCLSGYAGPLCETVTTLSFGSNGFLWVTSGSHTGIGPECNISLRFHTVQPNALLLIRGNKDVSMKLELLNGCVHLSIEVWNQLKVLLSISHNTSDGEWHFVEVTIAETLTLALVGGSCKEKCTTKSSVPVENHQSICALQDSFLGGLPMGTANNSVSVLNIYNVPSTPSFVGCLQDIRFDLNHITLENVSSGLSSNVKAGCLGKDWCESQPCQNRGRCINLWQGYQCECDRPYTGSNCLKEYVAGRFGQDDSTGYAAFSVNDNYGQNFSLSMFVRTRQPLGLLLALENSTYQYVSVWLEHGSLALQTPGSPKFMVNFFLSDGNVHLISLRIKPNEIELYQSSQNLGFISVPTWTIRRGDVIFIGGLPDREKTEVYGGFFKGCVQDVRLNSQTLEFFPNSTNNAYDDPILVNVTQGCPGDNTCKSNPCHNGGVCHSLWDDFSCSCPTNTAGRACEQVQWCQLSPCPPTAECQLLPQGFECIANAVFSGLSREILFRSNGNITRELTNITFAFRTHDTNVMILHAEKEPEFLNISIQDARLFFQLRSGNSFYTLHLMGSQLVNDGTWHQVTFSMIDPVAQTSRWQMEVNDQTPFVISEVATGSLNFLKDNTDIYVGDQSVDNPKGLQGCLSTIEIGGIYLSYFENLHGFPGKPQEEQFLKVSTNMVLTGCLPSNACHSSPCLHGGNCEDSYSSYRCACLSGWSGTHCEINIDECFSSPCIHGNCSDGVAAYHCRCEPGYTGVNCEVDVDNCKSHQCANGATCVPEAHGYSCLCFGNFTGRFCRHSRLPSTVCGNEKRNFTCYNGGSCSMFQEDWQCMCWPGFTGEWCEEDINECASDPCINGGLCRDLVNRFLCICDVAFAGERCELDVSGLSFYVSLLLWQNLFQLLSYLVLRMNDEPVVEWGAQENY

SEQ ID NO: 12 - DNA, 5801 bp, Mus musculus
  source 1..5801: mol_type = genomic DNA
tgttcacggaagcctgagggggacacgaatccaatccaggctggaaaaatctgctccaggattgactggttaccgtcttcctgtgcctgtaaggtgctgtgaaagagaagtgctttctgattctctgtctgtggaggagccctgggaggggtgggacagagatggcatcctggctctctgaggcacctgctcttctctgaaccacacaggagtcaagagccaaacagggatagcttcagcagcacttcagagggtgttctctaagtaagaacatgaagctcaagagaactgcctaccttctcttcctgtacctcagctcctcactgctcatctgcataaagaattcattttgcaataaaaacaataccaggtgcctttcaggtccttgccaaaacaattctacgtgcaagcattttccacaagacaacaattgttgcttagacacagccaataatttggacaaagactgtgaagatctgaaagacccttgcttctcgagtccctgccaaggaattgccacttgtgtgaaaatcccaggggaagggaacttcctgtgtcagtgtcctcctgggtacagcgggctgaactgtgaaactgccaccaattcctgtggagggaacctctgccaacatggaggcacctgccgtaaagaccctgagcaccctgtctgtatctgccctcctggatatgctggaaggttctgtgagactgatcacaatgagtgtgcttctagcccttgccacaatggggctatgtgccaggatggaatcaatggctactcctgcttctgtgtgcctggataccaaggcaggcattgtgacttggaagtggatgaatgtgtttctgatccctgcaagaatgaggctgtgtgcctcaatgagataggaagatacacttgtgtctgccctcaagagttttctggcgtgaactgtgagttggaaattgatgaatgcagatcccagccttgtctccacggtgccacatgtcaggacgctccagggggctactcctgtgactgtgcacctggattccttggagagcactgtgaactcagcgttaatgaatgtgaaagtcagccgtgtctccatggaggtctatgtgtggatggaagaaacagttaccactgtgactgcacaggtagtggattcacagggatgcactgtgagtccttgattcctctttgttggtcaaagccttgtcacaacgacgcgacatgtgaagatactgttgacagctatatttgtcactgccggcctggatacacaggtgccctgtgtgagacagacataaatgaatgcagtagcaacccctgccaattttggggggaatgtgtcgagctgtcctcagagggtctatatggaaacactgctggcctgccttcctccttcagctatgttggagcctcgggctatgtgtgtatctgtcagcctggattcacaggaattcactgtgaagaagacgttgatgaatgtttactgcacccttgcctaaatggtggtacttgtgagaacctgcctgggaattatgcctgtcactgtccctttgatgacacttctaggacattttatggaggagaaaactgctcagaaattctcctgggctgcactcatcaccagtgtctgaacaatggaaaatgtatccctcatttccaaaatggccagcatggattcacttgccagtgtctttctggctatgcggggcccctgtgtgaaactgtcaccacactttcatttgggagcaatggcttcctatgggtcacaagtggctcccatacaggcatagggccagaatgtaacatatccttgaggtttcacactgttcaaccaaacgcacttctcctcatccgaggcaacaaggacgtgtctatgaagctggagttgctgaatggttgtgttcacttatcaattgaagtctggaatcagttaaaggtgctcctgtctatttctcacaacaccagtgatggagaatggcatttcgtggaggtaacaatcgcagaaactctaacccttgccctagttggcggctcctgcaaggagaagtgcaccaccaagtcttctgttccagttgagaatcatcaatcaatatgtgctttgcaggactcttttttgggtggcttaccaatggggacagccaacaacagtgtgtctgtgcttaacatctataatgtgccgtccacaccttcctttgtaggctgtctccaagacattagatttgatttgaatcacattactctggagaacgtttcatctggcctgtcatcaaatgttaaagcaggctgcctgggaaaggactggtgtgaaagtcaaccctgtcaaaacagaggacgctgcatcaacttgtggcagggttatcagtgtgaatgtgacaggccctatacaggctccaactgcctgaaaggtgagaggagtggggtgccccagagtgctgtgcctctgagcagagccatctctaatcacccagggtgccgtcccctgttaggaaacataaggacccctcaggacttatgctggtatttgttcactaatgagataaaatggcatagtcatgatatgtattaattatgagtgggtttcataggatagctgagcttttttgggctgaaaagtaaaattaataataataacaataagcaaataactccaattaatgtggtgttttatctagttagcaaaatgctcttagcaatttgccattcattgtgtatcagaaatatatagaaaactttagttctttgtacaagatgtcatcttttagagaaaggggagttttggacagaaaaactagttactgccacgtactaataccacaccttgtgcttgctagagtctcagtgaataaaccctttgctgatctctctgtgtaactcatacttccgtaagaatcgtggttaagattagcatgttgacaagccatcagttctagtcaagactgtctcctaaaaggccttgttttctaaagaggagagatgtcttcagtcggaaaaagcaagaagacatgaactgtattatcaggaaaacttggtagttgtcacgcacagatccgtgattcctctagtgaatcagtttgaagtggatttccaatccctcacttttgacatcactctgaaggctgccatcaatagcgatcaatacatacctgctcacttttattattattatcatgtattgagagggctgaaagggaactctaacagactgctttgaagttcagctgcaatttactctagcattttagaatgagtccaagaagaacaacatatggcaaataggctacgctgttccgtatgaaggaaaattaaaaccagccgtagcctatactctactccatgttcaatggctaagaattattagaaactattcgcaggttttcccctaaccacaatattcattacaatcatggacctgcttgacaatgaggcatggcatctgctgctccgcgcaatattttaaatggcgtggcaagttgttttgtgattatttttaaaggtgaaattatgccgaggcaatggttcacgttttgaagaaaaatctaattgacccaaagcaatatttttactacatatacaaaataacaaacagatgcggactaattttgactggcttccagatgcggtgacccttgaggttagcactgacctgggaatgctgactgtcctaggaaatagattcgagagatgccaagccagcaggttctggttttctttacatttttttttccaacttggaaaataatgaacttttgaaacaaaattcctgatttggttacactttccatattcccccaaatagtgtgattacacccctccactcacaccgagtgtaactatatcccctacccttatacaaagtgtgattaaatcttctattttcacagtttgaaactgtttagctcaacatttgatatcaaatgattatagggcaggaatttcataaaacccttcacttctgaaaatttaggaggagaattttaaaagaaagttatatttttcatgtgaccctgagatcataaagttaaagtattctttattgctgagatccataaggaaatattttctgtattttattccttaaacacacacacacacacttaagactccaaatgagactctatatacatatagagccattctatatgcatattcaggatgaggcacactaaaaatcaagaggggaagccatcaaagtaacaattttttaaagatgtattttattattctatgtgtgtgggtctgagtgtatgcttctgcaccaggtgagtggagattcccctggaacaggagttaatgacagttttgagctgcctgatgtgggtattgggatcaaaccttggtcctctacaaggacagctcatacttttaaccactgagtcacctctccagttctcaaacaatgattttggaacaatgcttgccagtgttaaacccaatgaaagaagaaggcatgttgaataaagggtggagttatctgaatgatacaaaatgtagatagacattgccaatatcttgaaactgatctcaagtcatttatgccccccataaggtttctgtaacaacctgaactgcctgcagtgataacattgtatgtcttgcattatgtgttagaagaaggtcttctgggatattggtctaaagcagttgttctcaaccttcctaatgctgtgaccctttaatatagctccttgtgttttgctgacctcccaaccatacagttattttgttgctacttcacaactgtaacttttgctacttttgtgaattataatataaatgtctgtgttttccaatggtcttagacaagccctgtgacagccctgtgtcattcatctccaaaggcttacggcccacaggtcctaagagaacatgtaacgtacctctttctatgttcggaaagtctctaatttaaaaaaaaaaacaatttatatatgcttgtcttcctttgtacgcccagacttttagaatgctattatattagagtcagtgatagttaggtttgacagagcctcatcagcagctggatttcttatggaaccctctgctttgaacccacttcaggaatcgagaagtcactatcccatctggccccaaattttgaaacaattatttctgatgacgatttaacccagcttcccttttcccacacagttaccactgcggatattctcacttagggctttaacatcccctcttgaaaattcctaaatatttgaagaaaaatattccatgcatagcatccactcccagcatcctacacacattccttacctctagtatctctggaaggcacgtcccagtgggacatcattagctaccttacatgctcctttgccatacatttgcctctttctaacaggtggtatctaaatgtgcttgatgatgcactgacatggaaccacaacttccctctttctatataataggctctcatttatcatgttagcactacatttaatttttgggagagtttacacactgtcttttgtcagtcattgtcattgtgaagctagagagtcctcttctattgtatactgataagtcacatttaatatcaatgcctcctattaacctctcactaaacttcaccttatagtccatcagcattaaaatctctcaaattaaatttttttctccatacatctttagaacatatccactacctgtattagtatcaaatcttccatgtgtagagttgggtccttcctatgtggcttaccgtgtttctaaaatcaagtaacacatcacacactctgactacctgcttgtgatttctgaagaataggcttactggagagtcaagtttctaagg

SEQ ID NO: 13 - protein, 761 aa, Mus musculus
MKLKRTAYLLFLYLSSSLLICIKNSFCNKNNTRCLSGPCQNNSTCKHFPQDNNCCLDTANNLDKDCEDLKDPCFSSPCQGIATCVKIPGEGNFLCQCPPGYSGLNCETATNSCGGNLCQHGGTCRKDPEHPVCICPPGYAGRFCETDHNECASSPCHNGAMCQDGINGYSCFCVPGYQGRHCDLEVDECVSDPCKNEAVCLNEIGRYTCVCPQEFSGVNCELEIDECRSQPCLHGATCQDAPGGYSCDCAPGFLGEHCELSVNECESQPCLHGGLCVDGRNSYHCDCTGSGFTGMHCESLIPLCWSKPCHNDATCEDTVDSYICHCRPGYTGALCETDINECSSNPCQFWGECVELSSEGLYGNTAGLPSSFSYVGASGYVCICQPGFTGIHCEEDVDECLLHPCLNGGTCENLPGNYACHCPFDDTSRTFYGGENCSEILLGCTHHQCLNNGKCIPHFQNGQHGFTCQCLSGYAGPLCETVTTLSFGSNGFLWVTSGSHTGIGPECNISLRFHTVQPNALLLIRGNKDVSMKLELLNGCVHLSIEVWNQLKVLLSISHNTSDGEWHFVEVTIAETLTLALVGGSCKEKCTTKSSVPVENHQSICALQDSFLGGLPMGTANNSVSVLNIYNVPSTPSFVGCLQDIRFDLNHITLENVSSGLSSNVKAGCLGKDWCESQPCQNRGRCINLWQGYQCECDRPYTGSNCLKGERSGVPQSAVPLSRAISNHPGCRPLLGNIRTPQDLCWYLFTNEIKWHSHDMY

SEQ ID NO: 14 - protein, 42 aa, Homo sapiens
DVNECSSNPCQNGGTCENLPGNYTCHCPFDNLSRTFYGGRDC

SEQ ID NO: 15 - protein, 32 aa, Homo sapiens
CESQPCQSRGRCINLWLSYQCDCHRPYEGPNC

SEQ ID NO: 16 - protein, 35 aa, Homo sapiens
NSCKSNPCHNGGVCHSRWDDFSCSCPALTSGKACE

SEQ ID NO: 17 - protein, 30 aa, Homo sapiens
NPCLHGGNCEDIYSSYHCSCPLGWSGKHCE

SEQ ID NO: 18 - protein, 29 aa, Homo sapiens
NPCIHGNCSDRVAAYHCTCEPGYTGVNCE

SEQ ID NO: 19 - protein, 36 aa, Homo sapiens
DIDNCQSHQCANGATCISHTNGYSCLCFGNFTGKFC

SEQ ID NO: 20 - protein, 37 aa, Homo sapiens
DIDECASDPCVNGGLCQDLLNKFQCLCDVAFAGERCE

SEQ ID NO: 21 - protein, 136 aa, Homo sapiens
FQTVQPMALLLFRSNRDVFVKLELLSGYIHLSIQVNNQSKVLLFISHNTSDGEWHFVEVIFAEAVTLTLIDDSCKEKCIAKAPTPLESDQSICAFQNSFLGGLPVGMTSNGVALLNFYNMPSTPSFVGCLQDIKID

SEQ ID NO: 22 - protein, 118 aa, Homo sapiens
VRTLQPSGLLLALENSTYQYIRVWLERGRLAMLTPNSPKLVVKFVLNDGNVHLISLKIKPYKIELYQSSQNLGFISASTWKIEKGDVIYIGGLPDKQETELNGGFFKGCIQDVRLNNQ

SEQ ID NO: 23 - protein, 126 aa, Homo sapiens
FRTRDANVIILHAEKEPEFLNISIQDSRLFFQLQSGNSFYMLSLTSLQSVNDGTWHEVTLSMTDPLSQTSRWQMEVDNETPFVTSTIATGSLNFLKDNTDIYVGDRAIDNIKGLQGCLSTIEIGGI

SEQ ID NO: 24 - DNA, 292 bp, Homo sapiens
  source 1..292: mol_type = genomic DNA
ctcaggggattgtctttttctagcaccttcttgccactcctaagcgtcctccgtgaccccggctgggatttagcctggtgctgtgtcagccccgggctcccaggggcttcccagtggtccccaggaaccctcgacagggccagggcgtctctctcgtccagcaagggcagggacgggccacaggccaagggcagcagtcaggcctgctctgtctgtgaacgctcccggcttggcctcggctgatgggccctcacgcctgaagcgggcaggaagctccgggatggatttcggg

SEQ ID NO: 25 - DNA, 235 bp, Mus musculus
  source 1..235: mol_type = genomic DNA
caattaggccccggtggcagcagtgggattagcgttagtatgatatctcgcggatgctgaatcagcctctggcttagggagagaaggtcactttataagggtctggggggggtcagtgcctggagttgcgctgtgggagccgtcagtggctgagctcgccaagcagccttggtctctgtctacgaagagcccgtggggcagcctcgagagccgcagccatgaacggcacagaggg

SEQ ID NO: 26 - DNA, 508 bp, synthetic construct
  source 1..508: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 27 - DNA, 584 bp, synthetic construct
  misc_feature 1..584: note = Synthetic- chicken beta-actin (CBA promoter)
  source 1..584: mol_type = other DNA
gcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 28 - DNA, 20 bp, synthetic construct
  misc_feature 1..20: note = Synthetic- CRISPR gRNA primer for making Crb1 AB and B mouse
  source 1..20: mol_type = other DNA
gaataagtacccgttccttg

SEQ ID NO: 29 - DNA, 20 bp, synthetic construct
  misc_feature 1..20: note = Synthetic- CRISPR gRNA primer for making Crb1 B mouse
  source 1..20: mol_type = other DNA
aaagcgattaggtgatgccc

SEQ ID NO: 30 - DNA, 20 bp, synthetic construct
  misc_feature 1..20: note = Synthetic- CRISPR gRNA primer for making Crb1 AB mouse
  source 1..20: mol_type = other DNA
tgtccgaacacgtcaacccc

SEQ ID NO: 31 - DNA, 21 bp, synthetic construct
  misc_feature 1..21: note = Synthetic- RT-PCR primer
  source 1..21: mol_type = other DNA
gcttgctcactcgttctcagt

SEQ ID NO: 32 - DNA, 20 bp, synthetic construct
  misc_feature 1..20: note = Synthetic- RT-PCR primer
  source 1..20: mol_type = other DNA
agctctctccttccaaaccc

SEQ ID NO: 33 - DNA, 20 bp, synthetic construct
  misc_feature 1..20: note = Synthetic- RT-PCR primer
  source 1..20: mol_type = other DNA
acccacaagcgtttgctaag

SEQ ID NO: 34 - DNA, 20 bp, synthetic construct
  misc_feature 1..20: note = Synthetic- genotyping primer
  source 1..20: mol_type = other DNA
cagtatcccaggagcattcc

SEQ ID NO: 35 - DNA, 21 bp, synthetic construct
  misc_feature 1..21: note = Synthetic- genotyping primer
  source 1..21: mol_type = other DNA
tttttcagtgtgccaggaagt

SEQ ID NO: 36 - DNA, 20 bp, synthetic construct
  misc_feature 1..20: note = Synthetic- genotyping primer
  source 1..20: mol_type = other DNA
aagactttccgaagccatga

SEQ ID NO: 37 - DNA, 20 bp, synthetic construct
  misc_feature 1..20: note = Synthetic- genotyping primer
  source 1..20: mol_type = other DNA
caagacacccaggaccaagt

SEQ ID NO: 38 - DNA, 20 bp, synthetic construct
  misc_feature 1..20: note = Synthetic- genotyping primer
  source 1..20: mol_type = other DNA
cttccctctttggacattgc

SEQ ID NO: 39 - DNA, 20 bp, synthetic construct
  misc_feature 1..20: note = Synthetic- genotyping primer
  source 1..20: mol_type = other DNA
aacttgggagagcctggagt

SEQ ID NO: 40 - DNA, 20 bp, synthetic construct
  misc_feature 1..20: note = Synthetic- qPCR primer
  source 1..20: mol_type = other DNA
gcctcgggctatgtgtgtat

SEQ ID NO: 41 - DNA, 20 bp, synthetic construct
  misc_feature 1..20: note = Synthetic- qPCR primer
  source 1..20: mol_type = other DNA
aaacggttcctgtcgaccta

SEQ ID NO: 42 - DNA, 20 bp, synthetic construct
  misc_feature 1..20: note = Synthetic- qPCR primer
  source 1..20: mol_type = other DNA
ggcaagggtgcagtaaacat

SEQ ID NO: 43 - DNA, 20 bp, synthetic construct
  misc_feature 1..20: note = Synthetic- qPCR primer
  source 1..20: mol_type = other DNA
tgcatcaatggaggactgtg

SEQ ID NO: 44 - DNA, 20 bp, synthetic construct
  misc_feature 1..20: note = Synthetic- qPCR primer
  source 1..20: mol_type = other DNA
tcatgcgcagtacgaggtag

SEQ ID NO: 45 - DNA, 20 bp, synthetic construct
  misc_feature 1..20: note = Synthetic- qPCR primer
  source 1..20: mol_type = other DNA
tgaagaacagggccaaagtt

SEQ ID NO: 46 - DNA, 20 bp, synthetic construct
  misc_feature 1..20: note = Synthetic- qPCR primer
  source 1..20: mol_type = other DNA
agaggacgctgcatcaactt

SEQ ID NO: 47 - DNA, 20 bp, synthetic construct
  misc_feature 1..20: note = Synthetic- qPCR primer
  source 1..20: mol_type = other DNA
tcatcttggccaaatcttcc

SEQ ID NO: 48 - DNA, 20 bp, synthetic construct
  misc_feature 1..20: note = Synthetic- qPCR primer
  source 1..20: mol_type = other DNA
gctccctcaagggtttgaat

SEQ ID NO: 49 - DNA, 20 bp, synthetic construct
  misc_feature 1..20: note = Synthetic- qPCR primer
  source 1..20: mol_type = other DNA
ccatcaggtgcagcgtataa

SEQ ID NO: 50 - DNA, 48 bp, synthetic construct
  misc_feature 1..48: note = Synthetic- BaseScope probe
  source 1..48: mol_type = other DNA
cacaaggttttcacattttaatggcagtgctcataggaattcactgtg

SEQ ID NO: 51 - DNA, 45 bp, synthetic construct
  misc_feature 1..45: note = Synthetic- BaseScope probe
  source 1..45: mol_type = other DNA
acctcagctcctcactgctcatctgcataaagaattcattttgca

SEQ ID NO: 52 - protein, 42 aa, Homo sapiens
ILLGCTHQQCLNNGTCIPHFQDGQHGFSCLCPSGYTGSLCEI

SEQ ID NO: 53 - protein, 16 aa, Mus musculus
RMNDEPVVEWGAQENY

SEQ ID NO: 54 - DNA, 17 bp, synthetic construct
  misc_feature 1..17: note = Megf11-specific primer
  source 1..17: mol_type = other DNA
ggctccggggtatagga

SEQ ID NO: 55 - DNA, 18 bp, synthetic construct
  misc_feature 1..18: note = Megf11-specific primer
  source 1..18: mol_type = other DNA
ctggctgcattgcattgg

SEQ ID NO: 56 - DNA, 16 bp, synthetic construct
  misc_feature 1..16: note = Megf11-specific primer
  source 1..16: mol_type = other DNA
ggtgtccaataaagtc

SEQ ID NO: 57 - DNA, 5764 bp, Mus musculus; identical to SEQ ID NO: 10
  source 1..5764: mol_type = genomic DNA

SEQ ID NO: 58 - protein, 1003 aa, Mus musculus; identical to SEQ ID NO: 11

SEQ ID NO: 59 - DNA, 6170 bp, Mus musculus; identical to SEQ ID NO: 8
  source 1..6170: mol_type = genomic DNA

SEQ ID NO: 60 - protein, 1405 aa, Mus musculus; identical to SEQ ID NO: 9

SEQ ID NO: 61 - DNA, 5894 bp, Mus musculus
  source 1..5894: mol_type = genomic DNA
gcctttccaggaggcattgttcacggaagcctgagggggacacgaatccaatccaggctggaaaaatctgctccaggattgactggttaccgtcttcctgtgcctgtaaggtgctgtgaaagagaagtgctttctgattctctgtctgtggaggagccctgggaggggtgggacagagatggcatcctggctctctgaggcacctgctcttctctgaaccacacaggagtcaagagccaaacagggatagcttcagcagcacttcagagggtgttctctaagtaagaacatgaagctcaagagaactgcctaccttctcttcctgtacctcagctcctcactgctcatctgcataaagaattcattttgcaataaaaacaataccaggtgcctttcaggtccttgccaaaacaattctacgtgcaagcattttccacaagacaacaattgttgcttagacacagccaataatttggacaaagactgtgaagatctgaaagacccttgcttctcgagtccctgccaaggaattgccacttgtgtgaaaatcccaggggaagggaacttcctgtgtcagtgtcctcctgggtacagcgggctgaactgtgaaactgccaccaattcctgtggagggaacctctgccaacatggaggcacctgccgtaaagaccctgagcaccctgtctgtatctgccctcctggatatgctggaaggttctgtgagactgatcacaatgagtgtgcttctagcccttgccacaatggggctatgtgccaggatggaatcaatggctactcctgcttctgtgtgcctggataccaaggcaggcattgtgacttggaagtggatgaatgtgtttctgatccctgcaagaatgaggctgtgtgcctcaatgagataggaagatacacttgtgtctgccctcaagagttttctggcgtgaactgtgagttggaaattgatgaatgcagatcccagccttgtctccacggtgccacatgtcaggacgctccagggggctactcctgtgactgtgcacctggattccttggagagcactgtgaactcagcgttaatgaatgtgaaagtcagccgtgtctccatggaggtctatgtgtggatggaagaaacagttaccactgtgactgcacaggtagtggattcacagggatgcactgtgagtccttgattcctctttgttggtcaaagccttgtcacaacgacgcgacatgtgaagatactgttgacagctatatttgtcactgccggcctggaattcactgtgaagaagacgttgatgaatgtttactgcacccttgcctaaatggtggtacttgtgagaacctgcctgggaattatgcctgtcactgtccctttgatgacacttctaggacattttatggaggagaaaactgctcagaaattctcctgggctgcactcatcaccagtgtctgaacaatggaaaatgtatccctcatttccaaaatggccagcatggattcacttgccagtgtctttctggctatgcggggcccctgtgtgaaactgtcaccacactttcatttgggagcaatggcttcctatgggtcacaagtggctcccatacaggcatagggccagaatgtaacatatccttgaggtttcacactgttcaaccaaacgcacttctcctcatccgaggcaacaaggacgtgtctatgaagctggagttgctgaatggttgtgttcacttatcaattgaagtctggaatcagttaaaggtgctcctgtctatttctcacaacaccagtgatggagaatggcatttcgtggaggtaacaatcgcagaaactctaacccttgccctagttggcggctcctgcaaggagaagtgcaccaccaagtcttctgttccagttgagaatcatcaatcaatatgtgctttgcaggactcttttttgggtggcttaccaatggggacagccaacaacagtgtgtctgtgcttaacatctataatgtgccgtccacaccttcctttgtaggctgtctccaagacattagatttgatttgaatcacattactctggagaacgtttcatctggcctgtcatcaaatgttaaagcaggctgcctgggaaaggactggtgtgaaagtcaaccctgtcaaaacagaggacgctgcatcaacttgtggcagggttatcagtgtgaatgtgacaggccctatacaggctccaactgcctgaaagagtatgtagcgggaagatttggccaagatgactccacaggatatgcggcctttagtgttaatgataattatggacagaacttcagtctttcaatgtttgtccgaacacgtcaacccctgggcttacttctggctttggaaaatagtacttaccagtatgtcagtgtctggctagagcacggcagcctagcactgcagactccaggctctcccaagttcatggtaaacttttttctcagtgatggaaatgttcacttaatatctttgagaatcaaaccaaatgaaattgaactgtatcagtcttcacaaaacctaggattcatttctgttcctacatggacaattcgaagaggagacgtcatcttcattggtggcttacctgacagagagaagactgaagtttatggtggcttcttcaaaggctgtgttcaagatgtcagattaaacagccagactctggaattctttcccaattcaacaaacaatgcatacgatgacccaattcttgtcaatgtgactcaaggctgtcccggagacaacacatgtaagtccaacccctgtcataatggaggtgtctgccactccctgtgggatgacttctcctgctcctgccctacaaacacagcggggagagcctgcgagcaagttcagtggtgtcaactcagcccatgtcctcccactgcagagtgccagctgctccctcaagggtttgaatgtatcgcaaacgctgttttcagcggattaagcagagaaatactcttcagaagcaatgggaacattaccagagaactcaccaatatcacatttgctttcagaacacatgatacaaatgtgatgatattgcatgcagaaaaagaaccagagtttcttaatattagcattcaagatgccagattattctttcaattgcgaagtggcaacagcttttatacgctgcacctgatgggttcccaattggtgaatgatggcacatggcaccaagtgactttctccatgatagacccagtggcccagacctcccggtggcaaatggaggtgaacgaccagacaccctttgtgataagtgaagttgctactggaagcctgaactttttgaaggacaatacagacatctatgtgggtgaccaatctgttgacaatccgaaaggcctgcagggctgtctgagcacaatagagattggaggcatatatctttcttactttgaaaatctacatggtttccctggtaagcctcaggaagagcaatttctcaaagtttctacaaatatggtacttactggctgtttgccatcaaatgcctgccactccagcccctgtttgcatggaggaaactgtgaagacagctacagttcttatcggtgtgcctgtctctcgggatggtcagggacacactgtgaaatcaacattgatgagtgcttttctagcccctgtatccatggcaactgctctgatggagttgcagcctaccactgcaggtgtgagcctggatacaccggtgtgaactgtgaggtggatgtagacaattgcaagagtcatcagtgtgcaaatggggccacctgtgttcctgaagctcatggctactcttgtctctgctttggaaattttaccgggagattttgcagacacagcagattaccctcaacagtctgtgggaatgagaagagaaacttcacttgctacaatggaggcagctgctccatgttccaggaggactggcaatgtatgtgctggccaggtttcactggagagtggtgtgaagaggacatcaacgagtgtgcctccgatccctgcatcaatggaggactgtgcagggacttggtcaacaggttcctatgcatctgtgatgtggccttcgctggcgagcgctgtgagctggacctggctgatgacaggctcctgggcattttcaccgctgttggctccggaactttggccctgttcttcatcctcttgcttgctggggttgcttctcttattgcctccaacaaaagggcgactcaaggaacctacagccccagcggtcaggagaaggctggccctcgagtggaaatgtggatcaggatgccgcccccggcactggaaaggctcatctaggagactgctgctcttctcaggacagagaagaacatgatgagtaccgggtcgtgcctgagtgaagatggctttacatcactagagatacatacagctgggactgtgggaaggaccttcctgtggagtcactgagtagttatgtcatccattcacagaagagtgtccctgtgtttgcctgtcagcctcagaattagcaaaacatctagcagacagagaacacagtatttcagaagaactccagaggctgccccttaaactctttactggttgatccacataaaatgcttagtagccaagtgccattaattatacagagccaagaagaaaaattagaatacaactttcactttttattttgtagggaaggttttatgttttggtttgttgttgttgttgtgacagtgacagtgactcattacatagaccaagctggcctcaaaatcacatggaccctcgggattacatgtgtccgaccatgttcatcttatttttgaatcttctgtcatatggtaaaagattccagtgggacctgaggagtgactagctaggtaaagcaagggctgtgtaagtgccagaactggtgtttgtgtcctcattatccacataagtgccaagtgagtgtggcccctgcctgtcatcctaggcctcaggagatatcactgctcactggagcaagccggttaaactgttagggcaggtaagttttgacttcaagtgagagaccctgactcaatatgaaaggcaattagtgagtcaagatgaccctgtatgctaacctcttgcctatacatgcatatacacacatttacatatgtgcccaaacatgaggacacaagcacacgcgcgcgcgcacacacacacacacacacacacacacacacacacacacacacacacgagtctaattgtatatagtgataacagtacactttcctccttctatttcggatttagagaaagccatgagaagcgtgtatggtttaaaccatgacccaagcataacaaataaagttgaaatagttgttctcctgtccaagcttgtctttattgttgtgcattctgtaagctggttgcttggttggctgatggatggcttctgtttgtttgttgttttttgtttgtttgtttgtctgggatattacatgtaagaaaaataactggtaagaacaatcaaagaactttgttatgaattaaatcttttgtctaagtcacttagagtcattattctttatgtagatttgcttccagtcaggacatttcctagacagaatttaagacagtaagaaaatgatttgtcacgtctgaaagaggttctttactttcagggacttttgataatgcccaacagagatggcatcgaaagaggagctcatagcgagatgggcatttgtgcatcctcaaggagaaaatattgtaccttctgtttgtatattgtctattctgtgatggctgtatcttacatatgttttgatgcatgtaacaatagtatcatatgaaataaattatatatatatataatatataatatatatcacaagataaaaattgaaattacataaactttaaatct

SEQ ID NO: 62 - protein, 1344 aa, Mus musculus
MKLKRTAYLLFLYLSSSLLICIKNSFCNKNNTRCLSGPCQNNSTCKHFPQDNNCCLDTANNLDKDCEDLKDPCFSSPCQGIATCVKIPGEGNFLCQCPPGYSGLNCETATNSCGGNLCQHGGTCRKDPEHPVCICPPGYAGRFCETDHNECASSPCHNGAMCQDGINGYSCFCVPGYQGRHCDLEVDECVSDPCKNEAVCLNEIGRYTCVCPQEFSGVNCELEIDECRSQPCLHGATCQDAPGGYSCDCAPGFLGEHCELSVNECESQPCLHGGLCVDGRNSYHCDCTGSGFTGMHCESLIPLCWSKPCHNDATCEDTVDSYICHCRPGIHCEEDVDECLLHPCLNGGTCENLPGNYACHCPFDDTSRTFYGGENCSEILLGCTHHQCLNNGKCIPHFQNGQHGFTCQCLSGYAGPLCETVTTLSFGSNGFLWVTSGSHTGIGPECNISLRFHTVQPNALLLIRGNKDVSMKLELLNGCVHLSIEVWNQLKVLLSISHNTSDGEWHFVEVTIAETLTLALVGGSCKEKCTTKSSVPVENHQSICALQDSFLGGLPMGTANNSVSVLNIYNVPSTPSFVGCLQDIRFDLNHITLENVSSGLSSNVKAGCLGKDWCESQPCQNRGRCINLWQGYQCECDRPYTGSNCLKEYVAGRFGQDDSTGYAAFSVNDNYGQNFSLSMFVRTRQPLGLLLALENSTYQYVSVWLEHGSLALQTPGSPKFMVNFFLSDGNVHLISLRIKPNEIELYQSSQNLGFISVPTWTIRRGDVIFIGGLPDREKTEVYGGFFKGCVQDVRLNSQTLEFFPNSTNNAYDDPILVNVTQGCPGDNTCKSNPCHNGGVCHSLWDDFSCSCPTNTAGRACEQVQWCQLSPCPPTAECQLLPQGFECIANAVFSGLSREILFRSNGNITRELTNITFAFRTHDTNVMILHAEKEPEFLNISIQDARLFFQLRSGNSFYTLHLMGSQLVNDGTWHQVTFSMIDPVAQTSRWQMEVNDQTPFVISEVATGSLNFLKDNTDIYVGDQSVDNPKGLQGCLSTIEIGGIYLSYFENLHGFPGKPQEEQFLKVSTNMVLTGCLPSNACHSSPCLHGGNCEDSYSSYRCACLSGWSGTHCEINIDECFSSPCIHGNCSDGVAAYHCRCEPGYTGVNCEVDVDNCKSHQCANGATCVPEAHGYSCLCFGNFTGRFCRHSRLPSTVCGNEKRNFTCYNGGSCSMFQEDWQCMCWPGFTGEWCEEDINECASDPCINGGLCRDLVNRFLCICDVAFAGERCELDLADDRLLGIFTAVGSGTLALFFILLLAGVASLIASNKRATQGTYSPSGQEKAGPRVEMWIRMPPPALERLI

SEQ ID NO: 63 - DNA, 5554 bp, Mus musculus
  source 1..5554: mol_type = genomic DNA
cccactgatccagcttgaagaggagtgaggcaaagctgaaccctcccactctccttgacaagtgcaagcccacacttttggaaaaaagcacaaagacgtcagaaacggttcctgtcgacctactaggctttggatggctaagtgtttttgctttgtatggaaatatgtttggacacaagacacaaggttttcacattttaatggcagtgctcataggaattcactgtgaagaagacgttgatgaatgtttactgcacccttgcctaaatggtggtacttgtgagaacctgcctgggaattatgcctgtcactgtccctttgatgacacttctaggacattttatggaggagaaaactgctcagaaattctcctgggctgcactcatcaccagtgtctgaacaatggaaaatgtatccctcatttccaaaatggccagcatggattcacttgccagtgtctttctggctatgcggggcccctgtgtgaaactgtcaccacactttcatttgggagcaatggcttcctatgggtcacaagtggctcccatacaggcatagggccagaatgtaacatatccttgaggtttcacactgttcaaccaaacgcacttctcctcatccgaggcaacaaggacgtgtctatgaagctggagttgctgaatggttgtgttcacttatcaattgaagtctggaatcagttaaaggtgctcctgtctatttctcacaacaccagtgatggagaatggcatttcgtggaggtaacaatcgcagaaactctaacccttgccctagttggcggctcctgcaaggagaagtgcaccaccaagtcttctgttccagttgagaatcatcaatcaatatgtgctttgcaggactcttttttgggtggcttaccaatggggacagccaacaacagtgtgtctgtgcttaacatctataatgtgccgtccacaccttcctttgtaggctgtctccaagacattagatttgatttgaatcacattactctggagaacgtttcatctggcctgtcatcaaatgttaaagcaggctgcctgggaaaggactggtgtgaaagtcaaccctgtcaaaacagaggacgctgcatcaacttgtggcagggttatcagtgtgaatgtgacaggccctatacaggctccaactgcctgaaagagtatgtagcgggaagatttggccaagatgactccacaggatatgcggcctttagtgttaatgataattatggacagaacttcagtctttcaatgtttgtccgaacacgtcaacccctgggcttacttctggctttggaaaatagtacttaccagtatgtcagtgtctggctagagcacggcagcctagcactgcagactccaggctctcccaagttcatggtaaacttttttctcagtgatggaaatgttcacttaatatctttgagaatcaaaccaaatgaaattgaactgtatcagtcttcacaaaacctaggattcatttctgttcctacatggacaattcgaagaggagacgtcatcttcattggtggcttacctgacagagagaagactgaagtttatggtggcttcttcaaaggctgtgttcaagatgtcagattaaacagccagactctggaattctttcccaattcaacaaacaatgcatacgatgacccaattcttgtcaatgtgactcaaggctgtcccggagacaacacatgtaagtccaacccctgtcataatggaggtgtctgccactccctgtgggatgacttctcctgctcctgccctacaaacacagcggggagagcctgcgagcaagttcagtggtgtcaactcagcccatgtcctcccactgcagagtgccagctgctccctcaagggtttgaataacacatgatacaaatgtgatgatattgcatgcagaaaaagaaccagagtttcttaatattagcattcaagatgccagattattctttcaattgcgaagtggcaacagcttttatacgctgcacctgatgggttcccaattggtgaatgatggcacatggcaccaagtgactttctccatgatagacccagtggcccagacctcccggtggcaaatggaggtgaacgaccagacaccctttgtgataagtgaagttgctactggaagcctgaactttttgaaggacaatacagacatctatgtgggtgaccaatctgttgacaatccgaaaggcctgcagggctgtctgagcacaatagagattggaggcatatatctttcttactttgaaaatctacatggtttccctggtaagcctcaggaagagcaatttctcaaagtttctacaaatatggtacttactggctgtttgccatcaaatgcctgccactccagcccctgtttgcatggaggaaactgtgaagacagctacagttcttatcggtgtgcctgtctctcgggatggtcagggacacactgtgaaatcaacattgatgagtgcttttctagcccctgtatccatggcaactgctctgatggagttgcagcctaccactgcaggtgtgagcctggatacaccggtgtgaactgtgaggtggatgtagacaattgcaagagtcatcagtgtgcaaatggggccacctgtgttcctgaagctcatggctactcttgtctctgctttggaaattttaccgggagattttgcagacacagcagattaccctcaacagtctgtgggaatgagaagagaaacttcacttgctacaatggaggcagctgctccatgttccaggaggactggcaatgtatgtgctggccaggtttcactggagagtggtgtgaagaggacatcaacgagtgtgcctccgatccctgcatcaatggaggactgtgcagggacttggtcaacaggttcctatgcatctgtgatgtggccttcgctggcgagcgctgtgagctggacgtaagcggcctttccttttatgtgtccctcttactatggcaaaacctctttcagctcctgtcctacctcgtactgcgcatgaatgatgagccagttgtagagtggggggcacaggaaaattattaatgtgcatgggagcattcacaagtgtaaaacattgacttgcaagaaacatcttgtctcagtgtaggtttctaggaaagacaaagggaacattagggaatagactccatctagagcactggttctcagtcttcctaatgctgcaaccctttagtacagctcttcctgttgtagtgatcgcagccataacattattttcattgccacttcataactgtaatccttctactgctgtgaatcacaatggaaatatttatgttttctgatggtcttaagcaacacctctgaaaaagtcattgaccccccccccaaaggggctgtgatccacaggttgagaaatgctcatctggaaggtaaccatgcatttaagtgtacctctagtagtttgggtctatagaagatattctcctattctacctttttagacacgccagaagagggcatctgattccattaaagatgattgggagccaccgtgtggttcctgagaactgtactcgggccctttggaagagcaatcagtgctctttccagcccctaagaatatttttaatacagccagaaaggtctcattacccagtgtactgagccctaaggcactttcatcctcaatcgttccatgttgaatggttttcattacatttggaaaatgttttctctccactctacctttacatgttcctattttcctattgacaatttgccccttcactgtaattctaatttggtgtggtccttcttctcataagtttatatgtgacatgaacatttaaaaatatctatgaatattttatagtcatgtatgtctttctgcaaagctattcaaatgaactatggacagttcttttctacacgaagaagagatgagtttaatccccagtaacatgagaaaaagatgagtgagggacagtgctcacagtatccctcactagcatcatttgtgattccatgggccatttttttccaccagcaaatagcagagagccctttccctattcgtttctcttacacttccccttttctgttacaactgaacactttacattagttactcctttgtagggggtttgacttttccaccgttttctctggttcactatttatgctaagtatctgtgcagggcgggtatatcagtccaacagaggtgtcattagtgttcattgaggaggaaatactttgcatgaattcatgacatcattgaagtagcagtggccagaaagatacccttctgcgaatgtgtctgtgtattcagaagctgccctggttagaaaacatgtgggtcacttttcctttgcatgttaccagtgctcactgggtcatgattgttttaagacagagcttttgctgtggcaatgaccaaggtgaatccagagatgcagatcagacaaaggacaagacaatgtactatctgagtaaaaccctgccttgacttactcctcagtacttagagattttacatagcaacctccaccctgtggcaacccgttcacactagcagtgatgctgagatttgcccttccttctcatcatcttcctcacatccaaagcattttgtgtccacactgctgtttcagataactgtttctaaagtgggattgttgtagccagaaaggtagggaaaatgttccccaaaatatttgcattcttaagtatgtgaagtaagtagattatagtcagagacaatatgtaaggtttcaggttcactcccttctacacatatcttcaactgtgtatttgcagaatattctgaatgtgacatactcccaacagaatatatttaaggagtatttatccacagtattgttctctgtacagttctagtgcttctattgtcactgcaattgtcaattgtttttctgctttccaactgtcttattatcatttaatagcatcttgctaaatgccctctttctattctccttatttctccatagttcatgtgtgtctgtgtgactaaggattctcctcatttttgcagaaaaataaaatcttttcttctttatgtcctgcttgtcattctctggtgacacatgtctttgcttacttggactgagggttgtacagtaagtacagaagcaggctcagtcacacagacagagacacaccaccaccagcagcagcagcaccaccaccaccaccaccaccaccagaaaacagtatgagtactcatctcttgattacatgtcatttcaagtaagcaccatgacaccgagggccaggttccatggactttctctgttaggcacgtgattctttagctgacctttgagaacagactccaacaacctcacttatttttactgttgacttatatcatctctgacaacactggacttcgtttgagctagtcaagaggaaagaccatgacacctaagggacagaaattcacacactcggtttttcataattcacacacattcctatgtatcaaatctctgtaatagatgacatttacttgaataaaaagtcatttccctttg

SEQ ID NO: 64 - protein, 574 aa, Mus musculus
MFGHKTQGFHILMAVLIGIHCEEDVDECLLHPCLNGGTCENLPGNYACHCPFDDTSRTFYGGENCSEILLGCTHHQCLNNGKCIPHFQNGQHGFTCQCLSGYAGPLCETVTTLSFGSNGFLWVTSGSHTGIGPECNISLRFHTVQPNALLLIRGNKDVSMKLELLNGCVHLSIEVWNQLKVLLSISHNTSDGEWHFVEVTIAETLTLALVGGSCKEKCTTKSSVPVENHQSICALQDSFLGGLPMGTANNSVSVLNIYNVPSTPSFVGCLQDIRFDLNHITLENVSSGLSSNVKAGCLGKDWCESQPCQNRGRCINLWQGYQCECDRPYTGSNCLKEYVAGRFGQDDSTGYAAFSVNDNYGQNFSLSMFVRTRQPLGLLLALENSTYQYVSVWLEHGSLALQTPGSPKFMVNFFLSDGNVHLISLRIKPNEIELYQSSQNLGFISVPTWTIRRGDVIFIGGLPDREKTEVYGGFFKGCVQDVRLNSQTLEFFPNSTNNAYDDPILVNVTQGCPGDNTCKSNPCHNGGVCHSLWDDFSCSCPTNTAGRACEQVQWCQLSPCPPTAECQLLPQGFE

SEQ ID NO: 65 - DNA, 4783 bp, Mus musculus
  source 1..4783: mol_type = genomic DNA
gatccagcttgaagaggagtgaggcaaagctgaaccctcccactctccttgacaagtgcaagcccacacttttggaaaaaagcacaaagacgtcagaaacggttcctgtcgacctactaggctttggatggctaagtgtttttgctttgtatggaaatatgtttggacacaagacacaaggttttcacattttaatggcagtgctcataggaattcactgtgaagaagacgttgatgaatgtttactgcacccttgcctaaatggtggtacttgtgagaacctgcctgggaattatgcctgtcactgtccctttgatgacacttctaggacattttatggaggagaaaactgctcagaaattctcctgggctgcactcatcaccagtgtctgaacaatggaaaatgtatccctcatttccaaaatggccagcatggattcacttgccagtgtctttctggctatgcggggcccctgtgtgaaactgtcaccacactttcatttgggagcaatggcttcctatgggtcacaagtggctcccatacaggcatagggccagaatgtaacatatccttgaggtttcacactgttcaaccaaacgcacttctcctcatccgaggcaacaaggacgtgtctatgaagctggagttgctgaatggttgtgttcacttatcaattgaagtctggaatcagttaaaggtgctcctgtctatttctcacaacaccagtgatggagaatggcatttcgtggaggtaacaatcgcagaaactctaacccttgccctagttggcggctcctgcaaggagaagtgcaccaccaagtcttctgttccagttgagaatcatcaatcaatatgtgctttgcaggactcttttttgggtggcttaccaatggggacagccaacaacagtgtgtctgtgcttaacatctataatgtgccgtccacaccttcctttgtaggctgtctccaagacattagatttgatttgaatcacattactctggagaacgtttcatctggcctgtcatcaaatgttaaagcaggctgcctgggaaaggactggtgtgaaagtcaaccctgtcaaaacagaggacgctgcatcaacttgtggcagggttatcagtgtgaatgtgacaggccctatacaggctccaactgcctgaaagagtatgtagcgggaagatttggccaagatgactccacaggatatgcggcctttagtgttaatgataattatggacagaacttcagtctttcaatgtttgtccgaacacgtcaacccctgggcttacttctggctttggaaaatagtacttaccagtatgtcagtgtctggctagagcacggcagcctagcactgcagactccaggctctcccaagttcatggtaaacttttttctcagtgatggaaatgttcacttaatatctttgagaatcaaaccaaatgaaattgaactgtatcagtcttcacaaaacctaggattcatttctgttcctacatggacaattcgaagaggagacgtcatcttcattggtggcttacctgacagagagaagactgaagtttatggtggcttcttcaaaggctgtgttcaagatgtcagattaaacagccagactctggaattctttcccaattcaacaaacaatgcatacgatgacccaattcttgtcaatgtgactcaaggctgtcccggagacaacacatgtaagtccaacccctgtcataatggaggtgtctgccactccctgtgggatgacttctcctgctcctgccctacaaacacagcggggagagcctgcgagcaagttcagtggtgtcaactcagcccatgtcctcccactgcagagtgccagctgctccctcaagggtttgaatgtatcgcaaacgctgttttcagcggattaagcagagaaatactcttcagaagcaatgggaacattaccagagaactcaccaatatcacatttgctttcagaacacatgatacaaatgtgatgatattgcatgcagaaaaagaaccagagtttcttaatattagcattcaagatgccagattattctttcaattgcgaagtggcaacagcttttatacgctgcacctgatgggttcccaattggtgaatgatggcacatggcaccaagtgactttctccatgatagacccagtggcccagacctcccggtggcaaatggaggtgaacgaccagacaccctttgtgataagtgaagttgctactggaagcctgaactttttgaaggacaatacagacatctatgtgggtgaccaatctgttgacaatccgaaaggcctgcagggctgtctgagcacaatagagattggaggcatatatctttcttactttgaaaatctacatggtttccctggtaagcctcaggaagagcaatttctcaaagtttctacaaatatggtacttactggctgtttgccatcaaatgcctgccactccagcccctgtttgcatggaggaaactgtgaagacagctacagttcttatcggtgtgcctgtctctcgggatggtcagggacacactgtgaaatcaacattgatgagtgcttttctagcccctgtatccatggcaactgctctgatggagttgcagcctaccactgcaggtgtgagcctggatacaccggtgtgaactgtgaggtggatgtagacaattgcaagagtcatcagtgtgcaaatggggccacctgtgttcctgaagctcatggctactcttgtctctgctttggaaattttaccgggagattttgcagacacagcagattaccctcaacagtctgtgggaatgagaagagaaacttcacttgctacaatggaggcagctgctccatgttccaggaggactggcaatgtatgtgctggccaggtttcactggagagtggtgtgaagaggacatcaacgagtgtgcctccgatccctgcatcaatggaggactgtgcagggacttggtcaacaggttcctatgcatctgtgatgtggccttcgctggcgagcgctgtgagctggacctggctgatgacaggctcctgggcattttcaccgctgttggctccggaactttggccctgttcttcatcctcttgcttgctggggttgcttctcttattgcctccaacaaaagggcgactcaaggaacctacagccccagcggtcaggagaaggctggccctcgagtggaaatgtggatcaggatgccgcccccggcactggaaaggctcatctaggagactgctgctcttctcaggacagagaagaacatgatgagtaccgggtcgtgcctgagtgaagatggctttacatcactagagatacatacagctgggactgtgggaaggaccttcctgtggagtcactgagtagttatgtcatccattcacagaagagtgtccctgtgtttgcctgtcagcctcagaattagcaaaacatctagcagacagagaacacagtatttcagaagaactccagaggctgccccttaaactctttactggttgatccacataaaatgcttagtagccaagtgccattaattatacagagccaagaagaaaaattagaatacaactttcactttttattttgtagggaaggttttatgttttggtttgttgttgttgttgtgacagtgacagtgactcattacatagaccaagctggcctcaaaatcacatggaccctcgggattacatgtgtccgaccatgttcatcttatttttgaatcttctgtcatatggtaaaagattccagtgggacctgaggagtgactagctaggtaaagcaagggctgtgtaagtgccagaactggtgtttgtgtcctcattatccacataagtgccaagtgagtgtggcccctgcctgtcatcctaggcctcaggagatatcactgctcactggagcaagccggttaaactgttagggcaggtaagttttgacttcaagtgagagaccctgactcaatatgaaaggcaattagtgagtcaagatgaccctgtatgctaacctcttgcctatacatgcatatacacacatttacatatgtgcccaaacatgaggacacaagcacacgcgcgcgcgcacacacacacacacacacacacacacacacacacacacacacacacgagtctaattgtatatagtgataacagtacactttcctccttctatttcggatttagagaaagccatgagaagcgtgtatggtttaaaccatgacccaagcataacaaataaagttgaaatagttgttctcctgtccaagcttgtctttattgttgtgcattctgtaagctggttgcttggttggctgatggatggcttctgtttgtttgttgttttttgtttgtttgtttgtctgggatattacatgtaagaaaaataactggtaagaacaatcaaagaactttgttatgaattaaatcttttgtctaagtcacttagagtcattattctttatgtagatttgcttccagtcaggacatttcctagacagaatttaagacagtaagaaaatgatttgtcacgtctgaaagaggttctttactttcagggacttttgataatgcccaacagagatggcatcgaaagaggagctcatagcgagatgggcatttgtgcatcctcaaggagaaaatattgtaccttctgtttgtatattgtctattctgtgatggctgtatcttacatatgttttgatgcatgtaacaatagtatcatatgaaataaattatatatatatataatatat

SEQ ID NO: 66 - protein, 1033 aa, Mus musculus
MFGHKTQGFHILMAVLIGIHCEEDVDECLLHPCLNGGTCENLPGNYACHCPFDDTSRTFYGGENCSEILLGCTHHQCLNNGKCIPHFQNGQHGFTCQCLSGYAGPLCETVTTLSFGSNGFLWVTSGSHTGIGPECNISLRFHTVQPNALLLIRGNKDVSMKLELLNGCVHLSIEVWNQLKVLLSISHNTSDGEWHFVEVTIAETLTLALVGGSCKEKCTTKSSVPVENHQSICALQDSFLGGLPMGTANNSVSVLNIYNVPSTPSFVGCLQDIRFDLNHITLENVSSGLSSNVKAGCLGKDWCESQPCQNRGRCINLWQGYQCECDRPYTGSNCLKEYVAGRFGQDDSTGYAAFSVNDNYGQNFSLSMFVRTRQPLGLLLALENSTYQYVSVWLEHGSLALQTPGSPKFMVNFFLSDGNVHLISLRIKPNEIELYQSSQNLGFISVPTWTIRRGDVIFIGGLPDREKTEVYGGFFKGCVQDVRLNSQTLEFFPNSTNNAYDDPILVNVTQGCPGDNTCKSNPCHNGGVCHSLWDDFSCSCPTNTAGRACEQVQWCQLSPCPPTAECQLLPQGFECIANAVFSGLSREILFRSNGNITRELTNITFAFRTHDTNVMILHAEKEPEFLNISIQDARLFFQLRSGNSFYTLHLMGSQLVNDGTWHQVTFSMIDPVAQTSRWQMEVNDQTPFVISEVATGSLNFLKDNTDIYVGDQSVDNPKGLQGCLSTIEIGGIYLSYFENLHGFPGKPQEEQFLKVSTNMVLTGCLPSNACHSSPCLHGGNCEDSYSSYRCACLSGWSGTHCEINIDECFSSPCIHGNCSDGVAAYHCRCEPGYTGVNCEVDVDNCKSHQCANGATCVPEAHGYSCLCFGNFTGRFCRHSRLPSTVCGNEKRNFTCYNGGSCSMFQEDWQCMCWPGFTGEWCEEDINECASDPCINGGLCRDLVNRFLCICDVAFAGERCELDLADDRLLGIFTAVGSGTLALFFILLLAGVASLIASNKRATQGTYSPSGQEKAGPRVEMWIRMPPPALERLI

SEQ ID NO: 67 - DNA, 6739 bp, Mus musculus
  source 1..6739: mol_type = genomic DNA
gcaatcaaggaccctatcctaaaacaaagcctttccaggaggcattgttcacggaagcctgagggggacacgaatccaatccaggctggaaaaatctgctccaggattgactggttaccgtcttcctgtgcctgtaaggtgctgtgaaagagaagtgctttctgattctctgtctgtggaggagccctgggaggggtgggacagagatggcatcctggctctctgaggcacctgctcttctctgaaccacacaggagtcaagagccaaacagggatagcttcagcagcacttcagagggtgttctctaagtaagaacatgaagctcaagagaactgcctaccttctcttcctgtacctcagctcctcactgctcatctgcataaagaattcattttgcaataaaaacaataccaggtgcctttcaggtccttgccaaaacaattctacgtgcaagcattttccacaagacaacaattgttgcttagacacagccaataatttggacaaagactgtgaagatctgaaagacccttgcttctcgagtccctgccaaggaattgccacttgtgtgaaaatcccaggggaagggaacttcctgtgtcagtgtcctcctgggtacagcgggctgaactgtgaaactgccaccaattcctgtggagggaacctctgccaacatggaggcacctgccgtaaagaccctgagcaccctgtctgtatctgccctcctggatatgctggaaggttctgtgagactgatcacaatgagtgtgcttctagcccttgccacaatggggctatgtgccaggatggaatcaatggctactcctgcttctgtgtgcctggataccaaggcaggcattgtgacttggaagtggatgaatgtgtttctgatccctgcaagaatgaggctgtgtgcctcaatgagataggaagatacacttgtgtctgccctcaagagttttctggcgtgaactgtgagttggaaattgatgaatgcagatcccagccttgtctccacggtgccacatgtcaggacgctccagggggctactcctgtgactgtgcacctggattccttggagagcactgtgaactcagcgttaatgaatgtgaaagtcagccgtgtctccatggaggtctatgtgtggatggaagaaacagttaccactgtgactgcacaggtagtggattcacagggatgcactgtgagtccttgattcctctttgttggtcaaagccttgtcacaacgacgcgacatgtgaagatactgttgacagctatatttgtcactgccggcctggaattcactgtgaagaagacgttgatgaatgtttactgcacccttgcctaaatggtggtacttgtgagaacctgcctgggaattatgcctgtcactgtccctttgatgacacttctaggacattttatggaggagaaaactgctcagaaattctcctgggctgcactcatcaccagtgtctgaacaatggaaaatgtatccctcatttccaaaatggccagcatggattcacttgccagtgtctttctggctatgcggggcccctgtgtgaaactgtcaccacactttcatttgggagcaatggcttcctatgggtcacaagtggctcccatacaggcatagggccagaatgtaacatatccttgaggtttcacactgttcaaccaaacgcacttctcctcatccgaggcaacaaggacgtgtctatgaagctggagttgctgaatggttgtgttcacttatcaattgaagtctggaatcagttaaaggtgctcctgtctatttctcacaacaccagtgatggagaatggcatttcgtggaggtaacaatcgcagaaactctaacccttgccctagttggcggctcctgcaaggagaagtgcaccaccaagtcttctgttccagttgagaatcatcaatcaatatgtgctttgcaggactcttttttgggtggcttaccaatggggacagccaacaacagtgtgtctgtgcttaacatctataatgtgccgtccacaccttcctttgtaggctgtctccaagacattagatttgatttgaatcacattactctggagaacgtttcatctggcctgtcatcaaatgttaaagcaggctgcctgggaaaggactggtgtgaaagtcaaccctgtcaaaacagaggacgctgcatcaacttgtggcagggttatcagtgtgaatgtgacaggccctatacaggctccaactgcctgaaagagtatgtagcgggaagatttggccaagatgactccacaggatatgcggcctttagtgttaatgataattatggacagaacttcagtctttcaatgtttgtccgaacacgtcaacccctgggcttacttctggctttggaaaatagtacttaccagtatgtcagtgtctggctagagcacggcagcctagcactgcagactccaggctctcccaagttcatggtaaacttttttctcagtgatggaaatgttcacttaatatctttgagaatcaaaccaaatgaaattgaactgtatcagtcttcacaaaacctaggattcatttctgttcctacatggacaattcgaagaggagacgtcatcttcattggtggcttacctgacagagagaagactgaagtttatggtggcttcttcaaaggctgtgttcaagatgtcagattaaacagccagactctggaattctttcccaattcaacaaacaatgcatacgatgacccaattcttgtcaatgtgactcaaggctgtcccggagacaacacatgtaagtccaacccctgtcataatggaggtgtctgccactccctgtgggatgacttctcctgctcctgccctacaaacacagcggggagagcctgcgagcaagttcagtggtgtcaactcagcccatgtcctcccactgcagagtgccagctgctccctcaagggtttgaatgtatcgcaaacgctgttttcagcggattaagcagagaaatactcttcagaagcaatgggaacattaccagagaactcaccaatatcacatttgctttcagaacacatgatacaaatgtgatgatattgcatgcagaaaaagaaccagagtttcttaatattagcattcaagatgccagattattctttcaattgcgaagtggcaacagcttttatacgctgcacctgatgggttcccaattggtgaatgatggcacatggcaccaagtgactttctccatgatagacccagtggcccagacctcccggtggcaaatggaggtgaacgaccagacaccctttgtgataagtgaagttgctactggaagcctgaactttttgaaggacaatacagacatctatgtgggtgaccaatctgttgacaatccgaaaggcctgcagggctgtctgagcacaatagagattggaggcatatatctttcttactttgaaaatctacatggtttccctggtaagcctcaggaagagcaatttctcaaagtttctacaaatatggtacttactggctgtttgccatcaaatgcctgccactccagcccctgtttgcatggaggaaactgtgaagacagctacagttcttatcggtgtgcctgtctctcgggatggtcagggacacactgtgaaatcaacattgatgagtgcttttctagcccctgtatccatggcaactgctctgatggagttgcagcctaccactgcaggtgtgagcctggatacaccggtgtgaactgtgaggtggatgtagacaattgcaagagtcatcagtgtgcaaatggggccacctgtgttcctgaagctcatggctactcttgtctctgctttggaaattttaccgggagattttgcagacacagcagattaccctcaacagtctgtgggaatgagaagagaaacttcacttgctacaatggaggcagctgctccatgttccaggaggactggcaatgtatgtgctggccaggtttcactggagagtggtgtgaagaggacatcaacgagtgtgcctccgatccctgcatcaatggaggactgtgcagggacttggtcaacaggttcctatgcatctgtgatgtggccttcgctggcgagcgctgtgagctggacgtaagcggcctttccttttatgtgtccctcttactatggcaaaacctctttcagctcctgtcctacctcgtactgcgcatgaatgatgagccagttgtagagtggggggcacaggaaaattattaatgtgcatgggagcattcacaagtgtaaaacattgacttgcaagaaacatcttgtctcagtgtaggtttctaggaaagacaaagggaacattagggaatagactccatctagagcactggttctcagtcttcctaatgctgcaaccctttagtacagctcttcctgttgtagtgatcgcagccataacattattttcattgccacttcataactgtaatccttctactgctgtgaatcacaatggaaatatttatgttttctgatggtcttaagcaacacctctgaaaaagtcattgaccccccccccaaaggggctgtgatccacaggttgagaaatgctcatctggaaggtaaccatgcatttaagtgtacctctagtagtttgggtctatagaagatattctcctattctacctttttagacacgccagaagagggcatctgattccattaaagatgattgggagccaccgtgtggttcctgagaactgtactcgggccctttggaagagcaatcagtgctctttccagcccctaagaatatttttaatacagccagaaaggtctcattacccagtgtactgagccctaaggcactttcatcctcaatcgttccatgttgaatggttttcattacatttggaaaatgttttctctccactctacctttacatgttcctattttcctattgacaatttgccccttcactgtaattctaatttggtgtggtccttcttctcataagtttatatgtgacatgaacatttaaaaatatctatgaatattttatagtcatgtatgtctttctgcaaagctattcaaatgaactatggacagttcttttctacacgaagaagagatgagtttaatccccagtaacatgagaaaaagatgagtgagggacagtgctcacagtatccctcactagcatcatttgtgattccatgggccatttttttccaccagcaaatagcagagagccctttccctattcgtttctcttacacttccccttttctgttacaactgaacactttacattagttactcctttgtagggggtttgacttttccaccgttttctctggttcactatttatgctaagtatctgtgcagggcgggtatatcagtccaacagaggtgtcattagtgttcattgaggaggaaatactttgcatgaattcatgacatcattgaagtagcagtggccagaaagatacccttctgcgaatgtgtctgtgtattcagaagctgccctggttagaaaacatgtgggtcacttttcctttgcatgttaccagtgctcactgggtcatgattgttttaagacagagcttttgctgtggcaatgaccaaggtgaatccagagatgcagatcagacaaaggacaagacaatgtactatctgagtaaaaccctgccttgacttactcctcagtacttagagattttacatagcaacctccaccctgtggcaacccgttcacactagcagtgatgctgagatttgcccttccttctcatcatcttcctcacatccaaagcattttgtgtccacactgctgtttcagataactgtttctaaagtgggattgttgtagccagaaaggtagggaaaatgttccccaaaatatttgcattcttaagtatgtgaagtaagtagattatagtcagagacaatatgtaaggtttcaggttcactcccttctacacatatcttcaactgtgtatttgcagaatattctgaatgtgacatactcccaacagaatatatttaaggagtatttatccacagtattgttctctgtacagttctagtgcttctattgtcactgcaattgtcaattgtttttctgctttccaactgtcttattatcatttaatagcatcttgctaaatgccctctttctattctccttatttctccatagttcatgtgtgtctgtgtgactaaggattctcctcatttttgcagaaaaataaaatcttttcttctttatgtcctgcttgtcattctctggtgacacatgtctttgcttacttggactgagggttgtacagtaagtacagaagcaggctcagtcacacagacagagacacaccaccaccagcagcagcagcaccaccaccaccaccaccaccaccagaaaacagtatgagtactcatctcttgattacatgtcatttcaagtaagcaccatgacaccgagggccaggttccatggactttctctgttaggcacgtgattctttagctgacctttgagaacagactccaacaacctcacttatttttactgttgacttatatcatctctgacaacactggacttcgtttgagctagtcaagaggaaagaccatgacacctaagggacagaaattcacacactcggtttttcataattcacacacattcctatgtatcaaatctctgtaatagatgacatttacttgaataaaaagtcatttcccttt

SEQ ID NO: 68 - protein, 1314 aa, Mus musculus
MKLKRTAYLLFLYLSSSLLICIKNSFCNKNNTRCLSGPCQNNSTCKHFPQDNNCCLDTANNLDKDCEDLKDPCFSSPCQGIATCVKIPGEGNFLCQCPPGYSGLNCETATNSCGGNLCQHGGTCRKDPEHPVCICPPGYAGRFCETDHNECASSPCHNGAMCQDGINGYSCFCVPGYQGRHCDLEVDECVSDPCKNEAVCLNEIGRYTCVCPQEFSGVNCELEIDECRSQPCLHGATCQDAPGGYSCDCAPGFLGEHCELSVNECESQPCLHGGLCVDGRNSYHCDCTGSGFTGMHCESLIPLCWSKPCHNDATCEDTVDSYICHCRPGIHCEEDVDECLLHPCLNGGTCENLPGNYACHCPFDDTSRTFYGGENCSEILLGCTHHQCLNNGKCIPHFQNGQHGFTCQCLSGYAGPLCETVTTLSFGSNGFLWVTSGSHTGIGPECNISLRFHTVQPNALLLIRGNKDVSMKLELLNGCVHLSIEVWNQLKVLLSISHNTSDGEWHFVEVTIAETLTLALVGGSCKEKCTTKSSVPVENHQSICALQDSFLGGLPMGTANNSVSVLNIYNVPSTPSFVGCLQDIRFDLNHITLENVSSGLSSNVKAGCLGKDWCESQPCQNRGRCINLWQGYQCECDRPYTGSNCLKEYVAGRFGQDDSTGYAAFSVNDNYGQNFSLSMFVRTRQPLGLLLALENSTYQYVSVWLEHGSLALQTPGSPKFMVNFFLSDGNVHLISLRIKPNEIELYQSSQNLGFISVPTWTIRRGDVIFIGGLPDREKTEVYGGFFKGCVQDVRLNSQTLEFFPNSTNNAYDDPILVNVTQGCPGDNTCKSNPCHNGGVCHSLWDDFSCSCPTNTAGRACEQVQWCQLSPCPPTAECQLLPQGFECIANAVFSGLSREILFRSNGNITRELTNITFAFRTHDTNVMILHAEKEPEFLNISIQDARLFFQLRSGNSFYTLHLMGSQLVNDGTWHQVTFSMIDPVAQTSRWQMEVNDQTPFVISEVATGSLNFLKDNTDIYVGDQSVDNPKGLQGCLSTIEIGGIYLSYFENLHGFPGKPQEEQFLKVSTNMVLTGCLPSNACHSSPCLHGGNCEDSYSSYRCACLSGWSGTHCEINIDECFSSPCIHGNCSDGVAAYHCRCEPGYTGVNCEVDVDNCKSHQCANGATCVPEAHGYSCLCFGNFTGRFCRHSRLPSTVCGNEKRNFTCYNGGSCSMFQEDWQCMCWPGFTGEWCEEDINECASDPCINGGLCRDLVNRFLCICDVAFAGERCELDVSGLSFYVSLLLWQNLFQLLSYLVLRMNDEPVVEWGAQENY

SEQ ID NO: 69 - DNA, 5481 bp, Mus musculus
  source 1..5481: mol_type = genomic DNA
gatccagcttgaagaggagtgaggcaaagctgaaccctcccactctccttgacaagtgcaagcccacacttttggaaaaaagcacaaagacgtcagaaacggttcctgtcgacctactaggctttggatggctaagtgtttttgctttgtatggaaatatgtttggacacaagacacaaggttttcacattttaatggcagtgctcataggaattcactgtgaagaagacgttgatgaatgtttactgcacccttgcctaaatggtggtacttgtgagaacctgcctgggaattatgcctgtcactgtccctttgatgacacttctaggacattttatggaggagaaaactgctcagaaattctcctgggctgcactcatcaccagtgtctgaacaatggaaaatgtatccctcatttccaaaatggccagcatggattcacttgccagtgtctttctggctatgcggggcccctgtgtgaaactgtcaccacactttcatttgggagcaatggcttcctatgggtcacaagtggctcccatacaggcatagggccagaatgtaacatatccttgaggtttcacactgttcaaccaaacgcacttctcctcatccgaggcaacaaggacgtgtctatgaagctggagttgctgaatggttgtgttcacttatcaattgaagtctggaatcagttaaaggtgctcctgtctatttctcacaacaccagtgatggagaatggcatttcgtggaggtaacaatcgcagaaactctaacccttgccctagttggcggctcctgcaaggagaagtgcaccaccaagtcttctgttccagttgagaatcatcaatcaatatgtgctttgcaggactcttttttgggtggcttaccaatggggacagccaacaacagtgtgtctgtgcttaacatctataatgtgccgtccacaccttcctttgtaggctgtctccaagacattagatttgatttgaatcacattactctggagaacgtttcatctggcctgtcatcaaatgttaaagcaggctgcctgggaaaggactggtgtgaaagtcaaccctgtcaaaacagaggacgctgcatcaacttgtggcagggttatcagtgtgaatgtgacaggccctatacaggctccaactgcctgaaagagtatgtagcgggaagatttggccaagatgactccacaggatatgcggcctttagtgttaatgataattatggacagaacttcagtctttcaatgtttgtccgaacacgtcaacccctgggcttacttctggctttggaaaatagtacttaccagtatgtcagtgtctggctagagcacggcagcctagcactgcagactccaggctctcccaagttcatggtaaacttttttctcagtgatggaaatgttcacttaatatctttgagaatcaaaccaaatgaaattgaactgtatcagtcttcacaaaacctaggattcatttctgttcctacatggacaattcgaagaggagacgtcatcttcattggtggcttacctgacagagagaagactgaagtttatggtggcttcttcaaaggctgtgttcaagatgtcagattaaacagccagactctggaattctttcccaattcaacaaacaatgcatacgatgacccaattcttgtcaatgtgactcaaggctgtcccggagacaacacatgtaaggtatcgcaaacgctgttttcagcggattaagcagagaaatactcttcagaagcaatgggaacattaccagagaactcaccaatatcacatttgctttcagaacacatgatacaaatgtgatgatattgcatgcagaaaaagaaccagagtttcttaatattagcattcaagatgccagattattctttcaattgcgaagtggcaacagcttttatacgctgcacctgatgggttcccaattggtgaatgatggcacatggcaccaagtgactttctccatgatagacccagtggcccagacctcccggtggcaaatggaggtgaacgaccagacaccctttgtgataagtgaagttgctactggaagcctgaactttttgaaggacaatacagacatctatgtgggtgaccaatctgttgacaatccgaaaggcctgcagggctgtctgagcacaatagagattggaggcatatatctttcttactttgaaaatctacatggtttccctggtaagcctcaggaagagcaatttctcaaagtttctacaaatatggtacttactggctgtttgccatcaaatgcctgccactccagcccctgtttgcatggaggaaactgtgaagacagctacagttcttatcggtgtgcctgtctctcgggatggtcagggacacactgtgaaatcaacattgatgagtgcttttctagcccctgtatccatggcaactgctctgatggagttgcagcctaccactgcaggtgtgagcctggatacaccggtgtgaactgtgaggtggatgtagacaattgcaagagtcatcagtgtgcaaatggggccacctgtgttcctgaagctcatggctactcttgtctctgctttggaaattttaccgggagattttgcagacacagcagattaccctcaacagtctgtgggaatgagaagagaaacttcacttgctacaatggaggcagctgctccatgttccaggaggactggcaatgtatgtgctggccaggtttcactggagagtggtgtgaagaggacatcaacgagtgtgcctccgatccctgcatcaatggaggactgtgcagggacttggtcaacaggttcctatgcatctgtgatgtggccttcgctggcgagcgctgtgagctggacgtaagcggcctttccttttatgtgtccctcttactatggcaaaacctctttcagctcctgtcctacctcgtactgcgcatgaatgatgagccagttgtagagtggggggcacaggaaaattattaatgtgcatgggagcattcacaagtgtaaaacattgacttgcaagaaacatcttgtctcagtgtaggtttctaggaaagacaaagggaacattagggaatagactccatctagagcactggttctcagtcttcctaatgctgcaaccctttagtacagctcttcctgttgtagtgatcgcagccataacattattttcattgccacttcataactgtaatccttctactgctgtgaatcacaatggaaatatttatgttttctgatggtcttaagcaacacctctgaaaaagtcattgaccccccccccaaaggggctgtgatccacaggttgagaaatgctcatctggaaggtaaccatgcatttaagtgtacctctagtagtttgggtctatagaagatattctcctattctacctttttagacacgccagaagagggcatctgattccattaaagatgattgggagccaccgtgtggttcctgagaactgtactcgggccctttggaagagcaatcagtgctctttccagcccctaagaatatttttaatacagccagaaaggtctcattacccagtgtactgagccctaaggcactttcatcctcaatcgttccatgttgaatggttttcattacatttggaaaatgttttctctccactctacctttacatgttcctattttcctattgacaatttgccccttcactgtaattctaatttggtgtggtccttcttctcataagtttatatgtgacatgaacatttaaaaatatctatgaatattttatagtcatgtatgtctttctgcaaagctattcaaatgaactatggacagttcttttctacacgaagaagagatgagtttaatccccagtaacatgagaaaaagatgagtgagggacagtgctcacagtatccctcactagcatcatttgtgattccatgggccatttttttccaccagcaaatagcagagagccctttccctattcgtttctcttacacttccccttttctgttacaactgaacactttacattagttactcctttgtagggggtttgacttttccaccgttttctctggttcactatttatgctaagtatctgtgcagggcgggtatatcagtccaacagaggtgtcattagtgttcattgaggaggaaatactttgcatgaattcatgacatcattgaagtagcagtggccagaaagatacccttctgcgaatgtgtctgtgtattcagaagctgccctggttagaaaacatgtgggtcacttttcctttgcatgttaccagtgctcactgggtcatgattgttttaagacagagcttttgctgtggcaatgaccaaggtgaatccagagatgcagatcagacaaaggacaagacaatgtactatctgagtaaaaccctgccttgacttactcctcagtacttagagattttacatagcaacctccaccctgtggcaacccgttcacactagcagtgatgctgagatttgcccttccttctcatcatcttcctcacatccaaagcattttgtgtccacactgctgtttcagataactgtttctaaagtgggattgttgtagccagaaaggtagggaaaatgttccccaaaatatttgcattcttaagtatgtgaagtaagtagattatagtcagagacaatatgtaaggtttcaggttcactcccttctacacatatcttcaactgtgtatttgcagaatattctgaatgtgacatactcccaacagaatatatttaaggagtatttatccacagtattgttctctgtacagttctagtgcttctattgtcactgcaattgtcaattgtttttctgctttccaactgtcttattatcatttaatagcatcttgctaaatgccctctttctattctccttatttctccatagttcatgtgtgtctgtgtgactaaggattctcctcatttttgcagaaaaataaaatcttttcttctttatgtcctgcttgtcattctctggtgacacatgtctttgcttacttggactgagggttgtacagtaagtacagaagcaggctcagtcacacagacagagacacaccaccaccagcagcagcagcaccaccaccaccaccaccaccaccagaaaacagtatgagtactcatctcttgattacatgtcatttcaagtaagcaccatgacaccgagggccaggttccatggactttctctgttaggcacgtgattctttagctgacctttgagaacagactccaacaacctcacttatttttactgttgacttatatcatctctgacaacactggacttcgtttgagctagtcaagaggaaagaccatgacacctaagggacagaaattcacacactcggtttttcataattcacacacattcctatgtatcaaatctctgtaatagatgacatttacttgaataaaaagtcatttcccttt

SEQ ID NO: 70 - protein, 528 aa, Mus musculus
MFGHKTQGFHILMAVLIGIHCEEDVDECLLHPCLNGGTCENLPGNYACHCPFDDTSRTFYGGENCSEILLGCTHHQCLNNGKCIPHFQNGQHGFTCQCLSGYAGPLCETVTTLSFGSNGFLWVTSGSHTGIGPECNISLRFHTVQPNALLLIRGNKDVSMKLELLNGCVHLSIEVWNQLKVLLSISHNTSDGEWHFVEVTIAETLTLALVGGSCKEKCTTKSSVPVENHQSICALQDSFLGGLPMGTANNSVSVLNIYNVPSTPSFVGCLQDIRFDLNHITLENVSSGLSSNVKAGCLGKDWCESQPCQNRGRCINLWQGYQCECDRPYTGSNCLKEYVAGRFGQDDSTGYAAFSVNDNYGQNFSLSMFVRTRQPLGLLLALENSTYQYVSVWLEHGSLALQTPGSPKFMVNFFLSDGNVHLISLRIKPNEIELYQSSQNLGFISVPTWTIRRGDVIFIGGLPDREKTEVYGGFFKGCVQDVRLNSQTLEFFPNSTNNAYDDPILVNVTQGCPGDNTCKVSQTLFSAD

SEQ ID NO: 71 - DNA, 6950 bp, Mus musculus
  source 1..6950: mol_type = genomic DNA
gtcagaagaaattaatttctctattaggagcaatcaaggaccctatcctaaaacaaagcctttccaggaggcattgttcacggaagcctgagggggacacgaatccaatccaggctggaaaaatctgctccaggattgactggttaccgtcttcctgtgcctgtaaggtgctgtgaaagagaagtgctttctgattctctgtctgtggaggagccctgggaggggtgggacagagatggcatcctggctctctgaggcacctgctcttctctgaaccacacaggagtcaagagccaaacagggatagcttcagcagcacttcagagggtgttctctaagtaagaacatgaagctcaagagaactgcctaccttctcttcctgtacctcagctcctcactgctcatctgcataaagaattcattttgcaataaaaacaataccaggtgcctttcaggtccttgccaaaacaattctacgtgcaagcattttccacaagacaacaattgttgcttagacacagccaataatttggacaaagactgtgaagatctgaaagacccttgcttctcgagtccctgccaaggaattgccacttgtgtgaaaatcccaggggaagggaacttcctgtgtcagtgtcctcctgggtacagcgggctgaactgtgaaactgccaccaattcctgtggagggaacctctgccaacatggaggcacctgccgtaaagaccctgagcaccctgtctgtatctgccctcctggatatgctggaaggttctgtgagactgatcacaatgagtgtgcttctagcccttgccacaatggggctatgtgccaggatggaatcaatggctactcctgcttctgtgtgcctggataccaaggcaggcattgtgacttggaagtggatgaatgtgtttctgatccctgcaagaatgaggctgtgtgcctcaatgagataggaagatacacttgtgtctgccctcaagagttttctggcgtgaactgtgagttggaaattgatgaatgcagatcccagccttgtctccacggtgccacatgtcaggacgctccagggggctactcctgtgactgtgcacctggattccttggagagcactgtgaactcagcgttaatgaatgtgaaagtcagccgtgtctccatggaggtctatgtgtggatggaagaaacagttaccactgtgactgcacaggtagtggattcacagggatgcactgtgagtccttgattcctctttgttggtcaaagccttgtcacaacgacgcgacatgtgaagatactgttgacagctatatttgtcactgccggcctggatacacaggtgccctgtgtgagacagacataaatgaatgcagtagcaacccctgccaattttggggggaatgtgtcgagctgtcctcagagggtctatatggaaacactgctggcctgccttcctccttcagctatgttggagcctcgggctatgtgtgtatctgtcagcctggattcacaggaattcactgtgaagaagacgttgatgaatgtttactgcacccttgcctaaatggtggtacttgtgagaacctgcctgggaattatgcctgtcactgtccctttgatgacacttctaggacattttatggaggagaaaactgctcagaaattctcctgggctgcactcatcaccagtgtctgaacaatggaaaatgtatccctcatttccaaaatggccagcatggattcacttgccagtgtctttctggctatgcggggcccctgtgtgaaactgtcaccacactttcatttgggagcaatggcttcctatgggtcacaagtggctcccatacaggcatagggccagaatgtaacatatccttgaggtttcacactgttcaaccaaacgcacttctcctcatccgaggcaacaaggacgtgtctatgaagctggagttgctgaatggttgtgttcacttatcaattgaagtctggaatcagttaaaggtgctcctgtctatttctcacaacaccagtgatggagaatggcatttcgtggaggtaacaatcgcagaaactctaacccttgccctagttggcggctcctgcaaggagaagtgcaccaccaagtcttctgttccagttgagaatcatcaatcaatatgtgctttgcaggactcttttttgggtggcttaccaatggggacagccaacaacagtgtgtctgtgcttaacatctataatgtgccgtccacaccttcctttgtaggctgtctccaagacattagatttgatttgaatcacattactctggagaacgtttcatctggcctgtcatcaaatgttaaagcaggctgcctgggaaaggactggtgtgaaagtcaaccctgtcaaaacagaggacgctgcatcaacttgtggcagggttatcagtgtgaatgtgacaggccctatacaggctccaactgcctgaaagagtatgtagcgggaagatttggccaagatgactccacaggatatgcggcctttagtgttaatgataattatggacagaacttcagtctttcaatgtttgtccgaacacgtcaacccctgggcttacttctggctttggaaaatagtacttaccagtatgtcagtgtctggctagagcacggcagcctagcactgcagactccaggctctcccaagttcatggtaaacttttttctcagtgatggaaatgttcacttaatatctttgagaatcaaaccaaatgaaattgaactgtatcagtcttcacaaaacctaggattcatttctgttcctacatggacaattcgaagaggagacgtcatcttcattggtggcttacctgacagagagaagactgaagtttatggtggcttcttcaaaggctgtgttcaagatgtcagattaaacagccagactctggaattctttcccaattcaacaaacaatgcatacgatgacccaattcttgtcaatgtgactcaaggctgtcccggagacaacacatgtaagtccaacccctgtcataatggaggtgtctgccactccctgtgggatgacttctcctgctcctgccctacaaacacagcggggagagcctgcgagcaagttcagtggtgtcaactcagcccatgtcctcccactgcagagtgccagctgctccctcaagggtttgaatgtatcgcaaacgctgttttcagcggattaagcagagaaatactcttcagaagcaatgggaacattaccagagaactcaccaatatcacatttgctttcagaacacatgatacaaatgtgatgatattgcatgcagaaaaagaaccagagtttcttaatattagcattcaagatgccagattattctttcaattgcgaagtggcaacagcttttatacgctgcacctgatgggttcccaattggtgaatgatggcacatggcaccaagtgactttctccatgatagacccagtggcccagacctcccggtggcaaatggaggtgaacgaccagacaccctttgtgataagtgaagttgctactggaagcctgaactttttgaaggacaatacagacatctatgtgggtgaccaatctgttgacaatccgaaaggcctgcagggctgtctgagcacaatagagattggaggcatatatctttcttactttgaaaatctacatggtttccctggtaagcctcaggaagagcaatttctcaaagtttctacaaatatggtacttactggctgtttgccatcaaatgcctgccactccagcccctgtttgcatggaggaaactgtgaagacagctacagttcttatcggtgtgcctgtctctcgggatggtcagggacacactgtgaaatcaacattgatgagtgcttttctagcccctgtatccatggcaactgctctgatggagttgcagcctaccactgcaggtgtgagcctggatacaccggtgtgaactgtgaggtggatgtagacaattgcaagagtcatcagtgtgcaaatggggccacctgtgttcctgaagctcatggctactcttgtctctgctttggaaattttaccgggagattttgcagacacagcagattaccctcaacagtctgtgggaatgagaagagaaacttcacttgctacaatggaggcagctgctccatgttccaggaggactggcaatgtatgtgctggccaggtttcactggagagtggtgtgaagaggacatcaacgagtgtgcctccgatccctgcatcaatggaggactgtgcagggacttggtcaacaggttcctatgcatctgtgatgtggccttcgctggcgagcgctgtgagctggacgtaagcggcctttccttttatgtgtccctcttactatggcaaaacctctttcagctcctgtcctacctcgtactgcgcatgaatgatgagccagttgtagagtggggggcacaggaaaattattaatgtgcatgggagcattcacaagtgtaaaacattgacttgcaagaaacatcttgtctcagtgtaggtttctaggaaagacaaagggaacattagggaatagactccatctagagcactggttctcagtcttcctaatgctgcaaccctttagtacagctcttcctgttgtagtgatcgcagccataacattattttcattgccacttcataactgtaatccttctactgctgtgaatcacaatggaaatatttatgttttctgatggtcttaagcaacacctctgaaaaagtcattgaccccccccccaaaggggctgtgatccacaggttgagaaatgctcatctggaaggtaaccatgcatttaagtgtacctctagtagtttgggtctatagaagatattctcctattctacctttttagacacgccagaagagggcatctgattccattaaagatgattgggagccaccgtgtggttcctgagaactgtactcgggccctttggaagagcaatcagtgctctttccagcccctaagaatatttttaatacagccagaaaggtctcattacccagtgtactgagccctaaggcactttcatcctcaatcgttccatgttgaatggttttcattacatttggaaaatgttttctctccactctacctttacatgttcctattttcctattgacaatttgccccttcactgtaattctaatttggtgtggtccttcttctcataagtttatatgtgacatgaacatttaaaaatatctatgaatattttatagtcatgtatgtctttctgcaaagctattcaaatgaactatggacagttcttttctacacgaagaagagatgagtttaatccccagtaacatgagaaaaagatgagtgagggacagtgctcacagtatccctcactagcatcatttgtgattccatgggccatttttttccaccagcaaatagcagagagccctttccctattcgtttctcttacacttccccttttctgttacaactgaacactttacattagttactcctttgtagggggtttgacttttccaccgttttctctggttcactatttatgctaagtatctgtgcagggcgggtatatcagtccaacagaggtgtcattagtgttcattgaggaggaaatactttgcatgaattcatgacatcattgaagtagcagtggccagaaagatacccttctgcgaatgtgtctgtgtattcagaagctgccctggttagaaaacatgtgggtcacttttcctttgcatgttaccagtgctcactgggtcatgattgttttaagacagagcttttgctgtggcaatgaccaaggtgaatccagagatgcagatcagacaaaggacaagacaatgtactatctgagtaaaaccctgccttgacttactcctcagtacttagagattttacatagcaacctccaccctgtggcaacccgttcacactagcagtgatgctgagatttgcccttccttctcatcatcttcctcacatccaaagcattttgtgtccacactgctgtttcagataactgtttctaaagtgggattgttgtagccagaaaggtagggaaaatgttccccaaaatatttgcattcttaagtatgtgaagtaagtagattatagtcagagacaatatgtaaggtttcaggttcactcccttctacacatatcttcaactgtgtatttgcagaatattctgaatgtgacatactcccaacagaatatatttaaggagtatttatccacagtattgttctctgtacagttctagtgcttctattgtcactgcaattgtcaattgtttttctgctttccaactgtcttattatcatttaatagcatcttgctaaatgccctctttctattctccttatttctccatagttcatgtgtgtctgtgtgactaaggattctcctcatttttgcagaaaaataaaatcttttcttctttatgtcctgcttgtcattctctggtgacacatgtctttgcttacttggactgagggttgtacagtaagtacagaagcaggctcagtcacacagacagagacacaccaccaccagcagcagcagcaccaccaccaccaccaccaccaccagaaaacagtatgagtactcatctcttgattacatgtcatttcaagtaagcaccatgacaccgagggccaggttccatggactttctctgttaggcacgtgattctttagctgacctttgagaacagactccaacaacctcacttatttttactgttgacttatatcatctctgacaacactggacttcgtttgagctagtcaagaggaaagaccatgacacctaagggacagaaattcacacactcggtttttcataattcacacacattcctatgtatcaaatctctgtaatagatgacatttacttgaataaaaagtcatttccctt

SEQ ID NO: 72 - protein, 1375 aa, Mus musculus
MKLKRTAYLLFLYLSSSLLICIKNSFCNKNNTRCLSGPCQNNSTCKHFPQDNNCCLDTANNLDKDCEDLKDPCFSSPCQGIATCVKIPGEGNFLCQCPPGYSGLNCETATNSCGGNLCQHGGTCRKDPEHPVCICPPGYAGRFCETDHNECASSPCHNGAMCQDGINGYSCFCVPGYQGRHCDLEVDECVSDPCKNEAVCLNEIGRYTCVCPQEFSGVNCELEIDECRSQPCLHGATCQDAPGGYSCDCAPGFLGEHCELSVNECESQPCLHGGLCVDGRNSYHCDCTGSGFTGMHCESLIPLCWSKPCHNDATCEDTVDSYICHCRPGYTGALCETDINECSSNPCQFWGECVELSSEGLYGNTAGLPSSFSYVGASGYVCICQPGFTGIHCEEDVDECLLHPCLNGGTCENLPGNYACHCPFDDTSRTFYGGENCSEILLGCTHHQCLNNGKCIPHFQNGQHGFTCQCLSGYAGPLCETVTTLSFGSNGFLWVTSGSHTGIGPECNISLRFHTVQPNALLLIRGNKDVSMKLELLNGCVHLSIEVWNQLKVLLSISHNTSDGEWHFVEVTIAETLTLALVGGSCKEKCTTKSSVPVENHQSICALQDSFLGGLPMGTANNSVSVLNIYNVPSTPSFVGCLQDIRFDLNHITLENVSSGLSSNVKAGCLGKDWCESQPCQNRGRCINLWQGYQCECDRPYTGSNCLKEYVAGRFGQDDSTGYAAFSVNDNYGQNFSLSMFVRTRQPLGLLLALENSTYQYVSVWLEHGSLALQTPGSPKFMVNFFLSDGNVHLISLRIKPNEIELYQSSQNLGFISVPTWTIRRGDVIFIGGLPDREKTEVYGGFFKGCVQDVRLNSQTLEFFPNSTNNAYDDPILVNVTQGCPGDNTCKSNPCHNGGVCHSLWDDFSCSCPTNTAGRACEQVQWCQLSPCPPTAECQLLPQGFECIANAVFSGLSREILFRSNGNITRELTNITFAFRTHDTNVMILHAEKEPEFLNISIQDARLFFQLRSGNSFYTLHLMGSQLVNDGTWHQVTFSMIDPVAQTSRWQMEVNDQTPFVISEVATGSLNFLKDNTDIYVGDQSVDNPKGLQGCLSTIEIGGIYLSYFENLHGFPGKPQEEQFLKVSTNMVLTGCLPSNACHSSPCLHGGNCEDSYSSYRCACLSGWSGTHCEINIDECFSSPCIHGNCSDGVAAYHCRCEPGYTGVNCEVDVDNCKSHQCANGATCVPEAHGYSCLCFGNFTGRFCRHSRLPSTVCGNEKRNFTCYNGGSCSMFQEDWQCMCWPGFTGEWCEEDINECASDPCINGGLCRDLVNRFLCICDVAFAGERCELDVSGLSFYVSLLLWQNLFQLLSYLVLRMNDEPVVEWGAQENY

SEQ ID NO: 73 - DNA, 5434 bp, Mus musculus
  source 1..5434: mol_type = genomic DNA
tcactgtgaagaagacgttgatgaatgtttactgcacccttgcctaaatggtggtacttgtgagaacctgcctgggaattatgcctgtcactgtccctttgatgacacttctaggacattttatggaggagaaaactgctcagaaattctcctgggctgcactcatcaccagtgtctgaacaatggaaaatgtatccctcatttccaaaatggccagcatggattcacttgccagtgtctttctggctatgcggggcccctgtgtgaaactgtcaccacactttcatttgggagcaatggcttcctatgggtcacaagtggctcccatacaggcatagggccagaatgtaacatatccttgaggtttcacactgttcaaccaaacgcacttctcctcatccgaggcaacaaggacgtgtctatgaagctggagttgctgaatggttgtgttcacttatcaattgaagtctggaatcagttaaaggtgctcctgtctatttctcacaacaccagtgatggagaatggcatttcgtggaggtaacaatcgcagaaactctaacccttgccctagttggcggctcctgcaaggagaagtgcaccaccaagtcttctgttccagttgagaatcatcaatcaatatgtgctttgcaggactcttttttgggtggcttaccaatggggacagccaacaacagtgtgtctgtgcttaacatctataatgtgccgtccacaccttcctttgtaggctgtctccaagacattagatttgatttgaatcacattactctggagaacgtttcatctggcctgtcatcaaatgttaaagcaggctgcctgggaaaggactggtgtgaaagtcaaccctgtcaaaacagaggacgctgcatcaacttgtggcagggttatcagtgtgaatgtgacaggccctatacaggctccaactgcctgaaagagtatgtagcgggaagatttggccaagatgactccacaggatatgcggcctttagtgttaatgataattatggacagaacttcagtctttcaatgtttgtccgaacacgtcaacccctgggcttacttctggctttggaaaatagtacttaccagtatgtcagtgtctggctagagcacggcagcctagcactgcagactccaggctctcccaagttcatggtaaacttttttctcagtgatggaaatgttcacttaatatctttgagaatcaaaccaaatgaaattgaactgtatcagtcttcacaaaacctaggattcatttctgttcctacatggacaattcgaagaggagacgtcatcttcattggtggcttacctgacagagagaagactgaagtttatggtggcttcttcaaaggctgtgttcaagatgtcagattaaacagccagactctggaattctttcccaattcaacaaacaatgcatacgatgacccaattcttgtcaatgtgactcaaggctgtcccggagacaacacatgtaagtccaacccctgtcataatggaggtgtctgccactccctgtgggatgacttctcctgctcctgccctacaaacacagcggggagagcctgcgagcaagttcagtggtgtcaactcagcccatgtcctcccactgcagagtgccagctgctccctcaagggtttgaatgtatcgcaaacgctgttttcagcggattaagcagagaaatactcttcagaagcaatgggaacattaccagagaactcaccaatatcacatttgctttcagaacacatgatacaaatgtgatgatattgcatgcagaaaaagaaccagagtttcttaatattagcattcaagatgccagattattctttcaattgcgaagtggcaacagcttttatacgctgcacctgatgggttcccaattggtgaatgatggcacatggcaccaagtgactttctccatgatagacccagtggcccagacctcccggtggcaaatggaggtgaacgaccagacaccctttgtgataagtgaagttgctactggaagcctgaactttttgaaggacaatacagacatctatgtgggtgaccaatctgttgacaatccgaaaggcctgcagggctgtctgagcacaatagagattggaggcatatatctttcttactttgaaaatctacatggtttccctggtaagcctcaggaagagcaatttctcaaagtttctacaaatatggtacttactggctgtttgccatcaaatgcctgccactccagcccctgtttgcatggaggaaactgtgaagacagctacagttcttatcggtgtgcctgtctctcgggatggtcagggacacactgtgaaatcaacattgatgagtgcttttctagcccctgtatccatggcaactgctctgatggagttgcagcctaccactgcaggtgtgagcctggatacaccggtgtgaactgtgaggtggatgtagacaattgcaagagtcatcagtgtgcaaatggggccacctgtgttcctgaagctcatggctactcttgtctctgctttggaaattttaccgggagattttgcagacacagcagattaccctcaacagtctgtgggaatgagaagagaaacttcacttgctacaatggaggcagctgctccatgttccaggaggactggcaatgtatgtgctggccaggtttcactggagagtggtgtgaagaggacatcaacgagtgtgcctccgatccctgcatcaatggaggactgtgcagggacttggtcaacaggttcctatgcatctgtgatgtggccttcgctggcgagcgctgtgagctggacgtaagcggcctttccttttatgtgtccctcttactatggcaaaacctctttcagctcctgtcctacctcgtactgcgcatgaatgatgagccagttgtagagtggggggcacaggaaaattattaatgtgcatgggagcattcacaagtgtaaaacattgacttgcaagaaacatcttgtctcagtgtaggtttctaggaaagacaaagggaacattagggaatagactccatctagagcactggttctcagtcttcctaatgctgcaaccctttagtacagctcttcctgttgtagtgatcgcagccataacattattttcattgccacttcataactgtaatccttctactgctgtgaatcacaatggaaatatttatgttttctgatggtcttaagcaacacctctgaaaaagtcattgaccccccccccaaaggggctgtgatccacaggttgagaaatgctcatctggaaggtaaccatgcatttaagtgtacctctagtagtttgggtctatagaagatattctcctattctacctttttagacacgccagaagagggcatctgattccattaaagatgattgggagccaccgtgtggttcctgagaactgtactcgggccctttggaagagcaatcagtgctctttccagcccctaagaatatttttaatacagccagaaaggtctcattacccagtgtactgagccctaaggcactttcatcctcaatcgttccatgttgaatggttttcattacatttggaaaatgttttctctccactctacctttacatgttcctattttcctattgacaatttgccccttcactgtaattctaatttggtgtggtccttcttctcataagtttatatgtgacatgaacatttaaaaatatctatgaatattttatagtcatgtatgtctttctgcaaagctattcaaatgaactatggacagttcttttctacacgaagaagagatgagtttaatccccagtaacatgagaaaaagatgagtgagggacagtgctcacagtatccctcactagcatcatttgtgattccatgggccatttttttccaccagcaaatagcagagagccctttccctattcgtttctcttacacttccccttttctgttacaactgaacactttacattagttactcctttgtagggggtttgacttttccaccgttttctctggttcactatttatgctaagtatctgtgcagggcgggtatatcagtccaacagaggtgtcattagtgttcattgaggaggaaatactttgcatgaattcatgacatcattgaagtagcagtggccagaaagatacccttctgcgaatgtgtctgtgtattcagaagctgccctggttagaaaacatgtgggtcacttttcctttgcatgttaccagtgctcactgggtcatgattgttttaagacagagcttttgctgtggcaatgaccaaggtgaatccagagatgcagatcagacaaaggacaagacaatgtactatctgagtaaaaccctgccttgacttactcctcagtacttagagattttacatagcaacctccaccctgtggcaacccgttcacactagcagtgatgctgagatttgcccttccttctcatcatcttcctcacatccaaagcattttgtgtccacactgctgtttcagataactgtttctaaagtgggattgttgtagccagaaaggtagggaaaatgttccccaaaatatttgcattcttaagtatgtgaagtaagtagattatagtcagagacaatatgtaaggtttcaggttcactcccttctacacatatcttcaactgtgtatttgcagaatattctgaatgtgacatactcccaacagaatatatttaaggagtatttatccacagtattgttctctgtacagttctagtgcttctattgtcactgcaattgtcaattgtttttctgctttccaactgtcttattatcatttaatagcatcttgctaaatgccctctttctattctccttatttctccatagttcatgtgtgtctgtgtgactaaggattctcctcatttttgcagaaaaataaaatcttttcttctttatgtcctgcttgtcattctctggtgacacatgtctttgcttacttggactgagggttgtacagtaagtacagaagcaggctcagtcacacagacagagacacaccaccaccagcagcagcagcaccaccaccaccaccaccaccaccagaaaacagtatgagtactcatctcttgattacatgtcatttcaagtaagcaccatgacaccgagggccaggttccatggactttctctgttaggcacgtgattctttagctgacctttgagaacagactccaacaacctcacttatttttactgttgacttatatcatctctgacaacactggacttcgtttgagctagtcaagaggaaagaccatgacacctaagggacagaaattcacacactcggtttttcataattcacacacattcctatgtatcaaatctctgtaatagatgacatttacttgaataaaaagtcatttccctttg

SEQ ID NO: 74 - protein, 844 aa, Mus musculus
MKLELLNGCVHLSIEVWNQLKVLLSISHNTSDGEWHFVEVTIAETLTLALVGGSCKEKCTTKSSVPVENHQSICALQDSFLGGLPMGTANNSVSVLNIYNVPSTPSFVGCLQDIRFDLNHITLENVSSGLSSNVKAGCLGKDWCESQPCQNRGRCINLWQGYQCECDRPYTGSNCLKEYVAGRFGQDDSTGYAAFSVNDNYGQNFSLSMFVRTRQPLGLLLALENSTYQYVSVWLEHGSLALQTPGSPKFMVNFFLSDGNVHLISLRIKPNEIELYQSSQNLGFISVPTWTIRRGDVIFIGGLPDREKTEVYGGFFKGCVQDVRLNSQTLEFFPNSTNNAYDDPILVNVTQGCPGDNTCKSNPCHNGGVCHSLWDDFSCSCPTNTAGRACEQVQWCQLSPCPPTAECQLLPQGFECIANAVFSGLSREILFRSNGNITRELTNITFAFRTHDTNVMILHAEKEPEFLNISIQDARLFFQLRSGNSFYTLHLMGSQLVNDGTWHQVTFSMIDPVAQTSRWQMEVNDQTPFVISEVATGSLNFLKDNTDIYVGDQSVDNPKGLQGCLSTIEIGGIYLSYFENLHGFPGKPQEEQFLKVSTNMVLTGCLPSNACHSSPCLHGGNCEDSYSSYRCACLSGWSGTHCEINIDECFSSPCIHGNCSDGVAAYHCRCEPGYTGVNCEVDVDNCKSHQCANGATCVPEAHGYSCLCFGNFTGRFCRHSRLPSTVCGNEKRNFTCYNGGSCSMFQEDWQCMCWPGFTGEWCEEDINECASDPCINGGLCRDLVNRFLCICDVAFAGERCELDVSGLSFYVSLLLWQNLFQLLSYLVLRMNDEPVVEWGAQENY

SEQ ID NO: 75 - DNA, 5678 bp, Mus musculus
  source 1..5678: mol_type = genomic DNA
acgaatccaatccaggctggaaaaatctgctccaggattgactggttaccgtcttcctgtgcctgtaaggtgctgtgaaagagaagtgctttctgattctctgtctgtggaggagccctgggaggggtgggacagagatggcatcctggctctctgaggcacctgctcttctctgaaccacacaggagtcaagagccaaacagggatagcttcagcagcacttcagagggtgttctctaagtaagaacatgaagctcaagagaactgcctaccttctcttcctgtacctcagctcctcactgctcatctgcataaagaattcattttgcaataaaaacaataccaggtgcctttcaggtccttgccaaaacaattctacgtgcaagcattttccacaagacaacaattgttgcttagacacagccaataatttggacaaagactgtgaagatctgaaagacccttgcttctcgagtccctgccaaggaattgccacttgtgtgaaaatcccaggggaagggaacttcctgtgtcagtgtcctcctgggtacagcgggctgaactgtgaaactgccaccaattcctgtggagggaacctctgccaacatggaggcacctgccgtaaagaccctgagcaccctgtctgtatctgccctcctggatatgctggaaggttctgtgagactgatcacaatgagtgtgcttctagcccttgccacaatggggctatgtgccaggatggaatcaatggctactcctgcttctgtgtgcctggataccaaggcaggcattgtgacttggaagtggatgaatgtgtttctgatccctgcaagaatgaggctgtgtgcctcaatgagataggaagatacacttgtgtctgccctcaagagttttctggcgtgaactgtgagttggaaattgatgaatgcagatcccagccttgtctccacggtgccacatgtcaggacgctccagggggctactcctgtgactgtgcacctggattccttggagagcactgtgaactcagcgttaatgaatgtgaaagtcagccgtgtctccatggaggtctatgtgtggatggaagaaacaggaattcactgtgaagaagacgttgatgaatgtttactgcacccttgcctaaatggtggtacttgtgagaacctgcctgggaattatgcctgtcactgtccctttgatgacacttctaggacattttatggaggagaaaactgctcagaaattctcctgggctgcactcatcaccagtgtctgaacaatggaaaatgtatccctcatttccaaaatggccagcatggattcacttgccagtgtctttctggctatgcggggcccctgtgtgaaactgtcaccacactttcatttgggagcaatggcttcctatgggtcacaagtggctcccatacaggcatagggccagaatgtaacatatccttgaggtttcacactgttcaaccaaacgcacttctcctcatccgaggcaacaaggacgtgtctatgaagctggagttgctgaatggttgtgttcacttatcaattgaagtctggaatcagttaaaggtgctcctgtctatttctcacaacaccagtgatggagaatggcatttcgtggaggtaacaatcgcagaaactctaacccttgccctagttggcggctcctgcaaggagaagtgcaccaccaagtcttctgttccagttgagaatcatcaatcaatatgtgctttgcaggactcttttttgggtggcttaccaatggggacagccaacaacagtgtgtctgtgcttaacatctataatgtgccgtccacaccttcctttgtaggctgtctccaagacattagatttgatttgaatcacattactctggagaacgtttcatctggcctgtcatcaaatgttaaagcaggctgcctgggaaaggactggtgtgaaagtcaaccctgtcaaaacagaggacgctgcatcaacttgtggcagggttatcagtgtgaatgtgacaggccctatacaggctccaactgcctgaaagagtatgtagcgggaagatttggccaagatgactccacaggatatgcggcctttagtgttaatgataattatggacagaacttcagtctttcaatgtttgtccgaacacgtcaacccctgggcttacttctggctttggaaaatagtacttaccagtatgtcagtgtctggctagagcacggcagcctagcactgcagactccaggctctcccaagttcatggtaaacttttttctcagtgatggaaatgttcacttaatatctttgagaatcaaaccaaatgaaattgaactgtatcagtcttcacaaaacctaggattcatttctgttcctacatggacaattcgaagaggagacgtcatcttcattggtggcttacctgacagagagaagactgaagtttatggtggcttcttcaaaggctgtgttcaagatgtcagattaaacagccagactctggaattctttcccaattcaacaaacaatgcatacgatgacccaattcttgtcaatgtgactcaaggctgtcccggagacaacacatgtaagtccaacccctgtcataatggaggtgtctgccactccctgtgggatgacttctcctgctcctgccctacaaacacagcggggagagcctgcgagcaagttcagtggtgtcaactcagcccatgtcctcccactgcagagtgccagctgctccctcaagggtttgaatgtatcgcaaacgctgttttcagcggattaagcagagaaatactcttcagaagcaatgggaacattaccagagaactcaccaatatcacatttgctttcagaacacatgatacaaatgtgatgatattgcatgcagaaaaagaaccagagtttcttaatattagcattcaagatgccagattattctttcaattgcgaagtggcaacagcttttatacgctgcacctgatgggttcccaattggtgaatgatggcacatggcaccaagtgactttctccatgatagacccagtggcccagacctcccggtggcaaatggaggtgaacgaccagacaccctttgtgataagtgaagttgctactggaagcctgaactttttgaaggacaatacagacatctatgtgggtgaccaatctgttgacaatccgaaaggcctgcagggctgtctgagcacaatagagattggaggcatatatctttcttactttgaaaatctacatggtttccctggtaagcctcaggaagagcaatttctcaaagtttctacaaatatggtacttactggctgtttgccatcaaatgcctgccactccagcccctgtttgcatggaggaaactgtgaagacagctacagttcttatcggtgtgcctgtctctcgggatggtcagggacacactgtgaaatcaacattgatgagtgcttttctagcccctgtatccatggcaactgctctgatggagttgcagcctaccactgcaggtgtgagcctggatacaccggtgtgaactgtgaggtggatgtagacaattgcaagagtcatcagtgtgcaaatggggccacctgtgttcctgaagctcatggctactcttgtctctgctttggaaattttaccgggagattttgcagacacagcagattaccctcaacagtctgtgggaatgagaagagaaacttcacttgctacaatggaggcagctgctccatgttccaggaggactggcaatgtatgtgctggccaggtttcactggagagtggtgtgaagaggacatcaacgagtgtgcctccgatccctgcatcaatggaggactgtgcagggacttggtcaacaggttcctatgcatctgtgatgtggccttcgctggcgagcgctgtgagctggacctggctgatgacaggctcctgggcattttcaccgctgttggctccggaactttggccctgttcttcatcctcttgcttgctggggttgcttctcttattgcctccaacaaaagggcgactcaaggaacctacagccccagcggtcaggagaaggctggccctcgagtggaaatgtggatcaggatgccgcccccggcactggaaaggctcatctaggagactgctgctcttctcaggacagagaagaacatgatgagtaccgggtcgtgcctgagtgaagatggctttacatcactagagatacatacagctgggactgtgggaaggaccttcctgtggagtcactgagtagttatgtcatccattcacagaagagtgtccctgtgtttgcctgtcagcctcagaattagcaaaacatctagcagacagagaacacagtatttcagaagaactccagaggctgccccttaaactctttactggttgatccacataaaatgcttagtagccaagtgccattaattatacagagccaagaagaaaaattagaatacaactttcactttttattttgtagggaaggttttatgttttggtttgttgttgttgttgtgacagtgacagtgactcattacatagaccaagctggcctcaaaatcacatggaccctcgggattacatgtgtccgaccatgttcatcttatttttgaatcttctgtcatatggtaaaagattccagtgggacctgaggagtgactagctaggtaaagcaagggctgtgtaagtgccagaactggtgtttgtgtcctcattatccacataagtgccaagtgagtgtggcccctgcctgtcatcctaggcctcaggagatatcactgctcactggagcaagccggttaaactgttagggcaggtaagttttgacttcaagtgagagaccctgactcaatatgaaaggcaattagtgagtcaagatgaccctgtatgctaacctcttgcctatacatgcatatacacacatttacatatgtgcccaaacatgaggacacaagcacacgcgcgcgcgcacacacacacacacacacacacacacacacacacacacacacacacgagtctaattgtatatagtgataacagtacactttcctccttctatttcggatttagagaaagccatgagaagcgtgtatggtttaaaccatgacccaagcataacaaataaagttgaaatagttgttctcctgtccaagcttgtctttattgttgtgcattctgtaagctggttgcttggttggctgatggatggcttctgtttgtttgttgttttttgtttgtttgtttgtctgggatattacatgtaagaaaaataactggtaagaacaatcaaagaactttgttatgaattaaatcttttgtctaagtcacttagagtcattattctttatgtagatttgcttccagtcaggacatttcctagacagaatttaagacagtaagaaaatgatttgtcacgtctgaaagaggttctttactttcagggacttttgataatgcccaacagagatggcatcgaaagaggagctcatagcgagatgggcatttgtgcatcctcaaggagaaaatattgtaccttctgtttgtatattgtctattctgtgatggctgtatcttacatatgttttgatgcatgtaacaatagtatcatatgaaataaattatatatatatataatatataatatatatcac

SEQ ID NO: 76 - protein, 874 aa, Mus musculus
MKLELLNGCVHLSIEVWNQLKVLLSISHNTSDGEWHFVEVTIAETLTLALVGGSCKEKCTTKSSVPVENHQSICALQDSFLGGLPMGTANNSVSVLNIYNVPSTPSFVGCLQDIRFDLNHITLENVSSGLSSNVKAGCLGKDWCESQPCQNRGRCINLWQGYQCECDRPYTGSNCLKEYVAGRFGQDDSTGYAAFSVNDNYGQNFSLSMFVRTRQPLGLLLALENSTYQYVSVWLEHGSLALQTPGSPKFMVNFFLSDGNVHLISLRIKPNEIELYQSSQNLGFISVPTWTIRRGDVIFIGGLPDREKTEVYGGFFKGCVQDVRLNSQTLEFFPNSTNNAYDDPILVNVTQGCPGDNTCKSNPCHNGGVCHSLWDDFSCSCPTNTAGRACEQVQWCQLSPCPPTAECQLLPQGFECIANAVFSGLSREILFRSNGNITRELTNITFAFRTHDTNVMILHAEKEPEFLNISIQDARLFFQLRSGNSFYTLHLMGSQLVNDGTWHQVTFSMIDPVAQTSRWQMEVNDQTPFVISEVATGSLNFLKDNTDIYVGDQSVDNPKGLQGCLSTIEIGGIYLSYFENLHGFPGKPQEEQFLKVSTNMVLTGCLPSNACHSSPCLHGGNCEDSYSSYRCACLSGWSGTHCEINIDECFSSPCIHGNCSDGVAAYHCRCEPGYTGVNCEVDVDNCKSHQCANGATCVPEAHGYSCLCFGNFTGRFCRHSRLPSTVCGNEKRNFTCYNGGSCSMFQEDWQCMCWPGFTGEWCEEDINECASDPCINGGLCRDLVNRFLCICDVAFAGERCELDLADDRLLGIFTAVGSGTLALFFILLLAGVASLIASNKRATQGTYSPSGQEKAGPRVEMWIRMPPPALERLI

SEQ ID NO: 77 - DNA, 5277 bp, Mus musculus
  source 1..5277: mol_type = genomic DNA
gccccactgatccagcttgaagaggagtgaggcaaagctgaaccctcccactctccttgacaagtgcaagcccacacttttggaaaaaagcacaaagacgtcagaaacggttcctgtcgacctactaggctttggatggctaagtgtttttgctttgtatggaaatatgtttggacacaagacacaaggttttcacattttaatggcagtgctcataggaattcactgtgaagaagacgttgatgaatgtttactgcacccttgcctaaatggtggtacttgtgagaacctgcctgggaattatgcctgtcactgtccctttgatgacacttctaggacattttatggaggagaaaactgctcagaaattctcctgggctgcactcatcaccagtgtctgaacaatggaaaatgtatccctcatttccaaaatggccagcatggattcacttgccagtgtctttctggctatgcggggcccctgtgtgaaactgtcaccacactttcatttgggagcaatggcttcctatgggtcacaagtggctcccatacaggcatagggccagaatgtaacatatccttgaggtttcacactgttcaaccaaacgcacttctcctcatccgaggcaacaaggacgtgtctatgaagctggagttgctgaatggttgtgttcacttatcaattgaagtctggaatcagttaaaggtgctcctgtctatttctcacaacaccagtgatggagaatggcatttcgtggaggtaacaatcgcagaaactctaacccttgccctagttggcggctcctgcaaggagaagtgcaccaccaagtcttctgttccagttgagaatcatcaatcaatatgtgctttgcaggactcttttttgggtggcttaccaatggggacagccaacaacagtgtgtctgtgcttaacatctataatgtgccgtccacaccttcctttgtaggctgtctccaagacattagatttgatttgaatcacattactctggagaacgtttcatctggcctgtcatcaaatgttaaagcaggctgcctgggaaaggactggtgtgaaagtcaaccctgtcaaaacagaggacgctgcatcaacttgtggcagggttatcagtgtgaatgtgacaggccctatacaggctccaactgcctgaaagagtatgtagcgggaagatttggccaagatgactccacaggatatgcggcctttagtgttaatgataattatggacagaacttcagtctttcaatgtttgtccgaacacgtcaacccctgggcttacttctggctttggaaaatagtacttaccagtatgtcagtgtctggctagagcacggcagcctagcactgcagactccaggctctcccaagttcatggtaaacttttttctcagtgatggaaatgttcacttaatatctttgagaatcaaaccaaatgaaattgaactgtatcagtcttcacaaaacctaggattcatttctgttcctacatggacaattcgaagaggagacgtcatcttcattggtggcttacctgacagagagaagactgaagtttatggtggcttcttcaaaggctgtgttcaagatgtcagattaaacagccagactctggaattctttcccaattcaacaaacaatgcatacgatgacccaattcttgtcaatgtgactcaaggctgtcccggagacaacacatgtaagctttaaacatgcgggatgattatctctttatgagcttttgaagaatgagaagaaacatcactggcatgacttgaacatctatgagccccaataattacttccaacccctgtcataatggaggtgtctgccactccctgtgggatgacttctcctgctcctgccctacaaacacagcggggagagcctgcgagcaagttcagtggtgtcaactcagcccatgtcctcccactgcagagtgccagctgctccctcaagggtttgaatgtatcgcaaacgctgttttcagcggattaagcagagaaatactcttcagaagcaatgggaacattaccagagaactcaccaatatcacatttgctttcagaacacatgatacaaatgtgatgatattgcatgcagaaaaagaaccagagtttcttaatattagcattcaagatgccagattattctttcaattgcgaagtggcaacagcttttatacgctgcacctgatgggttcccaattggtgaatgatggcacatggcaccaagtgactttctccatgatagacccagtggcccagacctcccggtggcaaatggaggtgaacgaccagacaccctttgtgataagtgaagttgctactggaagcctgaactttttgaaggacaatacagacatctatgtgggtgaccaatctgttgacaatccgaaaggcctgcagggctgtctgagcacaatagagattggaggcatatatctttcttactttgaaaatctacatggtttccctggtaagcctcaggaagagcaatttctcaaagtttctacaaatatggtacttactggctgtttgccatcaaatgcctgccactccagcccctgtttgcatggaggaaactgtgaagacagctacagttcttatcggtgtgcctgtctctcgggatggtcagggacacactgtgaaatcaacattgatgagtgcttttctagcccctgtatccatggcaactgctctgatggagttgcagcctaccactgcaggtgtgagcctggatacaccggtgtgaactgtgaggtggatgtagacaattgcaagagtcatcagtgtgcaaatggggccacctgtgttcctgaagctcatggctactcttgtctctgctttggaaattttaccgggagattttgcagacacagcagattaccctcaacagtctgtgggaatgagaagagaaacttcacttgctacaatggaggcagctgctccatgttccaggaggactggcaatgtatgtgctggccaggtttcactggagagtggtgtgaagaggacatcaacgagtgtgcctccgatccctgcatcaatggaggactgtgcagggacttggtcaacaggttcctatgcatctgtgatgtggccttcgctggcgagcgctgtgagctggacgtaagcggcctttccttttatgtgtccctcttactatggcaaaacctctttcagctcctgtcctacctcgtactgcgcatgaatgatgagccagttgtagagtggggggcacaggaaaattattaatgtgcatgggagcattcacaagtgtaaaacattgacttgcaagaaacatcttgtctcagtgtaggtttctaggaaagacaaagggaacattagggaatagactccatctagagcactggttctcagtcttcctaatgctgcaaccctttagtacagctcttcctgttgtagtgatcgcagccataacattattttcattgccacttcataactgtaatccttctactgctgtgaatcacaatggaaatatttatgttttctgatggtcttaagcaacacctctgaaaaagtcattgaccccccccccaaaggggctgtgatccacaggttgagaaatgctcatctggaaggtaaccatgcatttaagtgtacctctagtagtttgggtctatagaagatattctcctattctacctttttagacacgccagaagagggcatctgattccattaaagatgattgggagccaccgtgtggttcctgagaactgtactcgggccctttggaagagcaatcagtgctctttccagcccctaagaatatttttaatacagccagaaaggtctcattacccagtgtactgagccctaaggcactttcatcctcaatcgttccatgttgaatggttttcattacatttggaaaatgttttctctccactctacctttacatgttcctattttcctattgacaatttgccccttcactgtaattctaatttggtgtggtccttcttctcataagtttatatgtgacatgaacatttaaaaatatctatgaatattttatagtcatgtatgtctttctgcaaagctattcaaatgaactatggacagttcttttctacacgaagaagagatgagtttaatccccagtaacatgagaaaaagatgagtgagggacagtgctcacagtatccctcactagcatcatttgtgattccatgggccatttttttccaccagcaaatagcagagagccctttccctattcgtttctcttacacttccccttttctgttacaactgaacactttacattagttactcctttgtagggggtttgacttttccaccgttttctctggttcactatttatgctaagtatctgtgcagggcgggtatatcagtccaacagaggtgtcattagtgttcattgaggaggaaatactttgcatgaattcatgacatcattgaagtagcagtggccagaaagatacccttctgcgaatgtgtctgtgtattcagaagctgccctggttagaaaacatgtgggtcacttttcctttgcatgttaccagtgctcactgggtcatgattgttttaagacagagcttttgctgtggcaatgaccaaggtgaatccagagatgcagatcagacaaaggacaagacaatgtactatctgagtaaaaccctgccttgacttactcctcagtacttagagattttacatagcaacctccaccctgtggcaacccgttcacactagcagtgatgctgagatttgcccttccttctcatcatcttcctcacatccaaagcattttgtgtccacactgctgtttcagataactgtttctaaagtgggattgttgtagccagaaaggtagggaaaatgttccccaaaatatttgcattcttaagtatgtgaagtaagtagattatagtcagagacaatatgtaaggtttcaggttcactcccttctacacatatcttcaactgtgtatttgcagaatattctgaatgtgacatactcccaacagaatatatttaaggagtatttatccacagtattgttctctgtacagttctagtgcttctattgtcactgcaattgtcaattgtttttctgctttccaactgtcttattatcatttaatagcatcttgctaaatgccctctttctattctccttatttctccatagttcatgtgtgtctgtgtgactaaggattctcctcatttttgcagaaaaataaaatcttttcttcttta

SEQ ID NO: 78 - protein, 520 aa, Mus musculus
MFGHKTQGFHILMAVLIGIHCEEDVDECLLHPCLNGGTCENLPGNYACHCPFDDTSRTFYGGENCSEILLGCTHHQCLNNGKCIPHFQNGQHGFTCQCLSGYAGPLCETVTTLSFGSNGFLWVTSGSHTGIGPECNISLRFHTVQPNALLLIRGNKDVSMKLELLNGCVHLSIEVWNQLKVLLSISHNTSDGEWHFVEVTIAETLTLALVGGSCKEKCTTKSSVPVENHQSICALQDSFLGGLPMGTANNSVSVLNIYNVPSTPSFVGCLQDIRFDLNHITLENVSSGLSSNVKAGCLGKDWCESQPCQNRGRCINLWQGYQCECDRPYTGSNCLKEYVAGRFGQDDSTGYAAFSVNDNYGQNFSLSMFVRTRQPLGLLLALENSTYQYVSVWLEHGSLALQTPGSPKFMVNFFLSDGNVHLISLRIKPNEIELYQSSQNLGFISVPTWTIRRGDVIFIGGLPDREKTEVYGGFFKGCVQDVRLNSQTLEFFPNSTNNAYDDPILVNVTQGCPGDNTCKL

SEQ ID NO: 79 - DNA, 5530 bp, Mus musculus
  source 1..5530: mol_type = genomic DNA
agccccactgatccagcttgaagaggagtgaggcaaagctgaaccctcccactctccttgacaagtgcaagcccacacttttggaaaaaagcacaaagacgtcagaaacggttcctgtcgacctactaggctttggatggctaagtgtttttgctttgtatggaaatatgtttggacacaagacacaaggttttcacattttaatggcagtgctcataggaattcactgtgaagaagacgttgatgaatgtttactgcacccttgcctaaatggtggtacttgtgagaacctgcctgggaattatgcctgtcactgtccctttgatgacacttctaggacattttatggaggagaaaactgctcagaaattctcctgggctgcactcatcaccagtgtctgaacaatggaaaatgtatccctcatttccaaaatggccagcatggattcacttgccagtgtctttctggctatgcggggcccctgtgtgaaactgtcaccacactttcatttgggagcaatggcttcctatgggtcacaagtggctcccatacaggcatagggccagaatgtaacatatccttgaggtttcacactgttcaaccaaacgcacttctcctcatccgaggcaacaaggacgtgtctatgaagctggagttgctgaatggttgtgttcacttatcaattgaagtctggaatcagttaaaggtgctcctgtctatttctcacaacaccagtgatggagaatggcatttcgtggaggtaacaatcgcagaaactctaacccttgccctagttggcggctcctgcaaggagaagtgcaccaccaagtcttctgttccagttgagaatcatcaatcaatatgtgctttgcaggactcttttttgggtggcttaccaatggggacagccaacaacagtgtgtctgtgcttaacatctataatgtgccgtccacaccttcctttgtaggctgtctccaagacattagatttgatttgaatcacattactctggagaacgtttcatctggcctgtcatcaaatgttaaagcaggctgcctgggaaaggactggtgtgaaagtcaaccctgtcaaaacagaggacgctgcatcaacttgtggcagggttatcagtgtgaatgtgacaggccctatacaggctccaactgcctgaaagagtatgtagcgggaagatttggccaagatgactccacaggatatgcggcctttagtgttaatgataattatggacagaacttcagtctttcaatgtttgtccgaacacgtcaacccctgggcttacttctggctttggaaaatagtacttaccagtatgtcagtgtctggctagagcacggcagcctagcactgcagactccaggctctcccaagttcatggtaaacttttttctcagtgatggaaatgttcacttaatatctttgagaatcaaaccaaatgaaattgaactgtatcagtcttcacaaaacctaggattcatttctgttcctacatggacaattcgaagaggagacgtcatcttcattggtggcttacctgacagagagaagactgaagtttatggtggcttcttcaaaggctgtgttcaagatgtcagattaaacagccagactctggaattctttcccaattcaacaaacaatgcatacgatgacccaattcttgtcaatgtgactcaaggctgtcccggagacaacacatgtaagtccaacccctgtcataatggaggtgtctgccactccctgtgggatgacttctcctgctcctgccctacaaacacagcggggagagcctgcgagcaagttcagtggtgtcaactcagcccatgtcctcccactgcagagtgccagctgctccctcaagggtttgaatgtatcgcaaacgctgttttcagcggattaagcagagaaatactcttcagaagcaatgggaacattaccagagaactcaccaatatcacatttgctttcagaacacatgatacaaatgtgatgatattgcatgcagaaaaagaaccagagtttcttaatattagcattcaagatgccagattattctttcaattgcgaagtggcaacagcttttatacgctgcacctgatgggttcccaattggtgaatgatggcacatggcaccaagtgactttctccatgatagacccagtggcccagacctcccggtggcaaatggaggtgaacgaccagacaccctttgtgataagtgaagttgctactggaagcctgaactttttgaaggacaatacagacatctatgtgggtgaccaatctgttgacaatccgaaaggcctgcagggctgtctgagcacaatagagattggaggcatatatctttcttactttgaaaatctacatggtttccctggtaagcctcaggaagagcaatttctcaaagtttctacaaatatggtacttactggctgtttgccatcaaatgcctgccactccagcccctgtttgcatggaggaaactgtgaagacagctacagttcttatcggtgtgcctgtctctcgggatggtcagggacacactgtgaaatcaacattgatgagtgcttttctagcccctgtatccatggcaactgctctgatggagttgcagcctaccactgcaggtgtgagcctggatacaccggtgtgaactgtgaggtggatgtagacaattgcaagagtcatcagtgtgcaaatggggccacctgtgttcctgaagctcatggctactcttgtctctgctttggaaattttaccgggagattttgcaggtgtgaagaggacatcaacgagtgtgcctccgatccctgcatcaatggaggactgtgcagggacttggtcaacaggttcctatgcatctgtgatgtggccttcgctggcgagcgctgtgagctggacgtaagcggcctttccttttatgtgtccctcttactatggcaaaacctctttcagctcctgtcctacctcgtactgcgcatgaatgatgagccagttgtagagtggggggcacaggaaaattattaatgtgcatgggagcattcacaagtgtaaaacattgacttgcaagaaacatcttgtctcagtgtaggtttctaggaaagacaaagggaacattagggaatagactccatctagagcactggttctcagtcttcctaatgctgcaaccctttagtacagctcttcctgttgtagtgatcgcagccataacattattttcattgccacttcataactgtaatccttctactgctgtgaatcacaatggaaatatttatgttttctgatggtcttaagcaacacctctgaaaaagtcattgaccccccccccaaaggggctgtgatccacaggttgagaaatgctcatctggaaggtaaccatgcatttaagtgtacctctagtagtttgggtctatagaagatattctcctattctacctttttagacacgccagaagagggcatctgattccattaaagatgattgggagccaccgtgtggttcctgagaactgtactcgggccctttggaagagcaatcagtgctctttccagcccctaagaatatttttaatacagccagaaaggtctcattacccagtgtactgagccctaaggcactttcatcctcaatcgttccatgttgaatggttttcattacatttggaaaatgttttctctccactctacctttacatgttcctattttcctattgacaatttgccccttcactgtaattctaatttggtgtggtccttcttctcataagtttatatgtgacatgaacatttaaaaatatctatgaatattttatagtcatgtatgtctttctgcaaagctattcaaatgaactatggacagttcttttctacacgaagaagagatgagtttaatccccagtaacatgagaaaaagatgagtgagggacagtgctcacagtatccctcactagcatcatttgtgattccatgggccatttttttccaccagcaaatagcagagagccctttccctattcgtttctcttacacttccccttttctgttacaactgaacactttacattagttactcctttgtagggggtttgacttttccaccgttttctctggttcactatttatgctaagtatctgtgcagggcgggtatatcagtccaacagaggtgtcattagtgttcattgaggaggaaatactttgcatgaattcatgacatcattgaagtagcagtggccagaaagatacccttctgcgaatgtgtctgtgtattcagaagctgccctggttagaaaacatgtgggtcacttttcctttgcatgttaccagtgctcactgggtcatgattgttttaagacagagcttttgctgtggcaatgaccaaggtgaatccagagatgcagatcagacaaaggacaagacaatgtactatctgagtaaaaccctgccttgacttactcctcagtacttagagattttacatagcaacctccaccctgtggcaacccgttcacactagcagtgatgctgagatttgcccttccttctcatcatcttcctcacatccaaagcattttgtgtccacactgctgtttcagataactgtttctaaagtgggattgttgtagccagaaaggtagggaaaatgttccccaaaatatttgcattcttaagtatgtgaagtaagtagattatagtcagagacaatatgtaaggtttcaggttcactcccttctacacatatcttcaactgtgtatttgcagaatattctgaatgtgacatactcccaacagaatatatttaaggagtatttatccacagtattgttctctgtacagttctagtgcttctattgtcactgcaattgtcaattgtttttctgctttccaactgtcttattatcatttaatagcatcttgctaaatgccctctttctattctccttatttctccatagttcatgtgtgtctgtgtgactaaggattctcctcatttttgcagaaaaataaaatcttttcttctttatgtcctgcttgtcattctctggtgacacatgtctttgcttacttggactgagggttgtacagtaagtacagaagcaggctcagtcacacagacagagacacaccaccaccagcagcagcagcaccaccaccaccaccaccaccaccagaaaacagtatgagtactcatctcttgattacatgtcatttcaagtaagcaccatgacaccgagggccaggttccatggactttctctgttaggcacgtgattctttagctgacctttgagaacagactccaacaacctcacttatttttactgttgacttatatcatctctgacaacactggacttcgtttgagctagtcaagaggaaagaccatgacacctaagggacagaaattcacacactcggtttttcataattcacacacattcctatgtatcaaatctctgtaatagatgacatttacttgaataaaaagtcatttccctttgct

SEQ ID NO: 80 - protein, 960 aa, Mus musculus
MFGHKTQGFHILMAVLIGIHCEEDVDECLLHPCLNGGTCENLPGNYACHCPFDDTSRTFYGGENCSEILLGCTHHQCLNNGKCIPHFQNGQHGFTCQCLSGYAGPLCETVTTLSFGSNGFLWVTSGSHTGIGPECNISLRFHTVQPNALLLIRGNKDVSMKLELLNGCVHLSIEVWNQLKVLLSISHNTSDGEWHFVEVTIAETLTLALVGGSCKEKCTTKSSVPVENHQSICALQDSFLGGLPMGTANNSVSVLNIYNVPSTPSFVGCLQDIRFDLNHITLENVSSGLSSNVKAGCLGKDWCESQPCQNRGRCINLWQGYQCECDRPYTGSNCLKEYVAGRFGQDDSTGYAAFSVNDNYGQNFSLSMFVRTRQPLGLLLALENSTYQYVSVWLEHGSLALQTPGSPKFMVNFFLSDGNVHLISLRIKPNEIELYQSSQNLGFISVPTWTIRRGDVIFIGGLPDREKTEVYGGFFKGCVQDVRLNSQTLEFFPNSTNNAYDDPILVNVTQGCPGDNTCKSNPCHNGGVCHSLWDDFSCSCPTNTAGRACEQVQWCQLSPCPPTAECQLLPQGFECIANAVFSGLSREILFRSNGNITRELTNITFAFRTHDTNVMILHAEKEPEFLNISIQDARLFFQLRSGNSFYTLHLMGSQLVNDGTWHQVTFSMIDPVAQTSRWQMEVNDQTPFVISEVATGSLNFLKDNTDIYVGDQSVDNPKGLQGCLSTIEIGGIYLSYFENLHGFPGKPQEEQFLKVSTNMVLTGCLPSNACHSSPCLHGGNCEDSYSSYRCACLSGWSGTHCEINIDECFSSPCIHGNCSDGVAAYHCRCEPGYTGVNCEVDVDNCKSHQCANGATCVPEAHGYSCLCFGNFTGRFCRCEEDINECASDPCINGGLCRDLVNRFLCICDVAFAGERCELDVSGLSFYVSLLLWQNLFQLLSYLVLRMNDEPVVEWGAQENY

SEQ ID NO: 81 - DNA, 5692 bp, Mus musculus
  source 1..5692: mol_type = genomic DNA
attaagccccactgatccagcttgaagaggagtgaggcaaagctgaaccctcccactctccttgacaagtgcaagcccacacttttggaaaaaagcacaaagacgtcagaaacggttcctgtcgacctactaggctttggatggctaagtgtttttgctttgtatggaaatatgtttggacacaagacacaaggttttcacattttaatggcagtgctcataggaattcactgtgaagaagacgttgatgaatgtttactgcacccttgcctaaatggtggtacttgtgagaacctgcctgggaattatgcctgtcactgtccctttgatgacacttctaggacattttatggaggagaaaactgctcagaaattctcctgggctgcactcatcaccagtgtctgaacaatggaaaatgtatccctcatttccaaaatggccagcatggattcacttgccagtgtctttctggctatgcggggcccctgtgtgaaactgtcaccacactttcatttgggagcaatggcttcctatgggtcacaagtggctcccatacaggcatagggccagaatgtaacatatccttgaggtttcacactgttcaaccaaacgcacttctcctcatccgaggcaacaaggacgtgtctatgaagctggagttgctgaatggttgtgttcacttatcaattgaagtctggaatcagttaaaggtgctcctgtctatttctcacaacaccagtgatggagaatggcatttcgtggaggtaacaatcgcagaaactctaacccttgccctagttggcggctcctgcaaggagaagtgcaccaccaagtcttctgttccagttgagaatcatcaatcaatatgtgctttgcaggactcttttttgggtggcttaccaatggggacagccaacaacagtgtgtctgtgcttaacatctataatgtgccgtccacaccttcctttgtaggctgtctccaagacattagatttgatttgaatcacattactctggagaacgtttcatctggcctgtcatcaaatgttaaagcaggctgcctgggaaaggactggtgtgaaagtcaaccctgtcaaaacagaggacgctgcatcaacttgtggcagggttatcagtgtgaatgtgacaggccctatacaggctccaactgcctgaaagagtatgtagcgggaagatttggccaagatgactccacaggatatgcggcctttagtgttaatgataattatggacagaacttcagtctttcaatgtttgtccgaacacgtcaacccctgggcttacttctggctttggaaaatagtacttaccagtatgtcagtgtctggctagagcacggcagcctagcactgcagactccaggctctcccaagttcatggtaaacttttttctcagtgatggaaatgttcacttaatatctttgagaatcaaaccaaatgaaattgaactgtatcagtcttcacaaaacctaggattcatttctgttcctacatggacaattcgaagaggagacgtcatcttcattggtggcttacctgacagagagaagactgaagtttatggtggcttcttcaaaggctgtgttcaagatgtcagattaaacagccagactctggaattctttcccaattcaacaaacaatgcatacgatgacccaattcttgtcaatgtgactcaaggctgtcccggagacaacacatgtaagtccaacccctgtcataatggaggtgtctgccactccctgtgggatgacttctcctgctcctgccctacaaacacagcggggagagcctgcgagcaagttcagtggtgtcaactcagcccatgtcctcccactgcagagtgccagctgctccctcaagggtttgaatgtaggtagcattcaaagctgtcatccatccaggtatcgcaaacgctgttttcagcggattaagcagagaaatactcttcagaagcaatgggaacattaccagagaactcaccaatatcacatttgctttcagaacacatgatacaaatgtgatgatattgcatgcagaaaaagaaccagagtttcttaatattagcattcaagatgccagattattctttcaattgcgaagtggcaacagcttttatacgctgcacctgatgggttcccaattggtgaatgatggcacatggcaccaagtgactttctccatgatagacccagtggcccagacctcccggtggcaaatggaggtgaacgaccagacaccctttgtgataagtgaagttgctactggaagcctgaactttttgaaggacaatacagacatctatgtgggtgaccaatctgttgacaatccgaaaggcctgcagggctgtctgagcacaatagagattggaggcatatatctttcttactttgaaaatctacatggtttccctggtaagcctcaggaagagcaatttctcaaagtttctacaaatatggtacttactggctgtttgccatcaaatgcctgccactccagcccctgtttgcatggaggaaactgtgaagacagctacagttcttatcggtgtgcctgtctctcgggatggtcagggacacactgtgaaatcaacattgatgagtgcttttctagcccctgtatccatggcaactgctctgatggagttgcagcctaccactgcaggtgtgagcctggatacaccggtgtgaactgtgaggtggatgtagacaattgcaagagtcatcagtgtgcaaatggggccacctgtgttcctgaagctcatggctactcttgtctctgctttggaaattttaccgggagattttgcagacacagcagattaccctcaacagtctgtgggaatgagaagagaaacttcacttgctacaatggaggcagctgctccatgttccaggaggactggcaatgtatgtgctggccaggtttcactggagagtggtgtgaagaggacatcaacgagtgtgcctccgatccctgcatcaatggaggactgtgcagggacttggtcaacaggttcctatgcatctgtgatgtggccttcgctggcgagcgctgtgagctggacgtaagcggcctttccttttatgtgtccctcttactatggcaaaacctctttcagctcctgtcctacctcgtactgcgcatgaatgatgagccagttgtagagtggggggcacaggaaaattattaatgtgcatgggagcattcacaagtgtaaaacattgacttgcaagaaacatcttgtctcagtgtaggtttctaggaaagacaaagggaacattagggaatagactccatctagagcactggttctcagtcttcctaatgctgcaaccctttagtacagctcttcctgttgtagtgatcgcagccataacattattttcattgccacttcataactgtaatccttctactgctgtgaatcacaatggaaatatttatgttttctgatggtcttaagcaacacctctgaaaaagtcattgaccccccccccaaaggggctgtgatccacaggttgagaaatgctcatctggaaggtaaccatgcatttaagtgtacctctagtagtttgggtctatagaagatattctcctattctacctttttagacacgccagaagagggcatctgattccattaaagatgattgggagccaccgtgtggttcctgagaactgtactcgggccctttggaagagcaatcagtgctctttccagcccctaagaatatttttaatacagccagaaaggtctcattacccagtgtactgagccctaaggcactttcatcctcaatcgttccatgttgaatggttttcattacatttggaaaatgttttctctccactctacctttacatgttcctattttcctattgacaatttgccccttcactgtaattctaatttggtgtggtccttcttctcataagtttatatgtgacatgaacatttaaaaatatctatgaatattttatagtcatgtatgtctttctgcaaagctattcaaatgaactatggacagttcttttctacacgaagaagagatgagtttaatccccagtaacatgagaaaaagatgagtgagggacagtgctcacagtatccctcactagcatcatttgtgattccatgggccatttttttccaccagcaaatagcagagagccctttccctattcgtttctcttacacttccccttttctgttacaactgaacactttacattagttactcctttgtagggggtttgacttttccaccgttttctctggttcactatttatgctaagtatctgtgcagggcgggtatatcagtccaacagaggtgtcattagtgttcattgaggaggaaatactttgcatgaattcatgacatcattgaagtagcagtggccagaaagatacccttctgcgaatgtgtctgtgtattcagaagctgccctggttagaaaacatgtgggtcacttttcctttgcatgttaccagtgctcactgggtcatgattgttttaagacagagcttttgctgtggcaatgaccaaggtgaatccagagatgcagatcagacaaaggacaagacaatgtactatctgagtaaaaccctgccttgacttactcctcagtacttagagattttacatagcaacctccaccctgtggcaacccgttcacactagcagtgatgctgagatttgcccttccttctcatcatcttcctcacatccaaagcattttgtgtccacactgctgtttcagataactgtttctaaagtgggattgttgtagccagaaaggtagggaaaatgttccccaaaatatttgcattcttaagtatgtgaagtaagtagattatagtcagagacaatatgtaaggtttcaggttcactcccttctacacatatcttcaactgtgtatttgcagaatattctgaatgtgacatactcccaacagaatatatttaaggagtatttatccacagtattgttctctgtacagttctagtgcttctattgtcactgcaattgtcaattgtttttctgctttccaactgtcttattatcatttaatagcatcttgctaaatgccctctttctattctccttatttctccatagttcatgtgtgtctgtgtgactaaggattctcctcatttttgcagaaaaataaaatcttttcttctttatgtcctgcttgtcattctctggtgacacatgtctttgcttacttggactgagggttgtacagtaagtacagaagcaggctcagtcacacagacagagacacaccaccaccagcagcagcagcaccaccaccaccaccaccaccaccagaaaacagtatgagtactcatctcttgattacatgtcatttcaagtaagcaccatgacaccgagggccaggttccatggactttctctgttaggcacgtgattctttagctgacctttgagaacagactccaacaacctcacttatttttactgttgacttatatcatctctgacaacactggacttcgtttgagctagtcaagaggaaagaccatgacacctaagggacagaaattcacacactcggtttttcataattcacacacattcctatgtatcaaatctctgtaatagatgacatttacttgaataaaaagtcatttcccttt

SEQ ID NO: 82 - protein, 576 aa, Mus musculus
MFGHKTQGFHILMAVLIGIHCEEDVDECLLHPCLNGGTCENLPGNYACHCPFDDTSRTFYGGENCSEILLGCTHHQCLNNGKCIPHFQNGQHGFTCQCLSGYAGPLCETVTTLSFGSNGFLWVTSGSHTGIGPECNISLRFHTVQPNALLLIRGNKDVSMKLELLNGCVHLSIEVWNQLKVLLSISHNTSDGEWHFVEVTIAETLTLALVGGSCKEKCTTKSSVPVENHQSICALQDSFLGGLPMGTANNSVSVLNIYNVPSTPSFVGCLQDIRFDLNHITLENVSSGLSSNVKAGCLGKDWCESQPCQNRGRCINLWQGYQCECDRPYTGSNCLKEYVAGRFGQDDSTGYAAFSVNDNYGQNFSLSMFVRTRQPLGLLLALENSTYQYVSVWLEHGSLALQTPGSPKFMVNFFLSDGNVHLISLRIKPNEIELYQSSQNLGFISVPTWTIRRGDVIFIGGLPDREKTEVYGGFFKGCVQDVRLNSQTLEFFPNSTNNAYDDPILVNVTQGCPGDNTCKSNPCHNGGVCHSLWDDFSCSCPTNTAGRACEQVQWCQLSPCPPTAECQLLPQGFECR

SEQ ID NO: 83 - DNA, 5801 bp, Mus musculus; identical to SEQ ID NO: 12
  source 1..5801: mol_type = genomic DNA

SEQ ID NO: 84 - protein, 761 aa, Mus musculus; identical to SEQ ID NO: 13

SEQ ID NO: 85 - DNA, 5547 bp, Mus musculus
  source 1..5547: mol_type = genomic DNA
acagctgactcttacattaagccccactgatccagcttgaagaggagtgaggcaaagctgaaccctcccactctccttgacaagtgcaagcccacacttttggaaaaaagcacaaagacgtcagaaacggttcctgtcgacctactaggctttggatggctaagtgtttttgctttgtatggaaatatgtttggacacaagacacaaggttttcacattttaatggcagtgctcataggaattcactgtgaagaagacgttgatgaatgtttactgcacccttgcctaaatggtggtacttgtgagaacctgcctgggaattatgcctgtcactgtccctttgatgacacttctaggacattttatggaggagaaaactgctcagaaattctcctgggctgcactcatcaccagtgtctgaacaatggaaaatgtatccctcatttccaaaatggccagcatggattcacttgccagtgtctttctggctatgcggggcccctgtgtgaaactgtcaccacactttcatttgggagcaatggcttcctatgggtcacaagtggctcccatacaggcatagggccagaatgtaacatatccttgaggtttcacactgttcaaccaaacgcacttctcctcatccgaggcaacaaggacgtgtctatgaagctggagttgctgaatggttgtgttcacttatcaattgaagtctggaatcagttaaaggtgctcctgtctatttctcacaacaccagtgatggagaatggcatttcgtggaggtaacaatcgcagaaactctaacccttgccctagttggcggctcctgcaaggagaagtgcaccaccaagtcttctgttccagttgagaatcatcaatcaatatgtgctttgcaggactcttttttgggtggcttaccaatggggacagccaacaacagtgtgtctgtgcttaacatctataatgtgccgtccacaccttcctttgtaggctgtctccaagacattagatttgatttgaatcacattactctggagaacgtttcatctggcctgtcatcaaatgttaaagcaggctgcctgggaaaggactggtgtgaaagtcaaccctgtcaaaacagaggacgctgcatcaacttgtggcagggttatcagtgtgaatgtgacaggccctatacaggctccaactgcctgaaagagtatgtagcgggaagatttggccaagatgactccacaggatatgcggcctttagtgttaatgataattatggacagaacttcagtctttcaatgtttgtccgaacacgtcaacccctgggcttacttctggctttggaaaatagtacttaccagtatgtcagtgtctggctagagcacggcagcctagcactgcagactccaggctctcccaagttcatgaggagacgtcatcttcattggtggcttacctgacagagagaagactgaagtttatggtggcttcttcaaaggctgtgttcaagatgtcagattaaacagccagactctggaattctttcccaattcaacaaacaatgcatacgatgacccaattcttgtcaatgtgactcaaggctgtcccggagacaacacatgtaagtccaacccctgtcataatggaggtgtctgccactccctgtgggatgacttctcctgctcctgccctacaaacacagcggggagagcctgcgagcaagttcagtggtgtcaactcagcccatgtcctcccactgcagagtgccagctgctccctcaagggtttgaatgtatcgcaaacgctgttttcagcggattaagcagagaaatactcttcagaagcaatgggaacattaccagagaactcaccaatatcacatttgctttcagaacacatgatacaaatgtgatgatattgcatgcagaaaaagaaccagagtttcttaatattagcattcaagatgccagattattctttcaattgcgaagtggcaacagcttttatacgctgcacctgatgggttcccaattggtgaatgatggcacatggcaccaagtgactttctccatgatagacccagtggcccagacctcccggtggcaaatggaggtgaacgaccagacaccctttgtgataagtgaagttgctactggaagcctgaactttttgaaggacaatacagacatctatgtgggtgaccaatctgttgacaatccgaaaggcctgcagggctgtctgagcacaatagagattggaggcatatatctttcttactttgaaaatctacatggtttccctggtaagcctcaggaagagcaatttctcaaagtttctacaaatatggtacttactggctgtttgccatcaaatgcctgccactccagcccctgtttgcatggaggaaactgtgaagacagctacagttcttatcggtgtgcctgtctctcgggatggtcagggacacactgtgaaatcaacattgatgagtgcttttctagcccctgtatccatggcaactgctctgatggagttgcagcctaccactgcaggtgtgagcctggatacaccggtgtgaactgtgaggtggatgtagacaattgcaagagtcatcagtgtgcaaatggggccacctgtgttcctgaagctcatggctactcttgtctctgctttggaaattttaccgggagattttgcagacacagcagattaccctcaacagtctgtgggaatgagaagagaaacttcacttgctacaatggaggcagctgctccatgttccaggaggactggcaatgtatgtgctggccaggtttcactggagagtggtgtgaagaggacatcaacgagtgtgcctccgatccctgcatcaatggaggactgtgcagggacttggtcaacaggttcctatgcatctgtgatgtggccttcgctggcgagcgctgtgagctggacgtaagcggcctttccttttatgtgtccctcttactatggcaaaacctctttcagctcctgtcctacctcgtactgcgcatgaatgatgagccagttgtagagtggggggcacaggaaaattattaatgtgcatgggagcattcacaagtgtaaaacattgacttgcaagaaacatcttgtctcagtgtaggtttctaggaaagacaaagggaacattagggaatagactccatctagagcactggttctcagtcttcctaatgctgcaaccctttagtacagctcttcctgttgtagtgatcgcagccataacattattttcattgccacttcataactgtaatccttctactgctgtgaatcacaatggaaatatttatgttttctgatggtcttaagcaacacctctgaaaaagtcattgaccccccccccaaaggggctgtgatccacaggttgagaaatgctcatctggaaggtaaccatgcatttaagtgtacctctagtagtttgggtctatagaagatattctcctattctacctttttagacacgccagaagagggcatctgattccattaaagatgattgggagccaccgtgtggttcctgagaactgtactcgggccctttggaagagcaatcagtgctctttccagcccctaagaatatttttaatacagccagaaaggtctcattacccagtgtactgagccctaaggcactttcatcctcaatcgttccatgttgaatggttttcattacatttggaaaatgttttctctccactctacctttacatgttcctattttcctattgacaatttgccccttcactgtaattctaatttggtgtggtccttcttctcataagtttatatgtgacatgaacatttaaaaatatctatgaatattttatagtcatgtatgtctttctgcaaagctattcaaatgaactatggacagttcttttctacacgaagaagagatgagtttaatccccagtaacatgagaaaaagatgagtgagggacagtgctcacagtatccctcactagcatcatttgtgattccatgggccatttttttccaccagcaaatagcagagagccctttccctattcgtttctcttacacttccccttttctgttacaactgaacactttacattagttactcctttgtagggggtttgacttttccaccgttttctctggttcactatttatgctaagtatctgtgcagggcgggtatatcagtccaacagaggtgtcattagtgttcattgaggaggaaatactttgcatgaattcatgacatcattgaagtagcagtggccagaaagatacccttctgcgaatgtgtctgtgtattcagaagctgccctggttagaaaacatgtgggtcacttttcctttgcatgttaccagtgctcactgggtcatgattgttttaagacagagcttttgctgtggcaatgaccaaggtgaatccagagatgcagatcagacaaaggacaagacaatgtactatctgagtaaaaccctgccttgacttactcctcagtacttagagattttacatagcaacctccaccctgtggcaacccgttcacactagcagtgatgctgagatttgcccttccttctcatcatcttcctcacatccaaagcattttgtgtccacactgctgtttcagataactgtttctaaagtgggattgttgtagccagaaaggtagggaaaatgttccccaaaatatttgcattcttaagtatgtgaagtaagtagattatagtcagagacaatatgtaaggtttcaggttcactcccttctacacatatcttcaactgtgtatttgcagaatattctgaatgtgacatactcccaacagaatatatttaaggagtatttatccacagtattgttctctgtacagttctagtgcttctattgtcactgcaattgtcaattgtttttctgctttccaactgtcttattatcatttaatagcatcttgctaaatgccctctttctattctccttatttctccatagttcatgtgtgtctgtgtgactaaggattctcctcatttttgcagaaaaataaaatcttttcttctttatgtcctgcttgtcattctctggtgacacatgtctttgcttacttggactgagggttgtacagtaagtacagaagcaggctcagtcacacagacagagacacaccaccaccagcagcagcagcaccaccaccaccaccaccaccaccagaaaacagtatgagtactcatctcttgattacatgtcatttcaagtaagcaccatgacaccgagggccaggttccatggactttctctgttaggcacgtgattctttagctgacctttgagaacagactccaacaacctcacttatttttactgttgacttatatcatctctgacaacactggacttcgtttgagctagtcaagaggaaagaccatgacacctaagggacagaaattcacacactcggtttttcataattcacacacattcctatgtatcaaatctctgtaatagatgacatttacttgaataaaaagtcatttcccttt

SEQ ID NO: 86 - protein, 420 aa, Mus musculus
MFGHKTQGFHILMAVLIGIHCEEDVDECLLHPCLNGGTCENLPGNYACHCPFDDTSRTFYGGENCSEILLGCTHHQCLNNGKCIPHFQNGQHGFTCQCLSGYAGPLCETVTTLSFGSNGFLWVTSGSHTGIGPECNISLRFHTVQPNALLLIRGNKDVSMKLELLNGCVHLSIEVWNQLKVLLSISHNTSDGEWHFVEVTIAETLTLALVGGSCKEKCTTKSSVPVENHQSICALQDSFLGGLPMGTANNSVSVLNIYNVPSTPSFVGCLQDIRFDLNHITLENVSSGLSSNVKAGCLGKDWCESQPCQNRGRCINLWQGYQCECDRPYTGSNCLKEYVAGRFGQDDSTGYAAFSVNDNYGQNFSLSMFVRTRQPLGLLLALENSTYQYVSVWLEHGSLALQTPGSPKFMRRRHLHWWLT

SEQ ID NO: 87 - protein, 18 aa, synthetic construct
  REGION 1..18: note = Consensus signal peptide generated in silico
  SITE 4: note = misc_feature - Xaa can be any naturally occurring amino acid
MFGXRTQGFHILMAMLIG

SEQ ID NO: 88 - protein, 41 aa, synthetic construct
  REGION 1..41: note = Consensus transmembrane domain generated in silico
  SITE 37: note = misc_feature - Xaa can be any naturally occurring amino acid
  SITE 40: note = misc_feature - Xaa can be any naturally occurring amino acid
VSGLSFYVSLLLWQNLFQLLSYLILRMNDEPVVEWGXQEXY

SEQ ID NO: 89 - protein, 18 aa, Homo sapiens
MFGARTHGFHILMAMLIG

SEQ ID NO: 90 - protein, 18 aa, Bos taurus
MFGARTQGFHILMAMLIG

SEQ ID NO: 91 - protein, 41 aa, Bos taurus
VSGLSFYVSLLLWQNLFQLLSYLILRLNDEPVVEWGDQDDY

SEQ ID NO: 92 - protein, 18 aa, Mus musculus
MFGHKTQGFHILMAVLIG

SEQ ID NO: 93 - protein, 41 aa, Mus musculus
VSGLSFYVSLLLWQNLFQLLSYLVLRMNDEPVVEWGAQENY

SEQ ID NO: 94 - protein, 18 aa, Rattus norvegicus
MFGHRTQGFYIFMAILIG

SEQ ID NO: 95 - protein, 41 aa, Rattus norvegicus
VSGLSFYVSLLLWQNLFQLLSYLILRMNDEPEVEWGAQENY

SEQ ID NO: 96 - protein, 16 aa, Danio rerio
MEVVVGIWSVLLLISG

SEQ ID NO: 97 - protein, 39 aa, Danio rerio
VSDLYFYMAALFWQNLFQFLSYLILRLDDEPEVDWGDNE

SEQ ID NO: 98 - DNA, 50 bp, Mus musculus
  source 1..50: mol_type = unassigned DNA
aaacaaaaactaaagcatcttattccctgggcttacttctggctttggaa

SEQ ID NO: 99 - DNA, 50 bp, Mus musculus
  source 1..50: mol_type = unassigned DNA
aagaagaataagtacccgttcccatcacctaatcgctttattctgataga

SEQ ID NO: 100 - DNA, 48 bp, synthetic construct
  misc_feature 1..48: note = Crb1 delB allele
  source 1..48: mol_type = other DNA
aagaagaataagtacccgttcatcacctaatcgctttattctgataga